(19) United States
(12) Patent Application Publication
(10) Pub. No.: US 2025/0382650 A1
(43) Pub. Date: Dec. 18, 2025

(54) NUCLEIC ACID LIGATION METHOD

(71) Applicant: Novartis AG, Basel (CH)

(72) Inventors: Gregory MANN, Basel (CH); Frédéric Valentin STANGER, Colmar (FR)

(21) Appl. No.: 19/141,592

(22) PCT Filed: Dec. 19, 2023
(86) PCT No.: PCT/IB2023/062952

§ 371 Date: Jun. 20, 2025

(30) Foreign Application Priority Data
Dec. 20, 2022 (EP) 22215207.6

(51) Int. Cl. C12P 19/34; C12N 9/00; C12N 9/12; C12N 15/62; C12N 15/70

(52) U.S. Cl. CPC C12P 19/34; C12N 9/1229; C12N 9/93; C12N 15/62; C12N 15/70; C07K 2319/00; C12Y 207/04001; C12Y 605/01001

(57) ABSTRACT

The present disclosure relates to biocatalytic ligation methods for producing oligonucleotides; and to fusion polypeptides for use in said methods. In particular, the present disclosure relates to biocatalytic ligation methods incorporating ATP regeneration and to fusion polypeptides comprising a polyphosphate kinase domain and an ATP-dependent nucleic acid ligase domain.

### CROSS-REFERENCE TO RELATED APPLICATIONS

This application claims priority to, and the benefit of, EP Application Serial No. 22215207.6, filed on Dec. 20, 2022, the content of which is incorporated herein by reference in its entirety.

### SEQUENCE LISTING

The instant application contains a Sequence Listing which has been submitted electronically in .XML format and is hereby incorporated by reference in its entirety. Said .XML copy, created on 29 Nov. 2023, is named PAT059356-WO-PCT_SL.xml and is 186 KB in size.

### TECHNICAL FIELD

The present disclosure relates to the field of biotechnology, in particular to biocatalytic ligation methods for producing oligonucleotides; and to fusion polypeptides for use in said methods. In particular, the present disclosure relates to biocatalytic ligation methods incorporating ATP regeneration; and to fusion polypeptides comprising a polyphosphate kinase domain and an ATP-dependent nucleic acid ligase domain.

### BACKGROUND ART

Therapeutic oligonucleotides, including small interfering RNA (siRNA) and inhibitory antisense oligonucleotides (ASOs) have the potential to treat a diverse range of life-threatening diseases. In recent years there has been a significant increase in the number of approved oligonucleotide-based drugs, and a large rise in the number of therapeutic oligonucleotides under clinical investigation (Roberts, T. C., Langer, R. & Wood, M. J. A. Nature Reviews Drug Discovery 2020 19:10 19, 673-694 (2020)).

In support of green synthesis initiatives throughout the pharmaceutical industry, there is a significant need for next-generation oligonucleotide synthesis methods that are both sustainable and economical at the scale required to reach wider patient populations (Mishra, M. et al. Current Research in Green and Sustainable Chemistry 4, (2021)).

To this end, biocatalysis is being more frequently applied in the manufacture of active pharmaceutical ingredients (APIs) since enzymes are capable of highly selective transformation under mild reaction conditions and in aqueous media (Mann, G. & Stanger, F. V. Chimia (Aarau) 74, 407-417 (2020)). The biocatalysis of short oligonucleotide fragments offers a sustainable and economical alternative to the solid phase chemical synthesis of full-length therapeutic oligonucleotides currently used.

Shorter oligonucleotides can be synthesized more easily and with higher purities than longer oligonucleotides, simplifying downstream processing and reducing solvent waste. These short oligonucleotide fragments can then be combined using nucleic acid ligases to produce oligonucleotide products. Nucleic acid ligases have shown remarkable tolerance towards unnatural DNA/RNA containing pharmaceutically relevant chemical modifications (Kestemont, D., Herdewijn, P. & Renders, M. Curr Protoc Chem Biol 11, e62 (2019); Kestemont, D. et al. Chemical Communications 54, 6408-6411 (2018); and Nandakumar, J. & Shuman, S. Molecular Cell 16, 211-221 (2004)), and the use of a dsRNA ligase to synthesize an siRNA product, starting from short fragments (≤9 nts), containing extensive chemical modification, including 2′-OMe, 2′-F modified nucleotides, phosphorothioate backbone modified nucleotides and a terminal fragment that is functionalized with a bulky N-acetyl galactosamine (GalNAc) moiety has previously been described (Mann, G. et al. Tetrahedron Letters 93, 153696 (2022)).

A major drawback of existing nucleic acid ligation reactions is their reliance on the expensive cofactor, ATP. One molecule of ATP is converted to AMP per ligation reaction, and so at increasing substrate (oligonucleotide fragment) concentrations, an increased concentration of ATP is required to achieve complete ligation. In practice, an excess (i.e. a higher than stoichiometric quantity) of ATP is typically required to achieve complete ligation. This requirement for high concentrations of ATP presents a number of limitations regarding sustainability, process costs, difficulties with downstream processing, and potentially cofactor by-product inhibition (Mordhorst, S. & Andexer, J. N. Natural Product Reports 37, 1316-1333 (2020)).

There exists an urgent and unmet need for efficient and cost-effective biocatalytic methods for producing oligonucleotides; and for enzymes for use in said methods.

### BRIEF DESCRIPTION OF THE DRAWINGS

FIG. 1. Chemical mechanism of ligation by ATP dependent ligases.

FIG. 2. Polyphosphate kinase 12 (PPK12) dose response demonstrating ATP synthesis activity starting from AMP via an ADP intermediate. Maximum conversion ˜ 65% ATP is achieved, which is consistent with previous reports.

FIG. 3. dsRNA ligase catalyzed ligation reaction of short, chemically modified oligonucleotides to generate an siRNA product. ATP is converted to AMP during the ligation reaction. Polyphosphate kinase converts the AMP generated into ATP using polyphosphate as a phosphate donor for the reaction.

FIG. 4. Enzyme dose response showing ligation activity with and without ATP recycling as indicated in the legend.

FIG. 5. Example chromatograms of ligation reaction analysis, highlighting how the identified substrate, intermediate and product peaks correspond to the calculated pseudo % conversion termed arbitrary units (AU). A) Product standard, B) 1.25 g/L ligase+10 mM ATP, C) 1.25 g/L Klignase 11+2.5 mM AMP+20 mM polyphosphate, D) 1.25 g/L ligase+1.25 g/L kinase+2.5 mM AMP+20 mM polyphosphate, E) 1.25 g/L Klignase 9+2.5 mM AMP+20 mM polyphosphate, F) 1.25 g/L Klignase 2+2.5 mM AMP+20 mM polyphosphate G) 1.25 g/L ligase+2.5 mM AMP+20 mM polyphosphate, H) substrate only (no enzyme control).

FIG. 6. SDS-PAGE of enzyme feedstocks diluted to 0.131 g/L (left panel) and 0.261 g/L (right panel). The bands observed at ˜37 kDa correspond with ligase/kinase enzymes; the bands at ˜76 kDa correspond the various Klignase constructs; the bands at ˜ 25 kDa correspond with chloramphenicol acetyl transferase (antibiotic resistance). The lanes contain the following proteins: R) protein standard; 1)-11) Klignases 1-11 respectively; 12) dsRNA ligase; 13) mix of dsRNA ligase and PPK; 14) PPK12

FIG. 7. Enzyme dose response comparing ligation and ATP synthesis activity of the Klignases and the non-fused enzymes.

FIG. 8. Enzyme dose response comparing ligation and ATP synthesis activity of Klignases 4 and 11 and the non-fused enzymes.

FIG. 9. Enzyme dose response comparing ligation activity of the optimized Bacteriophage RB69 RNA ligase 2 polypeptide of SEQ ID NO: 2 towards 1 mM substrate in the presence of 10 mM ATP and with an increasing concentration of polyphosphate as indicated by the legend.

FIG. 10. Comparison of ligation activity of the optimized Bacteriophage RB69 RNA ligase 2 polypeptides of SEQ ID NO: 2 and SEQ ID NO: 88 with 1 and 3 mM substrate.

FIG. 11. A Enzyme dose response of Klignase 4.2 of SEQ ID NO: 90 comparing the ligation and ATP synthesis activity in the presence of different polyphosphate salts with either 5 mM or 65 mM MgCl₂ as indicated by the legend. B Enzyme dose response of Klignase 4.2 of SEQ ID NO: 90 in the presence of 80 mM polyphosphate (Maddrell's salt) and an increasing concentration of MgCl₂ as indicated by the legend.

FIG. 12. Enzyme dose response comparing the ligation and ATP synthesis activity of the Klignases of SEQ ID NO: 90, SEQ ID NO: 92, SEQ ID NO: 94, SEQ ID NO: 96 and SEQ ID NO: 98 towards 2.5 mM of each substrate oligonucleotide (substrates 1-6, table 2) in the presence of 1 mM AMP, 80 mM polyphosphate, 40 mM MgCl₂, 5 mM DTT and 100 mM MOPS-buffer pH 7.2.

FIG. 13. Enzyme dose response comparing the ligation and ATP synthesis activity of the Klignases of SEQ ID NO: 11 and SEQ ID NO: 90 towards 6 mM of each substrate oligonucleotide (substrates 1-6, table 2) in the presence of 0.25 mM AMP, 80 mM polyphosphate, 40 mM MgCl₂, 5 mM DTT and 100 mM MOPS-buffer pH 7.2.

### SUMMARY OF THE DISCLOSURE

The present disclosure provides a ligation reaction method comprising an ATP regeneration system. The ATP regeneration system overcomes the requirement for high concentrations of ATP and enables the ligation reaction to be performed in the presence of the cheaper alternative, AMP. Beneficially, the methods described herein achieve complete ligation of oligonucleotide fragments in the presence of sub-stoichiometric quantities of ATP or AMP. The methods described herein benefit from significantly lower costs and improved sustainability as compared to methods performed in the absence of ATP regeneration which require significantly higher ATP concentrations to achieve complete ligation.

The present disclosure also provides bifunctional fusion polypeptides comprising a PPK domain and a ligase domain. These fusion polypeptides are particularly well suited to industrial biocatalysis ligation methods because they can be produced more quickly, more efficiently, and at a reduced cost as compared to the production of separate PPK and ligase enzymes. As demonstrated herein, linking the ligase and PPK enzymes unexpectedly provided a functional fusion polypeptide with retained ligase and PPK activity. Moreover, as demonstrated herein, linking the ligase and PPK enzymes unexpectedly improves ligase activity as compared to ligase activity in a reaction mixture containing unlinked enzymes.

The present disclosure provides a method of producing an oligonucleotide from two or more oligonucleotide fragments, wherein the method comprises contacting: i. two or more oligonucleotide fragments; ii. an ATP-dependent nucleic acid ligase; iii. a polyphosphate kinase (PPK); iv. adenosine triphosphate (ATP) and/or adenosine monophosphate (AMP); v. polyphosphate; and vi. a divalent cation; and thereby providing an oligonucleotide.

The present disclosure also provides use of an ATP-dependent nucleic acid ligase and a PPK in the production of an oligonucleotide from two or more oligonucleotide fragments.

In some embodiments, the two or more oligonucleotide fragments comprise two or more RNA oligonucleotide fragments. In some embodiments, the ATP-dependent nucleic acid ligase is an RNA ligase. In some embodiments, the RNA ligase is a double-stranded RNA ligase. In some embodiments, the RNA ligase is a member of the RNA ligase 2 family. In some embodiments, the RNA ligase is Bacteriophage RB69 RNA ligase 2.

In some embodiments, the two or more oligonucleotide fragments comprise two or more DNA oligonucleotide fragments. In some embodiments, the ATP-dependent nucleic acid ligase is a DNA ligase. In some embodiments, the DNA ligase is T4 DNA ligase.

In some embodiments, the PPK is PPK12 or ajPAP.

In some embodiments, the ATP-dependent nucleic acid ligase and the PPK are linked.

In some embodiments, the ATP-dependent nucleic acid ligase and the PPK are linked via a polypeptide linker. In some embodiments, the PPK is located at the N-terminus of the linker and the ATP-dependent nucleic acid ligase is located at the C-terminus of the linker.

In some embodiments, the ATP-dependent nucleic acid ligase comprises a purification tag. In some embodiments, the PPK comprises a purification tag. In some embodiments, the linker comprises a purification tag.

In some embodiments, the linker is a polypeptide linker comprising at least 3 amino acids, optionally at least 6 amino acids.

In some embodiments, the linker comprises an amino acid sequence selected from: a) HHHHHH (SEQ ID NO: 19), optionally HHHHHHHHHH (SEQ ID NO: 20); b) ENLYFQS (SEQ ID NO: 21); c) ENLYFQG (SEQ ID NO: 22); d) SSGSSG (SEQ ID NO: 23); e) GSAGSAAGSGEF (SEQ ID NO: 24); and/or f) GSSGSGSSSGGSSSSGSS (SEQ ID NO: 25).

In some embodiments, the polyphosphate is a polyphosphate salt. In some embodiments, the polyphosphate salt is sodium polyphosphate (Maddrell's salt) or sodium hexametaphosphate (Graham's salt).

In some embodiments, the divalent cation cofactor is Mg²⁺ or Mn²⁺. In some embodiments, the method is performed with a divalent cation concentration of 5-100 mM, optionally 30-50 mM.

In some embodiments, the method is performed with a sub-stoichiometric concentration of ATP and/or AMP.

In some embodiments, the method further comprises a step of purifying the oligonucleotide.

In some embodiments, the oligonucleotide is up to 60 nucleotides in length.

In some embodiments, each of the oligonucleotide fragments are 4-16 nucleotides in length, optionally 6-9 nucleotides in length.

In some embodiments, the oligonucleotide fragments are single-stranded.

In some embodiments, the oligonucleotide fragments are double-stranded, optionally wherein one or more of the double-stranded oligonucleotide fragments comprises one or two single-stranded overhang(s).

In some embodiments, one or more of the oligonucleotide fragments comprises a chemical modification. In some embodiments, the chemical modification is selected from: (a) a modified backbone, optionally selected from a phosphorothioate (e.g. chiral phosphorothioate) or methylphosphonate internucleotide linkage; (b) a modified nucleotide, optionally selected from 2′-O-methyl (2′-OMe), 2′-flouro (2′-F), 2′-deoxy, 2′-deoxy-2′-fluoro, 2′-O-methoxyethyl (2′-O-MOE), 2′-O-aminopropyl (2′-O-AP), 2′-O-dimethylaminoethyl (2′-O-DMAOE), 2′-O-dimethylaminopropyl (2′-O-DMAP), 2′-O-dimethylaminoethyloxyethyl (2′-O-DMAEOE), 2′-O—N-methylacetamido (2′-O-NMA), locked nucleic acid (LNA), glycol nucleic acid (GNA), phosphoramidate (e.g. mesyl phosphoramidate), 2′,3′-seco nucleotide mimic, 2′-F-arabino nucleotide, abasic nucleotide, 2′-amino modified nucleotide, 2′-alkyl-modified nucleotide, morpholino nucleotide, vinylphosphonate (e.g. 5′ vinylphosphonate), and cyclopropyl phosphonate deoxyribonucleotide; and/or (c) conjugation to a ligand, optionally wherein the ligand comprises one or more N-Acetylgalactosamine (GalNAc) derivatives.

In some embodiments, the ATP-dependent nucleic acid ligase and/or the PPK are immobilised. In some embodiments, the ATP-dependent nucleic acid ligase and/or the PPK are immobilised on a solid material by chemical bond or a physical adsorption method.

The disclosure also provides a composition comprising: i. an ATP-dependent nucleic acid ligase; ii. a PPK; iii. ATP and/or AMP; iv. a divalent cation; and v. polyphosphate. In some embodiments, the composition further comprises two or more oligonucleotide fragments.

The disclosure also provides a kit comprising: i. an ATP-dependent nucleic acid ligase; ii. a PPK; iii. ATP and/or AMP; iv. polyphosphate; v. a divalent cation; and vi. instructions for use in a method of producing an oligonucleotide from two or more oligonucleotide fragments.

In some embodiments, the polyphosphate is a polyphosphate salt. In some embodiments, the polyphosphate salt is Graham's salt or Maddrell's salt.

In some embodiments, the divalent cation is Mg²⁺ or Mn²⁺. In some embodiments, the concentration of divalent cation is 5-100 mM, optionally 30-50 mM.

The disclosure also provides a fusion polypeptide comprising: a) a PPK domain; and b) an ATP-dependent nucleic acid ligase domain.

In some embodiments, the fusion polypeptide comprises a linker.

In some embodiments, the PPK is PPK12 or ajPAP.

In some embodiments, the PPK domain comprises an amino acid sequence that has at least 85% identity with the amino acid sequence of any one of SEQ ID NOs: 5-7.

In some embodiments, the ATP-dependent nucleic acid ligase domain is an RNA ligase domain.

In some embodiments, the RNA ligase domain is a double-stranded RNA (dsRNA) ligase domain.

In some embodiments, the dsRNA ligase is a member of the RNA ligase 2 family.

In some embodiments, the dsRNA ligase is Bacteriophage RB69 RNA ligase 2.

In some embodiments, the ATP-dependent nucleic acid ligase domain is a DNA ligase domain.

In some embodiments, the DNA ligase domain is a T4 DNA ligase domain.

In some embodiments, the ATP-dependent nucleic acid ligase domain comprises an amino acid sequence that has at least 85% sequence identity with the amino acid sequence of any one of SEQ ID NOs: 1-4. In some embodiments, the ATP-dependent nucleic acid ligase domain comprises an amino acid sequence that has at least 85% sequence identity with the amino acid sequence of SEQ ID NO: 88. In some embodiments, the ATP-dependent nucleic acid ligase domain comprises an amino acid sequence that has at least 85% sequence identity with the amino acid sequence of any one of SEQ ID NOs: 1-4 or 88.

In some embodiments, the linker is located between the PPK domain and the ATP-dependent nucleic acid ligase domain.

In some embodiments, the PPK domain is located at the N-terminus of the linker and the ATP-dependent nucleic acid ligase domain is located at the C-terminus of the linker.

In some embodiments, the fusion polypeptide comprises a purification tag.

In some embodiments, the linker comprises a purification tag. In some embodiments, a purification tag is located at the N- and/or C-terminus of the fusion polypeptide.

In some embodiments, the linker is a polypeptide linker comprising at least 3 amino acids, optionally at least 6 amino acids. In some embodiments, the linker comprises an amino acid sequence selected from: a) HHHHHH (SEQ ID NO: 19), optionally HHHHHHHHHH (SEQ ID NO: 20); b) ENLYFQS (SEQ ID NO: 21); c) ENLYFQG (SEQ ID NO: 22); d) SSGSSG (SEQ ID NO: 23); e) GSAGSAAGSGEF (SEQ ID NO: 24); and/or f) GSSGSGSSSGGSSSSGSS (SEQ ID NO: 25).

In some embodiments, the fusion polypeptide comprises an amino acid sequence that has at least 85% sequence identity with the amino acid sequence of any one of SEQ ID NOs: 8-18. In some embodiments, the fusion polypeptide comprises an amino acid sequence that has at least 85% sequence identity with the amino acid sequence of any one of SEQ ID NOs: 90, 92, 94, 96, and 98. In some embodiments, the fusion polypeptide comprises an amino acid sequence that has at least 85% sequence identity with the amino acid sequence of any one of SEQ ID NOs: 8-18, 90, 92, 94, 96, and 98.

In some embodiments, the ATP-dependent nucleic acid ligase and the PPK are provided as a fusion polypeptide as described herein.

The disclosure also provides a nucleic acid molecule encoding the fusion polypeptide described herein.

In some embodiments, the nucleic acid molecule comprises a nucleic acid sequence that has at least 85% sequence identity with the nucleic acid sequence of any one of SEQ ID NOs: 34-36. SEQ ID NOs: 34-36 are nucleic acid sequences encoding the amino acid sequences of SEQ ID NOs: 5-7, respectively

In some embodiments, the nucleic acid molecule comprises a nucleic acid sequence that has at least 85% sequence identity with the nucleic acid sequence of any one of SEQ ID NOs: 30-33. In some embodiments, the nucleic acid molecule comprises a nucleic acid sequence that has at least 85% sequence identity with the nucleic acid sequence of SEQ ID NO: 87. In some embodiments, the nucleic acid molecule comprises a nucleic acid sequence that has at least 85% sequence identity with the nucleic acid sequence of any one of SEQ ID NOs: 30-33 or 87. SEQ ID NOs: 30-33 and 87 are nucleic acid sequences encoding the amino acid sequences of SEQ ID NOs: 1-4 and 88, respectively.

In some embodiments, the nucleic acid molecule comprises a nucleic acid sequence that has at least 85% sequence identity with the nucleic acid of any one of SEQ ID NOs: 37-47. In some embodiments, the nucleic acid molecule comprises a nucleic acid sequence that has at least 85% sequence identity with the nucleic acid of any one of SEQ ID NOs: 89, 91, 93, 95, or 97. In some embodiments, the nucleic acid molecule comprises a nucleic acid sequence that has at least 85% sequence identity with the nucleic acid of any one of SEQ ID NOs: 37-47, 89, 91, 93, 95, or 97. SEQ ID NOs: 37-47, 89, 91, 93, 95, and 97 are nucleic acid sequences encoding the amino acid sequences of SEQ ID NOs: 8-18, 90, 92, 94, 96, and 98, respectively.

The disclosure also provides a vector comprising the nucleic acid described herein.

In some embodiments, the vector is selected from a plasmid, a cosmid, a bacteriophage or a viral vector.

The disclosure also provides a host cell comprising the nucleic acid molecule described herein or the vector described herein. In some embodiments, the host cell is E. coli.

The disclosure also provides use of a fusion polypeptide described herein in an ATP-dependent nucleic acid ligation reaction. In some embodiments, the ATP-dependent nucleic acid ligation reaction is an ATP-dependent RNA ligation reaction (e.g, wherein the ATP-dependent nucleic acid ligase domain is a dsRNA ligase domain).

In some embodiments, the rate of nucleic acid ligation exceeds the rate of nucleic acid ligation of a control; wherein the control comprises: (a) a first protein consisting of the PPK domain of the fusion polypeptide; and (b) a second protein consisting of the ATP-dependent nucleic acid ligase domain of the fusion polypeptide; wherein said first and second proteins are not linked.

In some embodiments, the rate of RNA ligation exceeds the rate of RNA ligation of a control; wherein the control comprises: (a) a first protein consisting of the PPK domain of the fusion polypeptide; and (b) a second protein consisting of the ATP-dependent nucleic acid ligase domain of the fusion polypeptide; wherein the ATP-dependent nucleic acid ligase domain is a dsRNA ligase domain and said first and second proteins are not linked.

The disclosure also provides use of a fusion polypeptide described herein in a method of producing an oligonucleotide from two or more oligonucleotide fragments.

In some embodiments, the oligonucleotide is a therapeutic oligonucleotide.

In some embodiments, the oligonucleotide product is at least 80% pure, optionally wherein the oligonucleotide product is at least 85% pure, at least 90% pure, at least 95% pure, optionally wherein the oligonucleotide product is at least 98% pure.

### DETAILED DESCRIPTION

### Definitions

Unless expressly defined otherwise, technical and scientific terms used in this disclosure have the meanings that are commonly understood by people skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

As used throughout this disclosure, articles such as “a” and “an” refer to one or more than one (at least one) of the grammatical object of the article.

The term “and/or” means either “and” or “or” unless indicated otherwise.

As used herein, the term “about” typically refers to the value which immediately follows the term ‘about’. For example, “about 15 or more nucleotides” typically refers to 15 or more nucleotides. In some aspects and embodiments, the term “about” embraces values which are +/−1, 2 or 3 of the stated value. For example, “about 15 or more nucleotides” may refer to 15+/−3 nucleotides, e.g. 12, 13, 14, 15, 16, 17 or 18 nucleotides.

As used herein, “ligation” refers to the enzymatic linking of two adjacent nucleotides, e.g. via a phosphodiester bond. An ATP-dependent nucleic acid ligase is an enzyme that uses ATP to catalyze the formation of a covalent bond between two adjacent nucleotides.

As used herein, the term “oligonucleotide” refers to a nucleic acid, typically comprising up to 100 nucleotides. Unless expressly defined otherwise, the term “oligonucleotide” embraces both single-stranded oligonucleotides and double-stranded oligonucleotides. The oligonucleotide may comprise DNA and/or RNA. For example, a portion of the oligonucleotide may be double-stranded DNA, while another portion is double-stranded RNA, forming a DNA-RNA chimera.

The term “therapeutic oligonucleotide” refers to an oligonucleotide that can provide a therapeutic effect, e.g. by interacting with a biomolecule and/or by regulating gene expression. Therapeutic oligonucleotides include, but are not limited to, RNA interference (RNAi) agents and antisense oligonucleotides (ASO). RNAi is a post-transcriptional, targeted gene-silencing technique that uses RNAi agents to degrade messenger RNA (mRNA) containing the same sequence as the RNAi agent. ASOs are single-stranded nucleic acids that can be used to target mRNA derived from a gene of interest. ASOs can alter gene expression via a number of mechanisms including direct steric blockage of mRNA and ribonuclease H (RNase H) mediated degradation of mRNA.

RNAi agents include, as non-limiting examples, siRNAs (small interfering RNAs), dsRNAs (double-stranded RNAs), shRNAs (short hairpin RNAs) and miRNAs (micro RNAs). RNAi agents also include, as additional non-limiting examples, locked nucleic acid (LNA), Morpholino, UNA, threose nucleic acid (TNA), glycol nucleic acid (GNA), peptide nucleic acid (PNA) and fluoro-arabinonucleic acid (FANA). RNAi agents also include molecules in which one or more strands are a mixture of RNA, DNA, LNA, Morpholino, UNA (unlocked nucleic acid), TNA, GNA, and/or FANA. As a non-limiting example, one or both strands of an RNAi agent could be, for example, RNA, except that one or more RNA nucleotides is replaced by DNA, LNA, Morpholino, UNA, TNA, GNA, and/or FANA, etc. In some embodiments, one or both strands of the RNAi agent can be nicked, and both strands can be the same length, or one strand can be shorter than the other. The oligonucleotide of the invention may be any of the RNAi agents described herein.

The term “oligonucleotide fragment” herein refers to a nucleic acid that can be ligated to one or more additional oligonucleotide fragments to provide an oligonucleotide product. Unless expressly defined otherwise, the term “oligonucleotide fragment” embraces both single-stranded oligonucleotide fragments and double-stranded oligonucleotide fragments. Each oligonucleotide fragment corresponds to a portion of the oligonucleotide product. A “terminal oligonucleotide fragment” herein refers to a nucleic acid that corresponds to an end (e.g. 5′ or 3′ end) portion of the oligonucleotide product.

The term “overhang” or “nucleotide overhang” herein refers to at least one unpaired nucleotide that protrudes from the end of at least one of the two strands of a double-stranded oligonucleotide. In some embodiments, when a 3′-end of one strand extends beyond the 5′-end of the other strand, or vice versa, this forms a nucleotide overhang, e.g., the unpaired nucleotide(s) form the overhang. An overhang that is complementary to the overhang of a second oligonucleotide fragment may be referred to as a “sticky end”. The oligonucleotide fragments of described herein may have one or two sticky ends.

“Blunt” or “blunt end” means that there are no unpaired nucleotides at that end of the double-stranded nucleic acid, i.e., no nucleotide overhang. A “blunt ended” oligonucleotide or oligonucleotide fragment is an oligonucleotide that is double stranded over its entire length, i.e., no nucleotide overhang at either end of the molecule.

Double-stranded nucleic acids comprise two anti-parallel and substantially complementary nucleic acid strands which are referred to as “sense” and “antisense” strands. In the context of double-stranded RNAi agents, the “antisense strand” refers to the strand of an RNAi which includes a region that is substantially complementary to a target sequence, e.g. an mRNA sequence. The “sense strand” refers to the strand of an RNAi that includes a region that is substantially complementary to a region of the antisense strand. The sense and antisense strands of an RNAi agent may be referred to as the passenger and guide strands, respectively.

Sequences that are “substantially complementary” may be fully complementary or may contain one or more mismatches upon hybridization, while retaining the ability to hybridize under the conditions most relevant to their ultimate application.

The stoichiometric concentration of cofactor is the theoretical concentration required to achieve complete ligation in a given ligation reaction. The skilled person can readily derive the stoichiometric concentration of ATP required to achieve complete ligation based on the concentration of oligonucleotide fragments and the number of ligation reactions required to produce the oligonucleotide product. For example, a ligation reaction using 1 mM substrate which requires four ligation reactions has a stoichiometric ATP concentration of 4 mM.

“Conversion” refers to the enzymatic transformation of a substrate to the corresponding product. “Percent conversion” or “conversion” refers to the percentage of oligonucleotide fragment(s) that is converted to oligonucleotide within a period of time under the specified conditions. Thus, “enzymatic activity” or “activity” of a ligase can be expressed as the “percent conversion” of the oligonucleotide fragment(s) to oligonucleotide product.

Ideally to compare the activity between ligation reactions and account for natural variation in peak intensity between injections, the % conversion to product would be calculated for each sample analyzed using the following equation:

$$\% Conv = \frac{( \frac{A 260 Product ( s )}{{ε}_{p}} )}{\begin{matrix}( \frac{A 260 Product ( s )}{{ε}_{p}} ) + ( \frac{A 260 Substrate ( s )}{{ε}_{s}} ) + \\ ( \frac{A 260 Intermediate ( s )}{{ε}_{i}} )\end{matrix}} \times 100$$

whereby εₚ, εₛ and εᵢ=the extinction coefficient of the product, substrate, and intermediate oligonucleotides respectively. In some instances, such as using the analytical methods described herein, it is not possible to resolve all substrates, reaction intermediates and products. Therefore, the % conversion according to the above equation cannot be determined. However, in some instances, it is possible to resolve at least one substrate, reaction intermediate and product, such as well-defined GalNAc-containing oligonucleotides, including GalNAc containing substrate fragments (e.g. the GalNAc containing substrate fragment (F21), reaction intermediate of F21 ligated to F20 (int. F20/F21) and the product sense strand (SS) used in the examples described herein). Therefore, a pseudo-% conversion can be calculated, denoted with arbitrary units (AU), which considers only these well resolved species according to the following equation:

$$AU = \frac{( \frac{A 260 SS}{{ε}_{SS}} )}{( \frac{A 260 SS}{{ε}_{S S}} ) + ( \frac{A 260 F 21}{{ε}_{F 21}} ) + ( \frac{A 260 Int . F 20 / F 21}{{ε}_{int . F 20 / F 21}} )}$$

whereby εₛₛ, ε_{F21} and ε_{int.F20/F21}=the extinction coefficient of the SS, F21, and Int. F20/F21 oligonucleotides respectively (see Table 4). Using such a calculation an AU=1.0 would imply that no more GalNAc-containing substrate or intermediate oligonucleotides are present in the reaction and that they have all be converted to GalNAc-containing product (SS), i.e. AU=1.0 corresponds to 100% conversion of the sense strand. In reality, for samples where AU=1.0 the only other peak present in the chromatogram corresponds with the product antisense strand (AS), and no other intermediates or starting materials can be identified. Furthermore, the ratio of the SS and AS peaks are consistent with that of the authentic product standard. Taken together AU=1.0 is considered to be essentially equivalent to 100% conversion of both strands.

“Improved enzyme properties” and the like refer to an enzyme property that is better or more desirable for a specific purpose as compared to a reference, such as a ligase which is not linked to a PPK. As used herein, “improved enzyme properties” and the like typically refer to the properties of the ligase domain of the fusion polypeptides described herein. Enzyme properties that are expected to be improved include, but are not limited to, enzyme activity (which can be expressed as a percentage of substrate conversion or in arbitrary units as described herein), thermal stability, solvent stability, pH activity characteristics, cofactor requirements, and tolerance to inhibitors (e.g., reaction component, substrate or product inhibition). The fusion polypeptides described herein also demonstrate improved ligase activity when immobilized as compared to immobilized ligase polypeptides that are not linked to a PPK domain. The fusion polypeptides described herein may also exhibit improved soluble yield from host cells which results in increased enzyme activity when using crude extracts (e.g. cell free lyophilized extracts or cell lysates).

An “isolated polypeptide” (e.g. an “isolated fusion polypeptide”) refers to a polypeptide that is substantially separated from other substances with which it is naturally associated, such as proteins, lipids, and polynucleotides. The term comprises polypeptides that have been removed or purified from their naturally occurring environment or expression system (e.g., in host cells or in vitro synthesis). Polypeptides (e.g. fusion polypeptides) may be present in the cell, in the cell culture medium, or prepared in various forms, such as lysates or isolated preparations. As such, in some embodiments, the fusion polypeptide is an isolated fusion polypeptide.

As used herein, “crude extract” is the solution produced by lysing cells expressing a polypeptide of interest and removing cell debris, e.g. by centrifugation. Crude extract described herein may be cell free lyophilized extract or cell lysate.

“Naturally occurring” or “wild-type” refers to the form found in nature. For example, a naturally-occurring or wild-type polypeptide or polynucleotide sequence is a sequence that is present in an organism that can be isolated from sources in nature, and which has not been intentionally modified by manual procedures.

“Polyphosphate kinase” or “PPK” as used herein, refers to a wild-type or engineered enzyme having polyphosphate kinase activity, i.e. an enzyme that catalyzes the formation of ATP from AMP and polyphosphate. PPKs are also be described herein as phosphotransferases or polyphosphate-nucleotide phosphotransferases.

The terms “polynucleotide”, “nucleic acid molecule” and “nucleic acid” are used interchangeably herein.

The terms “protein”, “polypeptide” and “peptide” are used interchangeably herein to denote a polymer of at least two amino acids covalently linked by an amide bond, regardless of length or post-translational modification (e.g., glycosylation, phosphorylation, lipidation, myristoylation, ubiquitination, etc.). This definition includes D-amino acids and L-amino acids, as well as mixtures of D-amino acids and L-amino acids. Preferably, the amino acids have L configuration.

“Recombinant”, “engineered” or “non-naturally occurring” when used with reference to, for example, a cell, nucleic acid, or polypeptide, refers to a material or material corresponding to the native form of the material that has been modified in a manner that would not otherwise exist in nature, or is identical thereto but produced or derived from synthetic material and/or by manipulation using recombinant techniques.

As used herein, an oligonucleotide or oligonucleotide fragment comprising a chemical modification refers to oligonucleotides and oligonucleotide fragments having a modified nucleotide, oligonucleotides and oligonucleotide fragments having a modified backbone, and/or oligonucleotides and oligonucleotide fragments that are conjugated to a ligand.

As used herein, an “unmodified nucleotide” is a nucleotide that has a deoxyribose or ribose sugar and a nucleobase selected from adenine, cytosine, guanine, thymine and uracil. As used herein, “modified nucleotide” refers to a nucleotide comprising a modified sugar and/or a modified base. A modified sugar may be a modified deoxyribose sugar or modified ribose sugar that is substituted at one or more positions with a non-hydrogen substituent. A modified base refers to any base other than adenine, cytosine, guanine, thymine and uracil. Exemplary modified sugars and modified bases are described herein.

As used herein, an “unmodified backbone” consists of 3′ to 5′ phosphodiester bonds. As used herein, a “modified backbone” may comprise any non-natural internucleoside linkage, e.g. a phosphororthioate linkage (e.g. a chiral phosphorothioate linkage) and a phosphorodithioate linkage. Exemplary backbone modifications are described herein.

The abbreviations used for the genetically encoded amino acids are conventional and are as follows:

| Amino Acid | Three-Letter | One-Letter Abbreviation |
|---|---|---|
| Alanine | Ala | A |
| Arginine | Arg | R |
| Asparagine | Asn | N |
| Aspartate | Asp | D |
| Cysteine | Cys | C |
| Glutamate | Glu | E |
| Glutamine | Gln | Q |
| Glycine | Gly | G |
| Histidine | HIS | H |
| Isoleucine | Ile | I |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |

For a deletion of an amino acid a “-” is used and for a Stop Codon a “*” is used. When the three-letter abbreviations are used, unless specifically preceded by an “L” or a “D” or clear from the context in which the abbreviation is used, the amino acid may be in either the L- or D-configuration about α-carbon (C_{α}). For example, whereas “Ala” designates alanine without specifying the configuration about the α-carbon, “D-Ala” and “L-Ala” designate D-alanine and L-alanine, respectively.

When the one-letter abbreviations are used, upper case letters designate amino acids in the L-configuration about the α-carbon and lower case letters designate amino acids in the D-configuration about the α-carbon. For example, “A” designates L-alanine and “a” designates D-alanine. When polypeptide sequences are presented as a string of one-letter or three-letter abbreviations (or mixtures thereof), the sequences are presented in the amino (N) to carboxy (C) direction in accordance with common convention.

The abbreviations used for the genetically encoding nucleotides are conventional and are as follows: adenosine (A); guanosine (G); cytidine (C); thymidine (T); and uridine (U). Unless specifically delineated, the abbreviated nucleotides may be either ribonucleotides or 2′-deoxyribonucleotides. The nucleotides may be specified as being either ribonucleotides or 2′-deoxyribonucleotides on an individual basis or on an aggregate basis. When nucleic acid sequences are presented as a string of one-letter abbreviations, the sequences are presented in the 5′ to 3′ direction in accordance with common convention, and the phosphodiester bonds are not indicated.

The skilled person is well aware that guanine, cytosine, adenine, and uracil may be replaced by other moieties without substantially altering the base pairing properties of an oligonucleotide comprising a nucleotide bearing such replacement moiety. For example, without limitation, a nucleotide comprising inosine as its base may base pair with nucleotides containing adenine, cytosine, or uracil. Hence, nucleotides containing uracil, guanine, or adenine may be replaced in the nucleotide sequences of oligonucleotides featured in the present disclosure by a nucleotide containing, for example, inosine. In another example, adenine and cytosine anywhere in the oligonucleotide can be replaced with guanine and uracil, respectively to form Wobble base pairing with the target mRNA.

Methods of determining percentage sequence identity are known in the art. By way of example, when assessing sequence identity, a sequence having a defined number of contiguous nucleotides or amino acids may be aligned with a nucleic acid or peptide sequence (having the same number of contiguous nucleotides or amino acids) from the corresponding portion of a nucleic acid or peptide sequence disclosed herein. The percentage sequence identity can be calculated by determining the number of positions at which either the identical nucleic acid base or amino acid residue occurs in both sequences, or a nucleic acid base or amino acid residue is aligned with a gap to yield the number of matched positions, dividing the number of matched positions by the total number of positions in the sequence and multiplying the result by 100 to yield the percentage of sequence identity. Those skilled in the art will appreciate that there are many established algorithms available to align two sequences. The optimal alignment of sequences for comparison can be conducted, for example, by the local homology algorithm of Smith and Waterman, 1981, Adv. Appl. Math. 2:482, by the Homology alignment algorithm of Needleman and Wunsch, 1970, J. Mol. Biol. 48:443, by the search for similarity method of Pearson and Lipman, 1988, Proc. Natl. Acad. Sci. USA 85:2444, by computerized implementations of these algorithms (GAP, BESTFIT, FASTA, and TFASTA in the GCG Wisconsin Package) or by visual inspection (see generally, Current Protocols in Molecular Biology, FM Ausubel et al. eds., Current Protocols, a Joint Venture between Greene Publishing Associates, Inc. and John Wiley & Sons, Inc., (1995 Supplement) (Ausubel)). Examples of algorithms that are suitable for determining the percent sequence identity and percent sequence similarity are the BLAST and BLAST 2.0 algorithms, which are described in Altschul et al., 1990, J. Mol. Biol. 215:403-410 and Altschul et al., 1977, Nucleic Acids Res. 3389-3402, respectively. Software for performing BLAST analysis is publicly available through the National Center for Biotechnology Information website. The algorithm involves first identifying high scoring sequence pairs (HSPs) by identifying short words of length W in the query sequence, which either match or satisfy some positive-valued threshold scores T when aligned with a word of the same length in the database sequence. T is referred to as, the neighborhood word score threshold (Altschul et al., Supra). These initial neighborhood word hits serve as seeds for initiating searches to find longer HSPs that contain them. The word hits are then extended in both directions along each sequence for as far as the cumulative alignment score can be increased. For nucleotide sequences, the cumulative scores are calculated using the parameters M (reward score for matched pair of residues; always>0) and N (penalty score for mismatched residues; always <0). For amino acid sequences, a scoring matrix is used to calculate the cumulative score. The extension of the word hits in each direction are halted when: the cumulative alignment score falls off by the quality X from its maximum achieved value; the cumulative score goes 0 or below, due to the accumulation of one or more negative-scoring residue alignments; or the end of either sequence is reached. The BLAST algorithm parameters W, T and X determine the sensitivity and speed of the alignment. The BLASTN program (for nucleotide sequences) uses as defaults a word length (W) of 11, the expected value (E) of 10, M=5, N=−4, and a comparison of both strands as a default value. For amino acid sequences, the BLASTP program uses as defaults the word length (W) of 3, the expected value (E) of 10 and the BLOSUM62 scoring matrix (see Henikoff and Henikoff, 1989, Proc Natl Acad Sci USA 89:10915). Exemplary determination of sequence alignments and % sequence identity can employ the BESTFIT or GAP programs in the GCG Wisconsin Software package (Accelrys, Madison WI), using the default parameters provided.

It will be appreciated that, regardless of the percent sequence identity to a reference sequence, an ATP-dependent nucleic acid ligase or an ATP-dependent nucleic acid ligase domain (e.g. dsRNA ligase domain) possesses ATP-dependent nucleic acid ligase activity. It will likewise be appreciated that, regardless of the percent sequence identity to a reference sequence, a PPK or a PPK domain possesses PPK activity.

“Suitable reaction conditions” refer to those conditions (e.g., enzyme loading, substrate loading, temperature, pH, etc.) in the reaction system, under which the substrate is converted to the desired product. Suitable reaction conditions can be readily identified by the person skilled in the art. Exemplary “suitable reaction conditions” are provided in the present disclosure and illustrated by examples.

### Ligation Reactions

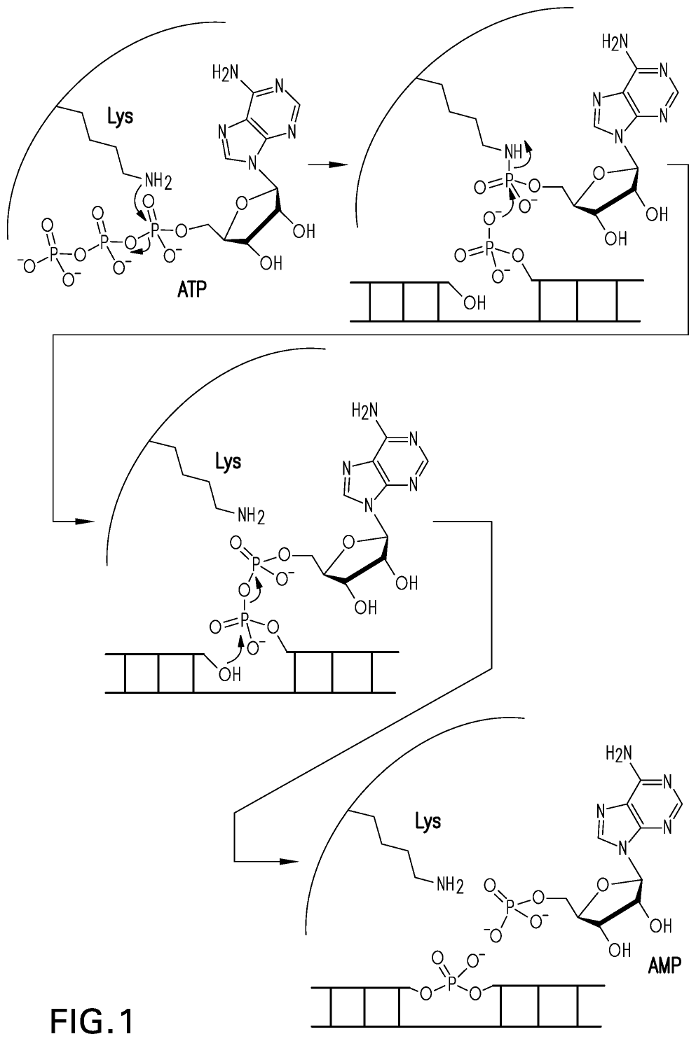

The enzymatic ligation of short oligonucleotide fragments offers a sustainable and economical alternative to solid phase chemical synthesis of full-length (e.g. therapeutic) oligonucleotides. Herein, “nucleic acid ligases” refers to ATP-dependent nucleic acid ligases (rather than e.g. NAD-dependent nucleic acid ligases). A major drawback of ligation reactions catalysed by ATP-dependent nucleic acid ligases is the need for a stoichiometric quantity of an expensive cofactor, ATP. ATP-dependent nucleic acid ligases are ATP-dependent enzymes, and their catalytic mechanism has been well characterized. First, the active-site lysine attacks the α-phosphate of ATP, forming a lysine-AMP intermediate and releasing pyrophosphate. Next, the AMP is transferred from the active site lysine to the 5′-phosphate of the 3′ ligation fragment, forming an adenylated oligonucleotide intermediate. Finally, the 3′-OH of the 5′ ligation fragment attacks the 5′ phosphate of the adenylated intermediate, releasing AMP (FIG. 1) (Nandakumar, J., Shuman, S. & Lima, C. D. Cell 127, 71-84 (2006)). In this way, one molecule of ATP is converted to AMP per ligation reaction. In practice, an excess of ATP in required to achieve complete ligation.

As demonstrated herein, the requirement for high concentrations of ATP in ligation reactions can be overcome by incorporating an ATP regeneration system into the reaction. The ATP regeneration system described herein comprises PPK and polyphosphate. PPK generates ATP from AMP using polyphosphate as a phosphate donor. ATP that is converted to AMP during the ligation reaction can be regenerated to ATP by PPK and used as a cofactor in a subsequent ligation reaction. This cycling of ATP obviates the need for high ATP concentration in the starting reaction. Instead, the reaction can be performed using sub-stoichiometric concentrations of ATP, and/or using the cheaper alternative, AMP.

In some embodiments, the oligonucleotide product is obtained with a percent conversion of at least 10%, at least 20%, at least 30%, at least 40%, at least 50%, at least 60%, at least 70%, at least 75%, at least 80%, at least 85%, at least 90%, at least 95%, or 100%. Percent conversion may be represented in arbitrary units (AU), calculated as described herein.

### Oligonucleotides and Oligonucleotide Fragments

Methods of the invention produce oligonucleotides by ligating two or more oligonucleotide fragments. The produced oligonucleotides (also referred to herein as “oligonucleotide products”) are nucleic acids, which typically comprise up to 100 nucleotides. Oligonucleotide fragments may be referred to herein as “substrates” of the ligation reaction.

In some embodiments, the oligonucleotide is a therapeutic oligonucleotide. In some embodiments, the therapeutic oligonucleotide is a small interfering RNA (siRNA) or an antisense oligonucleotide (ASO). In some embodiments, the oligonucleotide is an aptamer.

In some embodiments, the oligonucleotide is a double-stranded oligonucleotide. In some embodiments, the oligonucleotide comprises an overhang. In some embodiments, the oligonucleotide comprises a 3′ overhang. In some embodiments, the oligonucleotide comprises a 5′ overhang. In some embodiments, the overhang comprises 1, 2, 3, 4, 5, 6, 7, or 8 nucleotides. In some embodiments, the oligonucleotide comprises a blunt end. In some embodiments, the oligonucleotide comprises two blunt ends.

In some embodiments, the oligonucleotide is a single-stranded oligonucleotide.

In some embodiments, the oligonucleotide is up to 20 nucleotides in length. In some embodiments, the oligonucleotide is up to 25, up to 30, up to 35, up to 40, up to 45, up to 50, up to 55, up to 60, up to 65, up to 70, up to 75, up to 80, up to 85, up to 90, up to 95, or up to 100 nucleotides in length. In some embodiments, the oligonucleotide is up to 60 nucleotides in length.

In some embodiments, the oligonucleotide is at least 20 nucleotides in length. In some embodiments, the oligonucleotide is at least 25, at least 30, at least 35, at least 40, at least 45, at least 50, at least 55, at least 60, at least 65, at least 70, at least 75, at least 80, at least 85, at least 90, at least 95, or 100 nucleotides in length.

In some embodiments, the oligonucleotide is 10-100 nucleotides in length. In some embodiments, the oligonucleotide is 10-80, 10-70, 10-60, 10-50, 10-40, 10-30, 10-25, 15-80, 15-70, 15-60, 15-50, 15-40, 15-30, or 15-25 nucleotides in length. In some embodiments, the oligonucleotide is 15-25 nucleotides in length.

In some embodiments, the two or more oligonucleotide fragments comprise single-stranded oligonucleotide fragments.

In some embodiments, the two or more oligonucleotide fragments comprise double-stranded oligonucleotide fragments. In some embodiments, one or more of the oligonucleotide fragments comprises one or more mismatches. In some embodiments, one or more of the oligonucleotide fragments comprise an overhang. In some embodiments, one or more of the oligonucleotide fragments comprise a 3′ overhang. In some embodiments, one or more of the oligonucleotide fragments comprise a 5′ overhang. In some embodiments, one or more of the oligonucleotide fragments comprise a 3′ overhang and a 5′ overhang. In some embodiments, the overhang comprises 1, 2, 3, 4, 5, 6, 7, or 8 nucleotides.

In some embodiments, the two or more oligonucleotide fragments comprise a first oligonucleotide fragment having an overhang that is complementary to the overhang of a second oligonucleotide fragment. In some embodiments, the two or more oligonucleotide fragments comprise a first oligonucleotide fragment having a 3′ overhang and a 5′ overhang, wherein the 3′ overhang is complementary to the 5′ overhang of a second oligonucleotide fragment and the 5′ overhang is complementary to the 3′ overhang of a third oligonucleotide.

In some embodiments, one or more of the oligonucleotide fragments comprise a blunt end. In some embodiments, one or more of the oligonucleotide fragments comprise a 3′ overhang and a 5′ blunt end. In some embodiments, one or more of the oligonucleotide fragments comprise a 5′ overhang and a 3′ blunt end.

In some embodiments, two or more oligonucleotide fragments comprise two or more RNA oligonucleotide fragments. In some embodiments, the two or more RNA oligonucleotide fragments comprises double-stranded RNA (dsRNA) oligonucleotide fragments. In some embodiments, the two or more RNA oligonucleotide fragments comprise single-stranded RNA (ssRNA) oligonucleotide fragments.

In some embodiments, two or more oligonucleotide fragments comprise two or more DNA oligonucleotide fragments. In some embodiments, the two or more DNA oligonucleotide fragments comprises double-stranded DNA (dsDNA) oligonucleotide fragments. In some embodiments, the two or more DNA oligonucleotide fragments comprise single-stranded DNA (ssDNA) oligonucleotide fragments.

In some embodiments, the two or more oligonucleotide fragments comprise DNA oligonucleotide fragments and RNA oligonucleotide fragments. In some embodiments, the two or more oligonucleotide fragments comprise dsDNA oligonucleotide fragments and dsRNA oligonucleotide fragments.

In some embodiments, one or more of the oligonucleotide fragments comprise one or two strands which are RNA, or a mixture of RNA, DNA, LNA, Morpholino, UNA (unlocked nucleic acid), TNA (threose nucleic acid), GNA (glycol nucleic acid), and/or FANA (Fluoro-arabino nucleic acid), modified RNA, etc. As a non-limiting example, one or both strand(s) could be, for example, RNA except that one or more nucleotide(s) is replaced by DNA, LNA, Morpholino, UNA, TNA, GNA, and/or FANA, and/or modified RNA (e.g., any modified RNA disclosed herein or known in the art, such as 2′ modified RNA, including but not limited to 2′-F, 2′-OMe, 2′-O-MOE RNA, etc.).

In some embodiments, the two or more oligonucleotide fragments are the same length. In some embodiments, the two or more oligonucleotide fragments are different lengths. In some embodiments, each of the two or more oligonucleotide fragments are 3-20 nucleotides in length. In some embodiments, each of the two or more oligonucleotide fragments are 4-16 nucleotides in length. In some embodiments, each of the two or more oligonucleotide fragments are 4-16, 4-15, 5-15, 6-15, 4-14, 4-13, 4-12, 4-11, 4-10, 4-9, 5-9, or 6-9 nucleotides in length.

In some embodiments, each of the two or more oligonucleotide fragments are at least 3 nucleotides in length. In some embodiments, each of the two or more oligonucleotide fragments are at least 4, at least 5, at least 6, at least 7, at least 8, at least 9, at least 10, at least 11, at least 12, at least 13, at least 14, or at least 15 nucleotides in length.

In some embodiments, the two or more oligonucleotide fragments comprise 3 or more, 4 or more, 5 or more, 6 or more, 7 or more, 8 or more, 9 or more, or 10 or more oligonucleotide fragments.

In some embodiments, one or more ligation reactions are required to generate the oligonucleotide product. In some embodiments, 2 or more, 3 or more, 4 or more, 5 or more, 6 or more, 7 or more, 8 or more, 9 or more, or 10 or more ligation reactions are required to generate the oligonucleotide product.

In some embodiments, one or more of the oligonucleotide fragments comprises a chemical modification. In some embodiments, one or more of the oligonucleotide fragments comprises at least one backbone modification. In some embodiments, one or more of the oligonucleotide fragments comprises at least one nucleotide modification. In some embodiments, one or more of the oligonucleotide fragments comprises at least one sugar modification (e.g. at the 2′-position or 4′-position). In some embodiments, one or more of the oligonucleotide fragments comprises: (i) at least one backbone modification; (ii) at least one nucleotide modification; and/or (iii) at least one sugar modification. In some embodiments, the oligonucleotide comprises a chemical modification. In some embodiments, the oligonucleotide comprises at least one backbone modification. In some embodiments, the oligonucleotide comprises at least one nucleotide modification. In some embodiments, the oligonucleotide comprises at least one sugar modification (e.g. at the 2′-position or 4′-position). In some embodiments, the oligonucleotide comprises: (i) at least one backbone modification; (ii) at least one nucleotide modification; and/or (iii) at least one sugar modification.

In some embodiments, one or more of the oligonucleotide fragments comprise a modification selected from the group consisting of: 2′-O-methyl (2′-OMe), 2′-flouro (2′-F), 2′-deoxy, 2′-deoxy-2′-fluoro, 2′-O-methoxyethyl (2′-O-MOE), 2′-O-aminopropyl (2′-O-AP), 2′-O-dimethylaminoethyl (2′-O-DMAOE), 2′-O-dimethylaminopropyl (2′-O-DMAP), 2′-O-dimethylaminoethyloxyethyl (2′-O-DMAEOE), 2′-O—N-methylacetamido (2′-O-NMA), locked nucleic acid (LNA), glycol nucleic acid (GNA), phosphoramidate (e.g. mesyl phosphoramidate), 2′,3′-seco nucleotide mimic, 2′-F-arabino nucleotide, abasic nucleotide, 2′-amino modified nucleotide, 2′-alkyl-modified nucleotide, morpholino nucleotide, vinylphosphonate (e.g. 5′ vinylphosphonate), and cyclopropyl phosphonate deoxyribonucleotide. In some embodiments, one or more of the oligonucleotide fragments comprise a 2′-modification selected from the group consisting of: 2′-OMe, 2′-F, and 2′-deoxy.

In some embodiments, the oligonucleotide comprises a modification selected from the group consisting of: 2′-O-methyl (2′-OMe), 2′-flouro (2′-F), 2′-deoxy, 2′-deoxy-2′-fluoro, 2′-O-methoxyethyl (2′-O-MOE), 2′-O-aminopropyl (2′-O-AP), 2′-O-dimethylaminoethyl (2′-O-DMAOE), 2′-O-dimethylaminopropyl (2′-O-DMAP), 2′-O-dimethylaminoethyloxyethyl (2′-O-DMAEOE), 2′-O—N-methylacetamido (2′-O-NMA), locked nucleic acid (LNA), glycol nucleic acid (GNA), phosphoramidate (e.g. mesyl phosphoramidate), 2′,3′-seco nucleotide mimic, 2′-F-arabino nucleotide, abasic nucleotide, 2′-amino modified nucleotide, 2′-alkyl-modified nucleotide, morpholino nucleotide, vinylphosphonate (e.g. 5′ vinylphosphonate), and cyclopropyl phosphonate deoxyribonucleotide. In some embodiments, the oligonucleotide comprises a 2′-modification selected from the group consisting of: 2′-OMe, 2′-F, and 2′-deoxy.

In some embodiments, one or more of the oligonucleotide fragments comprises at least one phosphorothioate or methylphosphonate internucleotide linkage. In some embodiments, the oligonucleotide comprises at least one phosphorothioate or methylphosphonate internucleotide linkage. In some embodiments, the oligonucleotide comprises at least one chiral phosphorothioate linkage.

In some embodiments, one or more of the oligonucleotide fragments is conjugated to at least one ligand. The ligand may be conjugated to the sense strand, antisense strand or both strands, in any configuration e.g. at the 3′-end, 5′-end, non-end or a combination.

In some embodiments, the oligonucleotide is conjugated to at least one ligand. The ligand may be conjugated to the sense strand, antisense strand or both strands, in any configuration e.g. at the 3′-end, 5′-end, non-end or a combination.

In some embodiments, the ligand comprises one or more N-Acetylgalactosamine (GalNAc) derivatives. GalNAc is an amino sugar derivative of galactose which may be used as a targeting ligand in oligonucleotides intended for targeting to the liver, where it binds to the asialoglycoprotein receptors on hepatocytes. In some embodiments, the ligand comprises one or more GalNAc derivatives conjugated through a bivalent or trivalent branched carrier. In some embodiments, the ligand is a peptide or a peptidomimetic.

In some embodiments, the ligand is conjugated to the sense strand. In some embodiments, the ligand is conjugated to the 3′ end of the sense strand. In some embodiments, the ligand is conjugated to the 5′ end of the sense strand. In some embodiments, the ligand is conjugated to a non-end of the sense strand.

In some embodiments, the ligand is conjugated to the antisense strand. In some embodiments, the ligand is conjugated to the 3′ end of the antisense strand. In some embodiments, the ligand is conjugated to a non-end of the antisense strand.

In some embodiments, one or more of the oligonucleotide fragments comprises at least one 2′-modified nucleotide selected from a group consisting of 2′-OMe, 2′-F, 2′-deoxy, 2′-deoxy-2′-fluoro, and 2′-O-MOE. In some embodiments, one or more of the oligonucleotide fragments is a dsRNA wherein the sense strand is conjugated to one or more GalNAc ligand(s).

In some embodiments, the oligonucleotide comprises at least one 2′-modified nucleotide selected from a group consisting of 2′-OMe, 2′-F, 2′-deoxy, 2′-deoxy-2′-fluoro, and 2′-O-MOE. In some embodiments, the oligonucleotide is a dsRNA wherein the sense strand is conjugated to one or more GalNAc ligand(s).

In some embodiments, the oligonucleotide is an RNAi agent comprising at least one 2′-modified nucleotide selected from a group consisting of 2′-OMe, 2′-F, 2′-deoxy, 2′-deoxy-2′-fluoro, and 2′-O-MOE. In some embodiments, the oligonucleotide is an RNAi agent wherein the sense strand is conjugated to one or more GalNAc ligand(s).

In some embodiments, the method is performed with an oligonucleotide fragment concentration of at least 1 mM, at least 2 mM, at least 3 mM, at least 4 mM, at least 5 mM, at least 6 mM, at least 7 mM, at least 8 mM, at least 9 mM, or at least 10 mM. In some embodiments, the method is performed with at least 1 mM, at least 2 mM, at least 3 mM, at least 4 mM, at least 5 mM, at least 6 mM, at least 7 mM, at least 8 mM, at least 9 mM, or at least 10 mM of each oligonucleotide fragment. In some embodiments, the method is performed with equimolar amounts of each of the two or more oligonucleotide fragments.

In some embodiments, the method produces at least 15 g of oligonucleotide product per litre of reaction mixture. In some embodiments, the method produces at least 16 g, at least 17 g, at least 18 g, at least 19 g, at least 20 g, at least 30 g, at least 40 g, at least 50 g, at least 60 g, at least 70 g, at least 80 g, at least 90, or at least 100 g of oligonucleotide product per litre of reaction mixture.

In some embodiments, the method further comprises purifying the oligonucleotide product from the reaction mixture. In some embodiments, the oligonucleotide product is at least 80% pure, optionally wherein the oligonucleotide product is at least 85% pure, at least 90% pure, at least 95% pure, optionally wherein the oligonucleotide product is at least 98% pure, optionally wherein the oligonucleotide product is at least 99% pure, optionally wherein the oligonucleotide product is at least 99.5% pure, optionally wherein the oligonucleotide product is at least 99.9% pure. An oligonucleotide product that is pure typically does not contain oligonucleotide fragments, intermediate ligation products, or side products arising from non-specific ligation. The oligonucleotide product may be purified or isolated using any method known in the art, for example by separating oligonucleotides based on their size, e.g. using gel extractions, using cellulose-based matrices, ultrafiltration and/or chromatography.

The disclosure also provides an oligonucleotide produced by a method described herein.

### ATP-Dependent Nucleic Acid Ligase

ATP-dependent nucleic acid ligases are a family of enzymes which use ATP as a cofactor to catalyze the ligation of oligonucleotide fragments. The catalytic mechanism of ATP-dependent nucleic acid ligases has been well characterized, as summarised above and in FIG. 1.

In some embodiments, the ATP-dependent nucleic acid ligase is an RNA ligase. In some embodiments, the RNA ligase is a dsRNA ligase. In some embodiments, the RNA ligase is a member of RNA ligase 2 family. In some embodiments, the RNA ligase is Bacteriophage RB69 RNA ligase 2 (UniProt ID: Q7Y4V8). In some embodiments, the RNA ligase is Bacteriophage T4 RNA ligase 2 (UniProt ID: P32277).

In some embodiments, the RNA ligase comprises an amino acid sequence having at least 70% sequence identity to SEQ ID NO: 1, which is the amino acid sequence of Bacteriophage RB69 RNA ligase 2 (UniProt ID: Q7Y4V8). In some embodiments, the RNA ligase comprises an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% sequence identity to SEQ ID NO: 1.

In some embodiments, the RNA ligase comprises an amino acid sequence having at least 70% sequence identity to SEQ ID NO: 2, which is the amino acid sequence of an optimized Bacteriophage RB69 RNA ligase 2. In some embodiments, the RNA ligase comprises an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity to SEQ ID NO: 2.

In some embodiments, the RNA ligase comprises an amino acid sequence having at least 70% sequence identity to SEQ ID NO: 88, which is the amino acid sequence of an optimized Bacteriophage RB69 RNA ligase 2. In some embodiments, the RNA ligase comprises an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity to SEQ ID NO: 88.

In some embodiments, the RNA ligase comprises an amino acid sequence having at least 70% sequence identity to SEQ ID NO: 3, which is the amino acid sequence of Bacteriophage T4 RNA ligase 2 (UniProt ID: P32277). In some embodiments, the RNA ligase comprises an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity to SEQ ID NO: 3.

In some embodiments, the ATP-dependent nucleic acid ligase is a DNA ligase. In some embodiments, the DNA ligase is T4 DNA ligase or a variant thereof.

In some embodiments, the DNA ligase comprises an amino acid sequence having at least 70% sequence identity to SEQ ID NO: 4, which is the amino acid sequence of Bacteriophage T4 DNA ligase (UniProt ID: P00970). In some embodiments, the RNA ligase comprises an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity to SEQ ID NO: 4.

In some embodiments, the ATP-dependent nucleic acid ligase is used in the form of whole cell, crude extract, isolated polypeptide, or purified polypeptide. In some embodiments, the ATP-dependent nucleic acid ligase polypeptide is used in an immobilized form as described herein, such as immobilized on a solid support material.

### Polyphosphate Kinase

“Polyphosphate kinases” or “PPKs” are a family of enzymes which catalyze the formation of ATP from AMP and polyphosphate.

In some embodiments, the PPK is PPK12. In some embodiments, the PPK comprises an amino acid sequence having at least 70% sequence identity to SEQ ID NO: 5, which is the amino acid sequence of PPK12. In some embodiments, the PPK comprises an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity to SEQ ID NO: 5.

In some embodiments, the PPK comprises an amino acid sequence having at least 70% sequence identity to SEQ ID NO: 6, which is the amino acid sequence of an optimized PPK12. In some embodiments, the PPK comprises an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity to SEQ ID NO: 6.

In some embodiments, the PPK is Acinetobacter johnsonii polyphosphate: AMP phosphotransferase (AjPAP) (UniProt ID: Q83XD3). In some embodiments, the PPK comprises an amino acid sequence having at least 70% sequence identity to SEQ ID NO: 7, which is the amino acid sequence of AjPAP. In some embodiments, the PPK comprises an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity to SEQ ID NO: 7.

In some embodiments, the PPK is used in the form of whole cell, crude extract, isolated polypeptide, or purified polypeptide. In some embodiments, the PPK polypeptide is used in an immobilized form as described herein, such as immobilized on a resin.

### Fusion Polypeptides

Methods described herein may be performed using an ATP-dependent nucleic acid ligase and a PPK, wherein the ATP-dependent nucleic acid ligase and the PPK are provided as separate polypeptides.

Methods described herein may also be performed using a fusion polypeptide comprising an ATP-dependent nucleic acid ligase linked to a PPK. Herein, the ATP-dependent nucleic acid ligase portion of the fusion polypeptide is denoted the ATP-dependent nucleic acid ligase ‘domain’, and the PPK portion of the fusion polypeptide is denoted the PPK ‘domain’.

An important consideration for the economic viability and sustainability of biocatalytic methods is the production costs associated with the enzymes used therein. To counteract potential increases in ligation reaction costs associated with inclusion of an ATP regeneration system, bifunctional fusion polypeptides were developed having a kinase domain and a ligase domain. The fusion polypeptides exhibit both kinase and ligase activity and can be produced via a single reaction (e.g. by recombinant expression) which saves time, effort and money as compared to production of separate kinase and ligase enzymes.

Beneficially, fusion polypeptides were unexpectedly found to exhibit higher ligase activity as compared to the unlinked enzymes. Without wishing to be bound by theory, the inventors believe that this improvement in ligase activity may be due to the production of a localized supply of ATP by the kinase and/or improved stability of the enzymes as compared to unlinked enzymes.

The disclosure also provides a fusion polypeptide comprising: (a) a PPK domain; and (b) an ATP-dependent nucleic acid ligase domain. Fusion polypeptides described herein may be used in an ATP-dependent RNA ligation reaction described herein.

In some embodiments, the ATP-dependent nucleic acid ligase domain is an RNA ligase domain. In some embodiments, the RNA ligase domain is a dsRNA ligase domain. In some embodiments, the dsRNA ligase domain is a member of RNA ligase 2 family. In some embodiments, the dsRNA ligase domain comprises Bacteriophage RB69 RNA ligase 2 (UniProt ID: Q7Y4V8). In some embodiments, the dsRNA ligase domain comprises an amino acid sequence having at least 70% sequence identity to SEQ ID NO: 1, which is the amino acid sequence of Bacteriophage RB69 RNA ligase 2 (UniProt ID: Q7Y4V8). In some embodiments, the dsRNA ligase domain comprises an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity to SEQ ID NO: 1.

In some embodiments, the dsRNA ligase domain comprises an amino acid sequence having at least 70% sequence identity to SEQ ID NO: 2, which is the amino acid sequence of an optimized Bacteriophage RB69 RNA ligase 2. In some embodiments, the dsRNA ligase domain comprises an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity to SEQ ID NO: 2.

In some embodiments, the dsRNA ligase domain comprises an amino acid sequence having at least 70% sequence identity to SEQ ID NO: 88, which is the amino acid sequence of an optimized Bacteriophage RB69 RNA ligase 2. In some embodiments, the dsRNA ligase domain comprises an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity to SEQ ID NO: 88.

In some embodiments, the ATP-dependent nucleic acid ligase domain comprises an amino acid sequence having at least 70% sequence identity to SEQ ID NO: 3, which is the amino acid sequence of Bacteriophage T4 RNA ligase 2 (UniProt ID: P32277). In some embodiments, the ATP-dependent nucleic acid ligase domain comprises an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity to SEQ ID NO: 3.

In some embodiments, the ATP-dependent nucleic acid ligase domain is a DNA ligase domain. In some embodiments, the DNA ligase domain is a T4 DNA ligase domain. In some embodiments, the T4 DNA ligase domain comprises an amino acid sequence having at least 70% sequence identity to SEQ ID NO: 4, which is the amino acid sequence of Bacteriophage T4 RNA ligase 2 (UniProt ID: P32277). In some embodiments, the ATP-dependent nucleic acid ligase domain comprises an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity to SEQ ID NO: 4.

In some embodiments, the PPK domain is PPK12. In some embodiments, the PPK domain comprises an amino acid sequence having at least 70% sequence identity to SEQ ID NO: 5, which is the amino acid sequence of PPK12. In some embodiments, the PPK domain comprises an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity to SEQ ID NO: 5.

In some embodiments, the PPK domain comprises an amino acid sequence having at least 70% sequence identity to SEQ ID NO: 6, which is the amino acid sequence of an optimized PPK12. In some embodiments, the PPK domain comprises an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity to SEQ ID NO: 6.

In some embodiments, the PPK domain is AjPAP (UniProt ID: Q83XD3). In some embodiments, the PPK domain comprises an amino acid sequence having at least 70% sequence identity to SEQ ID NO: 7, which is the amino acid sequence of AjPAP. In some embodiments, the PPK domain comprises an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity to SEQ ID NO: 7.

In some embodiments, the fusion polypeptide comprises an amino acid sequence having at least 70% sequence identity to a sequence selected from SEQ ID NOs: 8-18. In some embodiments, the fusion polypeptide comprises an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity to a sequence selected from SEQ ID NOs: 8-18.

In some embodiments, the fusion polypeptide comprises an amino acid sequence having at least 70% sequence identity to a sequence selected from SEQ ID NOs: 90, 92, 94, 96 or 98. In some embodiments, the fusion polypeptide comprises an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity to a sequence selected from SEQ ID NOs: 90, 92, 94, 96 or 98.

In some embodiments, the fusion polypeptide comprises an amino acid sequence having at least 70% sequence identity to a sequence selected from SEQ ID NOs: 8-18, 90, 92, 94, 96 or 98. In some embodiments, the fusion polypeptide comprises an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity to a sequence selected from SEQ ID NOs: 8-18, 90, 92, 94, 96 or 98.

In some embodiments, the fusion polypeptide comprises an amino acid sequence having at least 70% sequence identity to SEQ ID NO: 17. In some embodiments, the fusion polypeptide comprises an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity to SEQ ID NO: 17.

In some embodiments, the fusion polypeptide comprises an amino acid sequence having at least 70% sequence identity to SEQ ID NO: 18. In some embodiments, the fusion polypeptide comprises an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity to SEQ ID NO: 18.

In some embodiments, the fusion polypeptide comprises an amino acid sequence having at least 70% sequence identity to SEQ ID NO: 98. In some embodiments, the fusion polypeptide comprises an amino acid sequence having at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity to SEQ ID NO: 98.

In some embodiments, the method described herein is performed using a fusion polypeptide described herein.

In some embodiments, the composition described herein comprises a fusion polypeptide described herein. In some embodiments, the kit described herein comprises a fusion polypeptide described herein.

In some embodiments, the fusion polypeptide is used in the form of whole cell, crude extract, isolated fusion polypeptide, or purified fusion polypeptide. In some embodiments, the fusion polypeptide is used in an immobilized form as described herein, such as immobilized on a solid support.

In some embodiments, the rate of nucleic acid ligation exceeds the rate of nucleic acid ligation of a control; wherein the control comprises: (a) a first protein consisting of the PPK domain of the fusion polypeptide; and (b) a second protein consisting of the ATP-dependent nucleic acid ligase domain of the fusion polypeptide; wherein said first and second proteins are not linked. In some embodiments, the rate of nucleic acid ligation exceeds the rate of nucleic acid ligation of the control by at least 5%, at least 10%, at least 15%, at least 20%, at least 25%, at least 30%, at least 35%, at least 40%, at least 45% or at least 50%.

In some embodiments, the rate of RNA ligation exceeds the rate of RNA ligation of a control; wherein the control comprises: (a) a first protein consisting of the PPK domain of the fusion polypeptide; and (b) a second protein consisting of the dsRNA ligase domain of the fusion polypeptide; wherein said first and second proteins are not linked. In some embodiments, the rate of RNA ligation exceeds the rate of RNA ligation of the control by at least 5%, at least 10%, at least 15%, at least 20%, at least 25%, at least 30%, at least 35%, at least 40%, at least 45% or at least 50%.

In some embodiments, the rate of nucleic acid ligation is calculated as the percent conversion or using arbitrary units (AU) (calculated as described herein) over a defined incubation time (e.g. 12 hours, 18 hours or 24 hours). Comparisons between rates of RNA ligation are typically made between ligation reactions performed using similar or identical molar amounts of ATP-dependent nucleic acid ligase or ATP-dependent nucleic acid ligase domain.

### Linkers and Purification Tags

A purification tag is typically appended to a polypeptide to allow purification from crude biological source using affinity techniques. Purification tags include, but are not limited to, poly-histidine, chitin binding protein (CBP), maltose binding protein (MBP), Strep-tag and glutathione-S-transferase (GST). Poly-histidine tags bind to matrices bearing immobilized metal ions thereby allowing immobilization of the polypeptide attached thereto by metal affinity.

Linkers are typically short peptide sequences present between protein domains. Linkers may be configured to allow adjacent protein domains to move relative to one another or may be rigid to prevent unwanted interactions between the protein domains.

In some embodiments, the ATP-dependent nucleic acid ligase and PPK are linked via a linker, e.g. a peptide linker. In some embodiments, the PPK is at the N-terminus of the linker and the ATP-dependent nucleic acid ligase is at the C-terminus of the linker. In some embodiments, the ATP-dependent nucleic acid ligase is at the N-terminus of the linker and the PPK is at the C-terminus of the linker.

In some embodiments, the linker is a polypeptide linker. In some embodiments, the linker comprises at least 3 amino acids, at least 4 amino acids, at least 5 amino acids, at least 6 amino acids, at least 7 amino acids, at least 8 amino acids, at least 9 amino acids, or at least 10 amino acids. In some embodiments, the linker comprises at least 6 amino acids.

In some embodiments, the linker is linear. In some embodiments, the linker comprises at least 3 amino acids, wherein each amino acid is selected from glycine, serine, or alanine. In some embodiments, the linker comprises at least one amino acid selected from glutamic acid, aspartic acid, lysine or arginine.

In some embodiments, the linker comprises a poly-histidine tag. In some embodiments, the linker comprises the amino acid recognition sequence for the tobacco etch virus (TEV) protease.

In some embodiments, the linker comprises an amino acid sequence selected from: (a) HHHHHH (SEQ ID NO: 19), optionally HHHHHHHHHH (SEQ ID NO: 20); (b) ENLYFQS (SEQ ID NO: 21); (c) ENLYFQG (SEQ ID NO: 22); (d) SSGSSG (SEQ ID NO: 23); (e) GSAGSAAGSGEF (SEQ ID NO: 24); and/or (f) GSSGSGSSSGGSSSSGSS (SEQ ID NO: 25).

In some embodiments, the fusion polypeptide comprises a purification tag. In some embodiments, the linker comprises a purification tag. In some embodiments, the purification tag is located at the N- or C-terminus of the fusion polypeptide. In some embodiments, the purification tag comprises a poly-histidine tag. In some embodiments, the purification tag comprises MHHHHHHENLYFQS (SEQ ID NO: 26). In some embodiments, the purification tag comprises GQTGHHHHHH (SEQ ID NO: 27). In some embodiments, the purification tag comprises a Myc-tag (EQKLISEEDL (SEQ ID NO: 28)). In some embodiments, the purification tag comprises a FLAG-tag (DYKDDDDK (SEQ ID NO: 29)).

### Immobilization

In some embodiments, the PPK, ATP-dependent nucleic acid ligase and/or fusion polypeptide are immobilized. In some embodiments, the PPK, ATP-dependent nucleic acid ligase and/or fusion polypeptide are immobilized using affinity immobilization. In some embodiments, the PPK, ATP-dependent nucleic acid ligase and/or the fusion polypeptide are immobilized using metal affinity immobilization, e.g. by contacting His-tagged PPK, ATP-dependent nucleic acid ligase and/or fusion polypeptide with immobilized metal such as nickel, zinc, cobalt, or copper.

In some embodiments, the PPK, ATP-dependent nucleic acid ligase and/or fusion polypeptide is immobilised on a solid material by chemical bond or a physical adsorption method. The terms “solid support”, “solid material”, and “solid support material” are used interchangeably herein. Immobilization of a polypeptide by physical absorption typically involves the polypeptide being physically adsorbed or attached onto a solid support material. Adsorption can occur through weak non-specific forces such as van der Waals, hydrophobic interactions and hydrogen bonds. Physical adsorption may be achieved by soaking the solid support material in a solution of the polypeptide and incubating to allow time for physical adsorption to occur. Immobilization of a polypeptide by chemical bonding typically involves the attachment of the polypeptide to the solid support material via a covalent bond.

Wherein the ATP-dependent nucleic acid ligase is immobilized, the inventors believe that (owing to the size of the oligonucleotide fragment substrate), the catalytic activity of the ATP-dependent ligase is optimal when a spacer is present between the ATP-dependent ligase and the immobilization moiety. Wherein the fusion polypeptide is immobilized, the inventors believe that, the catalytic activity of the ATP-dependent ligase domain is optimal when the PPK domain is linked to the immobilization moiety (optionally via a spacer). In some embodiments, the PPK is immobilized (optionally via a spacer), and the ATP-dependent nucleic acid ligase is not immobilized. In some embodiments, the spacer is a polypeptide (e.g. a polypeptide comprising 2 or more, 3 or more, 4 or more, 5 or more, 10 or more, 15 or more, 20 or more, 25 or more, 30 or more, 35 or more, 40 or more, 45 or more, 50 or more, 75 or more, or 100 or more amino acids).

In some embodiments, the PPK, ATP-dependent nucleic acid ligase and/or the fusion polypeptide are immobilized to a solid support, such as a membrane, resin, solid carrier, or other solid phase material. A solid support can be composed of organic polymers such as polystyrene, polyethylene, polypropylene, polyfluoroethylene, polyethyleneoxy, polymethacrylate, and polyacrylamide, as well as co-polymers and grafts thereof. A solid support can also be inorganic, such as glass, silica, controlled pore glass (CPG), reverse phase silica or metal, such as gold or platinum. The configuration of a solid support can be in the form of beads, spheres, particles, granules, a gel, a membrane or a surface. Surfaces can be planar, substantially planar, or non-planar. Solid supports can be porous or non-porous and can have swelling or non-swelling characteristics. A solid support can be configured in the form of a well, depression, or other container, vessel, feature, or location. Solid supports useful for immobilizing the PPK, ATP-dependent nucleic acid ligase and/or the fusion polypeptide for carrying out the reaction include but are not limited to beads or resins such as polymethacrylate, e.g., polymethacrylates with epoxy functional groups, polymethacrylates with amino epoxy functional groups, polymethacrylates, styrene/DVB copolymer or polymethacrylates with octadecyl functional groups.

Exemplary solid supports include, but are not limited to, chitosan beads, Eupergit C, IB-150, IB-350, IB—C435, IB-A369, IB-A161, IB-A171, IB—S500, IB—S861, SEPABEADS (Mitsubishi), e.g., Sepabeads EC-EP, Sepabeads EC-HFA, Sepabeads EC-HG, Sepabeads EC-BU, Sepabeads EC-OD, Sepabeads EC-CM, Sepabeads EC-IDA, Sepabeads EC-EA, Sepabeads EC-HA, Sepabeads EC-QA, Sepabeads EXE, Sepabeads EXA, Dilbeads-TA, Amberzyme Oxirane, Amberlite XAD-7HP, Amberlite FPA98C1, Amberlite IRA958Cl, Amberlite IRA67, Amberlite FPA90Cl, Amberlite FPA40Cl, Amberlite XAD18, Accurel EP100, ECR8206F/5730, ECR8206/5803, ECR8206M/5749, ReliZyme EP403, ReliZymeEP113, Lewatit VP OC 1600, Diaion WA20, Diaion WA21J, Diaion WA30, Dowex 66, Diaion HPA-25L, Lewatit VP OC 1064 MD PH, Lewatit VP OC 1163, Lifetech ECR8304F. Lifetech ECR8309F, Lifetech ECR8315F, Lifetech ECR8204F, Lifetech ECR8285, Lifetech ECR1090M, Lifetech ECR1030M, Lifetech ECR8806M, Chromalite (MAM2/F) D6591, Chromalite MIDA/M, Chromalite MIDA/M/Fe, Chromalite MIDA/M/Co, Chromalite MIDA/M/Ni, Chromalite MIDA/M/Cu and Chromalite MIDA/M/Zn.

### Cofactors

The enzymatic activity of ATP-dependent nucleic acid ligases requires ATP as a cofactor. One molecule of ATP is converted to AMP per ligation reaction. The catalytic mechanism of ATP-dependent nucleic acid ligase and the role of ATP in nucleic acid ligation reactions are described above.

In some embodiments, the method is performed using the cofactor, ATP. In some embodiments, the method is performed using a sub-stoichiometric concentration of the cofactor, ATP

In some embodiments, the method is performed using the cofactor, AMP. In some embodiments, the method is performed using a sub-stoichiometric concentration of the cofactor, AMP.

In some embodiments, the method is performed using a mixture of the cofactors, ATP and AMP. In some embodiments, the method is performed using a sub-stoichiometric concentration of the cofactors, ATP and AMP.

The skilled person can readily determine the stoichiometric concentration of cofactor required for a given ligation based on the concentration of the oligonucleotide fragments and the number of ligation reactions required to produce the oligonucleotide product. For example, for a ligation reaction using 1 mM oligonucleotide fragments which requires four ligation reactions to produce the oligonucleotide product, the stoichiometric concentration of ATP is 4 mM.

In some embodiments, the method is performed using an ATP and/or AMP concentration of about 0.5 mM, about 1 mM, about 2 mM, about 3 mM, about 4 mM, about 5 mM, about 6 mM, about 7 mM, about 8 mM, about 9 mM, about 10 mM, about 12 mM, about 14 mM, about 16 mM, about 18 mM, or about 20 mM.

### Polyphosphate

Polyphosphate is used as a phosphate donor by PPKs in the catalytic formation of ATP from AMP.

In some embodiments, the polyphosphate is a polyphosphate salt. In some embodiments, the polyphosphate salt is sodium polyphosphate (Maddrell's salt) or sodium hexametaphosphate (Graham's salt). In some embodiments, the method is performed using a stoichiometric excess of polyphosphate. In some embodiments, the method is performed using a polyphosphate concentration of at least 5 mM, at least 10 mM, at least 15 mM, at least 20 mM, at least 25 mM, at least 30 mM, at least 35 mM, at least 40 mM, at least 45 mM, at least 50 mM, 55 mM, at least 60 mM, at least 65 mM, at least 70 mM, at least 75 mM, at least 80 mM, at least 85 mM, at least 90 mM, at least 95 mM, or at least 100 mM.

### Divalent Cation

The enzymatic activity of ATP-dependent nucleic acid ligases and PPKs requires the presence of a divalent cation.

In some embodiments, the divalent cation comprises Mg²⁺ and/or Mn²⁺. In some embodiments, the method is performed with a divalent cation concentration of 5-100 mM, 10-100 mM, 15-100 mM, 20-100 mM, 30-100 mM, 5-90 mM, 5-80 mM, 5-70 mM, 5-60 mM, 5-50 mM, or 30-50 mM. In some embodiments, the method is performed with a divalent cation concentration of at least 5 mM, at least 10 mM, at least 15 mM, at least 20 mM, at least 25 mM, at least 30 mM, at least 35 mM, at least 40 mM, at least 45 mM, at least 50 mM, 55 mM, at least 60 mM, at least 65 mM, at least 70 mM, at least 75 mM, at least 80 mM, at least 85 mM, at least 90 mM, at least 95 mM, or at least 100 mM.

The divalent cation concentration will typically depend on the amount of ATP required to achieve complete ligation (which in turn depends on the starting concentration of ATP/AMP and the concentration and number of different oligonucleotide fragments).

### Nucleic Acids

The disclosure further provides a nucleic acid molecule encoding a fusion polypeptide described herein. The nucleic acid molecule encoding a fusion polypeptide described herein can be linked to one or more heterologous regulatory sequences that control gene expression to produce recombinant polynucleotides that are capable of expressing the fusion polypeptides. Expression constructs comprising a heterologous polynucleotide encoding a fusion polypeptide may be introduced into a suitable host cell to express the corresponding fusion polypeptide.

As is apparent to one skilled in the art, the availability of protein sequences and knowledge of codons corresponding to a variety of amino acids provide an illustration of all possible nucleic acid molecules that encode the protein sequence of interest. The degeneracy of the genetic code, in which the same amino acid is encoded by selectable or synonymous codons, allows for the production of an extremely large number of nucleic acid molecules, all of which encode the fusion polypeptides disclosed herein. Thus, upon determination of a particular amino acid sequence, one skilled in the art can generate any number of different nucleic acid molecules by modifying one or more codons in a manner that does not alter the amino acid sequence of the protein. In this regard, this disclosure specifically contemplates each and every possible alteration of a nucleic acid molecules that can be made by selecting a combination based on possible codon selections, for any of the polypeptides disclosed herein, comprising those amino acid sequences of exemplary fusion polypeptides listed in Example 1, and any of the fusion polypeptides of SEQ ID NOs: 8-18, 90, 92, 94, 96 and 98 in the Sequence Listing incorporated by reference.

In various embodiments, the codons are preferably selected to accommodate the host cell in which the recombinant protein is produced. For example, codons preferred for bacteria are used to express genes in bacteria; codons preferred for yeast are used to express genes in yeast; and codons preferred for mammals are used for gene expression in mammalian cells.

In some embodiments, the nucleic acid molecule encodes a fusion polypeptide comprising an amino acid sequence having at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity to SEQ ID NO: 1, which is the amino acid sequence of Bacteriophage RB69 RNA ligase 2 (UniProt ID: Q7Y4V8).

In some embodiments, the nucleic acid molecule comprises a nucleic acid sequence having at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity to SEQ ID NO: 30, which is a nucleic acid sequence encoding the amino acid sequence of SEQ ID NO: 1.

In some embodiments, the nucleic acid molecule encodes a fusion polypeptide comprising an amino acid sequence having at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity to SEQ ID NO: 2, which is the amino acid sequence of an optimized Bacteriophage RB69 RNA ligase 2.

In some embodiments, the nucleic acid molecule comprises a nucleic acid sequence having at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity to SEQ ID NO: 31, which is a nucleic acid sequence encoding the amino acid sequence of SEQ ID NO: 2.

In some embodiments, the nucleic acid molecule encodes a fusion polypeptide comprising an amino acid sequence having at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity to SEQ ID NO: 88, which is the amino acid sequence of an optimized Bacteriophage RB69 RNA ligase 2.

In some embodiments, the nucleic acid molecule comprises a nucleic acid sequence having at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity to SEQ ID NO: 87, which is a nucleic acid sequence encoding the amino acid sequence of SEQ ID NO: 88.

In some embodiments, the nucleic acid molecule encodes a fusion polypeptide comprising an amino acid sequence having at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity to SEQ ID NO: 3, which is the amino acid sequence of Bacteriophage T4 RNA ligase 2 (UniProt ID: P32277).

In some embodiments, the nucleic acid molecule comprises a nucleic acid sequence having at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity to SEQ ID NO: 32, which is a nucleic acid sequence encoding the amino acid sequence of SEQ ID NO: 3.

In some embodiments, the nucleic acid molecule encodes a fusion polypeptide comprising an amino acid sequence having at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity to SEQ ID NO: 4, which is the amino acid sequence of Bacteriophage T4 DNA ligase (UniProt ID: P00970).

In some embodiments, the nucleic acid molecule comprises a nucleic acid sequence having at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity to SEQ ID NO: 33, which is a nucleic acid sequence encoding the amino acid sequence of SEQ ID NO: 4.

In some embodiments, the nucleic acid molecule encodes a fusion polypeptide comprising an amino acid sequence having at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity to SEQ ID NO: 5, which is the amino acid sequence of PPK12.

In some embodiments, the nucleic acid molecule comprises a nucleic acid sequence having at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity to SEQ ID NO: 34, which is a nucleic acid sequence encoding the amino acid sequence of SEQ ID NO: 5.

In some embodiments, the nucleic acid molecule encodes a fusion polypeptide comprising an amino acid sequence having at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity to SEQ ID NO: 6, which is the amino acid sequence of an optimized PPK12.

In some embodiments, the nucleic acid molecule comprises a nucleic acid sequence having at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity to SEQ ID NO: 35, which is a nucleic acid sequence encoding the amino acid sequence of SEQ ID NO: 6.

In some embodiments, the nucleic acid molecule encodes a fusion polypeptide comprising an amino acid sequence having at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity to SEQ ID NO: 7, which is the amino acid sequence of AjPAP (UniProt ID: Q83XD3).

In some embodiments, the nucleic acid molecule comprises a nucleic acid sequence having at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity to SEQ ID NO: 36, which is a nucleic acid sequence encoding the amino acid sequence of SEQ ID NO: 7.

In some embodiments, the nucleic acid molecule encodes a fusion polypeptide comprising an amino acid sequence having at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity to any one of SEQ ID NOs: 8-18.

In some embodiments, the nucleic acid molecule comprises a nucleic acid sequence having at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity to any one of SEQ ID NOs: 37-47.

In some embodiments, the nucleic acid molecule encodes a fusion polypeptide comprising an amino acid sequence having at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity to any one of SEQ ID NOs: 90, 92, 94, 96 or 98.

In some embodiments, the nucleic acid molecule comprises a nucleic acid sequence having at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity to any one of SEQ ID NOs: 89, 91, 93, 95 or 97.

In some embodiments, the nucleic acid molecule encodes a fusion polypeptide comprising an amino acid sequence having at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity to any one of SEQ ID NOs: 8-18, 90, 92, 94, 96 or 98.

In some embodiments, the nucleic acid molecule comprises a nucleic acid sequence having at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity to any one of SEQ ID NOs: 37-47, 89, 91, 93, 95 or 97.

In some embodiments, the nucleic acid molecule encodes a fusion polypeptide comprising an amino acid sequence having at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity to any one of SEQ ID NO: 17.

In some embodiments, the nucleic acid molecule comprises a nucleic acid sequence having at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity to any one of SEQ ID NO: 46.

In some embodiments, the nucleic acid molecule encodes a fusion polypeptide comprising an amino acid sequence having at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity to any one of SEQ ID NO: 18.

In some embodiments, the nucleic acid molecule comprises a nucleic acid sequence having at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity to any one of SEQ ID NO: 47.

In some embodiments, the nucleic acid molecule encodes a fusion polypeptide comprising an amino acid sequence having at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity to any one of SEQ ID NO: 98.

In some embodiments, the nucleic acid molecule comprises a nucleic acid having at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% sequence identity to any one of SEQ ID NO: 97.

The nucleic acid molecule(s) encoding fusion polypeptide(s) can be manipulated to enable expression of the fusion polypeptide(s) in a variety of ways, such as by codon optimization to improve expression in the host cell, insertion into suitable expression elements with or without additional control sequences, and transformation into a host cell suitable for expression and production of the fusion polypeptides.

Depending on the expression vector, manipulation of the nucleic acid molecule prior to insertion into the vector may be desirable or necessary. Techniques for modifying nucleic acid sequences using recombinant DNA methods are well known in the art. Guidance is provided in e.g. Sambrook et al., 2001, Molecular Cloning: A Laboratory Manual, Third Edition, Cold Spring Harbor Laboratory Press; and Current Protocols in Molecular Biology, Ausubel. F. Eds., Greene Pub. Associates, 1998, 2010 Year update.

When the sequence of a polypeptide is known, the encoding polynucleotide may be prepared by standard solid-phase methods according to known synthetic methods. In some embodiments, fragments of up to about 100 bases can be synthesized separately and then ligated (e.g., by enzymatic or chemical ligation methods or polymerase-mediated methods) to form any desired contiguous sequence. For example, the nucleic acid molecules of the present disclosure can be prepared by chemical synthesis using, for example, the classic phosphoramidite methods described by Beaucage et al., 1981, Tet Lett 22:1859-69, or Matthes et al. People, 1984, EMBO J. 3:801-05, as typically practiced in automated synthesis methods. According to the phosphoramidite method, nucleic acid molecules are synthesized, purified, annealed, ligated, and cloned into a suitable vector, for example, in an automated DNA synthesizer. In addition, essentially any nucleic acid is available from any of a variety of commercial sources.

### Vectors

The disclosure provides a recombinant expression vector comprising the nucleic acid molecule encoding a PPK, ATP-dependent nucleic acid ligase and/or fusion polypeptide described herein. In some embodiments, the vector is selected from a plasmid, a cosmid, a bacteriophage, or a viral vector. Recombinant expression vectors typically comprise one or more expression regulatory regions, such as promoters and terminators, origin of replication and the like.

Nucleic acid molecules encoding a PPK, ATP-dependent nucleic acid ligase and/or fusion polypeptide described herein can be expressed by inserting the nucleic acid sequence or the nucleic acid construct comprising the sequence into an appropriate expression vector. In generating the expression vector, the coding sequence is located in the vector such that the coding sequence is linked to a suitable control sequence for expression. The recombinant expression vector can be any vector (e.g., a plasmid or virus) that can be conveniently used in recombinant DNA procedures and can result in the expression of a polynucleotide sequence. The choice of vector will generally depend on the compatibility of the vector with the host cell to be introduced into. The vector can be linear or closed circular plasmid. The expression vector may be an autonomously replicating vector, i.e., a vector that exists as an extrachromosomal entity whose replication is independent of chromosomal replication, such as a plasmid, extrachromosomal element, minichromosome, or artificial chromosome. The vector may contain any tools for ensuring self-copying. Alternatively, the vector may be a vector that, when introduced into a host cell, integrates into the genome and replicates with the chromosome into which it is integrated. Moreover, a single vector or plasmid or two or more vectors or plasmids that together comprise the total DNA to be introduced into the genome of the host cell may be used.

Many expression vectors useful to the embodiments of the present disclosure are commercially available. An exemplary expression vector can be prepared by inserting a polynucleotide encoding a fusion polypeptide to plasmid pACYC-Duet-1 (Novagen), pBR322 Vector (New England Biolabs), pUC19 Vector (New England Biolabs) or pET T7 Expression Vectors (Novagen).

### Host Cells

The disclosure also provides a host cell capable of expressing a fusion polypeptide described herein. In some embodiments, the host cell comprises the nucleic acid molecule described herein or the vector described herein. In some embodiments, the host cell is Escherichia coli

In some embodiments, the nucleic acid molecule encoding the polypeptide is linked to one or more control sequences for expression of polypeptides in the host cell. Host cells for expression of polypeptides encoded by the expression vectors of the present disclosure are well known in the art, including, but not limited to, bacterial cells such as E. coli, Streptomyces, and Salmonella typhimurium; fungal cells (e.g., Saccharomyces cerevisiae or Pichia pastoris); insect cells such as Drosophila S2 and Spodoptera Sf9; animal cells such as CHO, COS, BHK, 293 and Bowes melanoma cells; and plant cells. An exemplary host cell is E. coli BL21 (DE3). The above host cells may be wild-type or may be engineered cells through genomic edition. Suitable media and growth conditions for the above host cells are well known in the art.

Nucleic acid molecules or vectors used to express polypeptides can be introduced into cells by a variety of methods known in the art. Techniques comprise, among others, electroporation, bio-particle bombardment, liposome-mediated transfection, calcium chloride transfection, and protoplast fusion. Different methods of introducing polynucleotides into cells are known to those skilled in the art.

The host cell may be used to express and isolate the polypeptide described herein.

In some embodiments, the present disclosure also provides a process for producing a fusion polypeptide described herein, wherein the process comprises culturing a host cell capable of expressing a polynucleotide encoding the fusion polypeptide under culture conditions suitable for the expression of the fusion polypeptide. In some embodiments, the process of preparing a fusion polypeptide further comprises isolating the fusion polypeptide. Fusion polypeptides may be expressed in suitable cells and isolated (or recovered) from the host cell and/or culture medium using any one or more of the well-known techniques for protein purification, the techniques for protein purification include, among others, lysozyme treatment, sonication, filtration, salting out, ultracentrifugation and chromatography.

### Reaction Conditions

As disclosed herein and exemplified in the examples, the present disclosure contemplates a range of suitable reaction conditions that may be used in the methods described herein, including but not limited to pH, temperature, buffers, substrate loadings, enzyme loading, cofactor loading, pressure, and reaction time. Additional suitable reaction conditions for ligation reactions described herein can be readily optimized by routine experimentation, e.g. performing the method described herein under experimental reaction conditions of varying reagent concentration, pH, temperature, and detecting the rate of oligonucleotide product formation.

In any of the embodiments of the process disclosed herein, the reaction conditions may include a suitable pH. As noted above, the desired pH or desired pH range can be maintained by using an acid or base, a suitable buffer, or a combination of buffer and added acid or base. The pH of the reaction mixture can be controlled before and/or during the reaction. In some embodiments, suitable reaction conditions include a solution pH of about 4 to about 8, a pH of about 5 to about 7, a pH of about 6 to about 8, or a pH of about 7 to about 8. In some embodiments, the reaction conditions include a solution pH of about 4, 4.5, 5, 5.5, 6, 6.5, 7, 7.5 or 8.

In any of the embodiments of the process disclosed herein, suitable temperatures can be used for the reaction conditions, taking into consideration of, for example, the increase in reaction rate at higher temperatures, the activity of the enzyme for sufficient duration of the reaction. Accordingly, in some embodiments, suitable reaction conditions include a temperature of about 10° C. to about 60° C., about 10° C. to about 50° C., about 25° C. to about 50° C., about 25° C. to about 40° C., about 25° C. to about 30° C., or about 10° C. to about 30° C. In some embodiments, suitable reaction temperatures include a temperature of about 10° C., 15° C., 20° C., 25° C., 30° C., 35° C., 40° C., 45° C., 50° C., 55° C., or 60° C. In some embodiments, the temperature during the enzymatic reaction can be maintained at a certain temperature throughout the reaction. In some embodiments, the temperature during the enzymatic reaction may be adjusted over a temperature profile during the course of the reaction.

The reaction may be performed in any suitable buffer solution. In some embodiments, the buffer solution is selected from Tris buffer (e.g. Tris-HCl), phosphate buffer, HEPES, MOPS (3-(N-morpholino) propanesulfonic acid), and triethanolamine (TEOA) buffer. In some embodiments, the buffer solution comprises acetate, citrate, prolamine, carbonate, or phosphate, or any combination thereof. In some embodiments, the buffer solution is phosphate buffered saline (PBS), e.g. PBS having an NaCl concentration of <100 mM.

In some embodiments, the reaction mixture further comprises a reducing agent, optionally DTT (Dithiothreitol).

Suitable solvents include water, aqueous buffer solutions, organic solvents, and/or co-solvent systems, which generally include aqueous solvents and organic solvents. The aqueous solutions (water or aqueous co-solvent systems) can be pH-buffered or unbuffered.

Suitable reaction conditions can include a combination of reaction parameters that provide for the biocatalytic conversion of the oligonucleotide fragments to the corresponding oligonucleotide product.

In carrying out the ligation reactions described herein, the PPK, ATP-dependent nucleic acid ligase and/or the fusion polypeptide may be added to the reaction mixture in different formulation forms, as frozen or lyophilized whole cells (FWC or LWC) transformed with the gene encoding the PPK, ATP-dependent nucleic acid ligase and/or the fusion polypeptide and/or as cell lysate or lyophilized cell lysate of such cells, so called shake flask powder (SFP), where the cell debris was removed and/or further purified as fermentation powder (FP). Whole cells transformed with gene(s) encoding the PPK, ATP-dependent nucleic acid ligase and/or the fusion polypeptide or cell extracts, lysates thereof, and isolated enzymes can be used in a wide variety of different forms, including solids (e.g., lyophilized, spray dried, or the like) or semisolid (e.g., a crude paste). The cell extract or cell lysate may be partially purified by precipitation (e.g., ammonium sulfate, polyethyleneimine, heat treatment or the like), followed by desalting procedures (e.g., ultrafiltration, dialysis, and the like) prior to lyophilization. Any of the enzyme preparations can be immobilized to a solid phase material (such as a resin).

In any of the embodiments of the process disclosed herein, wherein an engineered polypeptide is expressed in the form of a secreted polypeptide, a culture medium containing the secreted polypeptide can be used in the process herein.

In any of the embodiments of the process disclosed herein, the solid reactants (e.g., enzymes, salts, etc.) can be provided to the reaction in a variety of different forms, including powders (e.g., lyophilized, spray dried, etc.), solutions, emulsions, suspensions, and the like. The reactants can be readily lyophilized or spray-dried using methods and instrumentation known to one skilled in the art. For example, the protein solution can be frozen at −80° C. in small aliquots, and then added to the pre-chilled lyophilization chamber, followed by the application of a vacuum.

In any of the embodiments of the process disclosed herein, the order of addition of reactants is not critical. The reactants may be added together to the solvent at the same time or alternatively, some reactants may be added separately, and some may be added together at different time points.

The methods of performing a ligation reaction may comprise the further step of isolating the oligonucleotide product of the enzymatic reaction. In particular, this step is typically performed after completion of the enzymatic reaction. The product is in particular typically separated from one or more, in particular essentially all of the other components of the reaction mixture. For example, the product is typically separated from the remaining substrate, side products, and/or enzymes. Isolation of the product may be achieved by means and techniques known in the art, e.g. by separating oligonucleotides based on their size such as by gel electrophoresis, gel extractions, using cellulose-based matrices, ultrafiltration, and/or chromatography.

### Stoichiometry

Ligation methods may be performed under any suitable reaction conditions. Such conditions can be readily identified by the person skilled in the art.

The skilled person will readily understand that the concentration of reagents will vary depending on the concentration of oligonucleotide fragments used. For example, as the concentration of oligonucleotide fragments increases, a higher number of ATP molecules is required to achieve complete ligation and therefore higher concentrations of polyphosphate and divalent cation are required to enable ATP regeneration from AMP.

The methods described herein are typically performed using a concentration of AMP, a concentration of ATP, or a combined concentration of AMP and ATP that is less than the stoichiometric concentration. In some embodiments, the method is performed using an ATP/AMP concentration that is lower than the concentration of oligonucleotide fragments.

In some embodiments, wherein one ligation reaction is required to generate the oligonucleotide product, the method is performed using about 1 mM oligonucleotide fragments and less than about 1 mM ATP and/or AMP, optionally ≤0.5 mM ATP and/or AMP.

In some embodiments, wherein one ligation reaction is required to generate the oligonucleotide product, the method is performed using about 2 mM oligonucleotide fragments and less than about 2 mM ATP and/or AMP, optionally ≤1.5 mM, ≤1 mM or ≤0.5 mM ATP and/or AMP.

In some embodiments, wherein one ligation reaction is required to generate the oligonucleotide product, the method is performed using about 3 mM oligonucleotide fragments and less than about 3 mM ATP and/or AMP, optionally ≤2.5 mM, ≤2 mM, ≤1.5 mM, ≤1 mM or ≤0.5 mM ATP and/or AMP.

In some embodiments, wherein one ligation reaction is required to generate the oligonucleotide product, the method is performed using about 4 mM oligonucleotide fragments and less than about 4 mM ATP and/or AMP, optionally ≤3.5 mM, ≤3 mM, ≤2.5 mM, ≤2 mM, ≤1.5 mM, ≤1 mM or ≤0.5 mM ATP and/or AMP.

In some embodiments, wherein one ligation reaction is required to generate the oligonucleotide product, the method is performed using about 5 mM oligonucleotide fragments and less than about 5 mM ATP and/or AMP, optionally ≤4.5 mM, ≤4 mM, ≤3.5 mM, ≤3 mM, ≤2.5 mM, ≤2 mM, ≤1.5 mM, ≤1 mM or ≤0.5 mM ATP and/or AMP.

In some embodiments, wherein one ligation reaction is required to generate the oligonucleotide product, the method is performed using about 6 mM oligonucleotide fragments and less than about 6 mM ATP and/or AMP, optionally ≤5.5 mM, ≤5 mM, ≤4.5 mM, ≤4 mM, ≤3.5 mM, ≤3 mM, ≤2.5 mM, ≤2 mM, ≤1.5 mM, ≤1 mM or ≤0.5 mM ATP and/or AMP.

In some embodiments, wherein two ligation reactions are required to generate the oligonucleotide product, the method is performed using about 1 mM oligonucleotide fragments and less than about 2 mM ATP and/or AMP, optionally ≤1.5 mM, ≤1 mM or ≤0.5 mM ATP and/or AMP.

In some embodiments, wherein two ligation reactions are required to generate the oligonucleotide product, the method is performed using about 2 mM oligonucleotide fragments and less than about 4 mM ATP and/or AMP, optionally ≤3.5 mM, ≤3 mM, ≤2.5 mM, ≤2 mM, ≤1.5 mM, ≤1 mM or ≤0.5 mM ATP and/or AMP.

In some embodiments, wherein two ligation reactions are required to generate the oligonucleotide product, the method is performed using about 3 mM oligonucleotide fragments and less than about 6 mM ATP and/or AMP, optionally ≤5.5 mM, ≤5 mM, ≤4.5 mM, ≤4 mM, ≤3.5 mM, ≤3 mM, ≤2.5 mM, ≤2 mM, ≤1.5 mM, ≤1 mM or ≤0.5 mM ATP and/or AMP.

In some embodiments, wherein two ligation reactions are required to generate the oligonucleotide product, the method is performed using about 4 mM oligonucleotide fragments and less than about 8 mM ATP and/or AMP, optionally ≤7.5 mM, ≤7 mM, ≤6.5 mM, ≤6 mM, ≤5.5 mM, ≤5 mM, ≤4.5 mM, ≤4 mM, ≤3.5 mM, ≤3 mM, ≤2.5 mM, ≤2 mM, ≤1.5 mM, ≤1 mM or ≤0.5 mM ATP and/or AMP.

In some embodiments, wherein two ligation reactions are required to generate the oligonucleotide product, the method is performed using about 5 mM oligonucleotide fragments and less than about 10 mM ATP and/or AMP, optionally ≤9.5 mM, ≤9 mM, ≤8.5 mM, ≤8 mM, ≤7.5 mM, ≤7 mM, ≤6.5 mM, ≤6 mM, ≤5.5 mM, ≤5 mM, ≤4.5 mM, ≤4 mM, ≤3.5 mM, ≤3 mM, ≤2.5 mM, ≤2 mM, ≤1.5 mM, ≤1 mM or ≤0.5 mM ATP and/or AMP.

In some embodiments, wherein two ligation reactions are required to generate the oligonucleotide product, the method is performed using about 6 mM oligonucleotide fragments and less than about 12 mM ATP and/or AMP, optionally ≤11.5 mM, ≤11 mM, ≤10.5 mM, ≤10 mM, ≤9.5 mM, ≤9 mM, ≤8.5 mM, ≤8 mM, ≤7.5 mM, ≤7 mM, ≤6.5 mM, ≤6 mM, ≤5.5 mM, ≤5 mM, ≤4.5 mM, ≤4 mM, ≤3.5 mM, ≤3 mM, ≤2.5 mM, ≤2 mM, ≤1.5 mM, ≤1 mM or ≤0.5 mM ATP and/or AMP.

In some embodiments, wherein three ligation reactions are required to generate the oligonucleotide product, the method is performed using about 1 mM oligonucleotide fragments and less than about 3 mM ATP and/or AMP, optionally ≤2.5 mM, ≤2 mM, ≤1.5 mM, ≤1 mM or ≤0.5 mM ATP and/or AMP.

In some embodiments, wherein three ligation reactions are required to generate the oligonucleotide product, the method is performed using about 2 mM oligonucleotide fragments and less than about 6 mM ATP and/or AMP, optionally ≤5.5 mM, ≤5 mM, ≤4.5 mM, ≤4 mM, ≤3.5 mM, ≤3 mM, ≤2.5 mM, ≤2 mM, ≤1.5 mM, ≤1 mM or ≤0.5 mM ATP and/or AMP.

In some embodiments, wherein three ligation reactions are required to generate the oligonucleotide product, the method is performed using about 3 mM oligonucleotide fragments and less than about 9 mM ATP and/or AMP, optionally ≤8.5 mM, ≤8 mM, ≤7.5 mM, ≤7 mM, ≤6.5 mM, ≤6 mM, ≤5.5 mM, ≤5 mM, ≤4.5 mM, ≤4 mM, ≤3.5 mM, ≤3 mM, ≤2.5 mM, ≤2 mM, ≤1.5 mM, ≤1 mM or ≤0.5 mM ATP and/or AMP.

In some embodiments, wherein three ligation reactions are required to generate the oligonucleotide product, the method is performed using about 4 mM oligonucleotide fragments and less than about 12 mM ATP and/or AMP, optionally ≤11.5 mM, ≤11 mM, ≤10.5 mM, ≤10 mM, ≤9.5 mM, ≤9 mM, ≤8.5 mM, ≤8 mM, ≤7.5 mM, ≤7 mM, ≤6.5 mM, ≤6 mM, ≤5.5 mM, ≤5 mM, ≤4.5 mM, ≤4 mM, ≤3.5 mM, ≤3 mM, ≤2.5 mM, ≤2 mM, ≤1.5 mM, ≤1 mM or ≤0.5 mM ATP and/or AMP.

In some embodiments, wherein three ligation reactions are required to generate the oligonucleotide product, the method is performed using about 5 mM oligonucleotide fragments and less than about 15 mM ATP and/or AMP, optionally ≤14.5 mM, ≤14 mM, ≤13.5 mM, ≤13 mM, ≤12.5 mM, ≤12 mM, ≤11.5 mM, ≤11 mM, ≤10.5 mM, ≤10 mM, ≤9.5 mM, ≤9 mM, ≤8.5 mM, ≤8 mM, ≤7.5 mM, ≤7 mM, ≤6.5 mM, ≤6 mM, ≤5.5 mM, ≤5 mM, ≤4.5 mM, ≤4 mM, ≤3.5 mM, ≤3 mM, ≤2.5 mM, ≤2 mM, ≤1.5 mM, ≤1 mM or ≤0.5 mM ATP and/or AMP.

In some embodiments, wherein three ligation reactions are required to generate the oligonucleotide product, the method is performed using about 6 mM oligonucleotide fragments and less than about 18 mM ATP and/or AMP, optionally ≤17.5 mM, ≤17 mM, ≤16.5 mM, ≤16 mM, ≤15.5 mM, ≤15 mM, ≤14.5 mM, ≤14 mM, ≤13.5 mM, ≤13 mM, ≤12.5 mM, ≤12 mM, ≤11.5 mM, ≤11 mM, ≤10.5 mM, ≤10 mM, ≤9.5 mM, ≤9 mM, ≤8.5 mM, ≤8 mM, ≤7.5 mM, ≤7 mM, ≤6.5 mM, ≤6 mM, ≤5.5 mM, ≤5 mM, ≤4.5 mM, ≤4 mM, ≤3.5 mM, ≤3 mM, ≤2.5 mM, ≤2 mM, ≤1.5 mM, ≤1 mM or ≤0.5 mM ATP and/or AMP.

In some embodiments, the method is performed using about 1 g/L PPK, optionally 1.1 g/L, 1.15 g/L, 1.2 g/L, 1.25 g/L, 1.3 g/L, 1.35 g/L, 1.4 g/L, 1.45 g/L, 1.5 g/L, 1.55 g/L, 1.6 g/L, 1.65 g/L, 1.7 g/L, 1.75 g/L, 1.8 g/L, 1.85 g/L, 1.9 g/L, 1.95 g/L, 2 g/L, 2.1 g/L, 2.2 g/L, 2.3 g/L, 2.4 g/L, 2.5 g/L, 2.6 g/L, 2.7 g/L, 2.8 g/L, 2.9 g/L, 3 g/L, 3.25 g/L, 3.5 g/L, 3.75 g/L, 4 g/L, 4.5 g/L or 5 g/L PPK.

In some embodiments, the method is performed using about 1 g/L ATP-dependent nucleic acid ligase, optionally 1.1 g/L, 1.15 g/L, 1.2 g/L, 1.25 g/L, 1.3 g/L, 1.35 g/L, 1.4 g/L, 1.45 g/L, 1.5 g/L, 1.55 g/L, 1.6 g/L, 1.65 g/L, 1.7 g/L, 1.75 g/L, 1.8 g/L, 1.85 g/L, 1.9 g/L, 1.95 g/L, 2 g/L, 2.1 g/L, 2.2 g/L, 2.3 g/L, 2.4 g/L, 2.5 g/L, 2.6 g/L, 2.7 g/L, 2.8 g/L, 2.9 g/L, 3 g/L, 3.25 g/L, 3.5 g/L, 3.75 g/L, 4 g/L, 4.5 g/L or 5 g/L ATP-dependent nucleic acid ligase.

In some embodiments, the method is performed using about 1 g/L fusion polypeptide, optionally 1.1 g/L, 1.15 g/L, 1.2 g/L, 1.25 g/L, 1.3 g/L, 1.35 g/L, 1.4 g/L, 1.45 g/L, 1.5 g/L, 1.55 g/L, 1.6 g/L, 1.65 g/L, 1.7 g/L, 1.75 g/L, 1.8 g/L, 1.85 g/L, 1.9 g/L, 1.95 g/L, 2 g/L, 2.1 g/L, 2.2 g/L, 2.3 g/L, 2.4 g/L, 2.5 g/L, 2.6 g/L, 2.7 g/L, 2.8 g/L, 2.9 g/L, 3 g/L, 3.25 g/L, 3.5 g/L, 3.75 g/L, 4 g/L, 4.5 g/L or 5 g/L fusion polypeptide.

In some embodiments, wherein four ligation reactions are required to generate the oligonucleotide product, the method is performed using about 1.25 g/L ligase, about 1.25 g/L kinase, about 1 mM oligonucleotide fragments, about 2.5 mM ATP and/or AMP, and about 20 mM polyphosphate. In some embodiments, wherein four ligation reactions are required to generate the oligonucleotide product, the method is performed using about 1.25 g/L fusion polypeptide, about 1 mM oligonucleotide fragments, about 2.5 mM ATP and/or AMP, and about 20 mM polyphosphate.

In some embodiments, wherein four ligation reactions are required to generate the oligonucleotide product, the method is performed using about 1.25 g/L ligase, about 1.25 g/L kinase, about 3 mM oligonucleotide fragments, about 1 mM ATP and/or AMP, and about 20 mM polyphosphate. In some embodiments, wherein four ligation reactions are required to generate the oligonucleotide product, the method is performed using about 1.25 g/L fusion polypeptide, about 3 mM oligonucleotide fragments, about 1 mM ATP and/or AMP, and about 20 mM polyphosphate.

In some embodiments, wherein four ligation reactions are required to generate the oligonucleotide product, the method is performed using about 1.25 g/L ligase, about 1.25 g/L kinase, at least 3 mM (e.g. at least 3.1 mM, at least 3.2 mM, at least 3.3 mM, at least 3.4 mM, at least 3.5 mM, at least 3.6 mM, at least 3.7 mM, at least 3.8 mM, at least 3.9 mM, at least 4 mM, at least 4.5 mM, or at least 5 mM) oligonucleotide fragments, up to 1 mM (e.g. ≤0.9 mM, ≤0.8 mM, ≤0.7 mM, ≤0.6 mM, or ≤0.5 mM) ATP and/or AMP, and about 20 mM polyphosphate. In some embodiments, wherein four ligation reactions are required to generate the oligonucleotide product, the method is performed using about 1.25 g/L fusion polypeptide, at least 3 mM (e.g. at least 3.1 mM, at least 3.2 mM, at least 3.3 mM, at least 3.4 mM, at least 3.5 mM, at least 3.6 mM, at least 3.7 mM, at least 3.8 mM, at least 3.9 mM, at least 4 mM, at least 4.5 mM, or at least 5 mM) oligonucleotide fragments, up to 1 mM (e.g. ≤0.9 mM, ≤0.8 mM, ≤0.7 mM, ≤0.6 mM, or ≤0.5 mM) ATP and/or AMP, and about 20 mM polyphosphate.

It will be understood that references to a concentration of ATP and/or AMP embrace the concentration of ATP alone, the concentration of AMP alone, or the combined concentration of ATP and AMP, e.g. “2.5 mM ATP and/or AMP” embraces: (i) an ATP concentration of 2.5 mM; (ii) an AMP concentration of 2.5 mM; and (iii) a combined ATP and AMP concentration of 2.5 mM.

### MODIFICATIONS

In some embodiments, the oligonucleotide fragment(s) and/or the oligonucleotide comprises a modification, e.g. a chemical modification. As used herein, the term “oligonucleotide fragment(s)” means one or more oligonucleotide fragments. It will be appreciated that modifications which are present in the oligonucleotide fragment(s) are typically present in the oligonucleotide produced from said oligonucleotide fragment(s). In some embodiments, modification(s) are introduced to and/or removed from the oligonucleotide product.

In some embodiments, the oligonucleotide fragment(s) and/or oligonucleotide comprises a chemical modification. In some embodiments, the oligonucleotide fragment(s) and/or oligonucleotide comprises at least one backbone modification. In some embodiments, the oligonucleotide fragment(s) and/or oligonucleotide comprises at least one nucleotide modification. In some embodiments, the oligonucleotide fragment(s) and/or oligonucleotide comprises at least one sugar modification (e.g. at the 2′-position or 4′-position). In some embodiments, the oligonucleotide fragment(s) and/or oligonucleotide comprises: (i) at least one backbone modification; (ii) at least one nucleotide modification; and/or (iii) at least one sugar modification.

Modifications include, but are not limited to, end modifications of the terminal oligonucleotide fragments, e.g., 5′-end modifications (phosphorylation, conjugation, inverted linkages) or 3′-end modifications (conjugation, inverted linkages, etc.); base modifications, e.g., replacement with stabilizing bases, destabilizing bases, or bases that base pair with an expanded repertoire of partners, removal of bases (abasic nucleotides), or conjugated bases; sugar modifications (e.g., at the 2′-position or 4′-position) or replacement of the sugar; or backbone modifications, including modification or replacement of the phosphodiester linkages.

In some embodiments, a terminal oligonucleotide fragment and/or oligonucleotide comprises a cap. The term “cap” and the like include a chemical moiety attached to the end of a double-stranded nucleotide duplex, but is used herein to exclude a chemical moiety that is a nucleotide or nucleoside. A “3′ cap” is attached at the 3′ end of a nucleotide or oligonucleotide and protects the molecule from degradation, e.g., from nucleases, such as those in blood serum or intestinal fluid. A non-nucleotidic 3′ cap is not a nucleotide and can replace a TT or UU dinucleotide at the end of a blunt-ended oligonucleotide. In some embodiments, non-nucleotidic 3′ end caps are as disclosed in, for example, WO 2005/021749 and WO 2007/128477; and U.S. Pat. Nos. 8,097,716; 8,084,600; and 8,344,128. A “5′ cap” is attached at the 5′ end of a nucleotide or oligonucleotide. A cap should not interfere (or unduly interfere) with oligonucleotide activity.

In some embodiments, the oligonucleotide fragment(s) and/or oligonucleotide comprises one or more mismatches. A mismatch is defined herein as a difference between the base sequence or length when two sequences are maximally aligned and compared. In the context of double-stranded oligonucleotides (in which two sequences are aligned antiparallel to each other) a mismatch is defined as a position wherein the base of one sequence is not complementary to the base of the other sequence. Thus, a mismatch is counted, for example, if a position in the first sequence has a particular base (e.g., A), and the corresponding position in the second sequence has a base which is not complementary to said base in the first sequence (e.g., G), when the first and second sequences are aligned antiparallel to each other. Note, however, that on a given RNA strand, a U can be replaced by T (either as RNA or, preferably, DNA, e.g., 2′-deoxy-thymidine); the replacement of a U with a T is not a mismatch as used herein, as either U or T can pair with A on the opposite strand. An RNA oligonucleotide can thus comprise one or more DNA bases, e.g., T. No mismatch is counted between a DNA portion(s) of an RNAi agent and the corresponding target mRNA if basepairing occurs (e.g., between A, G, C, or T in the DNA portion, and the corresponding U, C, G, or A, respectively in the mRNA).

A mismatch is also counted, e.g., if a position in one sequence has a base (e.g., A), and the corresponding position on the other sequence has no base (e.g., that position is an abasic nucleotide, which comprises a phosphate-sugar backbone but no base). A single-stranded nick in either sequence (or in the sense or anti-sense strand) is not counted as mismatch. Thus, as a non-limiting example, no mismatch would be counted if one sequence (in the 5′→3′ orientation) comprises the sequence AG, but the complementary sequence (in the 3′→5′ orientation) comprises the sequence TC with a single-stranded nick between the T and the C. A nucleotide modification in the sugar or phosphate is also not considered a mismatch. Thus, if one sequence comprises a G, and the complementary sequence comprises a modified C (e.g., 2′-modification) at the same position, no mismatch would be counted.

Thus, no mismatches are counted if modifications are made to the sugar, phosphate, or backbone of the oligonucleotide without modifying the base. Thus, in the context of double-stranded RNAi, a strand having a given sequence as an RNA would have zero mismatches from its complement sequence as a PNA; or morpholino; or LNA; or TNA; or GNA; or FANA; or a mix or chimera of RNA and DNA, TNA, GNA, FANA, Morpholino, UNA, LNA, and/or PNA, etc. No mismatch would occur between a nucleotide which is T, and a nucleotide which is A with a 5′ modification and/or a 2′-modification. The key feature of a mismatch (base replacement) is that it would not be able to base-pair with the corresponding base on the opposite strand. In addition, terminal overhangs such as “UU” or “dTdT” are not counted when counting the number of mismatches. In such cases, a mismatch is defined as a position wherein the base of one sequence does not match the base of the other sequence.

It is noted that dTdT (2′-deoxy-thymidine-5′-phosphate and 2′-deoxy-thymidine-5′-phosphate), or in some cases, TT or UU, can be added as a terminal dinucleotide cap or extension to one or both 3′-ends of the oligonucleotide, but this cap or extension is not included in the calculation of the total number of mismatches and is not considered part of the target sequence. This is because the terminal dinucleotide protects the ends from nuclease degradation but does not contribute to target specificity (Elbashir et al. 2001 Nature 411:494-498; Elbashir et al. 2001 EMBO J. 20:6877-6888; and Kraynack et al. 2006 RNA 12:163-176).

There are several examples in the art describing sugar, base, phosphate and backbone modifications that can be introduced into nucleic acid molecules with significant enhancement in their nuclease stability and efficacy. For example, oligonucleotides are modified to enhance stability and/or enhance biological activity by modification with nuclease resistant groups, for example, 2′-amino, 2′-C-allyl, 2′-flouro, 2′-O-methyl, 2′-O-allyl, 2′-H, nucleotide base modifications. Sugar modification of nucleic acid molecules are extensively described in the art.

Additional modifications and conjugations of oligonucleotides have been described. Soutschek et al. 2004 Nature 432:173-178 presented conjugation of cholesterol to the 3′-end of the sense strand of an siRNA molecule by means of a pyrrolidine linker, thereby generating a covalent and irreversible conjugate. Chemical modifications (including conjugation with other molecules) of oligonucleotides may also be made to improve the in vivo pharmacokinetic retention time and efficiency.

In some embodiments, the oligonucleotide fragment(s) and/or oligonucleotide comprises a modified base. The disclosure encompasses an oligonucleotide and oligonucleotide fragments with a substitution of a single nucleotide at a given position with a modified version of the same nucleotide. Thus a nucleotide (A, G, C or U) can be replaced by a modified base selected from 5-fluorouracil, 5-bromouracil, 5-chlorouracil, 5-iodouracil, hypoxanthine, xantine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, 2,6-diaminopurine, 5-hydroxymethyl cytosine, 2-aminoadenine, 6-methyl and other alkyl derivatives of adenine and guanine, 2-propyl and other alkyl derivatives of adenine and guanine, 2-thiothymine, 5-propynyl (—C═C—CH₃) uracil and cytosine and other alkynyl derivatives of pyrimidine bases, 6-azo uracil, cytosine and thymine, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methyladenine, 2-F-adenine, 8-azaguanine and 8-azaadenine, 7-deazaguanine and 7-deazaadenine and 3-deazaguanine and 3-deazaadenine.

Additional modified variants include the addition of any other moiety (e.g., a radiolabel or other tag or conjugate) to the oligonucleotide or oligonucleotide fragment; provided that the base sequence is identical, the addition of other moieties produces a “modified variant” (with no mismatches).

In addition to these modifications and patterns (e.g., formats) for modifications, other modifications or sets of modifications of the sequences provided can be generated using common knowledge of nucleic acid modification. These various embodiments and embodiments of the oligonucleotides of the present disclosure can be used in RNA interference.

In some embodiments, the oligonucleotide and/or oligonucleotide fragment(s) comprises a modification that causes the oligonucleotide to have increased stability in a biological sample or environment (e.g., cytoplasm, interstitial fluid, blood serum, lung or intestinal lavage).

In some embodiments, the oligonucleotide and/or oligonucleotide fragment(s) comprises a modification that promotes cleavage by the RNA-induced silencing complex (i.e. a “RISC cleavage site”). The RISC cleavage site is the site on the target at which cleavage occurs. In some embodiments, the antisense strand comprises a RISC cleavage site. For an RNAi agent having a duplex region of 17-23 nucleotide in length, the cleavage site of the antisense strand is typically around the 10, 11 and 12 positions from the 5′-end. As used herein, the term “cleavage region” refers to a region that is located immediately adjacent to the cleavage site. In some embodiments, the cleavage region comprises three bases on either end of, and immediately adjacent to, the cleavage site. In some embodiments, the cleavage region comprises two bases on either end of, and immediately adjacent to, the cleavage site. In some embodiments, the cleavage site specifically occurs at the site bound by nucleotides 10 and 11 of the antisense strand, and the cleavage region comprises nucleotides 11, 12 and 13 of the antisense strand.

In some embodiments, the oligonucleotide fragment(s) and/or oligonucleotide comprises a modified backbone. As used herein, an unmodified backbone consists of 3′ to 5′ phosphodiester bonds. A modified backbone may comprise non-natural internucleoside linkages. Oligonucleotide fragments and/or oligonucleotides having a modified backbone include those that retain a phosphorus atom in the backbone and those that do not have a phosphorus atom in the backbone.

Oligonucleotide fragments and/or oligonucleotides comprising a modified backbone include, but are not limited to, those that do not have a phosphorus atom in the backbone. Modified backbones include, but are not limited to, phosphorothioates, chiral phosphorothioates, phosphorodithioates, phosphotriesters, aminoalkylphosphotriesters, methyl and other alkyl phosphonates (e.g. 3′-alkylene phosphonates and chiral phosphonates), phosphinates, phosphoramidates (e.g. mesyl phosphoramidates, 3′-amino phosphoramidate and aminoalkylphosphoramidates), thionophosphoramidates, thionoalkylphosphonates, thionoalkylphosphotriesters, and boranophosphates having normal 3′-5′ linkages, 2′-5′-linked analogs of these, and those having inverted polarity wherein the adjacent pairs of nucleoside units are linked 3′-5′ to 5′-3′ or 2′-5′ to 5′-2′.

Oligonucleotide fragments and/or oligonucleotides comprising a modified backbone that does not include a phosphorus atom therein may have backbones that are formed by short chain alkyl or cycloalkyl internucleoside linkages, mixed heteroatoms and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH₂ component parts.

In some embodiments, the oligonucleotide and/or oligonucleotide fragment(s) comprises at least one phosphonate linkage, wherein the phosphonate is a modified phosphonate selected from the group consisting of: phosphorothioate (which may be an Rp isomer or an Sp isomer):

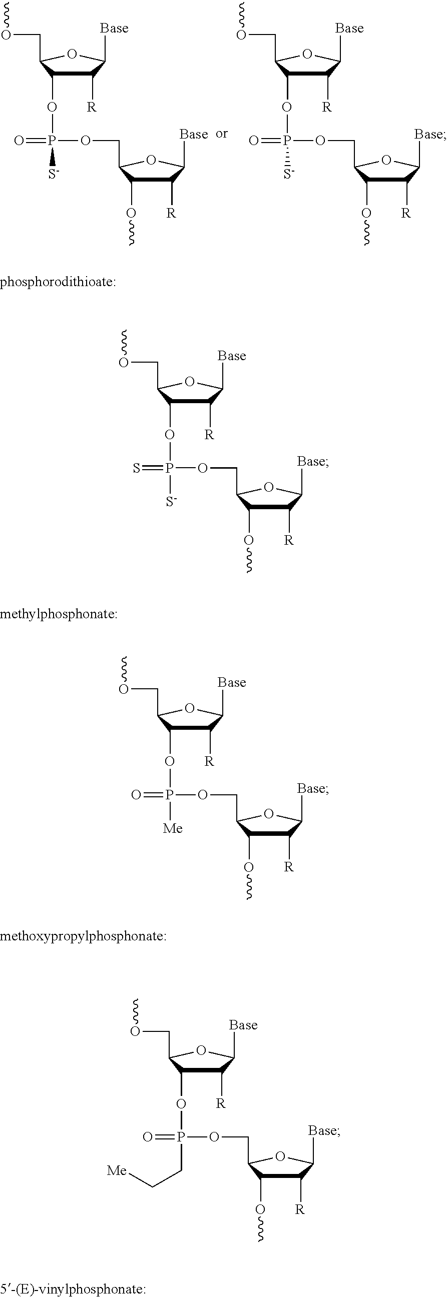

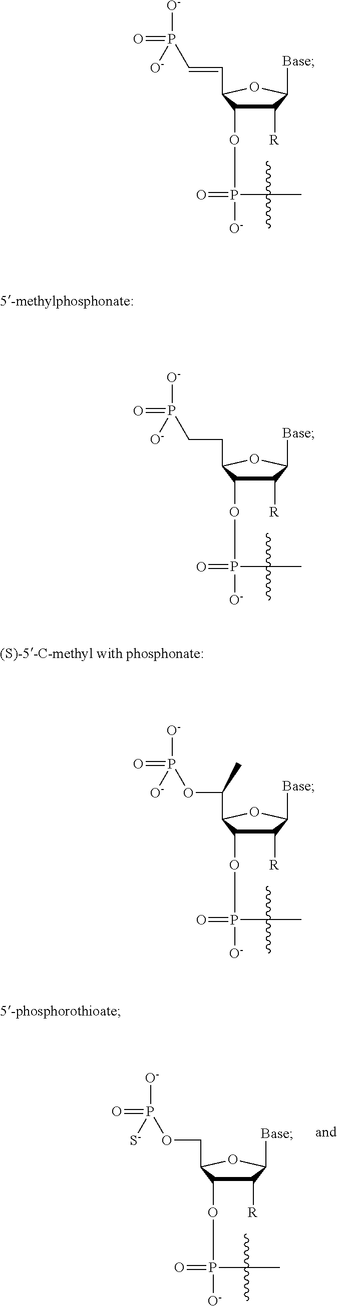

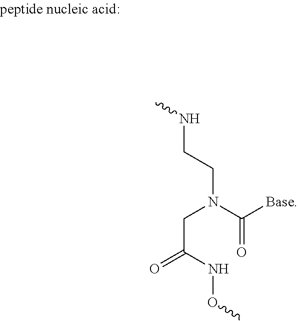

In some embodiments, the oligonucleotide and/or oligonucleotide fragment(s) comprises: at least one 5′-uridine-adenine-3′ (5′-ua-3′) dinucleotide, wherein the uridine is a 2′-modified nucleotide; at least one 5′-uridine-guanine-3′ (5′-ug-3′) dinucleotide, wherein the 5′-uridine is a 2′-modified nucleotide; at least one 5′-cytidine-adenine-3′ (5′-ca-3′) dinucleotide, wherein the 5′-cytidine is a 2′-modified nucleotide; or at least one 5′-uridine-uridine-3′ (5′-uu-3′) dinucleotide, wherein the 5′-uridine is a 2′-modified nucleotide. These dinucleotide motifs are particularly prone to serum nuclease degradation (e.g. RNase A). Chemical modification at the 2′-position of the first pyrimidine nucleotide in the motif prevents or slows down such cleavage. This modification recipe is also known under the term ‘endo light’.

In some embodiments, the oligonucleotide and/or oligonucleotide fragment(s) comprise a modified nucleobase, wherein the modified nucleobase is difluorotolyl, nitroindolyl, nitropyrrolyl, or nitroimidazolyl. In a particular embodiment, the modified nucleobase is difluorotolyl. In some embodiments, wherein the oligonucleotide and/or oligonucleotide fragment(s) is double-stranded, only one of the two strands contains a modified nucleobase. In some embodiments, wherein the oligonucleotide and/or oligonucleotide fragment(s) is double-stranded, both of the strands contain a modified nucleobase.

In some embodiments, the oligonucleotide fragment(s) and/or oligonucleotide comprises a modified sugar. Sugar modifications typically involve chemical modification of the sugar moiety of RNA or DNA. Sugar modifications include, but are not limited to, one of the following at the 2′-position: OH; F; O—, S—, or N-alkyl; O—, S—, or N-alkenyl; O—, S- or N-alkynyl; or O-alkyl-O-alkyl, wherein the alkyl, alkenyl and alkynyl can be substituted or unsubstituted C₁ to C₁₀ alkyl or C₂ to C₁₀ alkenyl and alkynyl. Exemplary modifications include O[(CH₂)ₙO]ₘCH₃, O(CH₂)·ₙOCH₃, O(CH₂)ₙNH₂, O(CH₂)ₙCH₃, O(CH₂)ₙONH₂, and O(CH₂)ₙON [(CH₂)ₙCH₃)]₂, where n and m are from 1 to about 10. Oligonucleotide fragments for use in the methods described herein may include one of the following at the 2′ position: C₁ to C₁₀ lower alkyl, substituted lower alkyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of a therapeutic RNA, or a group for improving the pharmacodynamic properties of a therapeutic RNA. In some embodiments, the modification comprises a 2′-methoxyethoxy (also known as 2′-O-(2-methoxyethyl) or 2′-O-MOE), 2′-dimethylaminooxyethoxy (also known as 2′-DMAOE), and 2′-dimethylaminoethoxyethoxy (also known in the art as 2′-O-dimethylaminoethoxyethyl or 2′-DMAEOE). Further exemplary modifications include: 5′-Me-2′-F nucleotides, 5′-Me-2′-OMe nucleotides, 5′-Me-2′-deoxynucleotides, 2′-alkoxyalkyl; and 2′-NMA (N-methylacetamide).

Other modifications include 2′-methoxy (2′-OCH₃), 2′-aminopropoxy (2′-OCH₂CH₂CH₂NH₂) and 2′-fluoro (2′-F). Similar modifications can also be made at other positions on an RNA, particularly the 3′ position of the sugar on the 3′ terminal nucleotide or in 2′-5′ linked dsRNA and the 5′ position of 5′ terminal nucleotide.

In some embodiments, the oligonucleotide fragment(s) and/or oligonucleotide comprises at least one 2′-modified nucleotide. In some embodiments, the 2′-modification is selected from the group consisting of: 2′-O-methyl (2′-OMe), 2′-flouro (2′-F), 2′-deoxy, 2′-deoxy-2′-fluoro, 2′-O-methoxyethyl (2′-O-MOE), 2′-O-aminopropyl (2′-O-AP), 2′-O-dimethylaminoethyl (2′-O-DMAOE), 2′-O-dimethylaminopropyl (2′-O-DMAP), 2′-O-dimethylaminoethyloxyethyl (2′-O-DMAEOE), 2′-O—N-methylacetamido (2′-O-NMA), locked nucleic acid (LNA), glycol nucleic acid (GNA), phosphoramidate (e.g. mesyl phosphoramidate), ‘2’,3′-seco nucleotide mimic, 2′-F-arabino nucleotide, abasic nucleotide, 2′-amino modified nucleotide, 2′-alkyl-modified nucleotide, morpholino nucleotide, vinylphosphonate (e.g. 5′ vinylphosphonate), and cyclopropyl phosphonate deoxyribonucleotide. In some embodiments, one or more of the oligonucleotide fragments comprises a 2′-modification selected from the group consisting of: 2′-OMe, 2′-F, and 2′-deoxy. In some embodiments, the oligonucleotide and/or oligonucleotide fragment(s) comprises one or more 3′-O-methyl nucleotide.

In some embodiments, the oligonucleotide and/or oligonucleotide fragment(s) comprise a 2′-modification selected from the group consisting of: 2′-O-methyl (2′-OMe), 2′-flouro (2′-F), 2′-deoxy, 2′-deoxy-2′-fluoro, 2′-O-methoxyethyl (2′-O-MOE), 2′-O-aminopropyl (2′-O-AP), 2′-O-dimethylaminoethyl (2′-O-DMAOE), 2′-O-dimethylaminopropyl (2′-O-DMAP), 2′-O-dimethylaminoethyloxyethyl (2′-O-DMAEOE), 2′-O—N-methylacetamido (2′-O-NMA), locked nucleic acid (LNA), glycol nucleic acid (GNA), phosphoramidate (e.g. mesyl phosphoramidate), 2′,3′-seco nucleotide mimic, 2′-F-arabino nucleotide, abasic nucleotide, 2′-amino modified nucleotide, 2′-alkyl-modified nucleotide, morpholino nucleotide, vinylphosphonate (e.g. 5′ vinylphosphonate), deoxyribonucleotide, and cyclopropyl phosphonate. In some embodiments, the oligonucleotide and/or oligonucleotide fragment(s) comprises one or more 3′-O-methyl nucleotide.

In some embodiments, the oligonucleotide and/or oligonucleotide fragment(s) comprises a bridged nucleic acid. In some embodiments, the bridged nucleic acid is locked nucleic acid. In some embodiments, the bridged nucleic acid is constrained ethyl bridged nucleic acid:

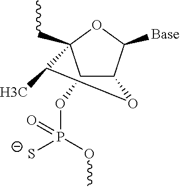

In some embodiments, all pyrimidines (uridine and cytidine) are 2′ O-methyl-modified nucleosides.

In some embodiments, the sense and/or antisense strand is conjugated to one or more diagnostic compound, reporter group, cross-linking agent, nuclease-resistance conferring moiety, modified or unmodified nucleobase, lipophilic molecule, cholesterol, lipid, lectin, steroid, uvaol, hecigenin, diosgenin, terpene, triterpene, sarsasapogenin, Friedelin, epifriedelanol-derivatized lithocholic acid, vitamin, carbohydrate, dextran, pullulan, chitin, chitosan, synthetic carbohydrate, oligo lactate 15-mer, natural polymer, low- or medium-molecular weight polymer, inulin, cyclodextrin, hyaluronic acid, protein, protein-binding agent, integrin-targeting molecule, polycationic, peptide, polyamine, peptide mimic, and/or transferrin.

In some embodiments, the antisense strand comprises at least one 2′-OMe modified nucleotide. In some embodiments, the antisense strand comprises at least one 2′-F modified nucleotide. In some embodiments, the antisense strand comprises at least one 2′-deoxy modified nucleotide. In some embodiments, the antisense strand comprises at least one 2′-OMe modified nucleotide, at least one 2′-F modified nucleotide, or at least one 2′-deoxy modified nucleotide, or any combination thereof. In some embodiments, the antisense strand comprises alternating 2′-OMe and 2′-F modified nucleotides. In some embodiments, the antisense strand comprises at least one 5′ vinylphosphonate. In some embodiments, the antisense strand comprises at least one chiral phosphorothioate linkage. In some embodiments, the antisense strand comprises at least one GNA. In some embodiments, the sense strand comprises at least one 2′-OMe modified nucleotide. In some embodiments, the sense strand comprises at least one 2′-F modified nucleotide. In some embodiments, the sense strand comprises at least one 2′-deoxy modified nucleotide. In some embodiments, the sense strand comprises at least one 2′-OMe modified nucleotide, at least one 2′-F modified nucleotide, or at least one 2′-deoxy modified nucleotide, or any combination thereof. In some embodiments, the sense strand comprises alternating 2′-OMe and 2′-F modified nucleotides. In some embodiments, the antisense strand and the sense strand each comprise at least one 2′-OMe modified nucleotide. In some embodiments, the antisense strand and the sense strand each comprise at least one 2′-F modified nucleotide. In some embodiments, the antisense strand and the sense strand each comprise alternating 2′-OMe and 2′-F modified nucleotides. In some embodiments, the sense strand comprises at least one 5′ vinylphosphonate. In some embodiments, the sense strand comprises at least one chiral phosphorothioate linkage. In some embodiments, the sense strand comprises at least one GNA.

In some embodiments, the sense strand comprises alternating 2′-OMe and 2′-F modified nucleotides over the full length of the sense strand. In some embodiments, the sense strand comprises alternating 2′-OMe and 2′-F modified nucleotides over part of the length of the sense strand e.g. over at least 2, 3, 4, 5, 6, 7, 8, 9, 10, 11, 12, 13, 14, 15, 16, 17, 18, 19, 20 or more nucleotides of the sense strand.

In some embodiments, the antisense strand comprises alternating 2′-OMe and 2′-F modified nucleotides over the full length of the antisense strand. In some embodiments, the antisense strand comprises alternating 2′-OMe and 2′-F modified nucleotides over part of the length of the antisense strand e.g. over at least 2, 3, 4, 5, 6, 7, 8, 9, 10, 11, 12, 13, 14, 15, 16, 17, 18, 19, 20 or more nucleotides of the antisense strand.

In some embodiments, the sense strand and antisense strand each comprise alternating 2′-OMe and 2′-F modified nucleotides over the full length of the sense strand and the antisense strand. In some embodiments, the sense strand and the antisense strand comprise alternating 2′-OMe and 2′-F modified nucleotides over part of the length of the sense strand and the antisense strand e.g. over at least 2, 3, 4, 5, 6, 7, 8, 9, 10, 11, 12, 13, 14, 15, 16, 17, 18, 19, 20 or more nucleotides of the sense strand and the antisense strand.

### Ligands

In some embodiments, one or more of the oligonucleotide fragments is conjugated to at least one ligand. In some embodiments, the oligonucleotide product is conjugated to at least one ligand. The ligand may be conjugated to the sense strand, antisense strand or both strands, in any configuration e.g. at the 3′-end, 5′-end, non-end or a combination.

In some embodiments, the ligand comprises one or more N-Acetylgalactosamine (GalNAc) derivatives. In some embodiments, the ligand comprises one or more GalNAc derivatives conjugated through a bivalent or trivalent branched carrier.

In some embodiments, the ligand is:

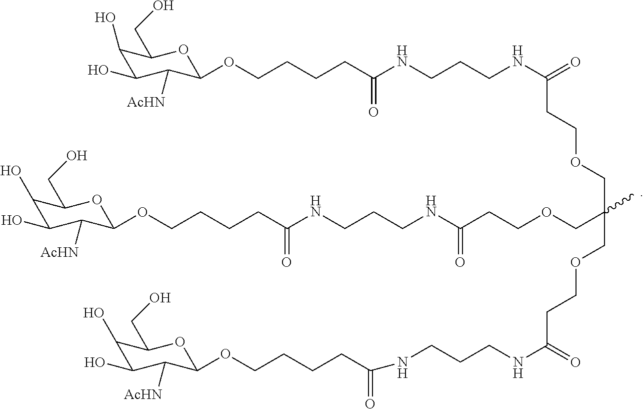 [SMILES: CC(=O)NC1C(OCCCCC(=O)NCCCNC(=O)CCOCC(C)(COCCC(=O)NCCCNC(=O)CCCCOC2OC(CO)C(O)C(O)C2NC(C)=O)COCCC(=O)NCCCNC(=O)CCCCOC2OC(CO)C(O)C(O)C2NC(C)=O)OC(CO)C(O)C1O]

In some embodiments, the ligand is:

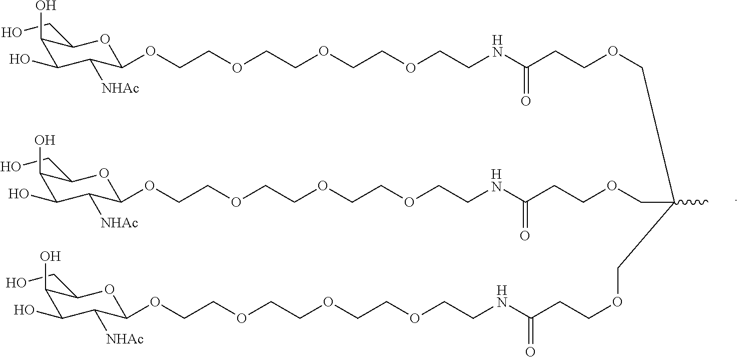 [SMILES: CC(=O)NC1C(OCCOCCOCCOCCNC(=O)CCOCC(C)(COCCC(=O)NCCOCCOCCOCCOC2OC(CO)C(O)C(O)C2NC(C)=O)COCCC(=O)NCCOCCOCCOCCOC2OC(CO)C(O)C(O)C2NC(C)=O)OC(CO)C(O)C1O]

In some embodiments, the ligand is:

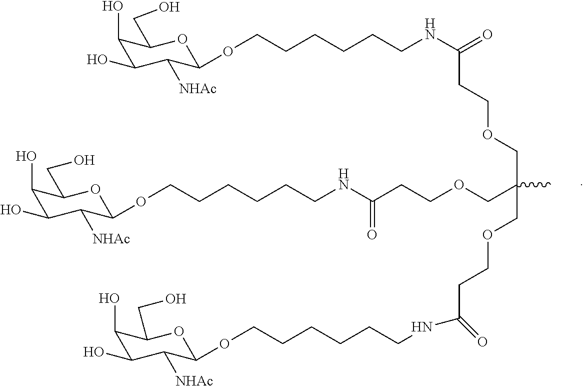 [SMILES: CC(=O)NC1C(OCCCCCCNC(=O)CCOCC(C)(COCCC(=O)NCCCCCCOC2OC(CO)C(O)C(O)C2NC(C)=O)COCCC(=O)NCCCCCCOC2OC(CO)C(O)C(O)C2NC(C)=O)OC(CO)C(O)C1O]

In some embodiments, the ligand is:

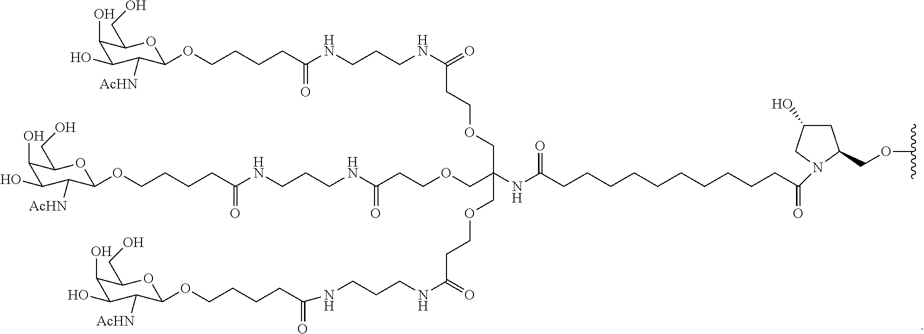 [SMILES: *OC[C@@H]1C[C@@H](O)CN1C(=O)CCCCCCCCCCC(=O)NC(COCCC(=O)NCCCNC(=O)CCCCOC1OC(CO)C(O)C(O)C1NC(C)=O)(COCCC(=O)NCCCNC(=O)CCCCOC1OC(CO)C(O)C(O)C1NC(C)=O)COCCC(=O)NCCCNC(=O)CCCCOC1OC(CO)C(O)C(O)C1NC(C)=O]

In some embodiments, the ligand is:

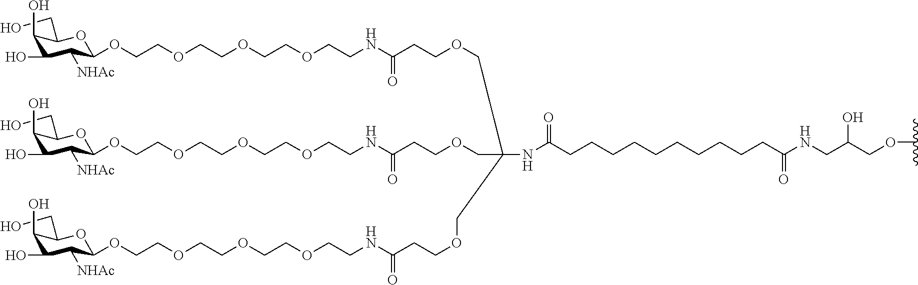

In some embodiments, the ligand is:

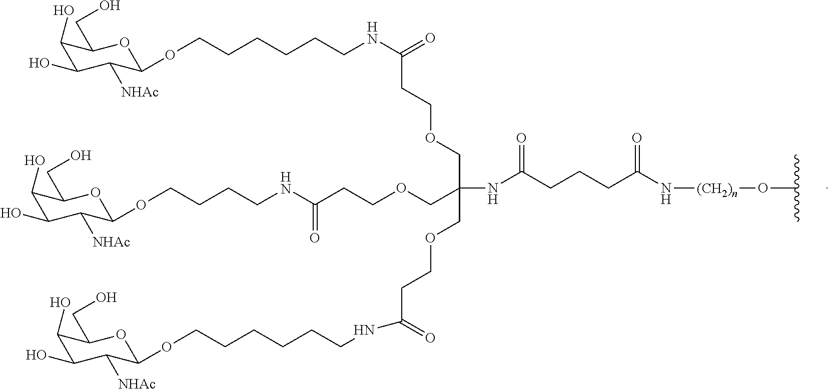

In some embodiments, a ligand alters the distribution, targeting or lifetime of the molecule into which it is incorporated. In some embodiments, a ligand provides an enhanced affinity for a selected target, e.g., molecule, cell or cell type, compartment, receptor e.g., a cellular or organ compartment, tissue, organ or region of the body, as, e.g., compared to a species absent such a ligand. Ligands providing enhanced affinity for a selected target are also termed targeting ligands.

Some ligands can have endosomolytic properties. The endosomolytic ligands promote the lysis of the endosome and/or transport of the oligonucleotide, or a composition comprising the oligonucleotide, from the endosome to the cytoplasm of the cell. The endosomolytic ligand may be a polyanionic peptide or peptidomimetic which shows pH-dependent membrane activity and fusogenicity. In some embodiments, the endosomolytic ligand assumes its active conformation at endosomal pH. The “active” conformation is that conformation in which the endosomolytic ligand promotes lysis of the endosome and/or transport of the oligonucleotide, or a composition comprising the oligonucleotide, from the endosome to the cytoplasm of the cell. Exemplary endosomolytic ligands include the GALA peptide (Subbarao et al., Biochemistry, 1987, 26:2964-2972), the EALA peptide (Vogel et al., J. Am. Chem. Soc., 1996, 118:1581-1586), and their derivatives (Turk et al., Biochem. Biophys. Acta, 2002, 1559:56-68). The endosomolytic component may contain a chemical group (e.g., an amino acid) which will undergo a change in charge or protonation in response to a change in pH. The endosomolytic component may be linear or branched.

Ligands can improve transport, hybridization, and specificity properties and may also improve nuclease resistance of the resultant natural or modified oligonucleotide.

Ligands in general can include therapeutic modifiers, e.g., for enhancing uptake; diagnostic compounds or reporter groups e.g., for monitoring distribution; cross-linking agents; and nuclease-resistance conferring moieties. General examples include lipids, steroids, vitamins, sugars, proteins, peptides, polyamines, and peptide mimics.

Ligands can include a naturally occurring substance, such as a protein (e.g., human serum albumin (HSA), low-density lipoprotein (LDL), high-density lipoprotein (HDL), or globulin); a carbohydrate (e.g., a dextran, pullulan, chitin, chitosan, inulin, cyclodextrin or hyaluronic acid); or a lipid. The ligand may also be a recombinant or synthetic molecule, such as a synthetic polymer, e.g., a synthetic polyamino acid, an oligonucleotide (e.g., an aptamer). Examples of polyamino acids include polylysine (PLL), poly L aspartic acid, poly L-glutamic acid, styrene-maleic acid anhydride copolymer, poly(L-lactide-co-glycolied) copolymer, divinyl ether-maleic anhydride copolymer, N-(2-hydroxypropyl) methacrylamide copolymer (HMPA), polyethylene glycol (PEG), polyvinyl alcohol (PVA), polyurethane, poly(2-ethylacryllic acid), N-isopropylacrylamide polymers, or polyphosphazine. Examples of polyamines include: polyethylenimine, polylysine (PLL), spermine, spermidine, polyamine, pseudopeptide-polyamine, peptidomimetic polyamine, dendrimer polyamine, arginine, amidine, protamine, cationic lipid, cationic porphyrin, quaternary salt of a polyamine, or an alpha helical peptide.

Ligands can also include targeting groups, e.g., a cell or tissue targeting agent, e.g., a lectin, glycoprotein, lipid or protein, e.g., an antibody, that binds to a specified cell type. A targeting group can be a thyrotropin, melanotropin, lectin, glycoprotein, surfactant protein A, Mucin carbohydrate, multivalent lactose, multivalent galactose, N-acetyl-galactosamine, N-acetyl-gulucosamine multivalent mannose, multivalent fucose, glycosylated polyaminoacids, multivalent galactose, transferrin, bisphosphonate, polyglutamate, polyaspartate, a lipid, cholesterol, a steroid, bile acid, folate, vitamin B12, biotin, an RGD peptide, an RGD peptidomimetic or an aptamer.

Other examples of ligands include dyes, intercalating agents (e.g., acridines), cross-linkers (e.g., psoralene, mitomycin C), porphyrins (TPPC4, texaphyrin, Sapphyrin), polycyclic aromatic hydrocarbons (e.g., phenazine, dihydrophenazine), artificial endonucleases or a chelator (e.g., EDTA), lipophilic molecules, e.g., cholesterol, cholic acid, adamantane acetic acid, 1-pyrene butyric acid, dihydrotestosterone, 1,3-Bis-O (hexadecyl)glycerol, geranyloxyhexyl group, hexadecylglycerol, borneol, menthol, 1,3-propanediol, heptadecyl group, palmitic acid, myristic acid, O3-(oleoyl) lithocholic acid, O3-(oleoyl) cholenic acid, dimethoxytrityl, or phenoxazine) and peptide conjugates (e.g., antennapedia peptide, Tat peptide), alkylating agents, phosphate, amino, mercapto, PEG (e.g., PEG-40K), MPEG, [MPEG]2, polyamino, alkyl, substituted alkyl, radiolabeled markers, enzymes, haptens (e.g., biotin), transport/absorption facilitators (e.g., aspirin, vitamin E, folic acid), synthetic ribonucleases (e.g., imidazole, bisimidazole, histamine, imidazole clusters, acridine-imidazole conjugates, Eu3+ complexes of tetraazamacrocycles), dinitrophenyl, HRP, or AP.

Ligands can be proteins, e.g., glycoproteins, or peptides, e.g., molecules having a specific affinity for a co-ligand, or antibodies e.g., an antibody, that binds to a specified cell type such as a cancer cell, endothelial cell, or bone cell. Ligands may also include hormones and hormone receptors. They can also include non-peptidic species, such as lipids, lectins, carbohydrates, vitamins, cofactors, multivalent lactose, multivalent galactose, N-acetyl-galactosamine, N-acetyl-gulucosamine multivalent mannose, multivalent fucose, or aptamers. The ligand can be, for example, a lipopolysaccharide, an activator of p38 MAP kinase, or an activator of NF-κB.

In some embodiments, the ligand is a lipid or lipid-based molecule. Such a lipid or lipid-based molecule preferably binds a serum protein, e.g., human serum albumin (HSA). An HSA binding ligand allows for distribution of the conjugate to a target tissue. A lipid or lipid-based ligand can (a) increase resistance to degradation of the conjugate, (b) increase targeting or transport into a target cell or cell membrane, and/or (c) can be used to adjust binding to a serum protein, e.g., HSA. A lipid based ligand can be used to modulate, e.g., control the binding of the conjugate to a target tissue.

In some embodiments, the ligand is a peptide or a peptidomimetic. A peptidomimetic is a molecule capable of folding into a defined three-dimensional structure similar to a natural peptide. The peptide or peptidomimetic moiety can be about 5-50 amino acids long, e.g., about 5, 10, 15, 20, 25, 30, 35, 40, 45, or 50 amino acids long. A peptide or peptidomimetic can be, for example, a cell permeation peptide, cationic peptide, amphipathic peptide, or hydrophobic peptide (e.g., consisting primarily of Tyr, Trp or Phe). The peptide moiety can be a dendrimer peptide, constrained peptide or crosslinked peptide. In another alternative, the peptide moiety can include a hydrophobic membrane translocation sequence (MTS). The peptide moiety can be a “delivery” peptide, which can carry large polar molecules including peptides, oligonucleotides, and protein across cell membranes. A peptide or peptidomimetic can be encoded by a random sequence of DNA, such as a peptide identified from a phage-display library, or one-bead-one-compound (OBOC) combinatorial library (Lam et al., Nature, 354:82-84, 1991).

As used herein, a “peptide moiety” can range in length from about 5 amino acids to about 50 amino acids. The peptide moieties can have a structural modification, such as to increase stability or direct conformational properties. Any of the structural modifications described below can be utilized. An arginine-glycine-aspartic acid (RGD)-peptide moiety can be used to target a tumor cell, such as an endothelial tumor cell or a breast cancer tumor cell (Zitzmann et al., Cancer Res., 62:5139-43, 2002). An RGD peptide can facilitate targeting of an oligonucleotide to tumors of a variety of other tissues, including the lung, kidney, spleen, or liver (Aoki et al., Cancer Gene Therapy 8:783-787, 2001). The RGD peptide can be linear or cyclic, and can be modified, e.g., glycosylated or methylated to facilitate targeting to specific tissues. Peptides that target markers enriched in proliferating cells can be used. For example, RGD containing peptides and peptidomimetics can target cancer cells, in particular cells that exhibit an integrin. Thus, the ligand may comprise RGD peptides, cyclic peptides containing RGD, RGD peptides that include D-amino acids, or synthetic RGD mimics.

Peptide and peptidomimetic ligands include those having naturally occurring or modified peptides, e.g., D or L peptides; a, B, or y peptides; N-methyl peptides; azapeptides; peptides having one or more amide, i.e., peptide, linkages replaced with one or more urea, thiourea, carbamate, or sulfonyl urea linkages; or cyclic peptides.

Ligands can be coupled to the oligonucleotide fragment(s) and/or oligonucleotide at various places, for example, 3′-end, 5′-end, and/or at an internal (“non-end”) position. In some embodiments, the ligand is attached via an intervening tether, e.g., a carrier described herein. The ligand or tethered ligand may be present on a monomer when the monomer is incorporated into the oligonucleotide fragment(s) and/or oligonucleotide. In some embodiments, the ligand may be incorporated via coupling to a “precursor” monomer after the “precursor” monomer has been incorporated into the oligonucleotide fragment and/or the oligonucleotide. For example, a monomer having, e.g., an amino-terminated tether (i.e., having no associated ligand), e.g., TAP-(CH2)nNH2 may be incorporated into a growing oligonucleotide fragment. In a subsequent operation, i.e., after incorporation of the precursor monomer into the oligonucleotide fragment, a ligand having an electrophilic group, e.g., a pentafluorophenyl ester or aldehyde group, can subsequently be attached to the precursor monomer by coupling the electrophilic group of the ligand with the terminal nucleophilic group of the precursor monomer's tether.

In another example, a monomer having a chemical group suitable for taking part in Click Chemistry reaction may be incorporated, e.g., an azide or alkyne terminated tether/linker. In a subsequent operation, i.e., after incorporation of the precursor monomer into the oligonucleotide fragment(s) and/or the oligonucleotide, a ligand having complementary chemical group, e.g. an alkyne or azide can be attached to the precursor monomer by coupling the alkyne and the azide together.

In some embodiments, the ligand is conjugated to nucleobases, sugar moieties, or internucleosidic linkages of oligonucleotide fragment(s) and/or oligonucleotide. Conjugation to purine nucleobases or derivatives thereof can occur at any position including, endocyclic and exocyclic atoms. In some embodiments, the 2-, 6-, 7-, or 8-positions of a purine nucleobase are attached to a conjugate moiety. Conjugation to pyrimidine nucleobases or derivatives thereof can also occur at any position. In some embodiments, the 2-, 5-, and 6-positions of a pyrimidine nucleobase can be substituted with a conjugate moiety. Conjugation to sugar moieties of nucleosides can occur at any carbon atom. Example carbon atoms of a sugar moiety that can be attached to a conjugate moiety include the 2′, 3′, and 5′ carbon atoms. The l′ position can also be attached to a conjugate moiety, such as in an abasic residue. Internucleosidic linkages can also bear conjugate moieties. For phosphorus-containing linkages (e.g., phosphodiester, phosphorothioate (e.g. chiral phosphorothioate), phosphorodithiotate, phosphoroamidate, and the like), the conjugate moiety can be attached directly to the phosphorus atom or to an O, N, or S atom bound to the phosphorus atom. For amine- or amide-containing internucleosidic linkages (e.g., PNA), the conjugate moiety can be attached to the nitrogen atom of the amine or amide or to an adjacent carbon atom.

In some embodiments, the ligand is conjugated to the sense strand. In some embodiments, the ligand is conjugated to the 3′ end of the sense strand. In some embodiments, the ligand is conjugated to the 5′ end of the sense strand. In some embodiments, the ligand is conjugated to a non-end of the sense strand.

In some embodiments, the ligand is conjugated to the antisense strand. In some embodiments, the ligand is conjugated to the 3′ end of the antisense strand. In some embodiments, the ligand is conjugated to a non-end of the antisense strand.

The ligand may be attached via a carrier. The carriers include (i) at least one “backbone attachment point,” preferably two “backbone attachment points” and (ii) at least one “tethering attachment point.” A “backbone attachment point” as used herein refers to a functional group, e.g. a hydroxyl group, or generally, a bond available for, and that is suitable for incorporation of the carrier into the backbone, e.g., the phosphate, or modified phosphate, e.g., sulfur containing, backbone, of a nucleic acid. A “tethering attachment point” (TAP) in some embodiments refers to a constituent ring atom of the cyclic carrier, e.g., a carbon atom or a heteroatom (distinct from an atom which provides a backbone attachment point), that connects a selected moiety. The moiety can be, e.g., a carbohydrate, e.g. monosaccharide, disaccharide, trisaccharide, tetrasaccharide, oligosaccharide and polysaccharide. Optionally, the selected moiety is connected by an intervening tether to the cyclic carrier. Thus, the cyclic carrier will often include a functional group, e.g., an amino group, or generally, provide a bond, that is suitable for incorporation or tethering of another chemical entity, e.g., a ligand to the constituent ring.

Wherein the oligonucleotide fragment is a dsRNA, the sense and/or antisense strand may be conjugated to a ligand via a carrier, wherein the carrier can be cyclic group or acyclic group; preferably, the cyclic group is selected from pyrrolidinyl, pyrazolinyl, pyrazolidinyl, imidazolinyl, imidazolidinyl, piperidinyl, piperazinyl, [1,3]dioxolane, oxazolidinyl, isoxazolidinyl, morpholinyl, thiazolidinyl, isothiazolidinyl, quinoxalinyl, pyridazinonyl, tetrahydrofuryl and and decalin; preferably, the acyclic group is selected from serinol backbone or diethanolamine backbone.

In some embodiments, one or more oligonucleotide fragments comprise the sequence “TT”, “dTdT”, “dTsdT” or “UU” as a single-stranded overhang at the 3′ end, also termed herein a terminal dinucleotide or 3′ terminal dinucleotide. dT is 2′-deoxy-thymidine-5′-phosphate and sdT is 2′-deoxy Thymidine 5′-phosphorothioate. Terminal dinucleotide “UU” is UU or 2′-OMe-U 2′-OMe-U, and the terminal TT and the terminal UU can be in the inverted/reverse orientation. The terminal dinucleotide (e.g., UU) is a modified variant of the dithymidine dinucleotide commonly placed as an overhang to protect the ends of siRNAs from nucleases (see, for example, Elbashir et al. 2001 Nature 411:494-498; Elbashir et al. 2001 EMBO J. 20:6877-6888; and Kraynack et al. 2006 RNA 12:163-176). A terminal dinucleotide is known from these references to enhance nuclease resistance but not contribute to target recognition.

In some embodiments, one or both terminal oligonucleotide fragments comprise a 3′ end cap instead of or in addition to a terminal dinucleotide to stabilize the end from nuclease degradation provided that the 3′ end cap is able to both stabilize the oligonucleotide (e.g., against nucleases) and not interfere excessively with its desired activity.

Also provided is an oligonucleotide produced by the method described herein.

The oligonucleotide may be in any suitable buffer solution. In some embodiments, the buffer solution is selected from Tris buffer (e.g. Tris-HCl), phosphate buffer, HEPES, MOPS (3-(N-morpholino) propanesulfonic acid), and triethanolamine (TEOA) buffer. In some embodiments, the buffer solution comprises acetate, citrate, prolamine, carbonate, or phosphate, or any combination thereof. In some embodiments, the buffer solution is phosphate buffered saline (PBS), e.g. PBS having an NaCl concentration of <100 mM. In some embodiments, the buffer solution further comprises an agent for controlling the osmolarity of the solution, such that the osmolarity is kept at a desired value, e.g., at the physiologic values of the human plasma. Solutes which can be added to the buffer solution to control the osmolarity include, but are not limited to, proteins, peptides, amino acids, non-metabolized polymers, vitamins, ions, sugars, metabolites, organic acids, lipids, or salts. In some embodiments, the agent for controlling the osmolarity of the solution is a salt. In some embodiments, the agent for controlling the osmolarity of the solution is sodium chloride or potassium chloride.

### Additional Embodiments

Embodiment 1. A method of producing an oligonucleotide from two or more oligonucleotide fragments, wherein the method comprises contacting:
   i. two or more oligonucleotide fragments;
   ii. an ATP-dependent nucleic acid ligase;
   iii. a polyphosphate kinase (PPK);
   iv. adenosine triphosphate (ATP) and/or adenosine monophosphate (AMP);
   v. polyphosphate; and
   vi. a divalent cation;
and thereby providing an oligonucleotide.
Embodiment 2. Use of an ATP-dependent nucleic acid ligase and a PPK in the production of an oligonucleotide from two or more oligonucleotide fragments.
Embodiment 3. The method of Embodiment 1 or the use of Embodiment 2, wherein the two or more oligonucleotide fragments comprise two or more RNA oligonucleotide fragments.
Embodiment 4. The method or use of Embodiment 3, wherein the ATP-dependent nucleic acid ligase is an RNA ligase.
Embodiment 5. The method or use of Embodiment 4, wherein the RNA ligase is a double-stranded RNA ligase.
Embodiment 6. The method or use of Embodiment 4 or Embodiment 5, wherein the RNA ligase is a member of the RNA ligase 2 family.
Embodiment 7. The method or use of Embodiment 6, wherein the RNA ligase is Bacteriophage RB69 RNA ligase 2.
Embodiment 8. The method of Embodiment 1 or the use of Embodiment 2, wherein the two or more oligonucleotide fragments comprise two or more DNA oligonucleotide fragments.
Embodiment 9. The method or use of Embodiment 8, wherein the ATP-dependent nucleic acid ligase is a DNA ligase.
Embodiment 10. The method or use of Embodiment 9, wherein the DNA ligase is T4 DNA ligase.
Embodiment 11. The method of any one of Embodiments 1 or 3-10 or the use of any one of Embodiments 2-10, wherein the PPK is PPK12 or ajPAP.
Embodiment 12. The method of any one of Embodiments 1 or 3-11 or the use of any one of Embodiments 2-11, wherein the ATP-dependent nucleic acid ligase and the PPK are linked.
Embodiment 13. The method or use of Embodiment 12, wherein the ATP-dependent nucleic acid ligase and the PPK are linked via a polypeptide linker.
Embodiment 14. The method or use of Embodiment 13, wherein the PPK is located at the N-terminus of the linker and the ATP-dependent nucleic acid ligase is located at the C-terminus of the linker.
Embodiment 15. The method of any one of Embodiments 1 or 3-14 or the use of any one of Embodiments 2-14, wherein:
   a. the ATP-dependent nucleic acid ligase comprises a purification tag;
   b. the PPK comprises a purification tag; and/or
   c. the linker comprises a purification tag.
Embodiment 16. The method or use of any of Embodiments 13-15, wherein the linker is a polypeptide linker comprising at least 3 amino acids, optionally at least 6 amino acids.
Embodiment 17. The method or use of Embodiment 16, wherein the linker comprises an amino acid sequence selected from:

a)
(SEQ ID NO: 19)
HHHHHH,
optionally
(SEQ ID NO: 20)
HHHHHHHHHH;
b)
(SEQ ID NO: 21)
ENLYFQS;
c)
(SEQ ID NO: 22)
ENLYFQG;
d)
(SEQ ID NO: 23)
SSGSSG;
e)
(SEQ ID NO: 24)
GSAGSAAGSGEF;
and/or
f)
(SEQ ID NO: 25)
GSSGSGSSSGGSSSSGSS.

Embodiment 18. The method of any one of Embodiments 1 or 3-17, wherein the polyphosphate is a polyphosphate salt.
Embodiment 19. The method of Embodiment 18, wherein the polyphosphate salt is sodium polyphosphate (Maddrell's salt) or sodium hexametaphosphate (Graham's salt).
Embodiment 20. The method of any one of Embodiments 1 or 3-19, wherein the divalent cation cofactor is Mg²⁺ or Mn²⁺.
Embodiment 21. The method of any one of Embodiments 1 or 3-20, wherein the method is performed with a divalent cation concentration of 5-100 mM, optionally 30-50 mM.
Embodiment 22. The method of any one of Embodiments 1 or 3-21, wherein the method is performed with a sub-stoichiometric concentration of ATP and/or AMP.
Embodiment 23. The method of any one of Embodiments 1 or 3-22, further comprising a step of purifying the oligonucleotide.
Embodiment 24. The method of any one of Embodiments 1 or 3-23 or the use according to any one of Embodiments 2-17, wherein the oligonucleotide is up to 60 nucleotides in length.
Embodiment 25. The method of any one of Embodiments 1 or 3-24 or the use of any one of Embodiments 2-17 or 24, wherein each of the oligonucleotide fragments are 4-16 nucleotides in length, optionally 6-9 nucleotides in length.
Embodiment 26. The method of any one of Embodiments 1 or 3-25 or the use of any one of Embodiments 2-17, 24 or 25, wherein the oligonucleotide fragments are single-stranded.
Embodiment 27. The method of any one of Embodiments 1 or 3-25 or the use of any one of Embodiments 2-17, 24 or 25, wherein the oligonucleotide fragments are double-stranded, optionally wherein one or more of the double-stranded oligonucleotide fragments comprises one or two single-stranded overhang(s).
Embodiment 28. The method of any one of Embodiments 1 or 3-27 or the use of any one of Embodiments 2-17 or 24-27, wherein one or more of the oligonucleotide fragments comprises a chemical modification.
Embodiment 29. The method or use of Embodiment 28, wherein the chemical modification is selected from:
   (a) a modified backbone, optionally selected from a phosphorothioate (e.g. chiral phosphorothioate) or methylphosphonate internucleotide linkage;
   (b) a modified nucleotide, optionally selected from 2′-O-methyl (2′-OMe), 2′-flouro (2′-F), 2′-deoxy, 2′-deoxy-2′-fluoro, 2′-O-methoxyethyl (2′-O-MOE), 2′-O-aminopropyl (2′-O-AP), 2′-O-dimethylaminoethyl (2′-O-DMAOE), 2′-O-dimethylaminopropyl (2′-O-DMAP), 2′-O-dimethylaminoethyloxyethyl (2′-O-DMAEOE), 2′-O—N-methylacetamido (2′-O-NMA), locked nucleic acid (LNA), glycol nucleic acid (GNA), phosphoramidate (e.g. mesyl phosphoramidate), 2′,3′-seco nucleotide mimic, 2′-F-arabino nucleotide, abasic nucleotide, 2′-amino modified nucleotide, 2′-alkyl-modified nucleotide, morpholino nucleotide, vinylphosphonate (e.g. 5′ vinylphosphonate), and cyclopropyl phosphonate deoxyribonucleotide; and/or
   (c) conjugation to a ligand, optionally wherein the ligand comprises one or more N-Acetylgalactosamine (GalNAc) derivatives.
Embodiment 30. The method of any one of Embodiments 1 or 3-29 or the use of any one of Embodiments 2-17 or 24-29, wherein the ATP-dependent nucleic acid ligase and/or the PPK are immobilised.
Embodiment 31. The method or use of Embodiment 30, wherein the ATP-dependent nucleic acid ligase and/or the PPK are immobilised on a solid material by chemical bond or a physical adsorption method.
Embodiment 32. A composition comprising:
   i. an ATP-dependent nucleic acid ligase;
   ii. a PPK;
   iii. ATP and/or AMP;
   iv. a divalent cation; and
   v. polyphosphate.
Embodiment 33. The composition of Embodiment 32, further comprising two or more oligonucleotide fragments.
Embodiment 34. A kit comprising:
   i. an ATP-dependent nucleic acid ligase;
   ii. a PPK;
   iii. ATP and/or AMP;
   iv. polyphosphate;
   v. a divalent cation; and
   vi. instructions for use in a method of producing an oligonucleotide from two or more oligonucleotide fragments.
Embodiment 35. The composition of Embodiment 32 or Embodiment 33 or the kit of Embodiment 34, wherein the polyphosphate is a polyphosphate salt.
Embodiment 36. The composition or kit of Embodiment 35, wherein the polyphosphate salt is selected from Graham's salt and Maddrell's salt.
Embodiment 37. The composition of any one of Embodiments 32, 33, 35 or 36 or the kit of any one of Embodiments 34-36, wherein the divalent cation is Mg²⁺ or Mn²⁺.
Embodiment 38. The composition of any one of Embodiments 32, 33 or 35-37 or the kit of any one of Embodiments 34-37, wherein the concentration of divalent cation is 5-100 mM, optionally 30-50 mM.
Embodiment 39. A fusion polypeptide comprising:
   a) a PPK domain; and
   b) an ATP-dependent nucleic acid ligase domain.
Embodiment 40. The fusion polypeptide of Embodiment 39, wherein the fusion polypeptide comprises a linker.
Embodiment 41. The fusion polypeptide of Embodiment 39 or Embodiment 40, wherein the PPK is PPK12 or ajPAP.
Embodiment 42. The fusion polypeptide of any one of Embodiments 39-41, wherein the PPK domain comprises an amino acid sequence that has at least 85% identity with the amino acid sequence of any one of SEQ ID NOs: 5-7.
Embodiment 43. The fusion polypeptide of any one of Embodiments 39-42, wherein the ATP-dependent nucleic acid ligase domain is an RNA ligase domain.
Embodiment 44. The fusion polypeptide of Embodiment 43, wherein the RNA ligase domain is a double-stranded RNA (dsRNA) ligase domain.
Embodiment 45. The fusion polypeptide of Embodiment 43 or Embodiment 44, wherein the dsRNA ligase is a member of the RNA ligase 2 family.
Embodiment 46. The fusion polypeptide of Embodiment 45, wherein the dsRNA ligase is Bacteriophage RB69 RNA ligase 2.
Embodiment 47. The fusion polypeptide of any one of Embodiments 39-42, wherein the ATP-dependent nucleic acid ligase domain is a DNA ligase domain.
Embodiment 48. The fusion polypeptide of Embodiment 47, wherein the DNA ligase domain is a T4 DNA ligase domain.
Embodiment 49. The fusion polypeptide of any one of Embodiments 39-48, wherein the ATP-dependent nucleic acid ligase domain comprises an amino acid sequence that has at least 85% sequence identity with the amino acid sequence of any one of SEQ ID NOs: 1-4.
Embodiment 50. The fusion polypeptide of any one of Embodiments 39-48, wherein the ATP-dependent nucleic acid ligase domain comprises an amino acid sequence that has at least 85% sequence identity with the amino acid sequence of any one of SEQ ID NOs: 1-4 or 88.
Embodiment 51. The fusion polypeptide of any one of Embodiments 40-50, wherein the linker is located between the PPK domain and the ATP-dependent nucleic acid ligase domain.
Embodiment 52. The fusion polypeptide of any one of Embodiments 40-51, wherein the PPK domain is located at the N-terminus of the linker and the ATP-dependent nucleic acid ligase domain is located at the C-terminus of the linker.
Embodiment 53. The fusion polypeptide of any one of Embodiments 39-52, wherein the fusion polypeptide comprises a purification tag.
Embodiment 54. The fusion polypeptide of any one of Embodiments 40-53, wherein the linker comprises a purification tag.
Embodiment 55. The fusion polypeptide of Embodiment 53 or Embodiment 54, wherein a purification tag is located at the N- and/or C-terminus of the fusion polypeptide.
Embodiment 56. The fusion polypeptide of any one of Embodiments 40-55, wherein the linker is a polypeptide linker comprising at least 3 amino acids, optionally at least 6 amino acids.
Embodiment 57. The fusion polypeptide of Embodiment 56, wherein the linker comprises an amino acid sequence selected from:

a)
(SEQ ID NO: 19)
HHHHHH,
optionally
(SEQ ID NO: 20)
HHHHHHHHHH;
b)
(SEQ ID NO: 21)
ENLYFQS;
c)
(SEQ ID NO: 22)
ENLYFQG;
d)
(SEQ ID NO: 23)
SSGSSG;
e)
(SEQ ID NO: 24)
GSAGSAAGSGEF;
and/or
f)
(SEQ ID NO: 25)
GSSGSGSSSGGSSSSGSS.

Embodiment 58. The fusion polypeptide of any one of Embodiments 39-57, wherein the fusion polypeptide comprises an amino acid sequence that has at least 85% sequence identity with the amino acid sequence of any one of SEQ ID NOs: 8-18.
Embodiment 59. The fusion polypeptide of any one of Embodiments 39-57, wherein the fusion polypeptide comprises an amino acid sequence that has at least 85% sequence identity with the amino acid sequence of any one of SEQ ID NOs: 8-18, 90, 92, 94, 96, or 98.
Embodiment 60. The method according to any one of Embodiments 1 or 3-31 or the use according to any one of Embodiments 2-17 or 24-31, wherein the ATP-dependent nucleic acid ligase and the PPK are provided as a fusion polypeptide as defined in any one of Embodiments 39-59.
Embodiment 61. A nucleic acid molecule encoding the fusion polypeptide of any one of the Embodiments 39-59.
Embodiment 62. The nucleic acid molecule of Embodiment 61, wherein the nucleic acid molecule comprises a nucleic acid sequence that has at least 85% sequence identity with the nucleic acid sequence of any one of SEQ ID NOs: 34-36.
Embodiment 63. The nucleic acid molecule of Embodiment 61 or Embodiment 62, wherein the nucleic acid molecule comprises a nucleic acid sequence that has at least 85% sequence identity with the nucleic acid sequence of any one of SEQ ID NOs: 30-33.
Embodiment 64. The nucleic acid molecule of Embodiment 61 or Embodiment 62, wherein the nucleic acid molecule comprises a nucleic acid sequence that has at least 85% sequence identity with the nucleic acid sequence of any one of SEQ ID NOs: 30-33 or 87.
Embodiment 65. A vector comprising the nucleic acid of any one of Embodiments 61-64.
Embodiment 66. The vector according to Embodiment 65, wherein the vector is selected from a plasmid, a cosmid, a bacteriophage or a viral vector.
Embodiment 67. A host cell comprising the nucleic acid molecule of any one of Embodiments 61-64 or the vector of Embodiment 65 or Embodiment 66.
Embodiment 68. The host cell of Embodiment 67, wherein the host cell is E. coli.
Embodiment 69. Use of a fusion polypeptide according to any one of Embodiments 39-59 in an ATP-dependent nucleic acid ligation reaction.
Embodiment 70. The use of Embodiment 69, wherein the rate of nucleic acid ligation exceeds the rate of nucleic acid ligation of a control; wherein the control comprises:
   (a) a first protein consisting of the PPK domain of Embodiment 39; and
   (b) a second protein consisting of the ATP-dependent nucleic acid ligase domain of Embodiment 39;
      wherein said first and second proteins are not linked.
Embodiment 71. Use of a fusion polypeptide according to any one of Embodiments 39-59 in a method of producing an oligonucleotide from two or more oligonucleotide fragments.
Embodiment 72. The method of any one of Embodiments 1, 3-31 or 60 or the use of any of Embodiments 2-17, 24-31, or 69-71, wherein the oligonucleotide is a therapeutic oligonucleotide.
Embodiment 73. The method of any one of Embodiments 1, 3-31, 60 or 72 or the use of any of Embodiments 2-17, 24-31 or 69-72, wherein the oligonucleotide product is at least 80% pure, optionally wherein the oligonucleotide product is at least 85% pure, at least 90% pure, at least 95% pure, optionally wherein the oligonucleotide product is at least 98% pure.

Different features and embodiments of the present disclosure are exemplified in the following representative examples, which are intended to be illustrative and not restrictive.

### EXAMPLES

The following Examples, including experiments and results achieved, are provided for illustrative purposes only and are not to be construed as limiting the present invention.

In the Examples below, the following abbreviations apply: ppm (parts per million); M (molar); mM (millimolar), uM and μM (micromolar); nM (nanomolar); mol (moles); gm and g (gram); mg (milligrams); ug and μg (micrograms); L and l (liter); ml and mL (milliliter); cm (centimeters); mm (millimeters); um and μm (micrometers); sec. (seconds); min(s) (minute(s)); h(s) and hr(s) (hour(s)); U (units); MW (molecular weight); rpm (rotations per minute); psi and PSI (pounds per square inch); ° C. (degrees Centigrade); RT and rt (room temperature); OD600 (Optical density at 600 nm), CAM and cam (chloramphenicol); DMSO (dimethylsulfoxide); FP (Fermentation powder); FWC (Frozen whole cells), LWC (Lyophilized whole cells), PMBS (polymyxin B sulfate); IPTG (isopropyl β-D-1-thiogalactopyranoside); AMP (adenosine-monophosphate); ADP (adenosine-diphosphate); ATP (adenosine-triphosphate), PolyP (polyphosphate); LB (Lysogeny broth); TB (Terrific Broth; 12 g/L bacto-tryptone, 24 g/L yeast extract, 4 mL/L glycerol, 65 mM potassium phosphate, pH 7.0, 1 mM MgSO₄); HEPES (HEPES zwitterionic buffer; 4-(2-hydroxyethyl)-piperazineethanesulfonic acid); SFP (shake flask powder); CDS (coding sequence); DNA (deoxyribonucleic acid); RNA (ribonucleic acid); E. coli W3110 (commonly used laboratory E. coli strain, available from the Coli Genetic Stock Center [CGSC], New Haven, CT); HTP (high throughput); HPLC (high pressure liquid chromatography); GC (gas chromatography), MS (mass spectrometer), RF (Rapid Fire), FIOP (fold improvements over positive control); Microfluidics (Microfluidics, Corp., Westwood, MA); Sigma-Aldrich (Sigma-Aldrich, St. Louis, MO; Difco (Difco Laboratories, BD Diagnostic Systems, Detroit, MI); Agilent (Agilent Technologies, Inc., Santa Clara, CA); Corning (Corning, Inc., Palo Alto, CA); Dow Corning (Dow Corning, Corp., Midland, MI); and Gene Oracle (Gene Oracle, Inc., Mountain View, CA).

### Example 1

### Results and Discussion

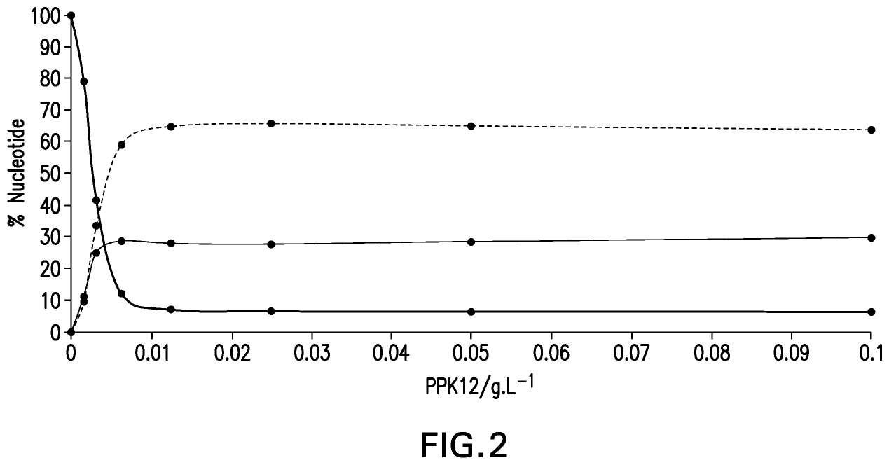

Polyphosphate kinase (PPK12) has previously been shown to convert AMP to ATP (via an ADP intermediate) using polyphosphate as a phosphate donor (Tavanti, M., Hosford, J., Lloyd, R. C. & Brown, M. J. B. Green Chemistry 23, 828-837 (2021)). To validate the activity of PPK12, wildtype PPK12 was produced in E. coli and applied in the reaction as lyophilized cell free extract, referred to herein as shake flask powder (SFP). Starting from AMP and 100 equivalents of polyphosphate, ATP production was monitored at different doses of PPK12; the reaction reached equilibrium with a maximum of 66% conversion to ATP (FIG. 2), which is consistent with previous reports.

To improve the catalytic activity of PPK12, saturation mutagenesis libraries were designed, constructed and screened. The best performing enzyme identified in screening (SEQ ID NO: 6) was approximately 2-fold more active than the wildtype enzyme and was used in all subsequent experiments.

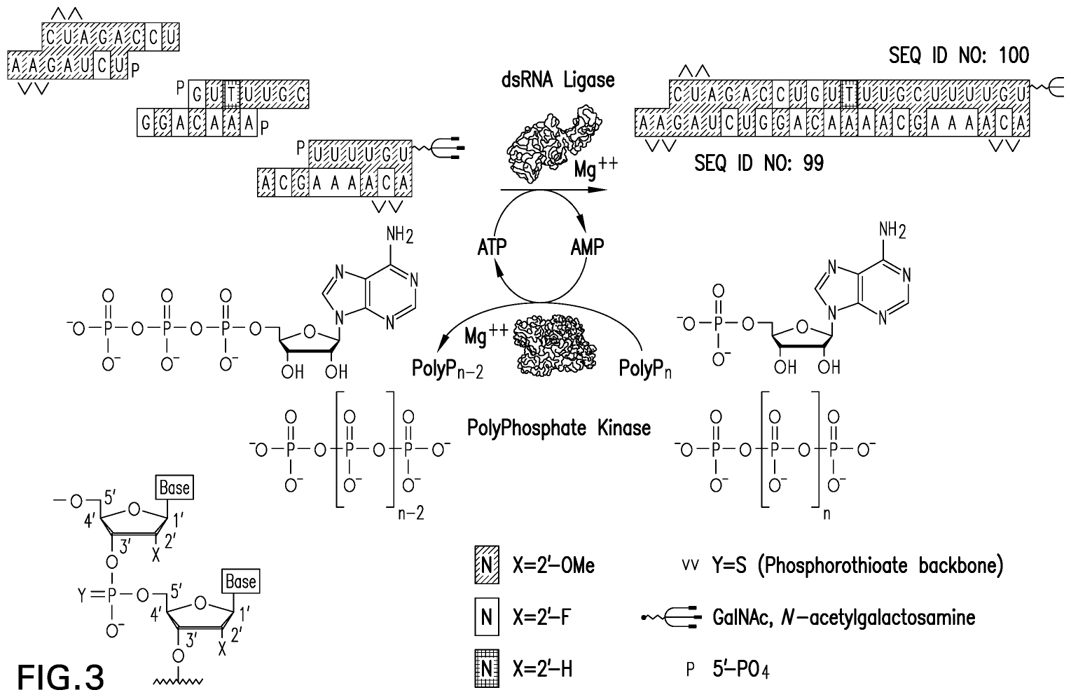

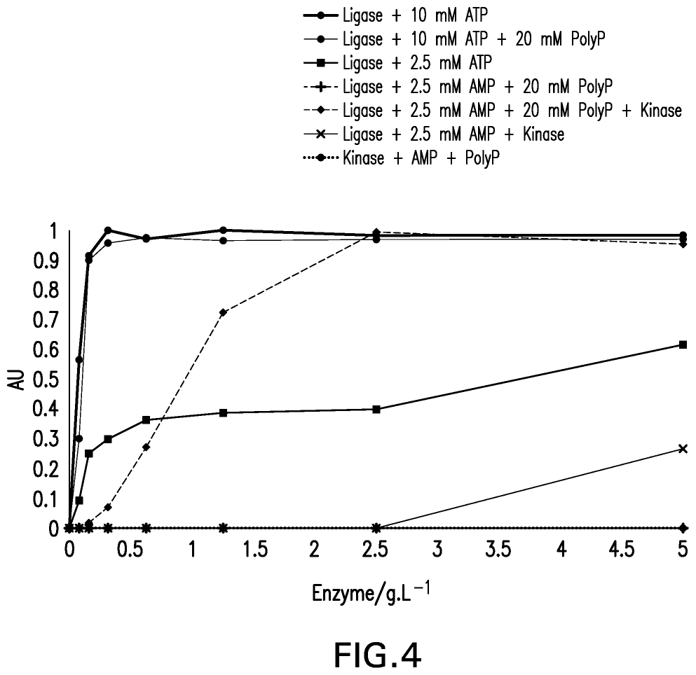

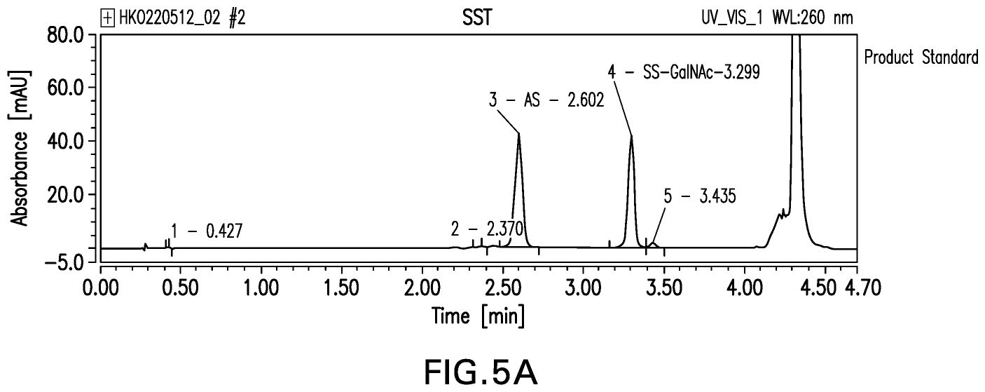

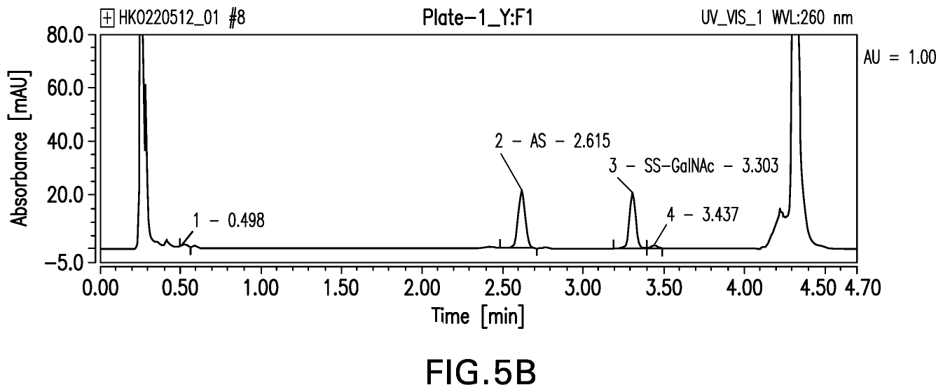

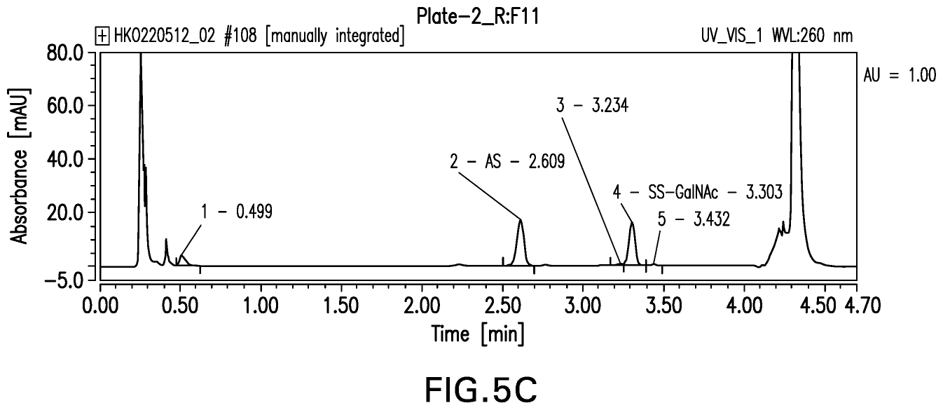

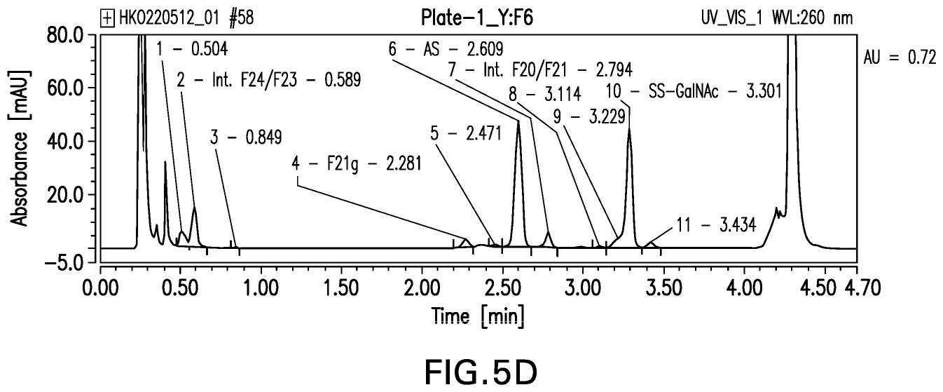

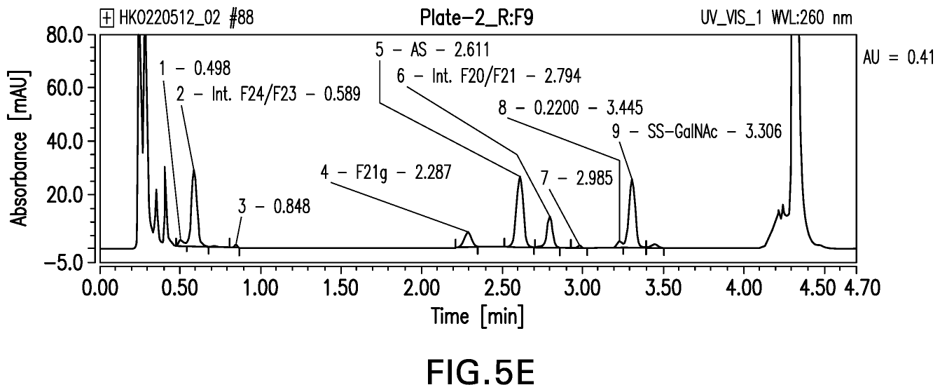

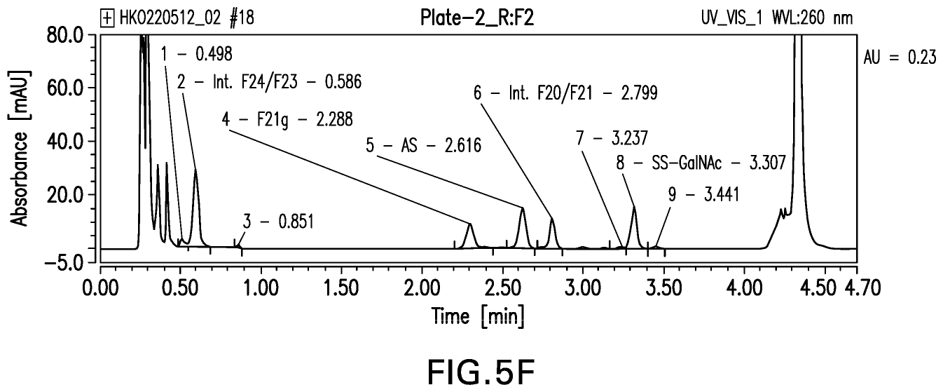

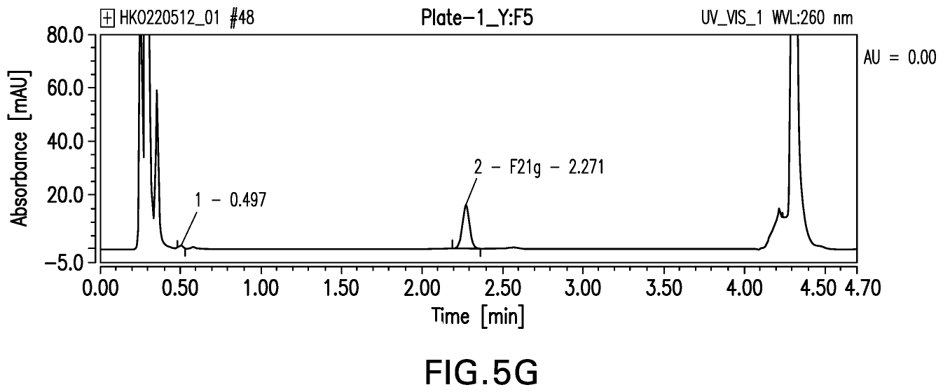

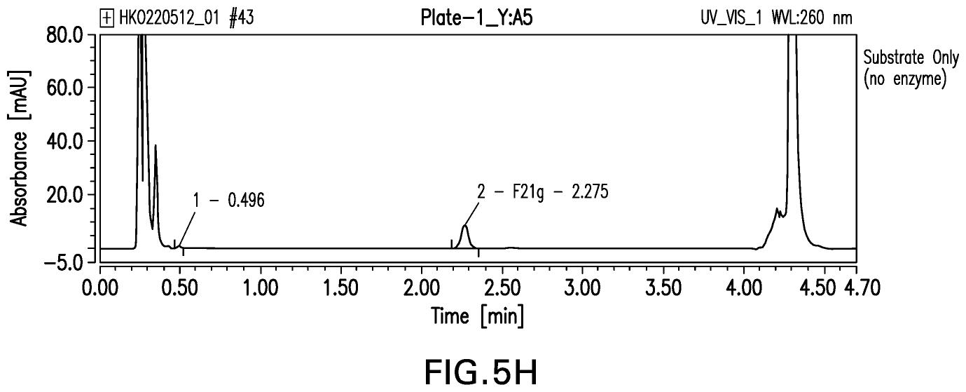

To develop an efficient biocatalytic process it is important to maximize the space-time yield of the ligation reaction. To this end, the ligation reaction was performed with a high substrate concentration of at least 1 mM. To generate the full-length oligonucleotide (in this example, siRNA) product from the oligonucleotide fragments as depicted in FIG. 3, the ligase must catalyze four ligation reactions. Therefore, to process 1 mM substrate, at least 4 mM ATP is required. In reality, an excess of ATP is necessary to achieve complete ligation of the substrate. Using a dsRNA ligase evolved to work at high substrate concentration (SEQ ID NO: 2) full conversion starting from 1 mM substrate oligonucleotides was obtained in the presence of 10 mM ATP (FIG. 2, FIG. 5). As expected, the addition of only 2.5 mM ATP resulted in only partial product formation (FIG. 4). In contrast, no products were observed when AMP was supplied in place of ATP (FIG. 4).

As demonstrated herein, ligation activity in the presence of AMP can be rescued by the addition of PPK and polyphosphate (FIG. 4, FIG. 5). In the presence of a sub-stoichiometric concentration of AMP (2.5 mM), the oligonucleotide fragments were fully converted to the oligonucleotide products, confirming that PPK12 converts AMP to ATP multiple times during the ligation reaction. These data indicate that PPK12 and polyphosphate function as an effective ATP regeneration system during the ligation reaction. Control experiments without polyphosphate, without kinase and without ligase indicate that each of these components must be present to achieve ligation (FIG. 4).

The use of a sub-stoichiometric quantity of AMP has a positive impact on the cost and the sustainability of the ligation reaction. To offset potential cost and resource demands associated with the use of two separate enzymes (i.e. a ligase and a kinase), genetic fusions of the kinase and ligase genes were generated and used to express a single fusion polypeptide comprising both a kinase and a ligase domain. A linker was included between the two enzymes to allow each of the enzymes to help maintain their activity without inference from the other enzyme. Production of a bifunctional biocatalyst from a single fermentation instead of two advantageously saves time, effort and money associated with enzyme production.

Eleven different fusion constructs were designed to investigate whether engineered PPK could be linked to engineered dsRNA ligase. These constructs, termed “Klignases” are provided in table 1.

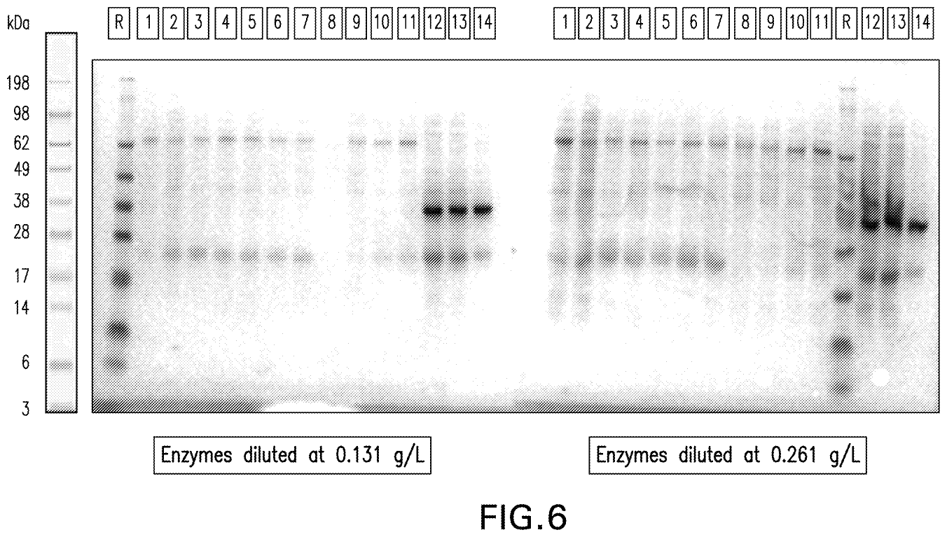

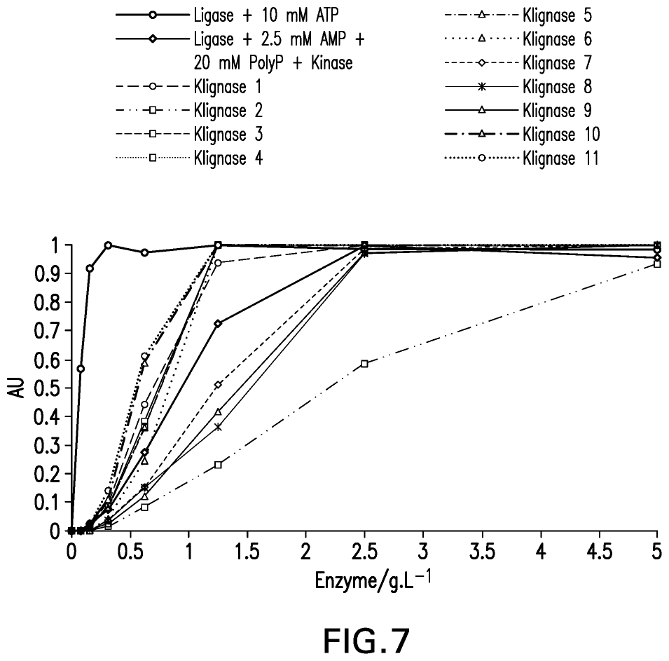

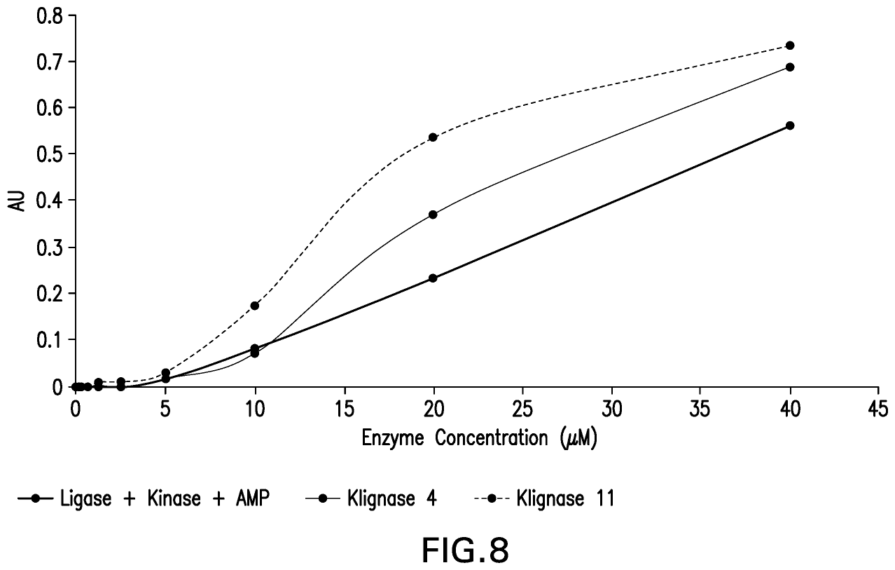

All Klignases were expressed as soluble proteins as determined via SDS-PAGE analysis (FIG. 6). To evaluate the activity of the Klignases compared to the respective non-linked enzymes, the formation of oligonucleotide product under the previously described ATP recycling conditions was monitored at various enzyme concentrations. Oligonucleotide product was identified for all Klignases, indicating that both ATP synthesis and ligation activity were maintained (FIG. 7, FIG. 5). The relative activity differed between the fusion constructs (FIG. 7, FIG. 5) with constructs containing the kinase at the N-terminus (Klignases 1, 3, 4, 5, 10 and 11) typically being more active than the non-linked ligase. The most active Klignase constructs (Klignases 10 and 11) exhibited approximately 1.5-2 fold higher ligase activity than the non-linked enzymes. Exemplary Klignases 4 and 11 exhibit higher ligase activity as compared to the non-linked ligase at equimolar amounts (FIG. 8). Without wishing to be bound by theory, it is possible that the linked enzymes are more stable. Alternatively, the close proximity of the kinase to the ligase could supply the ligase with a local supply of ATP, thereby accelerating the reaction.

TABLE 1
Design of PPK/dsRNA ligase fusion constructs exploring different orders
of the constituent subunits and various linkers, whereby His₆ is a
hexa-histidine tag, which can be used to purify or immobilize the
enzyme; TEV is the seven amino acid recognition sequence for the
tobacco etch virus protease (ENLYFQS (SEQ ID NO: 21)). N.B. It is
not intended that the Klignases are cleaved using TEV
protease, but the amino sequence is included in the designs as an
illustrative spacer that is tolerated in the original ligase construct.

| Klignase | Fusion construct design |
|---|---|
| 1 | Kinase-His₆-TEV-Ligase (SEQ ID NO: 8) |
| 2 | Kinase-His₁₀-Ligase (SEQ ID NO: 9) |
| 3 | His₆-TEV-Kinase-SSGSSG-Ligase (SEQ ID NO: 10) |
| 4 | His₆-TEV-Kinase-GSAGSAAGSGEF-Ligase (SEQ ID NO: 11) |
| 5 | His₆-TEV-Kinase-GSSGSGSSSGGSSSSGSS-Ligase (SEQ ID NO: 12) |
| 6 | His₆-TEV-Ligase-GSAGSAAGSGEF-Kinase (SEQ ID NO: 13) |
| 7 | His₆-TEV-Ligase-GSSGSGSSSGGSSSSGSS-Kinase (SEQ ID NO: 14) |
| 8 | Ligase-GSAGSAAGSGEF-Kinase-His₆ (SEQ ID NO: 15) |
| 9 | Ligase-GSSGSGSSSGGSSSSGSS-Kinase-His₆ (SEQ ID NO: 16) |
| 10 | Kinase-His₆-SSGSSG-His₆-TEV-Ligase (SEQ ID NO: 17) |
| 11 | Kinase-SSGSSG-His₆-SSGSSG-TEV-Ligase (SEQ ID NO: 18) |

### Conclusion

The enzymatic ligation of short oligonucleotide fragments offers a sustainable and economical alternative to the solid phase chemical synthesis of full-length (e.g. therapeutic) oligonucleotides. However, one drawback of the ligase catalyzed reaction is the need for a stoichiometric quantity of an expensive cofactor, ATP. As demonstrated herein, the addition of a second enzyme to the reaction, a polyphosphate kinase (e.g. PPK12 or an engineered variant of PPK12) and polyphosphate facilitates cofactor regeneration, enabling complete ligation at high substrate concentration using a ligase (e.g. a dsRNA ligase or an engineered dsRNA ligase) in the presence of a sub-stoichiometric quantity of AMP, which is significantly cheaper than ATP. The kinase and ligase (e.g. PPK and dsRNA ligase) can be expressed together as a single polypeptide, further simplifying and reducing the costs of the biocatalytic process.

Advantageously, linking the kinase and ligase can improve the activity of the ligase as compared to the activity of the non-linked ligase.

### Methods

### Oligonucleotide Synthesis

Substrate fragments and reference oligonucleotides were synthesized by commercial partners. Oligonucleotide substrates 1-6 and products AS (antisense strand) and SS (sense strand) are provided in table 2.

TABLE 2
Substrate and product oligonucleotide sequences.

| Sample | Sequence |
|---|---|
| Substrate 1 (F19) | mC-(s)-mU-(s)-mA-mG-mA-mC-fC-mU |
| Substrate 2 (F20) | pfG-mU-dT-mU-mU-mG-mC |
| Substrate 3 (F21) | pmU-mU-mU-mU-mG-mU-GaINAc |
| Substrate 4 (F22) | mA-(s)-fC-(s)-mA-fA-fA-fA-mG-fC-mA |
| Substrate 5 (F23) | pfA-mA-fA-mC-fA-mG-fG |
| Substrate 6 (F24) | pmU-fC-mU-mA-mG-(s)-mA-(s)-mA |
| Product AS | mA-(s)-fC-(s)-mA-fA-fA-fA-mG-fC-mA-fA-mA-fA- |
| | mC-fA-mG-fG-mU-fC-mU-mA-mG-(s)-mA-(s)-mA |
| | (SEQ ID NO: 99) |
| Product SS | mC-(s)-mU-(s)-mA-mG-mA-mC-fC-mU-fG-mU-dT-mU- |
| | mU-mG-mC-mU-mU-mU-mU-mG-mU-GalNAc |
| | (SEQ ID NO: 100) |

Sequences are denoted in the 5′ - 3′ direction.
mX = 2′-OMe modified nucleotides;
fX = 2′-F modified nucleotides;
dX = deoxyribonucleotides;
-(s)- = phosphorothioate bond;
-GalNAc = N-acetyl galactosamine;
AS = antisense strand;
SS = sense strand

### DNA Sequences Encoding Enzymes

>PPK12 with C-terminal tag (GQTGHHHHHH; SEQ ID NO: 27)
(SEQ ID NO: 48)
ATGATTAACATCTATAAAATTGATAAACTGAATAATTTTAACTTGAACAATCATAAG
ACCGACGACTACAGCCTGTGCAAGGATAAAGATACTGCGCTCGAACTGACCCAGAA
GAACATCCAGAAGATTTATGACTACCAACAGAAACTGTATGCCGAGAAGAAAGAAG
GCCTTATCATCGCATTCCAAGCTATGGATGCAGCGGGTAAAGATGGAACGATTCGC
GAAGTGTTAAAAGCGTTAGCTCCGCAGGGTGTTCACGAGAAACCATTTAAAAGTCC
GTCGTCGACAGAACTGGCCCATGATTACTTGTGGCGTGTCCACAATGCCGTACCTGA
GAAAGGCGAAATTACCATCTTCAACCGCTCCCATTATGAAGACGTGCTGATTGGGA
AAGTTAAAGAACTGTACAAGTTTCAGAACAAAGCGGATCGTATTGACGAAAATACG
GTGGTGGATAACCGCTATGAGGATATCCGTAATTTCGAGAAATATCTCTACAACAAC
TCTGTCCGCATCATCAAAATCTTTCTGAACGTCAGCAAGAAAGAACAAGCAGAACG
TTTTCTGAGCCGCATTGAAGAACCGGAGAAGAATTGGAAATTCAGTGACTCAGATTT
TGAAGAGCGTGTTTATTGGGACAAATATCAGCAGGCGTTCGAAGATGCCATTAATG
CGACCTCCACGAAAGATTGTCCCTGGTACGTAGTACCGGCTGATCGCAAATGGTACA
TGCGTTATGTGGTGAGCGAAATCGTTGTTAAGACCCTTGAAGAAATGAACCCAAAA
TACCCGACGGTGACTAAAGAGACATTGGAACGCTTTGAAGGCTATCGTACCAAACT
GCTGGAGGAGTATAATTATGACTTAGATACCATTCGCCCTATTGAAAAGGGCCAAA
CTGGCCACCATCACCATCACCATTAG
>Optimized PPK with C-terminal tag (GQTGHHHHHH;
SEQ ID NO: 27)
(SEQ ID NO: 49)
ATGATTAACATCTATAAAATTGATAAACTGAATAATTTTAACTTGAACAATCATAAG
ACCGACGACTACAGCCTGTGCAAGGATAAAGATACTGCGCTCGAACTGACCCAGAA
GAACATCCAGAAGATTTATGACTACCAACAGAAACTGTATGCCGAGAAGAAAGAAG
GCCTTATCATCGCATTCCAAGCTATGGATGCAGCGGGTAAAGATGGAACGATTCGC
GAAGTGTTAAAAGCGTTAGCTCCGCAGGGTGTTCACGAGAAACCATTTAAAAGTCC
GTCGTCGACAGAACTGGCCCATGATTACTTGTGGCGTGTCCACAATGCCGTACCTGA
GAAAGGCGAAATTACCATCTTCAACCGCTCCCATTATGAAGACGTGCTGATTGGGA
AAGTTAAAGAACTGTACAAGTTTCAGAACAAAGCGGATCGTATTGACGAAAATACG
GTGGTGGATAACCGCTATGAGGATATCCGTAATTTCGAGAAATATCTCTACAACAAC
TCTGTCCGCATCATCAAAATCTTTCTGAACGTCAGCAAGAAAGAACAAGCAGAACG
TTTTCTGAGCCGCATTGAAGAACCGGAGAAGAATTGGAAATTCAGTGACTCAGATTT
TGAAGAGCGTGTTTATTGGGACAAATATCAGCAGGCGTTCGAAGATGCCATTAATG
CGACCTCCACGAAAGATTGTCCCTGGTACGTAGTACCGGCTGATCGCAAATGGTACA
TGCGTTATGTGGTGAGCGAAATCGTTGTTAAGACCCTTGAAGAAATGAACCCAAAA
TACCCGACGGTGACTAAAGAGACATTGGAACGCTTTGAAGGCTATCGTACCAAACT
GCTGGAGGAGTATAATTATGGGTTAGATACCATTCGCCCTATTGAAAAGGGCCAAA
CTGGCCACCATCACCATCACCATTAG
>dsRNA ligase with N-terminal tag (MHHHHHHENLYFQS;
SEQ ID NO: 26)
(SEQ ID NO: 50)
ATGCATCATCATCACCATCACGAAAATCTGTATTTTCAGAGCATGTTCAAGAAATAC
TCCTCCTTGGAGAACCACTATAACTCGAAATTCATTGAAAAACTGTACACCAATGGC
CTTACGACGGGTGTGTGGGTTGCGCGCGAGAAGATCCATGGGACCAACTTTAGCCT
GATTATCGAACGTGATAATGTCACTTGCGCCAAACGTACAGGCCCGATTCTCCCAGC
TGAAGACTTCTATGGTTACGAAATCGTGCTGAAGAAATACGATAAAGCAATTAAGA
CCGTCCAAAAGTTTATGTATACAGCACGCGCCGTGTCTTACCAGGTATTTGGAGAAT
TTGCTGGTGGCGGCATCCAGAAAGGTGTTGATTATGGCGAGAAAGACTTCTATGTGT
TCGATATTTTAATCAACACCGAATCAGGTGACAATACGTATCTGACCGATTACGAGA
TGCAAGACTTCTGTAACGAATTTGGGTTTAAGATGGCGCCGATGTTAGGACGCGGCA
CCTTCGATAGTCTGATTATGATTCCTAACGATTTGGATAGTGTACTTGCGGCGTATA
ATGCCACTGCAAGCGAAGACCTGGTTGAAGCGAACAACTGCGTCTTTGATGCCAAT
GTGATCGGTGACAATACGGCTGAGGGCTACGTTCTGAAACCGTGTTTTCCCAAATGG
CTCCCGAACGGTACCCGTGTGGCGATCAAATGCAAGAACTCGAAATTCAGCGAGAA
AAAGAAATCTGACAAACCGATTAAGACTCAGGTACCACTGACGGAAATCGATAAGA
ATCTGTTGGATGTCCTGGCCTGTTATGTGACCTTAAACCGCGTCAATAACGTGATTTC
GAAGATTGGCACAGTTACCCCGAAAGATTTTGGGAAAGTTATGGGCCTGACTGTGC
AGGATATCCTCGAAGAAACCTCACGTGAAGGGATTGTACTTACGACGAGTGACAAC
CCTAATCTGGTGAAGAAAGAATTGGTTCGCATGGTCCAAGATGTGTTACGTCCCGCA
TGGATCGAGCTGGTTTCCTAA
>Optimized dsRNA ligase with N-terminal tag
(MHHHHHHENLYFQS; SEQ ID NO: 26)
(SEQ ID NO: 51)
ATGCATCATCATCACCATCACGAAAATCTGTATTTTCAGAGCATGTTCAAGAAATAC
TCCTCCTTGGAGAACCACTATAACTCGAAATTCATTGAAAAACTGTACACCAATGGC
CTTACGACGGGTGTGTGGGTTGCGCGCGAGAAGATTCATGGGGCGAACTTTAGCCT
GATTATCGAACGTGATAATGTCACTTGCGCCAAACGTACAGGCCCGATTCTCCCAGC
TGAAGACTTCTATGGTTACGAAATCGTGCTGAAGAAATACGATAAAGCAATTAAGA
CCGTCCAAAAGTTTATGTATACAGCACGCGCCGTGTCTTACCAGGTATTTGGAGAAT
TTGCTGGTGGTGGCATCCAGAAAGGTGTTGATTATGGCGAGAAAGACTTCTATGTGT
TCGATATTTTAATCAACACCGAATCAGGTGACAATACGTATCTGACCGATTACGAGA
TGCAAGACTTCTGTAACGAATTTGGGTTTAAGATGGCGCCTATGTTAGGACGCGGCA
CCTTCGATAGTCTGATTATGATTCCTAACGATTTGGATAGTGTACTTGCGGCGTATA
ATGCCACTGCAAGCGAAGACCTGGTTGAAGCGAACAACTGCGTCTTTGATGCCAAT
GTGATCGGTGACAATACGGCTGAGGGCTACGTTCTGAAACCGTGTTTTCCCAAATGG
CTCCCGAACGGTAACCGTGTGATTATCAAATGCAAGAACTCGAAATTCAGCGAGAA
AAAGAAATCTGACAAACCGATTAAGACTCAGGTACCACTGACGGAAATCGATAAGA
ATCTGTTGGATGTCCTGGCCTGTTATGTGACCTTAAACCGCGTCAATAACGTGATTTC
GAAGATTGGCACAGTTACCCCGAAAGATTTTGGGAAAGTTATGGGCCTGACTGTGC
AGGATATCCTCGAAGAAACCTCACGTGAAGGGATTGTACTTACGACGAGTGACAAC
CCTAATCTGGTGAAGAAAGAATTGGTTCGCATGGTCCAAGATGTGTTACGTCCCGCA
TGGATCGAGCTGGTTTCCTAA

### Enzyme Expression Shake Flask Powder Production

DNA encoding engineered enzymes were cloned into the pCK110900 vector and transformed into W3110 E. coli electro-competent cells. A single bacterial colony was picked and grown in 25 mL of LB media (Teknova) supplemented with 1% glucose and 30 μg·mL⁻¹ chloramphenicol, at 37° C., 200 rpm, 85% humidity overnight. Following overnight growth, 5 mL of the culture was used to inoculate a shake flask containing 250 mL of TB media (Teknova) supplemented with 30 μg·mL⁻¹ chloramphenicol and grown at 30° C., 200 rpm, 85% humidity until the optical density (OD₆₀₀) of the culture reached 0.7. Enzyme expression was induced by the addition of IPTG to a final concentration of 1 mM and the cultures further grown at 30° C., 200 rpm, 85% humidity for 16 h. Cells were harvested by centrifugation at 4 000 g at 4° C. for 5 min and the media discarded. Cell pellets were stored at −80° C. until ready for use.

Cell pellets were thawed and resuspended in 30 mL of 50 mM Tris·HCl pH 7.5 supplemented with 1 mM DTT and lysed via passage through a microfluidizer (Microfluidics, LM20). The lysate was clarified via centrifugation at 40 000 g at 4° C. for 30 min, frozen at −80° C. and then lyophilized. The resulting shake flask powder (SFP) was stored at −20° C. until used.

### ATP Synthesis Reactions

ATP synthesis activity of wild type PPK12 was investigated at different enzyme concentrations. A 5 g/L stock solution of wild-type PPK12 was prepared by dissolving 50 mg of SFP in 10 mL of 50 mM Tris·HCl pH 8.0. The stock solution was diluted accordingly to prepare a seven sample, two-fold serial dilution starting at 0.5 g/L PPK12. A blank control containing 0 g/L PPK12 was included as an eighth sample.

ATP synthesis reactions were set up using 0.3 mM AMP, 30 mM polyphosphate (Maddrell's salt), 5 mM MgCl₂, 5 mM DTT and 20% (v/v) of the PPK12 dilution series (final reaction concentration starting at 0.1 g/L PPK12) in 50 mM Tris·HCl pH 8.0. Reactions were set up in a BioRad Hardshell PCR plate and incubated in a thermocycler for 30° C. for 30 min. After 30 min, the reaction was quenched by diluting the reaction mixture 2.5-fold in 10 mM EDTA pH 7.0. Samples were analyzed via HPLC as described below.

### HPLC Analysis of ATP Synthesis Reactions

ATP synthesis reactions were analyzed via HPLC using an Agilent Infinity Lab Poroshell 120 HILIC-Z column (50×2.1 mm, 1.7 μm) on a Thermo Scientific UHPLC Vanquish Horizon. Mobile phase A (MPA) is composed of 20 mM ammonium acetate pH 9.0 (Honeywell HPLC grade) in Milli-Q grade water. Mobile phase B (MPB) is composed of acetonitrile (Superlco HPLC grade)/150 mM ammonium acetate pH 10.5. The elution starts with a linear gradient from 95% MPB to 72% MPB in 0.8 min, followed by a second linear gradient to 48% MPB in 0.1 min. 48% MPB is maintained for 0.3 min then changed to 95% MPB in 0.1 min, which is maintained for 0.45 min to re-equilibrate the column for the next sample resulting in a total run time of 1.75 min. The flow rate is set for 1.0 mL/min and the column temperature is set to 25° C. The detection is monitored by UV at λ=260 nm.

Ligation reactions and ATP recycling

Ligation activity and ATP recycling activity were investigated at different concentrations. 50 g/L stock solutions of dsRNA ligase, PPK and Klignase were prepared by dissolving 15 mg of SFP in 300 μL of 50 mM Tris·HCl pH 7.0. For ligation reactions using only dsRNA ligase or kinase, the stock solution was diluted to 25 g/L with 50 mM Tris·HCl pH 7.0. For ligation reactions requiring both ligase and kinase, the two stock solutions were combined 1:1 to give stock solution of 25 g/L of each enzyme. For ligation reactions using a Klignase, the stock solution was undiluted, to account for the fact that the Klignase is approximately twice the molecular weight of the ligase/kinase. The stock solutions were diluted accordingly to prepare a seven sample, two-fold serial dilution starting at 5 g/L ligase and/or kinase and 10 g/L Klignase. Blank controls containing 0 g/L enzyme were included as the eighth sample.

Ligation reactions were set up as follows: Oligonucleotide fragments 1-6 (table 2) were combined to a final concentration of 1 mM in 50 mM Tris·HCl pH 7.0, 5 mM DTT and 20% (v/v) of the appropriate enzyme dilution series. Reaction components including ATP/AMP, MgCl₂, and polyphosphate (Maddrell's salt) were added as incubated according to table 3. Otherwise, the remaining reaction volume was made up with 50 mM Tris·HCl pH 7.0. Reactions were prepared in a BioRad PCR plate and incubated at 30° C. for 24 h immediately followed by a heatshock at 95° C. for 20 min to inactivate the enzymes. The inactivated reactions were subsequently diluted 400-fold in 10 mM EDTA pH 7.0 and analyzed via HPLC as described below.

TABLE 3
Ligation reaction setup highlighting the enzyme(s), cofactor type and
concentration and amount of polyphosphate added to the reaction.

| | Enzyme SFP | | Cofactor | Polyphosphate / |
|---|---|---|---|---|
| Reaction | stock | Cofactor | Conc. / mM | mM |
| 1. | Ligase | ATP | 10 | 0 |
| 2. | Ligase | ATP | 10 | 20 |
| 3. | Ligase | ATP | 2.5 | 0 |
| 4. | Ligase | AMP | 2.5 | 0 |
| 5. | Ligase | AMP | 2.5 | 20 |
| 6. | Ligase / Kinase | AMP | 2.5 | 0 |
| 7. | Ligase / Kinase | AMP | 2.5 | 20 |
| 8. | Kinase | AMP | 2.5 | 20 |
| 9.-19. | Klignase 1-11 | AMP | 2.5 | 20 |

### HPLC Analysis of Ligation Reactions

Ligation reactions were analyzed using an IP—RP-HPLC (Ion-Pairing Reversed-Phase High-Performance Liquid Chromatography) analytical method focusing on the product formation using a Waters® column Acquity UHPLC® BEH C18 column (50×2.1 mm, 1.7 μm) on a Thermo Scientific UHPLC Vanquish Horizon. Mobile phases A (MPA) is composed of 200 mM HFIP (1,1,1,3,3,3-Hexafluoro-2-propanol, Sigma-Aldrich, Purity>99%) and 10 mM TEA (Triethylamine; Sigma-Aldrich BioUltra purity) in Milli-Q grade water. Mobile phase B (MPB) is composed of Methanol (Supelco, HPLC grade,). The elution starts with 16% MPB for 0.5 min, followed by a linear gradient from 16% MPB to 23% MPB in 3.0 min, followed by a second linear gradient from 23% MPB to 90% MPB in 0.1 min. 90% MPB is maintained for 0.20 min, then changed to 16% MPB in 0.20 min and maintained at 16% MPB for 0.70 min resulting in a run time of 4.7 min. The flow rate is set at 0.5 mL·min⁻¹ and the column temperature at 75° C. The detection is monitored by UV at 2=260 nm.

### Note on Using Arbitrary Units (AU) to Compare Ligation Activity

Ideally to compare the activity between samples and account for natural variation in peak intensity between injections, we would calculate the % conversion to product for each sample analyzed using the following equation:

$$\% Conv = \frac{( \frac{A 260 Product ( s )}{{ε}_{p}} )}{\begin{matrix}( \frac{A 260 Product ( s )}{{ε}_{p}} ) + ( \frac{A 260 Substrate ( s )}{{ε}_{s}} ) + \\ ( \frac{A 260 Intermediate ( s )}{{ε}_{i}} )\end{matrix}} \times 100$$

whereby εₚ, εₛ and εᵢ=the extinction coefficient of the product, substrate, and intermediate oligonucleotides respectively. Using the analytical method described herein, it is not possible to resolve all six substrates, four reaction intermediates and two products; all efforts to resolve all species has proven intractable. Therefore, the % conversion according to the above equation cannot be determined. However, using the analytical method described herein it is possible to resolve the well-defined GalNAc-containing oligonucleotides, including the GalNAc containing substrate fragment (F21), a reaction intermediate of F21 ligated to F20 (int. F20/F21) and the product sense strand (SS). Therefore, a pseudo-% conversion can be calculated denoted with arbitrary units (AU), which considers only these well resolved species according to the following equation:

$$AU = \frac{( \frac{A 260 SS}{{ε}_{SS}} )}{( \frac{A 260 SS}{{ε}_{S S}} ) + ( \frac{A 260 F 21}{{ε}_{F 21}} ) + ( \frac{A 260 Int . F 20 / F 21}{{ε}_{int . F 20 / F 21}} )}$$

whereby εₛₛ, ε_{F21} and ε_{int.F20/F21}=the extinction coefficient of the SS, F21, and Int. F20/F21 oligonucleotides respectively (table 4). Using such a calculation an AU=1.0 would imply that no more GalNAc-containing substrate or intermediate oligonucleotides are present in the reaction and that they have all be converted to GalNAc-containing product (SS) and so corresponds with 100% conversion of the sense strand. In reality, for samples where AU=1.0 the only other peak present in the chromatogram corresponds with the product AS, and no other intermediates or starting materials can be identified. Furthermore, the ratio of the SS and AS peaks are consistent with that of the authentic product standard. Taken together AU=1.0 is considered to be essentially equivalent to 100% conversion of both strands. Some example chromatographs illustrating this can be seen in FIG. 5.

TABLE 4
Extinction coefficient of the oligonucleotide
fragments used in the calculation of AU.

| Oligonucleotide | ε (L/mole · cm) |
|---|---|
| F21 | 58800 |
| int. F20/F21 | 123500 |
| SS | 197000 |

### Example 2

Described herein is the directed evolution of a PPK derived from Erysipelotrichaceae bacterium. A series of engineered polypeptides with improved activity are provided.

In this example, % conversion corresponds to the calculated conversion for each single sample expressed in percentage of ATP over the sum of substrate (AMP), intermediate product (ADP) and product (ATP). The amount of AMP, ADP and ATP in each single sample is quantified by HPLC.

### Preparation of Isolated Enzymes

Polynucleotides encoding the polypeptides having phosphotransferase activity, were cloned into the pCK110900 vector system (See e.g., US Pat. App. No. 2006/0195947A1, which is hereby incorporated by reference in its entirety) and subsequently expressed in E. coli W3110fhuA under the control of the lac promoter. The expression vector also contained the P15a origin of replication and the chloramphenicol (CAM) resistance gene.

E. coli W3110fhuA cells were transformed with the pCK110900 plasmid containing the phosphotransferase-encoding genes. Transformed cells were plated out on Lysogeny broth (LB) agar plates containing 1% glucose and 30 μg/mL CAM, and grown overnight at 37° C. Subsequently single colonies were inoculated into 25 mL of LB supplemented with 30 μg/mL CAM and 1% glucose in a 250 ml baffled shake flask. The culture was grown overnight (16-20 hours and optical density (OD₆₀₀)>3.8) in an incubator at 37° C., with shaking at 250 rpm. A 1 L shake flask containing 250 mL of Terrific Broth (TB) media with 30 μg/mL CAM, was inoculated with 5 mL of the grown overnight culture. The 250 mL culture was incubated at 30° C., 250 rpm, for 3-3.5 hours until OD₆₀₀ reached 0.6-0.8. Expression of the phosphotransferase gene was induced by the addition of isopropyl-β-D-thiogalactoside (IPTG) to a final concentration of 1 mM, and growth was continued for an additional 18-20 hours. Cells were harvested by transferring the culture into a centrifuge bottle, which was then centrifuged at 7,000 rpm for 5 minutes at 4° C. The supernatant was discarded, and the remaining cell pellet lysed. For lysis, the cell pellet was resuspended in 30 mL of 50 mM Tris-buffer at pH 7.5 and lysed using a LM20 MICROFLUIDIZER® processor system (Microfluidics). Cell debris was removed by centrifugation at 14,000 rpm for 30 minutes at 4° C. Phosphotransferase enzymes were then isolated from the clarified lysate using standard techniques known in the art, including immobilized metal affinity chromatography.

### Example 3

### Identification of Phosphotransferase Activity for the Production of ATP

To identify a single enzyme with phosphotransferase activity for the production of ATP, a set of phosphotransferases described in literature were first screened. Screening of the isolated phosphotransferases was performed in 200 μL reaction volumes in 1.5 mL Eppendorf tubes, each tube containing 100 mM Tris-buffer pH 7.5, 100 mM MgCl₂, 100 mM polyphosphate (Maddrell's salt) and 2 mM of AMP (1) with 50% (v/v) isolated phosphotransferase enzyme. Reactions were incubated at 30° C., 900 rpm, for 1 h and analyzed using standard techniques known in the art, including HPLC. A phosphotransferase with SEQ ID NO: 52 (PPK12) exhibited the highest activity towards the formation of ATP. The activity of SEQ ID NO: 52 for the production of ATP was subsequently confirmed using multiple enzyme preparations including isolated enzyme (example 2), clarified lysate (example 5) and shake flask powder (SFP; example 1).

### Example 4

### Preparation of Cell Pellets for HTP Screening

Single colonies were picked in a 96-well format and grown in 190 uL LB media containing 1% glucose and 30 μg/mL CAM, at 30° C., 200 rpm, and 85% humidity. Following overnight growth, 20 μL of the grown cultures were transferred into a deep well plate containing 380 μL of TB media with 30 μg/mL CAM. The cultures were grown at 30° C., 250 rpm, with 85% humidity for approximately 2.5 hours. When the OD₆₀₀ of the cultures reached 0.4-0.8, expression of the ligase gene was induced by the addition of IPTG to a final concentration of 1 mM. Following induction, growth continued for 18-20 hours at 30° C., 250 rpm with 85% humidity. Cells were harvested by centrifugation at 4,000 rpm and 4° C. for 10 minutes; the supernatant was then discarded. The cell pellets were stored at −80° C. until ready for use.

### Example 5

### Lysis and Preparation of Clarified Lysate

Prior to performing the assay, the cell pellets were thawed and resuspended in 300 μL of lysis buffer (containing 1 g/L lysozyme, 0.5 g/L PMBS and 0.1 μL/mL or 0.2 U/ml of commercial DNAse (New England BioLabs, M0303L) in 50 mM Tris-buffer at pH 7.5. The plates were agitated with medium-speed shaking for 2.5 hours on a microtiter plate shaker at room temperature. The plates were then centrifuged at 4,000 rpm for 10 minutes at 4° C., and the clarified supernatants were used in the HTP assay reaction for activity determination as described in the following examples.

### Example 6

### Analytical Method for Activity and Selectivity Evaluation

Activity improvements of the engineered phosphotransferases were analyzed by High Pressure Liquid Chromatography (HPLC) using the methods described in Table 5. HPLC methods with UV-detection were developed to analyze the formation of product ATP and resolve the substrate AMP (1) as well as the intermediate ADP. The analytical methods aim for the shortest run time enabling good resolution of the three compounds. The percentage conversion was calculated according to the following equation based on the peak area of each compound:

$$\% Conversion = \frac{ATP}{AMP + ADP + ATP}$$

TABLE 5
HPLC method 1 used for activity determination.

| Instrument | Agilent 1290 Infinity II UHPLC System | | |
|---|---|---|---|
| Column | Infinity Lab Poroshell 120 HILIC-Z (e.g. Agilent PN: | | |
| | 679775-924) | | |
| | Length 50 mm, internal diameter 2.1 mm column, particle | | |
| | size: 2.7 μm.. | | |
| Mobile Phase | A: 20 mM Ammonium Acetate pH 9.0 | | |
| | 1.54 g ammonium acetate is dissolved in 1 L water. The pH is | | |
| | adjusted to 9.0 with an ammonia solution (25%) | | |
| | B: Acetonitrile / 150 mM Ammonium Acetate pH 10.5 | | |
| | (90:10) | | |
| | 1.16 g ammonium acetate is dissolved in 100 mL water. The | | |
| | pH is adjusted to 10.5 with an ammonia solution (25%) and | | |
| | then mixed with 900 mL acetomitrile. | | |
| Gradient | Time (min.) | Phase B (%) | Flowrate (mL/min) |
| | 0.00 | 90 | 1.0 |
| | 0.80 | 74 | 1.0 |
| | 0.90 | 48 | 1.0 |
| | 1.20 | 48 | 1.0 |
| | 1.30 | 90 | 1.0 |
| | 1.75 | 90 | 1.0 |

| | |
|---|---|
| Run Time | 1.75 minutes |
| Compound elution | AMP = 0.817 min; ADP = 1.02 .886 min, ATP = 0.942 min |
| Column Temperature | 25° C. |
| Autosampler Temperature | 15° C. |
| Injection volume | 1 μL |
| Detection | UV 260 nm |

The method provided herein find use in analyzing the variants produced using the present invention. However, it is not intended that present invention be limited to the method described herein, as there are other suitable methods known in the art that are applicable to the analysis of the variants provided herein and/or produced using the methods provided herein.

### Example 7

Round 1 Evolution and Screening of Engineered Polypeptides Derived from SEQ ID NO: 52 for Improved Production of ATP Product

The engineered polynucleotide (SEQ ID NO: 48) encoding the polypeptide with phosphotransferase activity of SEQ ID NO: 52 was used to generate the engineered polypeptides of Table 6. These polypeptides displayed improved phosphotransferase activity under the desired conditions e.g., the improvement in the formation of ATP as compared to the starting polypeptide. The engineered polypeptides, having the amino acid sequences of even-numbered sequence identifiers were generated from the “backbone” amino acid sequence of SEQ ID NO: 52, as described below together with the analytical method described in Table 5.

Directed evolution began with the polynucleotide set forth in SEQ ID NO: 48. Libraries of engineered polypeptides were generated using various well-known techniques (e.g., saturation mutagenesis, recombination of previously identified beneficial amino acid differences) and screened using HTP assay and analysis methods, described below, that measured the polypeptides' ability to produce ATP.

The enzyme assays were carried out in 96-well PCR plates, in 80 μL total reaction volume per well. The reactions contained 0.0025% (v/v) of undiluted phosphotransferase lysate, prepared as described in Example 4, 0.3 mM AMP, 30 mM polyphosphate (Maddrell's salt), 50 mM Tris-buffer at pH 8.0, 5 mM MgCl₂ and 5 mM DTT. The reaction plates were heat-sealed and incubated in a thermocycler at 30° C. for 30 minutes.

After incubation the reactions were quenched by the addition of 120 μL of 10 mM EDTA solution into each well of plates. The plates were then centrifuged at 4,000 rpm for 5 min. Subsequently a 150 μL aliquot of the supernatant was removed from each well and added to a shallow well 96-well plate. The samples were analyzed via HPLC to determine the activity of the enzyme variants using the analytical method described in Table 5. Selected phosphotransferase (PPK12) variants showing an improved formation of ATP relative to SEQ ID NO: 52 are shown in Table 6. Levels of increased activity were determined as the mean of two replicates.

TABLE 6
Phosphotransferase Activity for the production
of ATP Relative to SEQ ID NO: 52

| SEQ | Amino | FIOP | FIOP |
|---|---|---|---|
| ID NO: | Acid | % Conversion | % Conversion |
| (nt/aa) | Differences | Mean | Standard Deviation |
| 53/54 | Y288R | +++ | low |
| 55/56 | E284G | +++ | low |
| 57/58 | K298* | +++ | medium |
| 59/60 | L178T | +++ | medium |
| 49/62 | D289G | +++ | high |
| 63/64 | L290V | ++ | medium |
| 65/66 | C232A | ++ | low |
| 67/68 | N287V | ++ | low |
| 69/70 | T301R | +++ | high |
| 71/72 | L290G | ++ | high |
| 73/74 | I293Q | ++ | medium |
| 75/76 | Y288Q | + | low |
| 77/78 | D291W | + | low |
| 79/80 | K298Y | + | medium |
| 81/82 | Y288* | ++ | medium |
| 83/84 | N141M | + | low |
| 85/86 | I296V | + | medium |
| | | Levels of increased | Levels of standard |
| | | activity were | deviation were defined |
| | | determined | as follows: “low” 0 to |
| | | relative to the reference | 0.2, “medium” > 0.2, |
| | | polypeptide of SEQ ID | “high” > 1.0 |
| | | NO: 52 and defined as | |
| | | follows: “+” > 1.0, | |
| | | “++” > 2.00, “+++” > | |
| | | 3.0 | |

### Example 8

Identification of Improved dsRNA Ligation Activity Under ATP Recycling Conditions with Increased Polyphosphate Concentration

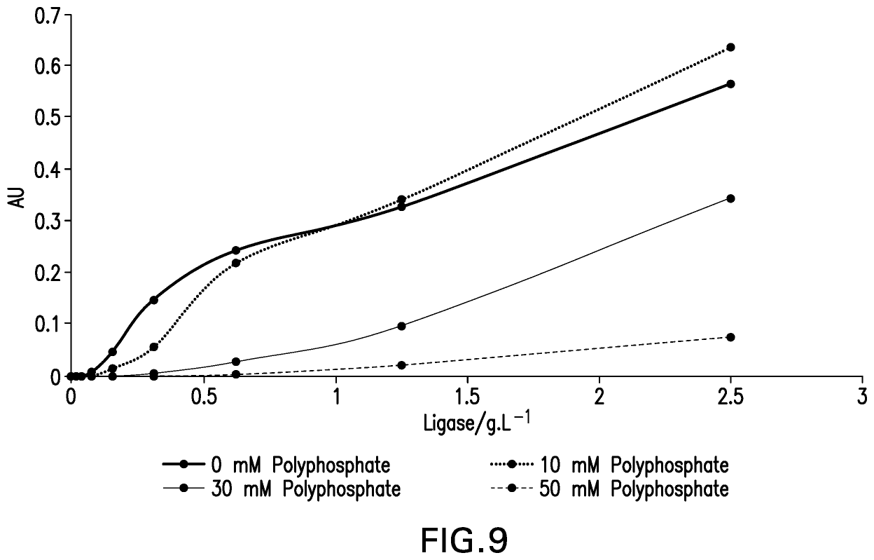

ATP is consumed during the ligation reaction. In the presence of PPK, polyphosphate is consumed to generate ATP from AMP. Therefore, to facilitate the ligation reaction with a higher substrate concentration under ATP recycling conditions, a high concentration of polyphosphate is needed. To process 6 mM of substrate oligonucleotides (substrates 1-6, table 2), either a stoichiometric quantity of ATP (24 mM), or a sub-stoichiometric quantity of ATP and a stoichiometric quantity of polyphosphate (48 mM) is required. However, increasing polyphosphate concentration has a negative effect on the ligation reaction (FIG. 9).

Therefore, to identify an enzyme with improved dsRNA ligation activity under ATP recycling conditions which can tolerate a higher polyphosphate concentration compared with the ligase of SEQ ID NO: 2, a collection of ligases was screened in the presence of AMP, polyphosphate and the optimized PPK of SEQ ID NO: 62.

The enzyme assays were carried in 96-well PCR plates, in 100 μL total reaction volume per well. The reaction contained 10% (v/v) of undiluted ligase lysate prepared as described in Example 5, 1 mM (each) of substrate oligonucleotides (substrates 1-6, table 2), 50 mM Tris-buffer at pH 7.0, 1 mM AMP, 5 mM MgCl₂, 5 mM DTT, 30 mM polyphosphate (Maddrell's salt) and 1 g/L PPK of SEQ ID NO: 62 SFP (prepared as described in Example 1). The reaction plates were heat-sealed and incubated in a thermocycler at 30° C. for 24 h.

After incubation the plates were subjected to a heat inactivation step (95° C., 20 min) to quench the reaction and precipitate proteinaceous content of the added lysate. The plates were then centrifuged at 4,000 rpm for 5 min. Subsequently, a 50 μL aliquot of the supernatant was removed from each well and added to the deep well 96-well plate containing 950 μL of 10 mM EDTA solution (pH 7.0). The samples were further diluted by transferring 50 μL of the diluted sample into a deep well 96-well plate containing 950 μL of 10 mM EDTA solution (pH 7.0). The samples were analyzed via HPLC to determine the activity of ligase variants using the analytical method described in Example 1.

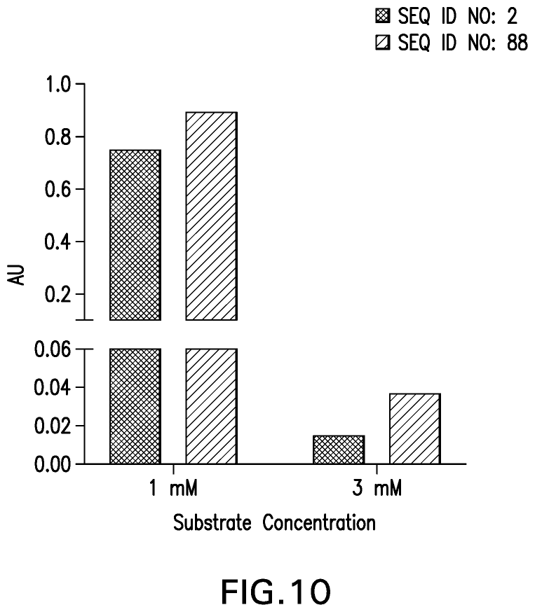

The ligase of SEQ ID NO: 88 was identified as having higher ligation activity compared to the ligase of SEQ ID NO: 2 under the desired reaction conditions. The higher activity of the ligase of SEQ ID NO: 88 was confirmed using SFP (Example 1) with both 1 mM and 3 mM substrate oligonucleotides (FIG. 10).

### Example 9

### Optimization of ATP Recycling Conditions

The polynucleotide sequence SEQ ID NO: 87 encoding the best performing polypeptide of SEQ ID NO: 88 with dsRNA ligation activity in the presence of a high concentration of polyphosphate was cloned in place of the ligase polynucleotide of SEQ ID NO: 31 at the C-terminus of the Klignase 4 polynucleotide construct SEQ ID NO: 40 to generate a new polynucleotide SEQ ID NO: 89 encoding a new fusion polypeptide termed Klignase 4.2 (SEQ ID NO: 90). To evaluate both the dsRNA ligation activity and the phosphotransferase activity of Klignase 4.2, ligation reactions were set up under various ATP recycling conditions.

The enzyme assays were carried in 96-well PCR plates, in 100 μL total reaction volume per well. The reaction contained 1 mM of each substrate oligonucleotide (substrates 1-6, table 2), 1 mM AMP, 5 mM DTT, either 50 mM Tris-buffer at pH 7.0, 100 mM Tris-buffer pH 7.0 or 100 mM MOPS buffer pH 7.2, either 5 mM, 20 mM, 40 mM, 60 mM, 80 mM or 100 mM MgCl₂, either 0% (v/v) or 10% (v/v) DMSO, either 30 mM polyphosphate (Graham's salt) or either 30 mM, 50 mM or 80 mM polyphosphate (Maddrell's salt) and either 0 g/L, 0.625 g/L, 1.25 g/L, 2.5 g/L, 5 g/L, 10 g/L, 20 g/L or 40 g/L of Klignase 4.2 (SEQ ID NO: 90) SFP (prepared as described in Example 1). The reaction plates were heat-sealed and incubated in a thermocycler at 30° C. for 24 h.

After incubation the plates were subjected to a heat inactivation step (95° C., 20 min) to quench the reaction and precipitate proteinaceous content of the added lysate. The plates were then centrifuged at 4,000 rpm for 5 min. Subsequently, a 50 μL aliquot of the supernatant was removed from each well and added to the deep well 96-well plate containing 950 μL of 10 mM EDTA solution (pH 7.0). The samples were further diluted by transferring 50 μL of the diluted sample into a deep well 96-well plate containing 950 μL of 10 mM EDTA solution (pH 7.0). The samples were analyzed via HPLC to determine the activity of ligases variants using the analytical method described in Example 1.

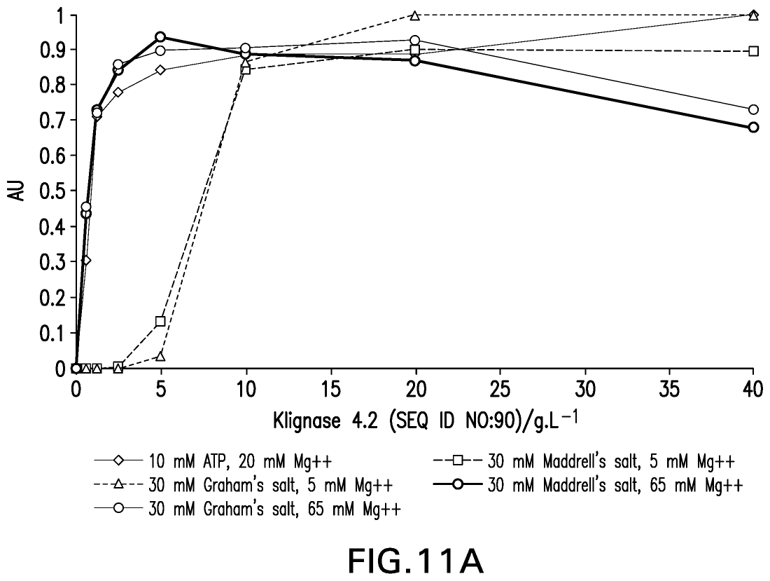

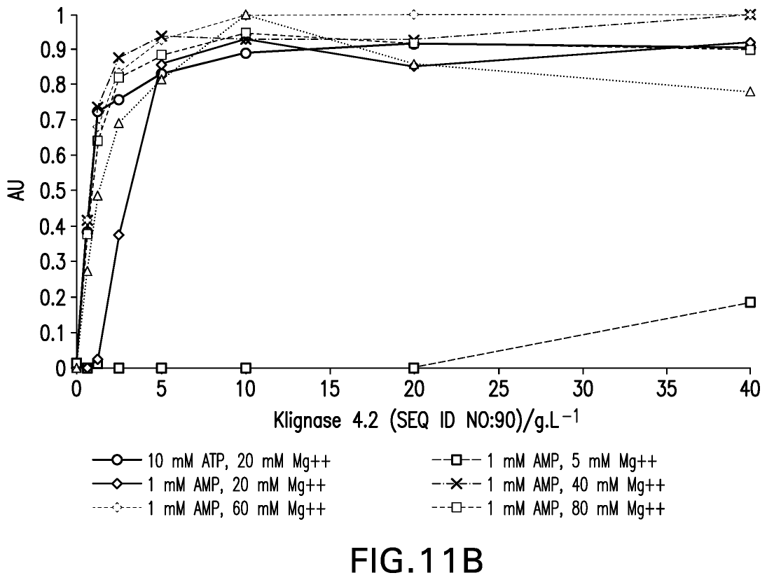

Klignase 4.2 was shown to accept both Maddrell's and Graham's salt of polyphosphate, (FIG. 11a). Increasing the concentration of MgCl₂ from 5 mM to >20 mM significantly improved dsRNA ligation activity (FIG. 11b).

### Example 10

Round 1 Evolution and Screening of Engineered Polypeptides Derived from SEQ ID NO: 90 for Improved Production of siRNA Product Under ATP Recycling Conditions.

The engineered polynucleotide of SEQ ID NO: 89 encoding the polypeptide with dsRNA ligation activity and phosphotransferase activity of SEQ ID NO: 90 was used to generate engineered polypeptides that displayed improved dsRNA ligase activity under ATP recycling conditions.

The engineered polypeptides, having amino acid sequences of even-numbered sequence identifiers were generated from the “backbone” amino acid sequence of SEQ ID NO: 90, as described below together with the analytical method described in Example 1. Directed evolution began with the polynucleotide set forth in SEQ ID NO: 89. Libraries of engineered polypeptides were generated using various well-known techniques (e.g., saturation mutagenesis, recombination of previously identified beneficial amino acid differences) and screened using HTP assay and analysis methods, described below, that measured the polypeptides' ability to produce the siRNA product.

The enzyme assays were carried in 96-well PCR plates, in 100 μL total reaction volume per well. The reaction contained either 10% (v/v) or 20% (v/v) of undiluted Klignase lysate prepared as described in Example 5, 2.5 mM (each) of substrate oligonucleotides (substrates 1-6, table 2), 50 mM Tris-buffer at pH 7.0, 1 mM AMP, 40 mM MgCl₂, 5 mM DTT, 80 mM polyphosphate (Maddrell's salt). The reaction plates were heat-sealed and incubated in a thermocycler at 30° C. for 24 h.

After incubation the plates were subjected to a heat inactivation step (95° C., 20 min) to quench the reaction and precipitate proteinaceous content of the added lysate. The plates were then centrifuged at 4,000 rpm for 5 min. Subsequently, a 50 μL aliquot of the supernatant was removed from each well and added to the deep well 96-well plate containing 950 μL of 10 mM EDTA solution (pH 7.0). The samples were further diluted by transferring 50 μL of the diluted sample into a deep well 96-well plate containing 950 μL of 10 mM EDTA solution (pH 7.0). The samples were further diluted by transferring 80 μL of the diluted sample into a deep well 96-well plate containing 120 μL of 10 mM EDTA solution (pH 7.0). The samples were analyzed via HPLC to determine the activity of Klignase variants using the analytical method described in Example 1.

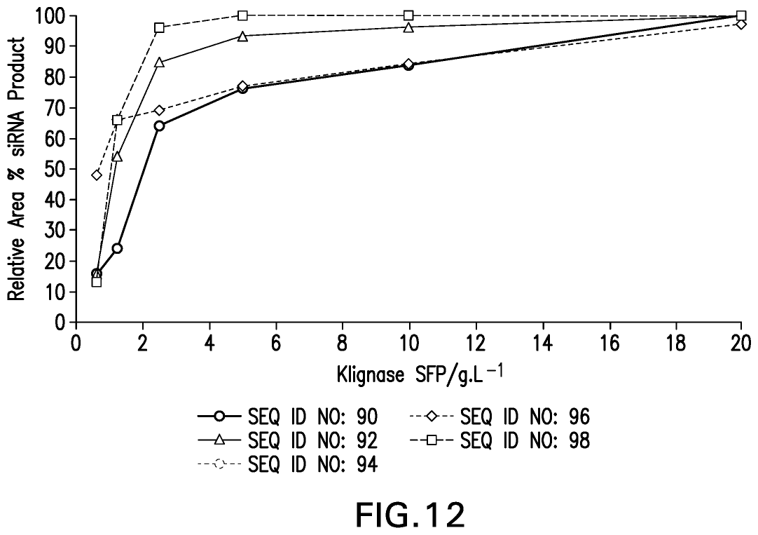

Lysates containing engineered polypeptides with SEQ ID NOs: 92, 94, 96 and 98 were identified as having increased dsRNA ligation activity under ATP recycling conditions as compared to the ligase of SEQ ID NO: 90. The higher activity of Klignases with SEQ ID NOs: 92, 94, 96 and 98 were confirmed using SFP prepared as described in Example 1 (FIG. 12).

### Example 11

### Comparison of the Catalytic Activity of the Engineered Polypeptide SEQ ID NO: 98 and Seq Id No: 11.

To further validate the improved dsRNA ligase activity of the new Klignase of SEQ ID NO: 98, compared to that of one of the best performing initial Klignase constructs, i.e. Klignase 4 of SEQ ID NO: 11, the two polypeptides were produced as SFP as described in Example 1 and evaluated for their ability to convert 6 mM of each substrate oligonucleotide (substrates 1-6, table 2) in the presence of 0.25 mM AMP, 40 mM MgCl₂, 80 mM Polyphosphate (Maddrell's salt), 5 mM DTT and 100 mM MOPS-buffer, pH 7.2. The enzyme assays were carried out in 96-well PCR plates, in 100 μL total reaction volume per well and contained either 0 g/L, 0.313 g/L, 0.625 g/L, 1.25 g/L, 2.5 g/L, 5 g/L, 10 g/L or 20 g/L of Klignase. The reaction plate was heat-sealed and incubated in a thermocycler at 30° C. for 24 h.

Following incubation, the plate was subjected to a heat inactivation step (95° C., 20 min) to quench the reaction and precipitate proteinaceous content of the added SFP. The plate was then centrifuged at 4,000 rpm for 5 min. A 50 μL aliquot of the supernatant from each well of each plate was removed and subsequently diluted in 950 μL of 10 mM EDTA solution. The samples were further diluted by transferring 20 μL of the diluted sample into a deep well 96-well plate containing 180 μL of 10 mM EDTA solution (pH 7.0). The samples were further diluted by transferring 18 μL of the diluted sample into a deep well 96-well plate containing 198 μL of 10 mM EDTA solution (pH 7.0). The samples were analyzed via HPLC using the analytical method described in Example 1.

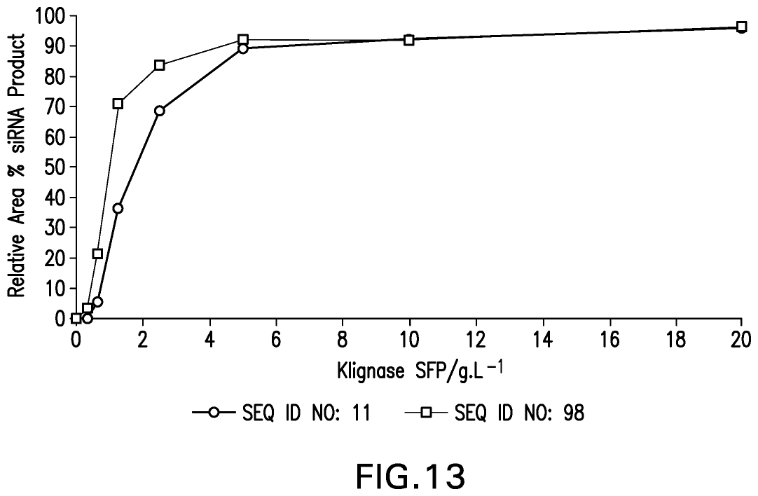

Comparative data in FIG. 13 shows that the Klignase of SEQ ID NO: 98 exhibits improved ligation activity under ATP recycling conditions compared with the Klignase of SEQ ID NO: 11.

1. A method of producing an oligonucleotide from two or more oligonucleotide fragments, wherein the method comprises contacting:
i. two or more oligonucleotide fragments;
ii. an ATP-dependent nucleic acid ligase; and
iii. a polyphosphate kinase (PPK);
and thereby providing an oligonucleotide.

2. The method of claim 1, further comprising contacting:
iv. adenosine triphosphate (ATP) and/or adenosine monophosphate (AMP);
v. polyphosphate; and
vi. a divalent cation.

3. The method of claim 1, wherein:
(a) the two or more oligonucleotide fragments comprise two or more RNA oligonucleotide fragments; optionally wherein the ATP-dependent nucleic acid ligase is an RNA ligase; optionally wherein:
(i) the RNA ligase is a double-stranded RNA ligase; and/or
(ii) the RNA ligase is a member of the RNA ligase 2 family, optionally wherein the RNA ligase is Bacteriophage RB69 RNA ligase 2;
(b) the two or more oligonucleotide fragments comprise two or more DNA oligonucleotide fragments; optionally wherein the ATP-dependent nucleic acid ligase is a DNA ligase; optionally wherein the DNA ligase is T4 DNA ligase; and/or
(c) the PPK is PPK12 or ajPAP.

4. The method of claim 1
(A) wherein the ATP-dependent nucleic acid ligase and the PPK are linked, optionally wherein the ATP-dependent nucleic acid ligase and the PPK are linked via a polypeptide linker; optionally wherein:
(i) the PPK is located at the N-terminus of the linker and the ATP-dependent nucleic acid ligase is located at the C-terminus of the linker; and/or
(ii) the linker is a polypeptide linker comprising at least 3 amino acids, optionally at least 6 amino acids; optionally wherein the linker comprises an amino acid sequence selected from:
a)
(SEQ ID NO: 19)
HHHHHH,
optionally
(SEQ ID NO: 20)
HHHHHHHHHH;
b)
(SEQ ID NO: 21)
ENLYFQS;
c)
(SEQ ID NO: 22)
ENLYFQG;
d)
(SEQ ID NO: 23)
SSGSSG;
e)
(SEQ ID NO: 24)
GSAGSAAGSGEF;
and/or
f)
(SEQ ID NO: 25)
GSSGSGSSSGGSSSSGSS;
and/or
(B) wherein:
(i) the ATP-dependent nucleic acid ligase comprises a purification tag;
(ii) the PPK comprises a purification tag; and/or
(iii) the linker comprises a purification tag.

5. The method of claim 2, wherein:
(a) the polyphosphate is a polyphosphate salt, optionally wherein the polyphosphate salt is sodium polyphosphate (Maddrell's salt) or sodium hexametaphosphate (Graham's salt); and/or
(b) the divalent cation cofactor is Mg²⁺ or Mn²⁺; and/or
(c) the method is performed with a divalent cation concentration of 5-100 mM, optionally 30-50 mM; and/or
(d) the method is performed with a sub-stoichiometric concentration of ATP and/or AMP; and/or
(e) the method further comprises a step of purifying the oligonucleotide.

6. The method of claim 1, wherein:
(A) the oligonucleotide is up to 60 nucleotides in length; optionally wherein each of the oligonucleotide fragments are 4-16 nucleotides in length, optionally 6-9 nucleotides in length; and/or
(B) the oligonucleotide fragments are:
(a) single-stranded; or
(b) double-stranded, optionally wherein one or more of the double-stranded oligonucleotide fragments comprises one or two single-stranded overhang(s); and/or
(C) one or more of the oligonucleotide fragments comprises a chemical modification; optionally wherein the chemical modification is selected from:
(a) a modified backbone, optionally selected from a phosphorothioate (e.g. chiral phosphorothioate) or methylphosphonate internucleotide linkage;
(b) a modified nucleotide, optionally selected from 2′-O-methyl (2′-OMe), 2′-flouro (2′-F), 2′-deoxy, 2′-deoxy-2′-fluoro, 2′-O-methoxyethyl (2′-O-MOE), 2′-O-aminopropyl (2′-O-AP), 2′-O-dimethylaminoethyl (2′-O-DMAOE), 2′-O-dimethylaminopropyl (2′-O-DMAP), 2′-O-dimethylaminoethyloxyethyl (2′-O-DMAEOE), 2′-O—N-methylacetamido (2′-O-NMA), locked nucleic acid (LNA), glycol nucleic acid (GNA), phosphoramidate (e.g. mesyl phosphoramidate), 2′,3′-seco nucleotide mimic, 2′-F-arabino nucleotide, abasic nucleotide, 2′-amino modified nucleotide, 2′-alkyl-modified nucleotide, morpholino nucleotide, vinylphosphonate (e.g. 5′ vinylphosphonate), and cyclopropyl phosphonate deoxyribonucleotide; and/or
(c) conjugation to a ligand, optionally wherein the ligand comprises one or more N-Acetylgalactosamine (GalNAc) derivatives; and/or
(D) the ATP-dependent nucleic acid ligase and/or the PPK are immobilised; optionally wherein the ATP-dependent nucleic acid ligase and/or the PPK are immobilised on a solid material by chemical bond or a physical adsorption method.

7. A composition comprising:
i. an ATP-dependent nucleic acid ligase;
ii. a PPK;
iii. ATP and/or AMP;
iv. a divalent cation; and
v. polyphosphate;
optionally wherein the composition further comprises two or more oligonucleotide fragments.

8. A kit comprising:
i. an ATP-dependent nucleic acid ligase;
ii. a PPK;
iii. ATP and/or AMP;
iv. polyphosphate;
v. a divalent cation; and
vi. instructions for use in a method of producing an oligonucleotide from two or more oligonucleotide fragments.

9. The composition of claim 7 or the kit of claim 8, wherein:
(a) the polyphosphate is a polyphosphate salt; optionally wherein the polyphosphate salt is selected from Graham's salt and Maddrell's salt; and/or
(b) the divalent cation is Mg²⁺ or Mn²⁺; and/or
(c) the concentration of divalent cation is 5-100 mM, optionally 30-50 mM.

10. A fusion polypeptide comprising:
a) a PPK domain; and
b) an ATP-dependent nucleic acid ligase domain.

11. The fusion polypeptide of claim 10, wherein:
(A) the PPK is PPK12 or ajPAP; and/or
(B) the PPK domain comprises an amino acid sequence that has at least 85% identity with the amino acid sequence of any one of SEQ ID NOs: 5-7; and/or
(C) the ATP-dependent nucleic acid ligase domain is:
(i) an RNA ligase domain; optionally wherein the RNA ligase domain is a double-stranded RNA (dsRNA) ligase domain; and/or the dsRNA ligase is a member of the RNA ligase 2 family, optionally wherein the dsRNA ligase is Bacteriophage RB69 RNA ligase 2; or
(ii) a DNA ligase domain; optionally wherein the DNA ligase domain is a T4 DNA ligase domain; and/or
(D) the ATP-dependent nucleic acid ligase domain comprises an amino acid sequence that has at least 85% sequence identity with the amino acid sequence of any one of SEQ ID NOs: 1-4 or 88; and/or
(E) the fusion polypeptide comprises a purification tag, optionally wherein a purification tag is located at the N- and/or C-terminus of the fusion polypeptide; and/or
(F) the fusion polypeptide comprises an amino acid sequence that has at least 85% sequence identity with the amino acid sequence of any one of SEQ ID NOs: 8-18, 90, 92, 94, 96 or 98; and/or
(G) the fusion polypeptide comprises a linker; optionally wherein:
(i) the linker is located between the PPK domain and the ATP-dependent nucleic acid ligase domain; and/or
(ii) the PPK domain is located at the N-terminus of the linker and the ATP-dependent nucleic acid ligase domain is located at the C-terminus of the linker; and/or
(iii) the linker comprises a purification tag; optionally wherein a purification tag is located at the N- and/or C-terminus of the fusion polypeptide; and/or
(iv) the linker is a polypeptide linker comprising at least 3 amino acids, optionally at least 6 amino acids, optionally wherein the linker comprises an amino acid sequence selected from:
a)
(SEQ ID NO: 19)
HHHHHH,
optionally
(SEQ ID NO: 20)
HHHHHHHHHH;
b)
(SEQ ID NO: 21)
ENLYFQS;
c)
(SEQ ID NO: 22)
ENLYFQG;
d)
(SEQ ID NO: 23)
SSGSSG;
e)
(SEQ ID NO: 24)
GSAGSAAGSGEF;
and/or
f)
(SEQ ID NO: 25)
GSSGSGSSSGGSSSSGSS.

12. The method according to claim 1, wherein the ATP-dependent nucleic acid ligase and the PPK are provided as a fusion polypeptide, wherein the fusion polypeptide comprising:
a) a PPK domain; and
b) an ATP-dependent nucleic acid ligase domain.

13. A nucleic acid molecule encoding the fusion polypeptide of claim 10; optionally wherein the nucleic acid molecule comprises a nucleic acid sequence that has at least 85% sequence identity with the nucleic acid sequence of:
(a) any one of SEQ ID NOs: 34-36; and/or
(b) any one of SEQ ID NOs: 30-33 or 87.

14. A vector comprising the nucleic acid of claim 13; optionally wherein the vector is selected from a plasmid, a cosmid, a bacteriophage or a viral vector.

15. A host cell comprising the nucleic acid molecule of claim 13; optionally wherein the host cell is E. coli.

16. (canceled)

17. The method of claim 1, wherein:
(a) the oligonucleotide is a therapeutic oligonucleotide; and/or
(b) the oligonucleotide product is at least 80% pure, optionally wherein the oligonucleotide product is at least 85% pure, at least 90% pure, at least 95% pure, optionally wherein the oligonucleotide product is at least 98% pure.


SEQUENCE LISTING (100 sequences)

SEQ ID NO: 1 - protein, 332 aa, unidentified
MFKKYSSLENHYNSKFIEKLYTNGLTTGVWVAREKIHGTNFSLIIERDNVTCAKRTGPILPAEDFYGYEIVLKKYDKAIKTVQKFMYTARAVSYQVFGEFAGGGIQKGVDYGEKDFYVFDILINTESGDNTYLTDYEMQDFCNEFGFKMAPMLGRGTFDSLIMIPNDLDSVLAAYNATASEDLVEANNCVFDANVIGDNTAEGYVLKPCFPKWLPNGTRVAIKCKNSKFSEKKKSDKPIKTQVPLTEIDKNLLDVLACYVTLNRVNNVISKIGTVTPKDFGKVMGLTVQDILEETSREGIVLTTSDNPNLVKKELVRMVQDVLRPAWIELVS

SEQ ID NO: 2 - protein, 332 aa, synthetic construct
MFKKYSSLENHYNSKFIEKLYTNGLTTGVWVAREKIHGANFSLIIERDNVTCAKRTGPILPAEDFYGYEIVLKKYDKAIKTVQKFMYTARAVSYQVFGEFAGGGIQKGVDYGEKDFYVFDILINTESGDNTYLTDYEMQDFCNEFGFKMAPMLGRGTFDSLIMIPNDLDSVLAAYNATASEDLVEANNCVFDANVIGDNTAEGYVLKPCFPKWLPNGNRVIIKCKNSKFSEKKKSDKPIKTQVPLTEIDKNLLDVLACYVTLNRVNNVISKIGTVTPKDFGKVMGLTVQDILEETSREGIVLTTSDNPNLVKKELVRMVQDVLRPAWIELVS

SEQ ID NO: 3 - protein, 334 aa, unidentified
MFKKYSSLENHYNSKFIEKLYSLGLTGGEWVAREKIHGTNFSLIIERDKVTCAKRTGPILPAEDFFGYEIILKNYADSIKAVQDIMETSAVVSYQVFGEFAGPGIQKNVDYCDKDFYVFDIIVTTESGDVTYVDDYMMESFCNTFKFKMAPLLGRGKFEELIKLPNDLDSVVQDYNFTVDHAGLVDANKCVWNAEAKGEVFTAEGYVLKPCYPSWLRNGNRVAIKCKNSKFSEKKKSDKPIKAKVELSEADNKLVGILACYVTLNRVNNVISKIGEIGPKDFGKVMGLTVQDILEETSREGITLTQADNPSLIKKELVKMVQDVLRPAWIELVS

SEQ ID NO: 4 - protein, 487 aa, synthetic construct
MILKILNEIASIGSTKQKQAILEKNKDNELLKRVYRLTYSRGLQYYIKKWPKPGIATQSFGMLTLTDMLDFIEFTLATRKLTGNAAIEELTGYITDGKKDDVEVLRRVMMRDLECGASVSIANKVWPGLIPEQPQMLASSYDEKGINKNIKFPAFAQLKADGARCFAEVRGDELDDVRLLSRAGNEYLGLDLLKEELIKMTAEARQIHPEGVLIDGELVYHEQVKKEPEGLDFLFDAYPENSKAKEFAEVAESRTASNGIANKSLKGTISEKEAQCMKFQVWDYVPLVEIYSLPAFRLKYDVRFSKLEQMTSGYDKVILIENQVVNNLDEAKVIYKKYIDQGLEGIILKNIDGLWENARSKNLYKFKEVIDVDLKIVGIYPHRKDPTKAGGFILESECGKIKVNAGSGLKDKAGVKSHELDRTRIMENQNYYIGKILECECNGWLKSDGRTDYVKLFLPIAIRLREDKTKANTFEDVFGDFHEVTGL

SEQ ID NO: 5 - protein, 298 aa, unidentified
MINIYKIDKLNNFNLNNHKTDDYSLCKDKDTALELTQKNIQKIYDYQQKLYAEKKEGLIIAFQAMDAAGKDGTIREVLKALAPQGVHEKPFKSPSSTELAHDYLWRVHNAVPEKGEITIFNRSHYEDVLIGKVKELYKFQNKADRIDENTVVDNRYEDIRNFEKYLYNNSVRIIKIFLNVSKKEQAERFLSRIEEPEKNWKFSDSDFEERVYWDKYQQAFEDAINATSTKDCPWYVVPADRKWYMRYVVSEIVVKTLEEMNPKYPTVTKETLERFEGYRTKLLEEYNYDLDTIRPIEK

SEQ ID NO: 6 - protein, 298 aa, synthetic construct
MINIYKIDKLNNFNLNNHKTDDYSLCKDKDTALELTQKNIQKIYDYQQKLYAEKKEGLIIAFQAMDAAGKDGTIREVLKALAPQGVHEKPFKSPSSTELAHDYLWRVHNAVPEKGEITIFNRSHYEDVLIGKVKELYKFQNKADRIDENTVVDNRYEDIRNFEKYLYNNSVRIIKIFLNVSKKEQAERFLSRIEEPEKNWKFSDSDFEERVYWDKYQQAFEDAINATSTKDCPWYVVPADRKWYMRYVVSEIVVKTLEEMNPKYPTVTKETLERFEGYRTKLLEEYNYGLDTIRPIEK

SEQ ID NO: 7 - protein, 475 aa, Acinetobacter johnsonii
MDTETIASAVLNEEQLSLDLIEAQYALMNTRDQSNAKSLVILVSGIELAGKGEAVKQLREWVDPRFLYVKADPPHLFNLKQPFWQPYTRFVPAEGQIMVWFGNWYGDLLATAMHASKPLDDTLFDEYVSNMRAFEQDLKNNNVDVLKVWFDLSWKSLQKRLDDMDPSEVHWHKLHGLDWRNKKQYDTLQKLRTRFTDDWQIIDGEDEDLRNHNFAQAILTALRHCPEHEKKAALKWQQAPIPDILTQFEVPQAEDANYKSELKKLTKQVADAMRCDDRKVVIAFEGMDAAGKGGAIKRIVKKLDPREYEIHTIAAPEKYELRRPYLWRFWSKLQSDDITIFDRTWYGRVLVERVEGFATEVEWQRAYAEINRFEKNLSSSQTVLIKFWLAIDKDEQAARFKARESTPHKRFKITEEDWRNRDKWDDYLKAAADMFAHTDTSYAPWYIISTNDKQQARIEVLRAILKQLKADRDTD

SEQ ID NO: 8 - protein, 643 aa, synthetic construct
MINIYKIDKLNNFNLNNHKTDDYSLCKDKDTALELTQKNIQKIYDYQQKLYAEKKEGLIIAFQAMDAAGKDGTIREVLKALAPQGVHEKPFKSPSSTELAHDYLWRVHNAVPEKGEITIFNRSHYEDVLIGKVKELYKFQNKADRIDENTVVDNRYEDIRNFEKYLYNNSVRIIKIFLNVSKKEQAERFLSRIEEPEKNWKFSDSDFEERVYWDKYQQAFEDAINATSTKDCPWYVVPADRKWYMRYVVSEIVVKTLEEMNPKYPTVTKETLERFEGYRTKLLEEYNYGLDTIRPIEKHHHHHHENLYFQSMFKKYSSLENHYNSKFIEKLYTNGLTTGVWVAREKIHGANFSLIIERDNVTCAKRTGPILPAEDFYGYEIVLKKYDKAIKTVQKFMYTARAVSYQVFGEFAGGGIQKGVDYGEKDFYVFDILINTESGDNTYLTDYEMQDFCNEFGFKMAPMLGRGTFDSLIMIPNDLDSVLAAYNATASEDLVEANNCVFDANVIGDNTAEGYVLKPCFPKWLPNGNRVIIKCKNSKFSEKKKSDKPIKTQVPLTEIDKNLLDVLACYVTLNRVNNVISKIGTVTPKDFGKVMGLTVQDILEETSREGIVLTTSDNPNLVKKELVRMVQDVLRPAWIELVS

SEQ ID NO: 9 - protein, 647 aa, synthetic construct
MINIYKIDKLNNFNLNNHKTDDYSLCKDKDTALELTQKNIQKIYDYQQKLYAEKKEGLIIAFQAMDAAGKDGTIREVLKALAPQGVHEKPFKSPSSTELAHDYLWRVHNAVPEKGEITIFNRSHYEDVLIGKVKELYKFQNKADRIDENTVVDNRYEDIRNFEKYLYNNSVRIIKIFLNVSKKEQAERFLSRIEEPEKNWKFSDSDFEERVYWDKYQQAFEDAINATSTKDCPWYVVPADRKWYMRYVVSEIVVKTLEEMNPKYPTVTKETLERFEGYRTKLLEEYNYGLDTIRPIEKHHHHHHHHHHENLYFQSMFKKYSSLENHYNSKFIEKLYTNGLTTGVWVAREKIHGANFSLIIERDNVTCAKRTGPILPAEDFYGYEIVLKKYDKAIKTVQKFMYTARAVSYQVFGEFAGGGIQKGVDYGEKDFYVFDILINTESGDNTYLTDYEMQDFCNEFGFKMAPMLGRGTFDSLIMIPNDLDSVLAAYNATASEDLVEANNCVFDANVIGDNTAEGYVLKPCFPKWLPNGNRVIIKCKNSKFSEKKKSDKPIKTQVPLTEIDKNLLDVLACYVTLNRVNNVISKIGTVTPKDFGKVMGLTVQDILEETSREGIVLTTSDNPNLVKKELVRMVQDVLRPAWIELVS

SEQ ID NO: 10 - protein, 653 aa, synthetic construct
MHHHHHHENLYFQSINIYKIDKLNNFNLNNHKTDDYSLCKDKDTALELTQKNIQKIYDYQQKLYAEKKEGLIIAFQAMDAAGKDGTIREVLKALAPQGVHEKPFKSPSSTELAHDYLWRVHNAVPEKGEITIFNRSHYEDVLIGKVKELYKFQNKADRIDENTVVDNRYEDIRNFEKYLYNNSVRIIKIFLNVSKKEQAERFLSRIEEPEKNWKFSDSDFEERVYWDKYQQAFEDAINATSTKDCPWYVVPADRKWYMRYVVSEIVVKTLEEMNPKYPTVTKETLERFEGYRTKLLEEYNYGLDTIRPIEKGQTGSSGSSGMFKKYSSLENHYNSKFIEKLYTNGLTTGVWVAREKIHGANFSLIIERDNVTCAKRTGPILPAEDFYGYEIVLKKYDKAIKTVQKFMYTARAVSYQVFGEFAGGGIQKGVDYGEKDFYVFDILINTESGDNTYLTDYEMQDFCNEFGFKMAPMLGRGTFDSLIMIPNDLDSVLAAYNATASEDLVEANNCVFDANVIGDNTAEGYVLKPCFPKWLPNGNRVIIKCKNSKFSEKKKSDKPIKTQVPLTEIDKNLLDVLACYVTLNRVNNVISKIGTVTPKDFGKVMGLTVQDILEETSREGIVLTTSDNPNLVKKELVRMVQDVLRPAWIELVS

SEQ ID NO: 11 - protein, 659 aa, synthetic construct
MHHHHHHENLYFQSINIYKIDKLNNFNLNNHKTDDYSLCKDKDTALELTQKNIQKIYDYQQKLYAEKKEGLIIAFQAMDAAGKDGTIREVLKALAPQGVHEKPFKSPSSTELAHDYLWRVHNAVPEKGEITIFNRSHYEDVLIGKVKELYKFQNKADRIDENTVVDNRYEDIRNFEKYLYNNSVRIIKIFLNVSKKEQAERFLSRIEEPEKNWKFSDSDFEERVYWDKYQQAFEDAINATSTKDCPWYVVPADRKWYMRYVVSEIVVKTLEEMNPKYPTVTKETLERFEGYRTKLLEEYNYGLDTIRPIEKGQTGGSAGSAAGSGEFMFKKYSSLENHYNSKFIEKLYTNGLTTGVWVAREKIHGANFSLIIERDNVTCAKRTGPILPAEDFYGYEIVLKKYDKAIKTVQKFMYTARAVSYQVFGEFAGGGIQKGVDYGEKDFYVFDILINTESGDNTYLTDYEMQDFCNEFGFKMAPMLGRGTFDSLIMIPNDLDSVLAAYNATASEDLVEANNCVFDANVIGDNTAEGYVLKPCFPKWLPNGNRVIIKCKNSKFSEKKKSDKPIKTQVPLTEIDKNLLDVLACYVTLNRVNNVISKIGTVTPKDFGKVMGLTVQDILEETSREGIVLTTSDNPNLVKKELVRMVQDVLRPAWIELVS

SEQ ID NO: 12 - protein, 665 aa, synthetic construct
MHHHHHHENLYFQSINIYKIDKLNNFNLNNHKTDDYSLCKDKDTALELTQKNIQKIYDYQQKLYAEKKEGLIIAFQAMDAAGKDGTIREVLKALAPQGVHEKPFKSPSSTELAHDYLWRVHNAVPEKGEITIFNRSHYEDVLIGKVKELYKFQNKADRIDENTVVDNRYEDIRNFEKYLYNNSVRIIKIFLNVSKKEQAERFLSRIEEPEKNWKFSDSDFEERVYWDKYQQAFEDAINATSTKDCPWYVVPADRKWYMRYVVSEIVVKTLEEMNPKYPTVTKETLERFEGYRTKLLEEYNYGLDTIRPIEKGQTGGSSGSGSSSGGSSSSGSSMFKKYSSLENHYNSKFIEKLYTNGLTTGVWVAREKIHGANFSLIIERDNVTCAKRTGPILPAEDFYGYEIVLKKYDKAIKTVQKFMYTARAVSYQVFGEFAGGGIQKGVDYGEKDFYVFDILINTESGDNTYLTDYEMQDFCNEFGFKMAPMLGRGTFDSLIMIPNDLDSVLAAYNATASEDLVEANNCVFDANVIGDNTAEGYVLKPCFPKWLPNGNRVIIKCKNSKFSEKKKSDKPIKTQVPLTEIDKNLLDVLACYVTLNRVNNVISKIGTVTPKDFGKVMGLTVQDILEETSREGIVLTTSDNPNLVKKELVRMVQDVLRPAWIELVS

SEQ ID NO: 13 - protein, 659 aa, synthetic construct
MHHHHHHENLYFQSMFKKYSSLENHYNSKFIEKLYTNGLTTGVWVAREKIHGANFSLIIERDNVTCAKRTGPILPAEDFYGYEIVLKKYDKAIKTVQKFMYTARAVSYQVFGEFAGGGIQKGVDYGEKDFYVFDILINTESGDNTYLTDYEMQDFCNEFGFKMAPMLGRGTFDSLIMIPNDLDSVLAAYNATASEDLVEANNCVFDANVIGDNTAEGYVLKPCFPKWLPNGNRVIIKCKNSKFSEKKKSDKPIKTQVPLTEIDKNLLDVLACYVTLNRVNNVISKIGTVTPKDFGKVMGLTVQDILEETSREGIVLTTSDNPNLVKKELVRMVQDVLRPAWIELVSGSAGSAAGSGEFINIYKIDKLNNFNLNNHKTDDYSLCKDKDTALELTQKNIQKIYDYQQKLYAEKKEGLIIAFQAMDAAGKDGTIREVLKALAPQGVHEKPFKSPSSTELAHDYLWRVHNAVPEKGEITIFNRSHYEDVLIGKVKELYKFQNKADRIDENTVVDNRYEDIRNFEKYLYNNSVRIIKIFLNVSKKEQAERFLSRIEEPEKNWKFSDSDFEERVYWDKYQQAFEDAINATSTKDCPWYVVPADRKWYMRYVVSEIVVKTLEEMNPKYPTVTKETLERFEGYRTKLLEEYNYGLDTIRPIEKGQTG

SEQ ID NO: 14 - protein, 665 aa, synthetic construct
MHHHHHHENLYFQSMFKKYSSLENHYNSKFIEKLYTNGLTTGVWVAREKIHGANFSLIIERDNVTCAKRTGPILPAEDFYGYEIVLKKYDKAIKTVQKFMYTARAVSYQVFGEFAGGGIQKGVDYGEKDFYVFDILINTESGDNTYLTDYEMQDFCNEFGFKMAPMLGRGTFDSLIMIPNDLDSVLAAYNATASEDLVEANNCVFDANVIGDNTAEGYVLKPCFPKWLPNGNRVIIKCKNSKFSEKKKSDKPIKTQVPLTEIDKNLLDVLACYVTLNRVNNVISKIGTVTPKDFGKVMGLTVQDILEETSREGIVLTTSDNPNLVKKELVRMVQDVLRPAWIELVSGSSGSGSSSGGSSSSGSSINIYKIDKLNNFNLNNHKTDDYSLCKDKDTALELTQKNIQKIYDYQQKLYAEKKEGLIIAFQAMDAAGKDGTIREVLKALAPQGVHEKPFKSPSSTELAHDYLWRVHNAVPEKGEITIFNRSHYEDVLIGKVKELYKFQNKADRIDENTVVDNRYEDIRNFEKYLYNNSVRIIKIFLNVSKKEQAERFLSRIEEPEKNWKFSDSDFEERVYWDKYQQAFEDAINATSTKDCPWYVVPADRKWYMRYVVSEIVVKTLEEMNPKYPTVTKETLERFEGYRTKLLEEYNYGLDTIRPIEKGQTG

SEQ ID NO: 15 - protein, 641 aa, synthetic construct
MFKKYSSLENHYNSKFIEKLYTNGLTTGVWVAREKIHGANFSLIIERDNVTCAKRTGPILPAEDFYGYEIVLKKYDKAIKTVQKFMYTARAVSYQVFGEFAGGGIQKGVDYGEKDFYVFDILINTESGDNTYLTDYEMQDFCNEFGFKMAPMLGRGTFDSLIMIPNDLDSVLAAYNATASEDLVEANNCVFDANVIGDNTAEGYVLKPCFPKWLPNGNRVIIKCKNSKFSEKKKSDKPIKTQVPLTEIDKNLLDVLACYVTLNRVNNVISKIGTVTPKDFGKVMGLTVQDILEETSREGIVLTTSDNPNLVKKELVRMVQDVLRPAWIELVSGSAGSAAGSGEFINIYKIDKLNNFNLNNHKTDDYSLCKDKDTALELTQKNIQKIYDYQQKLYAEKKEGLIIAFQAMDAAGKDGTIREVLKALAPQGVHEKPFKSPSSTELAHDYLWRVHNAVPEKGEITIFNRSHYEDVLIGKVKELYKFQNKADRIDENTVVDNRYEDIRNFEKYLYNNSVRIIKIFLNVSKKEQAERFLSRIEEPEKNWKFSDSDFEERVYWDKYQQAFEDAINATSTKDCPWYVVPADRKWYMRYVVSEIVVKTLEEMNPKYPTVTKETLERFEGYRTKLLEEYNYGLDTIRPIEK

SEQ ID NO: 16 - protein, 647 aa, synthetic construct
MFKKYSSLENHYNSKFIEKLYTNGLTTGVWVAREKIHGANFSLIIERDNVTCAKRTGPILPAEDFYGYEIVLKKYDKAIKTVQKFMYTARAVSYQVFGEFAGGGIQKGVDYGEKDFYVFDILINTESGDNTYLTDYEMQDFCNEFGFKMAPMLGRGTFDSLIMIPNDLDSVLAAYNATASEDLVEANNCVFDANVIGDNTAEGYVLKPCFPKWLPNGNRVIIKCKNSKFSEKKKSDKPIKTQVPLTEIDKNLLDVLACYVTLNRVNNVISKIGTVTPKDFGKVMGLTVQDILEETSREGIVLTTSDNPNLVKKELVRMVQDVLRPAWIELVSGSSGSGSSSGGSSSSGSSINIYKIDKLNNFNLNNHKTDDYSLCKDKDTALELTQKNIQKIYDYQQKLYAEKKEGLIIAFQAMDAAGKDGTIREVLKALAPQGVHEKPFKSPSSTELAHDYLWRVHNAVPEKGEITIFNRSHYEDVLIGKVKELYKFQNKADRIDENTVVDNRYEDIRNFEKYLYNNSVRIIKIFLNVSKKEQAERFLSRIEEPEKNWKFSDSDFEERVYWDKYQQAFEDAINATSTKDCPWYVVPADRKWYMRYVVSEIVVKTLEEMNPKYPTVTKETLERFEGYRTKLLEEYNYGLDTIRPIEK

SEQ ID NO: 17 - protein, 655 aa, synthetic construct
MINIYKIDKLNNFNLNNHKTDDYSLCKDKDTALELTQKNIQKIYDYQQKLYAEKKEGLIIAFQAMDAAGKDGTIREVLKALAPQGVHEKPFKSPSSTELAHDYLWRVHNAVPEKGEITIFNRSHYEDVLIGKVKELYKFQNKADRIDENTVVDNRYEDIRNFEKYLYNNSVRIIKIFLNVSKKEQAERFLSRIEEPEKNWKFSDSDFEERVYWDKYQQAFEDAINATSTKDCPWYVVPADRKWYMRYVVSEIVVKTLEEMNPKYPTVTKETLERFEGYRTKLLEEYNYGLDTIRPIEKHHHHHHSSGSSGHHHHHHENLYFQSMFKKYSSLENHYNSKFIEKLYTNGLTTGVWVAREKIHGANFSLIIERDNVTCAKRTGPILPAEDFYGYEIVLKKYDKAIKTVQKFMYTARAVSYQVFGEFAGGGIQKGVDYGEKDFYVFDILINTESGDNTYLTDYEMQDFCNEFGFKMAPMLGRGTFDSLIMIPNDLDSVLAAYNATASEDLVEANNCVFDANVIGDNTAEGYVLKPCFPKWLPNGNRVIIKCKNSKFSEKKKSDKPIKTQVPLTEIDKNLLDVLACYVTLNRVNNVISKIGTVTPKDFGKVMGLTVQDILEETSREGIVLTTSDNPNLVKKELVRMVQDVLRPAWIELVS

SEQ ID NO: 18 - protein, 659 aa, synthetic construct
MINIYKIDKLNNFNLNNHKTDDYSLCKDKDTALELTQKNIQKIYDYQQKLYAEKKEGLIIAFQAMDAAGKDGTIREVLKALAPQGVHEKPFKSPSSTELAHDYLWRVHNAVPEKGEITIFNRSHYEDVLIGKVKELYKFQNKADRIDENTVVDNRYEDIRNFEKYLYNNSVRIIKIFLNVSKKEQAERFLSRIEEPEKNWKFSDSDFEERVYWDKYQQAFEDAINATSTKDCPWYVVPADRKWYMRYVVSEIVVKTLEEMNPKYPTVTKETLERFEGYRTKLLEEYNYGLDTIRPIEKGQTGSSGSSGHHHHHHSSGSSGENLYFQSMFKKYSSLENHYNSKFIEKLYTNGLTTGVWVAREKIHGANFSLIIERDNVTCAKRTGPILPAEDFYGYEIVLKKYDKAIKTVQKFMYTARAVSYQVFGEFAGGGIQKGVDYGEKDFYVFDILINTESGDNTYLTDYEMQDFCNEFGFKMAPMLGRGTFDSLIMIPNDLDSVLAAYNATASEDLVEANNCVFDANVIGDNTAEGYVLKPCFPKWLPNGNRVIIKCKNSKFSEKKKSDKPIKTQVPLTEIDKNLLDVLACYVTLNRVNNVISKIGTVTPKDFGKVMGLTVQDILEETSREGIVLTTSDNPNLVKKELVRMVQDVLRPAWIELVS

SEQ ID NO: 19 - protein, 6 aa, synthetic construct
HHHHHH

SEQ ID NO: 20 - protein, 10 aa, synthetic construct
HHHHHHHHHH

SEQ ID NO: 21 - protein, 7 aa, synthetic construct
ENLYFQS

SEQ ID NO: 22 - protein, 7 aa, synthetic construct
ENLYFQG

SEQ ID NO: 23 - protein, 6 aa, synthetic construct
SSGSSG

SEQ ID NO: 24 - protein, 12 aa, synthetic construct
GSAGSAAGSGEF

SEQ ID NO: 25 - protein, 18 aa, synthetic construct
GSSGSGSSSGGSSSSGSS

SEQ ID NO: 26 - protein, 14 aa, synthetic construct
MHHHHHHENLYFQS

SEQ ID NO: 27 - protein, 10 aa, synthetic construct
GQTGHHHHHH

SEQ ID NO: 28 - protein, 10 aa, synthetic construct
EQKLISEEDL

SEQ ID NO: 29 - protein, 8 aa, synthetic construct
DYKDDDDK

SEQ ID NO: 30 - DNA, 999 bp, unidentified
  source 1..999: mol_type = unassigned DNA
atgttcaagaaatactcctccttggagaaccactataactcgaaattcattgaaaaactgtacaccaatggccttacgacgggtgtgtgggttgcgcgcgagaagatccatgggaccaactttagcctgattatcgaacgtgataatgtcacttgcgccaaacgtacaggcccgattctcccagctgaagacttctatggttacgaaatcgtgctgaagaaatacgataaagcaattaagaccgtccaaaagtttatgtatacagcacgcgccgtgtcttaccaggtatttggagaatttgctggtggcggcatccagaaaggtgttgattatggcgagaaagacttctatgtgttcgatattttaatcaacaccgaatcaggtgacaatacgtatctgaccgattacgagatgcaagacttctgtaacgaatttgggtttaagatggcgccgatgttaggacgcggcaccttcgatagtctgattatgattcctaacgatttggatagtgtacttgcggcgtataatgccactgcaagcgaagacctggttgaagcgaacaactgcgtctttgatgccaatgtgatcggtgacaatacggctgagggctacgttctgaaaccgtgttttcccaaatggctcccgaacggtacccgtgtggcgatcaaatgcaagaactcgaaattcagcgagaaaaagaaatctgacaaaccgattaagactcaggtaccactgacggaaatcgataagaatctgttggatgtcctggcctgttatgtgaccttaaaccgcgtcaataacgtgatttcgaagattggcacagttaccccgaaagattttgggaaagttatgggcctgactgtgcaggatatcctcgaagaaacctcacgtgaagggattgtacttacgacgagtgacaaccctaatctggtgaagaaagaattggttcgcatggtccaagatgtgttacgtcccgcatggatcgagctggtttcctaa

SEQ ID NO: 31 - DNA, 999 bp, synthetic construct
  source 1..999: mol_type = other DNA
atgttcaagaaatactcctccttggagaaccactataactcgaaattcattgaaaaactgtacaccaatggccttacgacgggtgtgtgggttgcgcgcgagaagattcatggggcgaactttagcctgattatcgaacgtgataatgtcacttgcgccaaacgtacaggcccgattctcccagctgaagacttctatggttacgaaatcgtgctgaagaaatacgataaagcaattaagaccgtccaaaagtttatgtatacagcacgcgccgtgtcttaccaggtatttggagaatttgctggtggtggcatccagaaaggtgttgattatggcgagaaagacttctatgtgttcgatattttaatcaacaccgaatcaggtgacaatacgtatctgaccgattacgagatgcaagacttctgtaacgaatttgggtttaagatggcgcctatgttaggacgcggcaccttcgatagtctgattatgattcctaacgatttggatagtgtacttgcggcgtataatgccactgcaagcgaagacctggttgaagcgaacaactgcgtctttgatgccaatgtgatcggtgacaatacggctgagggctacgttctgaaaccgtgttttcccaaatggctcccgaacggtaaccgtgtgattatcaaatgcaagaactcgaaattcagcgagaaaaagaaatctgacaaaccgattaagactcaggtaccactgacggaaatcgataagaatctgttggatgtcctggcctgttatgtgaccttaaaccgcgtcaataacgtgatttcgaagattggcacagttaccccgaaagattttgggaaagttatgggcctgactgtgcaggatatcctcgaagaaacctcacgtgaagggattgtacttacgacgagtgacaaccctaatctggtgaagaaagaattggttcgcatggtccaagatgtgttacgtcccgcatggatcgagctggtttcctaa

SEQ ID NO: 32 - DNA, 1002 bp, unidentified
  source 1..1002: mol_type = unassigned DNA
acttaccaactcaatccaagctggacgaagtacatcttgtaccattttaactaattcctttttaatcaaagaaggattatctgcttgagttagagtaataccttcacgagaagtttcttccaaaatatcttgaacagttagccccatcacctttccaaaatcctttggaccaatttcgccaattttagaaataacgttatttacgcggttcagtgtaacgtaacaagctaaaattcccaccaatttgttatcagcttctgatagctcaactttagctttaataggcttatcagactttttcttttcactaaatttagagttcttgcatttaatcgctacacgatttccattacgaagccaagaaggataacaaggtttcaatacatatccttcagcagtaaatacttcgccttttgcttcggcattccaaacgcatttatttgcatcaactaatccagcatggtctactgtaaaattataatcttggacgacagaatctaaatcatttggcaatttaataagctcttcaaatttaccgcgacctaaaagtggagccattttaaatttaaatgtattacagaatgattccatcatataatcatctacataagtcacatcaccgctttctgtagtaacaataatgtcaaatacataaaaatctttatcacaataatcaacattcttctgaatgccaggtccagcgaattcgccaaagacttgataagatacaaccgctgaggtttccataatatcttgtacagctttaatggaatcagcataattcttcaaaataatttcatacccaaagaaatcttcagcaggaagaatcggtccagtgcgtttagcgcaagtcactttatcacgctcaataatcaatgagaaatttgtgccgtgaatcttttcacgagctacccactccccaccagtcaatcccaagctataaagtttttcaataaatttagagttgtaatgattttcaagactgctatactttttaaacat

SEQ ID NO: 33 - DNA, 1464 bp, synthetic construct
  source 1..1464: mol_type = other DNA
atgattcttaaaattctgaacgaaatagcatctattggttcaactaaacagaagcaagcaattcttgaaaagaataaagataatgaattgcttaaacgagtatatcgtctgacttattctcgtgggttacagtattatatcaagaaatggcctaaacctggtattgctacccagagttttggaatgttgactcttaccgatatgcttgacttcattgaattcacattagctactcggaaattgactggaaatgcagcaattgaggaattaactggatatatcaccgatggtaaaaaagatgatgttgaagttttgcgtcgagtgatgatgcgagaccttgaatgtggtgcttcagtatctattgcaaacaaagtttggccaggtttaattcctgaacaacctcaaatgctcgcaagttcttatgatgaaaaaggcattaataagaatatcaaatttccagcctttgctcagttaaaagctgatggagctcggtgttttgctgaagttagaggtgatgaattagatgatgttcgtcttttatcacgagctggtaatgaatatctaggattagatcttcttaaggaagagttaattaaaatgaccgctgaagcccgccagattcatccagaaggtgtgttgattgatggcgaattggtataccatgagcaagttaaaaaggagccagaaggcctagattttctttttgatgcttatcctgaaaacagtaaagctaaagaattcgccgaagtagctgaatcacgtactgcttctaatggaatcgccaataaatctttaaagggaaccatttctgaaaaagaagcacaatgcatgaagtttcaggtctgggattatgtcccgttggtagaaatatacagtcttcctgcatttcgtttgaaatatgatgtacgtttttctaaactagaacaaatgacatctggatatgataaagtaattttaattgaaaaccaggtagtaaataacctagatgaagctaaggtaatttataaaaagtatattgaccaaggtcttgaaggtattattctcaaaaatatcgatggattatgggaaaatgctcgttcaaaaaatctttataaatttaaagaagtaattgatgttgatttaaaaattgtaggaatttatcctcaccgtaaagaccctactaaagcgggtggatttattcttgagtcagagtgtggaaaaattaaggtaaatgctggttcaggcttaaaagataaagccggtgtaaaatcgcatgaacttgaccgtactcgcattatggaaaaccaaaattattatattggaaaaattctagagtgcgaatgcaacggttggttaaaatctgatggccgcactgattacgttaaattatttcttccgattgcgattcgtttacgtgaagataaaactaaagctaatacattcgaagatgtatttggtgattttcatgaggtaactggtctatga

SEQ ID NO: 34 - DNA, 897 bp, unidentified
  source 1..897: mol_type = unassigned DNA
atgattaacatctataaaattgataaactgaataattttaacttgaacaatcataagaccgacgactacagcctgtgcaaggataaagatactgcgctcgaactgacccagaagaacatccagaagatttatgactaccaacagaaactgtatgccgagaagaaagaaggccttatcatcgcattccaagctatggatgcagcgggtaaagatggaacgattcgcgaagtgttaaaagcgttagctccgcagggtgttcacgagaaaccatttaaaagtccgtcgtcgacagaactggcccatgattacttgtggcgtgtccacaatgccgtacctgagaaaggcgaaattaccatcttcaaccgctcccattatgaagacgtgctgattgggaaagttaaagaactgtacaagtttcagaacaaagcggatcgtattgacgaaaatacggtggtggataaccgctatgaggatatccgtaatttcgagaaatatctctacaacaactctgtccgcatcatcaaaatctttctgaacgtcagcaagaaagaacaagcagaacgttttctgagccgcattgaagaaccggagaagaattggaaattcagtgactcagattttgaagagcgtgtttattgggacaaatatcagcaggcgttcgaagatgccattaatgcgacctccacgaaagattgtccctggtacgtagtaccggctgatcgcaaatggtacatgcgttatgtggtgagcgaaatcgttgttaagacccttgaagaaatgaacccaaaatacccgacggtgactaaagagacattggaacgctttgaaggctatcgtaccaaactgctggaggagtataattatgacttagataccattcgccctattgaaaagggc

SEQ ID NO: 35 - DNA, 897 bp, synthetic construct
  source 1..897: mol_type = other DNA
atgattaacatctataaaattgataaactgaataattttaacttgaacaatcataagaccgacgactacagcctgtgcaaggataaagatactgcgctcgaactgacccagaagaacatccagaagatttatgactaccaacagaaactgtatgccgagaagaaagaaggccttatcatcgcattccaagctatggatgcagcgggtaaagatggaacgattcgcgaagtgttaaaagcgttagctccgcagggtgttcacgagaaaccatttaaaagtccgtcgtcgacagaactggcccatgattacttgtggcgtgtccacaatgccgtacctgagaaaggcgaaattaccatcttcaaccgctcccattatgaagacgtgctgattgggaaagttaaagaactgtacaagtttcagaacaaagcggatcgtattgacgaaaatacggtggtggataaccgctatgaggatatccgtaatttcgagaaatatctctacaacaactctgtccgcatcatcaaaatctttctgaacgtcagcaagaaagaacaagcagaacgttttctgagccgcattgaagaaccggagaagaattggaaattcagtgactcagattttgaagagcgtgtttattgggacaaatatcagcaggcgttcgaagatgccattaatgcgacctccacgaaagattgtccctggtacgtagtaccggctgatcgcaaatggtacatgcgttatgtggtgagcgaaatcgttgttaagacccttgaagaaatgaacccaaaatacccgacggtgactaaagagacattggaacgctttgaaggctatcgtaccaaactgctggaggagtataattatgggttagataccattcgccctattgaaaagggc

SEQ ID NO: 36 - DNA, 1425 bp, Acinetobacter johnsonii
  source 1..1425: mol_type = genomic DNA
atggatacagaaacgatcgccagtgcagtgctgaatgaagaacagctttcactggacttaattgaagcgcaatatgcgttgatgaatacccgtgatcagagcaatgcaaaaagtttagtgattttggtcagtggaatcgaacttgcgggtaaaggcgaagcggtgaaacagctccgcgaatgggtcgatcctcgttttttatatgtcaaagccgatccaccgcatctgtttaatctaaaacagcctttttggcagccctatacccgatttgtgcctgccgaagggcaaattatggtgtggtttggtaattggtatggggatttgttggctacggccatgcatgcttcaaagcctttagatgacactttgtttgatgaatacgtcagcaatatgcgggcttttgaacaggacttaaaaaataacaacgtagatgtcttaaaagtttggttcgatttgtcgtggaagtctctgcaaaagcgtctagatgatatggacccgagcgaagtgcattggcataagttgcatgggctagactggcgcaataaaaaacaatatgacaccttacaaaagctacgtacgcgcttcaccgatgactggcaaatcattgatggtgaagatgaggatttgcgtaatcacaattttgcacaagcaattttaacggcactacgacactgcccagagcatgaaaaaaaggccgcgctaaaatggcagcaagcaccaataccagatattctgactcagtttgaagtccctcaagctgaggatgcgaactataaatcagaattgaaaaaactcaccaaacaagtggccgatgccatgcgctgtgatgaccgtaaagtggtgattgcttttgaaggtatggatgctgcgggtaaagggggggcgattaagcgtattgtgaaaaagctcgacccacgagaatatgaaattcataccattgccgcacctgaaaaatatgagttacgccgtccttatctgtggcgtttttggagcaaattgcagtcggatgacatcactatttttgatcggacgtggtatggacgcgttttagtcgagcgggtagaaggcttcgcaaccgaggtagagtggcaacgcgcttatgcggaaatcaatcgttttgaaaaaaacctcagtagcagccaaaccgtgctgattaagttttggctggcgattgataaagatgaacaagcagcgcgttttaaagcccgcgaaagtactccgcataaacgttttaaaattaccgaagaagattggcgcaatcgcgacaaatgggatgactatttaaaggcagccgcggatatgtttgcgcataccgacaccagctatgcgccttggtatattatttccaccaatgataaacaacaggcccgcattgaagtcttaagggcaattttaaaacagctcaaagcggatcgcgacacggat

SEQ ID NO: 37 - DNA, 1932 bp, synthetic construct
  source 1..1932: mol_type = other DNA
atgattaacatctataaaattgataaactgaataattttaacttgaacaatcataagaccgacgactacagcctgtgcaaggataaagatactgcgctcgaactgacccagaagaacatccagaagatttatgactaccaacagaaactgtatgccgagaagaaagaaggccttatcatcgcattccaagctatggatgcagcgggtaaagatggaacgattcgcgaagtgttaaaagcgttagctccgcagggtgttcacgagaaaccatttaaaagtccgtcgtcgacagaactggcccatgattacttgtggcgtgtccacaatgccgtacctgagaaaggcgaaattaccatcttcaaccgctcccattatgaagacgtgctgattgggaaagttaaagaactgtacaagtttcagaacaaagcggatcgtattgacgaaaatacggtggtggataaccgctatgaggatatccgtaatttcgagaaatatctctacaacaactctgtccgcatcatcaaaatctttctgaacgtcagcaagaaagaacaagcagaacgttttctgagccgcattgaagaaccggagaagaattggaaattcagtgactcagattttgaagagcgtgtttattgggacaaatatcagcaggcgttcgaagatgccattaatgcgacctccacgaaagattgtccctggtacgtagtaccggctgatcgcaaatggtacatgcgttatgtggtgagcgaaatcgttgttaagacccttgaagaaatgaacccaaaatacccgacggtgactaaagagacattggaacgctttgaaggctatcgtaccaaactgctggaggagtataattatgggttagataccattcgccctattgaaaagcaccatcaccatcaccatgaaaatctgtattttcagagcatgttcaagaaatactcctccttggagaaccactataactcgaaattcattgaaaaactgtacaccaatggccttacgacgggtgtgtgggttgcgcgcgagaagattcatggggcgaactttagcctgattatcgaacgtgataatgtcacttgcgccaaacgtacaggcccgattctcccagctgaagacttctatggttacgaaatcgtgctgaagaaatacgataaagcaattaagaccgtccaaaagtttatgtatacagcacgcgccgtgtcttaccaggtatttggagaatttgctggtggtggcatccagaaaggtgttgattatggcgagaaagacttctatgtgttcgatattttaatcaacaccgaatcaggtgacaatacgtatctgaccgattacgagatgcaagacttctgtaacgaatttgggtttaagatggcgcctatgttaggacgcggcaccttcgatagtctgattatgattcctaacgatttggatagtgtacttgcggcgtataatgccactgcaagcgaagacctggttgaagcgaacaactgcgtctttgatgccaatgtgatcggtgacaatacggctgagggctacgttctgaaaccgtgttttcccaaatggctcccgaacggtaaccgtgtgattatcaaatgcaagaactcgaaattcagcgagaaaaagaaatctgacaaaccgattaagactcaggtaccactgacggaaatcgataagaatctgttggatgtcctggcctgttatgtgaccttaaaccgcgtcaataacgtgatttcgaagattggcacagttaccccgaaagattttgggaaagttatgggcctgactgtgcaggatatcctcgaagaaacctcacgtgaagggattgtacttacgacgagtgacaaccctaatctggtgaagaaagaattggttcgcatggtccaagatgtgttacgtcccgcatggatcgagctggtttcctaa

SEQ ID NO: 38 - DNA, 1945 bp, synthetic construct
  source 1..1945: mol_type = other DNA
atgatcaatatttacaagatcgacaagttaaacaacttcaatctgaataaccacaaaacagatgattatagtttatgtaaagacaaggacaccgccctggagcttacgcagaagaatattcaaaagatatacgattatcagcaaaagttgtacgcagagaagaaggaagggctgataatagcctttcaggcaatggacgctgctgggaaggacggtactatacgtgaggtactgaaggctctggcaccacaaggagtccatgaaaagcctttcaagtcgcctagctctactgagttagctcacgactatctttggagagtacataacgcggtcccggaaaagggagagatcacgatatttaatcgttcacactacgaggatgtattgatcggtaaggtaaaggagctttataaattccaaaataaggccgaccggatcgatgagaacactgtcgtcgacaatagatacgaagacattcggaacttcgaaaagtacctgtataacaattcagttcggataataaagattttccttaatgtgtctaagaaggagcaggctgagcgattcttgtctcgaatcgaggagcccgaaaagaactggaagttttcggattcggacttcgaggaacgagtgtactgggataagtaccaacaagcctttgaggacgcgataaacgccacgtcaacaaaggactgcccatggtatgtggtgcctgcagaccgtaagtggtatatgcgctacgttgttagtgagattgtagtaaagactttagaggagatgaatccgaagtatccaaccgtcaccaaggaaacgctggagcgtttcgaggggtaccgcacgaagttactcgaagaatacaactacggcctggacactatccgtccaatcgagaaacatcaccatcaccatcaccaccaccatcacgagaacttatacttccagtctatgtttaagaagtatagcagcttagaaaatcattacaatagtaagttcatagagaagttgtacacgaacggtttaaccacaggcgtctgggtagccagagagaaaatccacggagcaaatttcagcttaatcattgagagagacaacgttacatgtgcaaagcgaacgggaccaatattacccgccgaggacttttacggctatgagattgtattaaagaagtatgacaaggccatcaaaacagttcagaaattcatgtacacggcccgtgcagtcagttatcaagtgttcggcgagttcgcgggtggcggtattcaaaagggcgtggactacggagaaaaggatttctacgtatttgacatccttataaatactgagtcgggagataacacctacttaacagactatgaaatgcaggatttctgcaatgagttcggattcaaaatggcacccatgctgggtcgtggtacgtttgacagcttgataatgatcccaaatgacctcgactccgtgctggcagcttacaacgcgaccgcgagtgaggatttggttgaggccaataattgtgtgttcgacgcaaacgttattggcgataacacagcagaaggatatgtcttaaagccatgcttcccgaagtggttacctaatgggaatcgggtcatcattaagtgtaagaatagtaagttttcggaaaagaagaagagcgataagcctataaagacacaagtcccgttaacagagattgacaagaaccttctggacgtgttggcttgctacgtcacactgaatcgagtaaacaatgttatctcaaagataggaaccgtgacacctaaggacttcggaaaggtcatgggactcacggttcaagacattctggaagagacgtcccgagagggtatagtgctgaccacatctgataatccgaacttggttaagaaggagcttgtgcggatggttcaggacgtcttgcgcccagcttggatagaattggtaagctgaa

SEQ ID NO: 39 - DNA, 1962 bp, synthetic construct
  source 1..1962: mol_type = other DNA
atgcaccatcaccatcaccatgaaaatctgtattttcagagcattaacatctataaaattgataaactgaataattttaacttgaacaatcataagaccgacgactacagcctgtgcaaggataaagatactgcgctcgaactgacccagaagaacatccagaagatttatgactaccaacagaaactgtatgccgagaagaaagaaggccttatcatcgcattccaagctatggatgcagcgggtaaagatggaacgattcgcgaagtgttaaaagcgttagctccgcagggtgttcacgagaaaccatttaaaagtccgtcgtcgacagaactggcccatgattacttgtggcgtgtccacaatgccgtacctgagaaaggcgaaattaccatcttcaaccgctcccattatgaagacgtgctgattgggaaagttaaagaactgtacaagtttcagaacaaagcggatcgtattgacgaaaatacggtggtggataaccgctatgaggatatccgtaatttcgagaaatatctctacaacaactctgtccgcatcatcaaaatctttctgaacgtcagcaagaaagaacaagcagaacgttttctgagccgcattgaagaaccggagaagaattggaaattcagtgactcagattttgaagagcgtgtttattgggacaaatatcagcaggcgttcgaagatgccattaatgcgacctccacgaaagattgtccctggtacgtagtaccggctgatcgcaaatggtacatgcgttatgtggtgagcgaaatcgttgttaagacccttgaagaaatgaacccaaaatacccgacggtgactaaagagacattggaacgctttgaaggctatcgtaccaaactgctggaggagtataattatgggttagataccattcgccctattgaaaagggccaaactggcagctctggttctagcggcatgttcaagaaatactcctccttggagaaccactataactcgaaattcattgaaaaactgtacaccaatggccttacgacgggtgtgtgggttgcgcgcgagaagattcatggggcgaactttagcctgattatcgaacgtgataatgtcacttgcgccaaacgtacaggcccgattctcccagctgaagacttctatggttacgaaatcgtgctgaagaaatacgataaagcaattaagaccgtccaaaagtttatgtatacagcacgcgccgtgtcttaccaggtatttggagaatttgctggtggtggcatccagaaaggtgttgattatggcgagaaagacttctatgtgttcgatattttaatcaacaccgaatcaggtgacaatacgtatctgaccgattacgagatgcaagacttctgtaacgaatttgggtttaagatggcgcctatgttaggacgcggcaccttcgatagtctgattatgattcctaacgatttggatagtgtacttgcggcgtataatgccactgcaagcgaagacctggttgaagcgaacaactgcgtctttgatgccaatgtgatcggtgacaatacggctgagggctacgttctgaaaccgtgttttcccaaatggctcccgaacggtaaccgtgtgattatcaaatgcaagaactcgaaattcagcgagaaaaagaaatctgacaaaccgattaagactcaggtaccactgacggaaatcgataagaatctgttggatgtcctggcctgttatgtgaccttaaaccgcgtcaataacgtgatttcgaagattggcacagttaccccgaaagattttgggaaagttatgggcctgactgtgcaggatatcctcgaagaaacctcacgtgaagggattgtacttacgacgagtgacaaccctaatctggtgaagaaagaattggttcgcatggtccaagatgtgttacgtcccgcatggatcgagctggtttcctaa

SEQ ID NO: 40 - DNA, 1980 bp, synthetic construct
  source 1..1980: mol_type = other DNA
atgcaccatcaccatcaccatgaaaatctgtattttcagagcattaacatctataaaattgataaactgaataattttaacttgaacaatcataagaccgacgactacagcctgtgcaaggataaagatactgcgctcgaactgacccagaagaacatccagaagatttatgactaccaacagaaactgtatgccgagaagaaagaaggccttatcatcgcattccaagctatggatgcagcgggtaaagatggaacgattcgcgaagtgttaaaagcgttagctccgcagggtgttcacgagaaaccatttaaaagtccgtcgtcgacagaactggcccatgattacttgtggcgtgtccacaatgccgtacctgagaaaggcgaaattaccatcttcaaccgctcccattatgaagacgtgctgattgggaaagttaaagaactgtacaagtttcagaacaaagcggatcgtattgacgaaaatacggtggtggataaccgctatgaggatatccgtaatttcgagaaatatctctacaacaactctgtccgcatcatcaaaatctttctgaacgtcagcaagaaagaacaagcagaacgttttctgagccgcattgaagaaccggagaagaattggaaattcagtgactcagattttgaagagcgtgtttattgggacaaatatcagcaggcgttcgaagatgccattaatgcgacctccacgaaagattgtccctggtacgtagtaccggctgatcgcaaatggtacatgcgttatgtggtgagcgaaatcgttgttaagacccttgaagaaatgaacccaaaatacccgacggtgactaaagagacattggaacgctttgaaggctatcgtaccaaactgctggaggagtataattatgggttagataccattcgccctattgaaaagggccaaactggcggtagcgcgggctctgcggcgggttctggcgaatttatgttcaagaaatactcctccttggagaaccactataactcgaaattcattgaaaaactgtacaccaatggccttacgacgggtgtgtgggttgcgcgcgagaagattcatggggcgaactttagcctgattatcgaacgtgataatgtcacttgcgccaaacgtacaggcccgattctcccagctgaagacttctatggttacgaaatcgtgctgaagaaatacgataaagcaattaagaccgtccaaaagtttatgtatacagcacgcgccgtgtcttaccaggtatttggagaatttgctggtggtggcatccagaaaggtgttgattatggcgagaaagacttctatgtgttcgatattttaatcaacaccgaatcaggtgacaatacgtatctgaccgattacgagatgcaagacttctgtaacgaatttgggtttaagatggcgcctatgttaggacgcggcaccttcgatagtctgattatgattcctaacgatttggatagtgtacttgcggcgtataatgccactgcaagcgaagacctggttgaagcgaacaactgcgtctttgatgccaatgtgatcggtgacaatacggctgagggctacgttctgaaaccgtgttttcccaaatggctcccgaacggtaaccgtgtgattatcaaatgcaagaactcgaaattcagcgagaaaaagaaatctgacaaaccgattaagactcaggtaccactgacggaaatcgataagaatctgttggatgtcctggcctgttatgtgaccttaaaccgcgtcaataacgtgatttcgaagattggcacagttaccccgaaagattttgggaaagttatgggcctgactgtgcaggatatcctcgaagaaacctcacgtgaagggattgtacttacgacgagtgacaaccctaatctggtgaagaaagaattggttcgcatggtccaagatgtgttacgtcccgcatggatcgagctggtttcctaa

SEQ ID NO: 41 - DNA, 1998 bp, synthetic construct
  source 1..1998: mol_type = other DNA
atgcaccatcaccatcaccatgaaaatctgtattttcagagcattaacatctataaaattgataaactgaataattttaacttgaacaatcataagaccgacgactacagcctgtgcaaggataaagatactgcgctcgaactgacccagaagaacatccagaagatttatgactaccaacagaaactgtatgccgagaagaaagaaggccttatcatcgcattccaagctatggatgcagcgggtaaagatggaacgattcgcgaagtgttaaaagcgttagctccgcagggtgttcacgagaaaccatttaaaagtccgtcgtcgacagaactggcccatgattacttgtggcgtgtccacaatgccgtacctgagaaaggcgaaattaccatcttcaaccgctcccattatgaagacgtgctgattgggaaagttaaagaactgtacaagtttcagaacaaagcggatcgtattgacgaaaatacggtggtggataaccgctatgaggatatccgtaatttcgagaaatatctctacaacaactctgtccgcatcatcaaaatctttctgaacgtcagcaagaaagaacaagcagaacgttttctgagccgcattgaagaaccggagaagaattggaaattcagtgactcagattttgaagagcgtgtttattgggacaaatatcagcaggcgttcgaagatgccattaatgcgacctccacgaaagattgtccctggtacgtagtaccggctgatcgcaaatggtacatgcgttatgtggtgagcgaaatcgttgttaagacccttgaagaaatgaacccaaaatacccgacggtgactaaagagacattggaacgctttgaaggctatcgtaccaaactgctggaggagtataattatgggttagataccattcgccctattgaaaagggccaaactggcggtagctctggctctggcagctctagcggcggttctagctctagcggtagctctatgttcaagaaatactcctccttggagaaccactataactcgaaattcattgaaaaactgtacaccaatggccttacgacgggtgtgtgggttgcgcgcgagaagattcatggggcgaactttagcctgattatcgaacgtgataatgtcacttgcgccaaacgtacaggcccgattctcccagctgaagacttctatggttacgaaatcgtgctgaagaaatacgataaagcaattaagaccgtccaaaagtttatgtatacagcacgcgccgtgtcttaccaggtatttggagaatttgctggtggtggcatccagaaaggtgttgattatggcgagaaagacttctatgtgttcgatattttaatcaacaccgaatcaggtgacaatacgtatctgaccgattacgagatgcaagacttctgtaacgaatttgggtttaagatggcgcctatgttaggacgcggcaccttcgatagtctgattatgattcctaacgatttggatagtgtacttgcggcgtataatgccactgcaagcgaagacctggttgaagcgaacaactgcgtctttgatgccaatgtgatcggtgacaatacggctgagggctacgttctgaaaccgtgttttcccaaatggctcccgaacggtaaccgtgtgattatcaaatgcaagaactcgaaattcagcgagaaaaagaaatctgacaaaccgattaagactcaggtaccactgacggaaatcgataagaatctgttggatgtcctggcctgttatgtgaccttaaaccgcgtcaataacgtgatttcgaagattggcacagttaccccgaaagattttgggaaagttatgggcctgactgtgcaggatatcctcgaagaaacctcacgtgaagggattgtacttacgacgagtgacaaccctaatctggtgaagaaagaattggttcgcatggtccaagatgtgttacgtcccgcatggatcgagctggtttcctaa

SEQ ID NO: 42 - DNA, 1980 bp, synthetic construct
  source 1..1980: mol_type = other DNA
atgcaccatcaccatcaccatgaaaatctgtattttcagagcatgttcaagaaatactcctccttggagaaccactataactcgaaattcattgaaaaactgtacaccaatggccttacgacgggtgtgtgggttgcgcgcgagaagattcatggggcgaactttagcctgattatcgaacgtgataatgtcacttgcgccaaacgtacaggcccgattctcccagctgaagacttctatggttacgaaatcgtgctgaagaaatacgataaagcaattaagaccgtccaaaagtttatgtatacagcacgcgccgtgtcttaccaggtatttggagaatttgctggtggtggcatccagaaaggtgttgattatggcgagaaagacttctatgtgttcgatattttaatcaacaccgaatcaggtgacaatacgtatctgaccgattacgagatgcaagacttctgtaacgaatttgggtttaagatggcgcctatgttaggacgcggcaccttcgatagtctgattatgattcctaacgatttggatagtgtacttgcggcgtataatgccactgcaagcgaagacctggttgaagcgaacaactgcgtctttgatgccaatgtgatcggtgacaatacggctgagggctacgttctgaaaccgtgttttcccaaatggctcccgaacggtaaccgtgtgattatcaaatgcaagaactcgaaattcagcgagaaaaagaaatctgacaaaccgattaagactcaggtaccactgacggaaatcgataagaatctgttggatgtcctggcctgttatgtgaccttaaaccgcgtcaataacgtgatttcgaagattggcacagttaccccgaaagattttgggaaagttatgggcctgactgtgcaggatatcctcgaagaaacctcacgtgaagggattgtacttacgacgagtgacaaccctaatctggtgaagaaagaattggttcgcatggtccaagatgtgttacgtcccgcatggatcgagctggtttccggtagcgcgggctctgcggcgggttctggcgaatttattaacatctataaaattgataaactgaataattttaacttgaacaatcataagaccgacgactacagcctgtgcaaggataaagatactgcgctcgaactgacccagaagaacatccagaagatttatgactaccaacagaaactgtatgccgagaagaaagaaggccttatcatcgcattccaagctatggatgcagcgggtaaagatggaacgattcgcgaagtgttaaaagcgttagctccgcagggtgttcacgagaaaccatttaaaagtccgtcgtcgacagaactggcccatgattacttgtggcgtgtccacaatgccgtacctgagaaaggcgaaattaccatcttcaaccgctcccattatgaagacgtgctgattgggaaagttaaagaactgtacaagtttcagaacaaagcggatcgtattgacgaaaatacggtggtggataaccgctatgaggatatccgtaatttcgagaaatatctctacaacaactctgtccgcatcatcaaaatctttctgaacgtcagcaagaaagaacaagcagaacgttttctgagccgcattgaagaaccggagaagaattggaaattcagtgactcagattttgaagagcgtgtttattgggacaaatatcagcaggcgttcgaagatgccattaatgcgacctccacgaaagattgtccctggtacgtagtaccggctgatcgcaaatggtacatgcgttatgtggtgagcgaaatcgttgttaagacccttgaagaaatgaacccaaaatacccgacggtgactaaagagacattggaacgctttgaaggctatcgtaccaaactgctggaggagtataattatgggttagataccattcgccctattgaaaagggccaaactggctaa

SEQ ID NO: 43 - DNA, 1998 bp, synthetic construct
  source 1..1998: mol_type = other DNA
atgcaccatcaccatcaccatgaaaatctgtattttcagagcatgttcaagaaatactcctccttggagaaccactataactcgaaattcattgaaaaactgtacaccaatggccttacgacgggtgtgtgggttgcgcgcgagaagattcatggggcgaactttagcctgattatcgaacgtgataatgtcacttgcgccaaacgtacaggcccgattctcccagctgaagacttctatggttacgaaatcgtgctgaagaaatacgataaagcaattaagaccgtccaaaagtttatgtatacagcacgcgccgtgtcttaccaggtatttggagaatttgctggtggtggcatccagaaaggtgttgattatggcgagaaagacttctatgtgttcgatattttaatcaacaccgaatcaggtgacaatacgtatctgaccgattacgagatgcaagacttctgtaacgaatttgggtttaagatggcgcctatgttaggacgcggcaccttcgatagtctgattatgattcctaacgatttggatagtgtacttgcggcgtataatgccactgcaagcgaagacctggttgaagcgaacaactgcgtctttgatgccaatgtgatcggtgacaatacggctgagggctacgttctgaaaccgtgttttcccaaatggctcccgaacggtaaccgtgtgattatcaaatgcaagaactcgaaattcagcgagaaaaagaaatctgacaaaccgattaagactcaggtaccactgacggaaatcgataagaatctgttggatgtcctggcctgttatgtgaccttaaaccgcgtcaataacgtgatttcgaagattggcacagttaccccgaaagattttgggaaagttatgggcctgactgtgcaggatatcctcgaagaaacctcacgtgaagggattgtacttacgacgagtgacaaccctaatctggtgaagaaagaattggttcgcatggtccaagatgtgttacgtcccgcatggatcgagctggtttccggtagctctggctctggcagctctagcggcggttctagctctagcggtagctctattaacatctataaaattgataaactgaataattttaacttgaacaatcataagaccgacgactacagcctgtgcaaggataaagatactgcgctcgaactgacccagaagaacatccagaagatttatgactaccaacagaaactgtatgccgagaagaaagaaggccttatcatcgcattccaagctatggatgcagcgggtaaagatggaacgattcgcgaagtgttaaaagcgttagctccgcagggtgttcacgagaaaccatttaaaagtccgtcgtcgacagaactggcccatgattacttgtggcgtgtccacaatgccgtacctgagaaaggcgaaattaccatcttcaaccgctcccattatgaagacgtgctgattgggaaagttaaagaactgtacaagtttcagaacaaagcggatcgtattgacgaaaatacggtggtggataaccgctatgaggatatccgtaatttcgagaaatatctctacaacaactctgtccgcatcatcaaaatctttctgaacgtcagcaagaaagaacaagcagaacgttttctgagccgcattgaagaaccggagaagaattggaaattcagtgactcagattttgaagagcgtgtttattgggacaaatatcagcaggcgttcgaagatgccattaatgcgacctccacgaaagattgtccctggtacgtagtaccggctgatcgcaaatggtacatgcgttatgtggtgagcgaaatcgttgttaagacccttgaagaaatgaacccaaaatacccgacggtgactaaagagacattggaacgctttgaaggctatcgtaccaaactgctggaggagtataattatgggttagataccattcgccctattgaaaagggccaaactggctaa

SEQ ID NO: 44 - DNA, 1923 bp, synthetic construct
  source 1..1923: mol_type = other DNA
atgttcaagaaatactcctccttggagaaccactataactcgaaattcattgaaaaactgtacaccaatggccttacgacgggtgtgtgggttgcgcgcgagaagattcatggggcgaactttagcctgattatcgaacgtgataatgtcacttgcgccaaacgtacaggcccgattctcccagctgaagacttctatggttacgaaatcgtgctgaagaaatacgataaagcaattaagaccgtccaaaagtttatgtatacagcacgcgccgtgtcttaccaggtatttggagaatttgctggtggtggcatccagaaaggtgttgattatggcgagaaagacttctatgtgttcgatattttaatcaacaccgaatcaggtgacaatacgtatctgaccgattacgagatgcaagacttctgtaacgaatttgggtttaagatggcgcctatgttaggacgcggcaccttcgatagtctgattatgattcctaacgatttggatagtgtacttgcggcgtataatgccactgcaagcgaagacctggttgaagcgaacaactgcgtctttgatgccaatgtgatcggtgacaatacggctgagggctacgttctgaaaccgtgttttcccaaatggctcccgaacggtaaccgtgtgattatcaaatgcaagaactcgaaattcagcgagaaaaagaaatctgacaaaccgattaagactcaggtaccactgacggaaatcgataagaatctgttggatgtcctggcctgttatgtgaccttaaaccgcgtcaataacgtgatttcgaagattggcacagttaccccgaaagattttgggaaagttatgggcctgactgtgcaggatatcctcgaagaaacctcacgtgaagggattgtacttacgacgagtgacaaccctaatctggtgaagaaagaattggttcgcatggtccaagatgtgttacgtcccgcatggatcgagctggtttccggtagcgcgggctctgcggcgggttctggcgaatttattaacatctataaaattgataaactgaataattttaacttgaacaatcataagaccgacgactacagcctgtgcaaggataaagatactgcgctcgaactgacccagaagaacatccagaagatttatgactaccaacagaaactgtatgccgagaagaaagaaggccttatcatcgcattccaagctatggatgcagcgggtaaagatggaacgattcgcgaagtgttaaaagcgttagctccgcagggtgttcacgagaaaccatttaaaagtccgtcgtcgacagaactggcccatgattacttgtggcgtgtccacaatgccgtacctgagaaaggcgaaattaccatcttcaaccgctcccattatgaagacgtgctgattgggaaagttaaagaactgtacaagtttcagaacaaagcggatcgtattgacgaaaatacggtggtggataaccgctatgaggatatccgtaatttcgagaaatatctctacaacaactctgtccgcatcatcaaaatctttctgaacgtcagcaagaaagaacaagcagaacgttttctgagccgcattgaagaaccggagaagaattggaaattcagtgactcagattttgaagagcgtgtttattgggacaaatatcagcaggcgttcgaagatgccattaatgcgacctccacgaaagattgtccctggtacgtagtaccggctgatcgcaaatggtacatgcgttatgtggtgagcgaaatcgttgttaagacccttgaagaaatgaacccaaaatacccgacggtgactaaagagacattggaacgctttgaaggctatcgtaccaaactgctggaggagtataattatgggttagataccattcgccctattgaaaag

SEQ ID NO: 45 - DNA, 1941 bp, synthetic construct
  source 1..1941: mol_type = other DNA
atgttcaagaaatactcctccttggagaaccactataactcgaaattcattgaaaaactgtacaccaatggccttacgacgggtgtgtgggttgcgcgcgagaagattcatggggcgaactttagcctgattatcgaacgtgataatgtcacttgcgccaaacgtacaggcccgattctcccagctgaagacttctatggttacgaaatcgtgctgaagaaatacgataaagcaattaagaccgtccaaaagtttatgtatacagcacgcgccgtgtcttaccaggtatttggagaatttgctggtggtggcatccagaaaggtgttgattatggcgagaaagacttctatgtgttcgatattttaatcaacaccgaatcaggtgacaatacgtatctgaccgattacgagatgcaagacttctgtaacgaatttgggtttaagatggcgcctatgttaggacgcggcaccttcgatagtctgattatgattcctaacgatttggatagtgtacttgcggcgtataatgccactgcaagcgaagacctggttgaagcgaacaactgcgtctttgatgccaatgtgatcggtgacaatacggctgagggctacgttctgaaaccgtgttttcccaaatggctcccgaacggtaaccgtgtgattatcaaatgcaagaactcgaaattcagcgagaaaaagaaatctgacaaaccgattaagactcaggtaccactgacggaaatcgataagaatctgttggatgtcctggcctgttatgtgaccttaaaccgcgtcaataacgtgatttcgaagattggcacagttaccccgaaagattttgggaaagttatgggcctgactgtgcaggatatcctcgaagaaacctcacgtgaagggattgtacttacgacgagtgacaaccctaatctggtgaagaaagaattggttcgcatggtccaagatgtgttacgtcccgcatggatcgagctggtttccggtagctctggctctggcagctctagcggcggttctagctctagcggtagctctattaacatctataaaattgataaactgaataattttaacttgaacaatcataagaccgacgactacagcctgtgcaaggataaagatactgcgctcgaactgacccagaagaacatccagaagatttatgactaccaacagaaactgtatgccgagaagaaagaaggccttatcatcgcattccaagctatggatgcagcgggtaaagatggaacgattcgcgaagtgttaaaagcgttagctccgcagggtgttcacgagaaaccatttaaaagtccgtcgtcgacagaactggcccatgattacttgtggcgtgtccacaatgccgtacctgagaaaggcgaaattaccatcttcaaccgctcccattatgaagacgtgctgattgggaaagttaaagaactgtacaagtttcagaacaaagcggatcgtattgacgaaaatacggtggtggataaccgctatgaggatatccgtaatttcgagaaatatctctacaacaactctgtccgcatcatcaaaatctttctgaacgtcagcaagaaagaacaagcagaacgttttctgagccgcattgaagaaccggagaagaattggaaattcagtgactcagattttgaagagcgtgtttattgggacaaatatcagcaggcgttcgaagatgccattaatgcgacctccacgaaagattgtccctggtacgtagtaccggctgatcgcaaatggtacatgcgttatgtggtgagcgaaatcgttgttaagacccttgaagaaatgaacccaaaatacccgacggtgactaaagagacattggaacgctttgaaggctatcgtaccaaactgctggaggagtataattatgggttagataccattcgccctattgaaaag

SEQ ID NO: 46 - DNA, 1968 bp, synthetic construct
  source 1..1968: mol_type = other DNA
atgattaacatctataaaattgataaactgaataattttaacttgaacaatcataagaccgacgactacagcctgtgcaaggataaagatactgcgctcgaactgacccagaagaacatccagaagatttatgactaccaacagaaactgtatgccgagaagaaagaaggccttatcatcgcattccaagctatggatgcagcgggtaaagatggaacgattcgcgaagtgttaaaagcgttagctccgcagggtgttcacgagaaaccatttaaaagtccgtcgtcgacagaactggcccatgattacttgtggcgtgtccacaatgccgtacctgagaaaggcgaaattaccatcttcaaccgctcccattatgaagacgtgctgattgggaaagttaaagaactgtacaagtttcagaacaaagcggatcgtattgacgaaaatacggtggtggataaccgctatgaggatatccgtaatttcgagaaatatctctacaacaactctgtccgcatcatcaaaatctttctgaacgtcagcaagaaagaacaagcagaacgttttctgagccgcattgaagaaccggagaagaattggaaattcagtgactcagattttgaagagcgtgtttattgggacaaatatcagcaggcgttcgaagatgccattaatgcgacctccacgaaagattgtccctggtacgtagtaccggctgatcgcaaatggtacatgcgttatgtggtgagcgaaatcgttgttaagacccttgaagaaatgaacccaaaatacccgacggtgactaaagagacattggaacgctttgaaggctatcgtaccaaactgctggaggagtataattatgggttagataccattcgccctattgaaaagcaccatcaccatcaccatagctctggttctagcggccaccatcaccatcaccatgaaaatctgtattttcagagcatgttcaagaaatactcctccttggagaaccactataactcgaaattcattgaaaaactgtacaccaatggccttacgacgggtgtgtgggttgcgcgcgagaagattcatggggcgaactttagcctgattatcgaacgtgataatgtcacttgcgccaaacgtacaggcccgattctcccagctgaagacttctatggttacgaaatcgtgctgaagaaatacgataaagcaattaagaccgtccaaaagtttatgtatacagcacgcgccgtgtcttaccaggtatttggagaatttgctggtggtggcatccagaaaggtgttgattatggcgagaaagacttctatgtgttcgatattttaatcaacaccgaatcaggtgacaatacgtatctgaccgattacgagatgcaagacttctgtaacgaatttgggtttaagatggcgcctatgttaggacgcggcaccttcgatagtctgattatgattcctaacgatttggatagtgtacttgcggcgtataatgccactgcaagcgaagacctggttgaagcgaacaactgcgtctttgatgccaatgtgatcggtgacaatacggctgagggctacgttctgaaaccgtgttttcccaaatggctcccgaacggtaaccgtgtgattatcaaatgcaagaactcgaaattcagcgagaaaaagaaatctgacaaaccgattaagactcaggtaccactgacggaaatcgataagaatctgttggatgtcctggcctgttatgtgaccttaaaccgcgtcaataacgtgatttcgaagattggcacagttaccccgaaagattttgggaaagttatgggcctgactgtgcaggatatcctcgaagaaacctcacgtgaagggattgtacttacgacgagtgacaaccctaatctggtgaagaaagaattggttcgcatggtccaagatgtgttacgtcccgcatggatcgagctggtttcctaa

SEQ ID NO: 47 - DNA, 1980 bp, synthetic construct
  source 1..1980: mol_type = other DNA
atgattaacatctataaaattgataaactgaataattttaacttgaacaatcataagaccgacgactacagcctgtgcaaggataaagatactgcgctcgaactgacccagaagaacatccagaagatttatgactaccaacagaaactgtatgccgagaagaaagaaggccttatcatcgcattccaagctatggatgcagcgggtaaagatggaacgattcgcgaagtgttaaaagcgttagctccgcagggtgttcacgagaaaccatttaaaagtccgtcgtcgacagaactggcccatgattacttgtggcgtgtccacaatgccgtacctgagaaaggcgaaattaccatcttcaaccgctcccattatgaagacgtgctgattgggaaagttaaagaactgtacaagtttcagaacaaagcggatcgtattgacgaaaatacggtggtggataaccgctatgaggatatccgtaatttcgagaaatatctctacaacaactctgtccgcatcatcaaaatctttctgaacgtcagcaagaaagaacaagcagaacgttttctgagccgcattgaagaaccggagaagaattggaaattcagtgactcagattttgaagagcgtgtttattgggacaaatatcagcaggcgttcgaagatgccattaatgcgacctccacgaaagattgtccctggtacgtagtaccggctgatcgcaaatggtacatgcgttatgtggtgagcgaaatcgttgttaagacccttgaagaaatgaacccaaaatacccgacggtgactaaagagacattggaacgctttgaaggctatcgtaccaaactgctggaggagtataattatgggttagataccattcgccctattgaaaagggccaaactggcagctctggttctagcggccaccatcaccatcaccatagctctggttctagcggcgaaaatctgtattttcagagcatgttcaagaaatactcctccttggagaaccactataactcgaaattcattgaaaaactgtacaccaatggccttacgacgggtgtgtgggttgcgcgcgagaagattcatggggcgaactttagcctgattatcgaacgtgataatgtcacttgcgccaaacgtacaggcccgattctcccagctgaagacttctatggttacgaaatcgtgctgaagaaatacgataaagcaattaagaccgtccaaaagtttatgtatacagcacgcgccgtgtcttaccaggtatttggagaatttgctggtggtggcatccagaaaggtgttgattatggcgagaaagacttctatgtgttcgatattttaatcaacaccgaatcaggtgacaatacgtatctgaccgattacgagatgcaagacttctgtaacgaatttgggtttaagatggcgcctatgttaggacgcggcaccttcgatagtctgattatgattcctaacgatttggatagtgtacttgcggcgtataatgccactgcaagcgaagacctggttgaagcgaacaactgcgtctttgatgccaatgtgatcggtgacaatacggctgagggctacgttctgaaaccgtgttttcccaaatggctcccgaacggtaaccgtgtgattatcaaatgcaagaactcgaaattcagcgagaaaaagaaatctgacaaaccgattaagactcaggtaccactgacggaaatcgataagaatctgttggatgtcctggcctgttatgtgaccttaaaccgcgtcaataacgtgatttcgaagattggcacagttaccccgaaagattttgggaaagttatgggcctgactgtgcaggatatcctcgaagaaacctcacgtgaagggattgtacttacgacgagtgacaaccctaatctggtgaagaaagaattggttcgcatggtccaagatgtgttacgtcccgcatggatcgagctggtttcctaa

SEQ ID NO: 48 - DNA, 927 bp, unidentified
  source 1..927: mol_type = unassigned DNA
atgattaacatctataaaattgataaactgaataattttaacttgaacaatcataagaccgacgactacagcctgtgcaaggataaagatactgcgctcgaactgacccagaagaacatccagaagatttatgactaccaacagaaactgtatgccgagaagaaagaaggccttatcatcgcattccaagctatggatgcagcgggtaaagatggaacgattcgcgaagtgttaaaagcgttagctccgcagggtgttcacgagaaaccatttaaaagtccgtcgtcgacagaactggcccatgattacttgtggcgtgtccacaatgccgtacctgagaaaggcgaaattaccatcttcaaccgctcccattatgaagacgtgctgattgggaaagttaaagaactgtacaagtttcagaacaaagcggatcgtattgacgaaaatacggtggtggataaccgctatgaggatatccgtaatttcgagaaatatctctacaacaactctgtccgcatcatcaaaatctttctgaacgtcagcaagaaagaacaagcagaacgttttctgagccgcattgaagaaccggagaagaattggaaattcagtgactcagattttgaagagcgtgtttattgggacaaatatcagcaggcgttcgaagatgccattaatgcgacctccacgaaagattgtccctggtacgtagtaccggctgatcgcaaatggtacatgcgttatgtggtgagcgaaatcgttgttaagacccttgaagaaatgaacccaaaatacccgacggtgactaaagagacattggaacgctttgaaggctatcgtaccaaactgctggaggagtataattatgacttagataccattcgccctattgaaaagggccaaactggccaccatcaccatcaccattag

SEQ ID NO: 49 - DNA, 927 bp, synthetic construct
  source 1..927: mol_type = other DNA
atgattaacatctataaaattgataaactgaataattttaacttgaacaatcataagaccgacgactacagcctgtgcaaggataaagatactgcgctcgaactgacccagaagaacatccagaagatttatgactaccaacagaaactgtatgccgagaagaaagaaggccttatcatcgcattccaagctatggatgcagcgggtaaagatggaacgattcgcgaagtgttaaaagcgttagctccgcagggtgttcacgagaaaccatttaaaagtccgtcgtcgacagaactggcccatgattacttgtggcgtgtccacaatgccgtacctgagaaaggcgaaattaccatcttcaaccgctcccattatgaagacgtgctgattgggaaagttaaagaactgtacaagtttcagaacaaagcggatcgtattgacgaaaatacggtggtggataaccgctatgaggatatccgtaatttcgagaaatatctctacaacaactctgtccgcatcatcaaaatctttctgaacgtcagcaagaaagaacaagcagaacgttttctgagccgcattgaagaaccggagaagaattggaaattcagtgactcagattttgaagagcgtgtttattgggacaaatatcagcaggcgttcgaagatgccattaatgcgacctccacgaaagattgtccctggtacgtagtaccggctgatcgcaaatggtacatgcgttatgtggtgagcgaaatcgttgttaagacccttgaagaaatgaacccaaaatacccgacggtgactaaagagacattggaacgctttgaaggctatcgtaccaaactgctggaggagtataattatgggttagataccattcgccctattgaaaagggccaaactggccaccatcaccatcaccattag

SEQ ID NO: 50 - DNA, 1041 bp, synthetic construct
  source 1..1041: mol_type = other DNA
atgcatcatcatcaccatcacgaaaatctgtattttcagagcatgttcaagaaatactcctccttggagaaccactataactcgaaattcattgaaaaactgtacaccaatggccttacgacgggtgtgtgggttgcgcgcgagaagatccatgggaccaactttagcctgattatcgaacgtgataatgtcacttgcgccaaacgtacaggcccgattctcccagctgaagacttctatggttacgaaatcgtgctgaagaaatacgataaagcaattaagaccgtccaaaagtttatgtatacagcacgcgccgtgtcttaccaggtatttggagaatttgctggtggcggcatccagaaaggtgttgattatggcgagaaagacttctatgtgttcgatattttaatcaacaccgaatcaggtgacaatacgtatctgaccgattacgagatgcaagacttctgtaacgaatttgggtttaagatggcgccgatgttaggacgcggcaccttcgatagtctgattatgattcctaacgatttggatagtgtacttgcggcgtataatgccactgcaagcgaagacctggttgaagcgaacaactgcgtctttgatgccaatgtgatcggtgacaatacggctgagggctacgttctgaaaccgtgttttcccaaatggctcccgaacggtacccgtgtggcgatcaaatgcaagaactcgaaattcagcgagaaaaagaaatctgacaaaccgattaagactcaggtaccactgacggaaatcgataagaatctgttggatgtcctggcctgttatgtgaccttaaaccgcgtcaataacgtgatttcgaagattggcacagttaccccgaaagattttgggaaagttatgggcctgactgtgcaggatatcctcgaagaaacctcacgtgaagggattgtacttacgacgagtgacaaccctaatctggtgaagaaagaattggttcgcatggtccaagatgtgttacgtcccgcatggatcgagctggtttcctaa

SEQ ID NO: 51 - DNA, 1041 bp, synthetic construct
  source 1..1041: mol_type = other DNA
atgcatcatcatcaccatcacgaaaatctgtattttcagagcatgttcaagaaatactcctccttggagaaccactataactcgaaattcattgaaaaactgtacaccaatggccttacgacgggtgtgtgggttgcgcgcgagaagattcatggggcgaactttagcctgattatcgaacgtgataatgtcacttgcgccaaacgtacaggcccgattctcccagctgaagacttctatggttacgaaatcgtgctgaagaaatacgataaagcaattaagaccgtccaaaagtttatgtatacagcacgcgccgtgtcttaccaggtatttggagaatttgctggtggtggcatccagaaaggtgttgattatggcgagaaagacttctatgtgttcgatattttaatcaacaccgaatcaggtgacaatacgtatctgaccgattacgagatgcaagacttctgtaacgaatttgggtttaagatggcgcctatgttaggacgcggcaccttcgatagtctgattatgattcctaacgatttggatagtgtacttgcggcgtataatgccactgcaagcgaagacctggttgaagcgaacaactgcgtctttgatgccaatgtgatcggtgacaatacggctgagggctacgttctgaaaccgtgttttcccaaatggctcccgaacggtaaccgtgtgattatcaaatgcaagaactcgaaattcagcgagaaaaagaaatctgacaaaccgattaagactcaggtaccactgacggaaatcgataagaatctgttggatgtcctggcctgttatgtgaccttaaaccgcgtcaataacgtgatttcgaagattggcacagttaccccgaaagattttgggaaagttatgggcctgactgtgcaggatatcctcgaagaaacctcacgtgaagggattgtacttacgacgagtgacaaccctaatctggtgaagaaagaattggttcgcatggtccaagatgtgttacgtcccgcatggatcgagctggtttcctaa

SEQ ID NO: 52 - protein, 308 aa, unidentified
MINIYKIDKLNNFNLNNHKTDDYSLCKDKDTALELTQKNIQKIYDYQQKLYAEKKEGLIIAFQAMDAAGKDGTIREVLKALAPQGVHEKPFKSPSSTELAHDYLWRVHNAVPEKGEITIFNRSHYEDVLIGKVKELYKFQNKADRIDENTVVDNRYEDIRNFEKYLYNNSVRIIKIFLNVSKKEQAERFLSRIEEPEKNWKFSDSDFEERVYWDKYQQAFEDAINATSTKDCPWYVVPADRKWYMRYVVSEIVVKTLEEMNPKYPTVTKETLERFEGYRTKLLEEYNYDLDTIRPIEKGQTGHHHHHH

SEQ ID NO: 53 - DNA, 927 bp, synthetic construct
  source 1..927: mol_type = other DNA
atgattaacatctataaaattgataaactgaataattttaacttgaacaatcataagaccgacgactacagcctgtgcaaggataaagatactgcgctcgaactgacccagaagaacatccagaagatttatgactaccaacagaaactgtatgccgagaagaaagaaggccttatcatcgcattccaagctatggatgcagcgggtaaagatggaacgattcgcgaagtgttaaaagcgttagctccgcagggtgttcacgagaaaccatttaaaagtccgtcgtcgacagaactggcccatgattacttgtggcgtgtccacaatgccgtacctgagaaaggcgaaattaccatcttcaaccgctcccattatgaagacgtgctgattgggaaagttaaagaactgtacaagtttcagaacaaagcggatcgtattgacgaaaatacggtggtggataaccgctatgaggatatccgtaatttcgagaaatatctctacaacaactctgtccgcatcatcaaaatctttctgaacgtcagcaagaaagaacaagcagaacgttttctgagccgcattgaagaaccggagaagaattggaaattcagtgactcagattttgaagagcgtgtttattgggacaaatatcagcaggcgttcgaagatgccattaatgcgacctccacgaaagattgtccctggtacgtagtaccggctgatcgcaaatggtacatgcgttatgtggtgagcgaaatcgttgttaagacccttgaagaaatgaacccaaaatacccgacggtgactaaagagacattggaacgctttgaaggctatcgtaccaaactgctggaggagtataatagggacttagataccattcgccctattgaaaagggccaaactggccaccatcaccatcaccattag

SEQ ID NO: 54 - protein, 308 aa, synthetic construct
MINIYKIDKLNNFNLNNHKTDDYSLCKDKDTALELTQKNIQKIYDYQQKLYAEKKEGLIIAFQAMDAAGKDGTIREVLKALAPQGVHEKPFKSPSSTELAHDYLWRVHNAVPEKGEITIFNRSHYEDVLIGKVKELYKFQNKADRIDENTVVDNRYEDIRNFEKYLYNNSVRIIKIFLNVSKKEQAERFLSRIEEPEKNWKFSDSDFEERVYWDKYQQAFEDAINATSTKDCPWYVVPADRKWYMRYVVSEIVVKTLEEMNPKYPTVTKETLERFEGYRTKLLEEYNRDLDTIRPIEKGQTGHHHHHH

SEQ ID NO: 55 - DNA, 927 bp, synthetic construct
  source 1..927: mol_type = other DNA
atgattaacatctataaaattgataaactgaataattttaacttgaacaatcataagaccgacgactacagcctgtgcaaggataaagatactgcgctcgaactgacccagaagaacatccagaagatttatgactaccaacagaaactgtatgccgagaagaaagaaggccttatcatcgcattccaagctatggatgcagcgggtaaagatggaacgattcgcgaagtgttaaaagcgttagctccgcagggtgttcacgagaaaccatttaaaagtccgtcgtcgacagaactggcccatgattacttgtggcgtgtccacaatgccgtacctgagaaaggcgaaattaccatcttcaaccgctcccattatgaagacgtgctgattgggaaagttaaagaactgtacaagtttcagaacaaagcggatcgtattgacgaaaatacggtggtggataaccgctatgaggatatccgtaatttcgagaaatatctctacaacaactctgtccgcatcatcaaaatctttctgaacgtcagcaagaaagaacaagcagaacgttttctgagccgcattgaagaaccggagaagaattggaaattcagtgactcagattttgaagagcgtgtttattgggacaaatatcagcaggcgttcgaagatgccattaatgcgacctccacgaaagattgtccctggtacgtagtaccggctgatcgcaaatggtacatgcgttatgtggtgagcgaaatcgttgttaagacccttgaagaaatgaacccaaaatacccgacggtgactaaagagacattggaacgctttgaaggctatcgtaccaaactgctgggtgagtataattatgacttagataccattcgccctattgaaaagggccaaactggccaccatcaccatcaccattag

SEQ ID NO: 56 - protein, 308 aa, synthetic construct
MINIYKIDKLNNFNLNNHKTDDYSLCKDKDTALELTQKNIQKIYDYQQKLYAEKKEGLIIAFQAMDAAGKDGTIREVLKALAPQGVHEKPFKSPSSTELAHDYLWRVHNAVPEKGEITIFNRSHYEDVLIGKVKELYKFQNKADRIDENTVVDNRYEDIRNFEKYLYNNSVRIIKIFLNVSKKEQAERFLSRIEEPEKNWKFSDSDFEERVYWDKYQQAFEDAINATSTKDCPWYVVPADRKWYMRYVVSEIVVKTLEEMNPKYPTVTKETLERFEGYRTKLLGEYNYDLDTIRPIEKGQTGHHHHHH

SEQ ID NO: 57 - DNA, 927 bp, synthetic construct
  source 1..927: mol_type = other DNA
atgattaacatctataaaattgataaactgaataattttaacttgaacaatcataagaccgacgactacagcctgtgcaaggataaagatactgcgctcgaactgacccagaagaacatccagaagatttatgactaccaacagaaactgtatgccgagaagaaagaaggccttatcatcgcattccaagctatggatgcagcgggtaaagatggaacgattcgcgaagtgttaaaagcgttagctccgcagggtgttcacgagaaaccatttaaaagtccgtcgtcgacagaactggcccatgattacttgtggcgtgtccacaatgccgtacctgagaaaggcgaaattaccatcttcaaccgctcccattatgaagacgtgctgattgggaaagttaaagaactgtacaagtttcagaacaaagcggatcgtattgacgaaaatacggtggtggataaccgctatgaggatatccgtaatttcgagaaatatctctacaacaactctgtccgcatcatcaaaatctttctgaacgtcagcaagaaagaacaagcagaacgttttctgagccgcattgaagaaccggagaagaattggaaattcagtgactcagattttgaagagcgtgtttattgggacaaatatcagcaggcgttcgaagatgccattaatgcgacctccacgaaagattgtccctggtacgtagtaccggctgatcgcaaatggtacatgcgttatgtggtgagcgaaatcgttgttaagacccttgaagaaatgaacccaaaatacccgacggtgactaaagagacattggaacgctttgaaggctatcgtaccaaactgctggaggagtataattatgacttagataccattcgccctattgaatagggccaaactggccaccatcaccatcaccattag

SEQ ID NO: 58 - protein, 297 aa, synthetic construct
MINIYKIDKLNNFNLNNHKTDDYSLCKDKDTALELTQKNIQKIYDYQQKLYAEKKEGLIIAFQAMDAAGKDGTIREVLKALAPQGVHEKPFKSPSSTELAHDYLWRVHNAVPEKGEITIFNRSHYEDVLIGKVKELYKFQNKADRIDENTVVDNRYEDIRNFEKYLYNNSVRIIKIFLNVSKKEQAERFLSRIEEPEKNWKFSDSDFEERVYWDKYQQAFEDAINATSTKDCPWYVVPADRKWYMRYVVSEIVVKTLEEMNPKYPTVTKETLERFEGYRTKLLEEYNYDLDTIRPIE

SEQ ID NO: 59 - DNA, 927 bp, synthetic construct
  source 1..927: mol_type = other DNA
atgattaacatctataaaattgataaactgaataattttaacttgaacaatcataagaccgacgactacagcctgtgcaaggataaagatactgcgctcgaactgacccagaagaacatccagaagatttatgactaccaacagaaactgtatgccgagaagaaagaaggccttatcatcgcattccaagctatggatgcagcgggtaaagatggaacgattcgcgaagtgttaaaagcgttagctccgcagggtgttcacgagaaaccatttaaaagtccgtcgtcgacagaactggcccatgattacttgtggcgtgtccacaatgccgtacctgagaaaggcgaaattaccatcttcaaccgctcccattatgaagacgtgctgattgggaaagttaaagaactgtacaagtttcagaacaaagcggatcgtattgacgaaaatacggtggtggataaccgctatgaggatatccgtaatttcgagaaatatctctacaacaactctgtccgcatcatcaaaatctttactaacgtcagcaagaaagaacaagcagaacgttttctgagccgcattgaagaaccggagaagaattggaaattcagtgactcagattttgaagagcgtgtttattgggacaaatatcagcaggcgttcgaagatgccattaatgcgacctccacgaaagattgtccctggtacgtagtaccggctgatcgcaaatggtacatgcgttatgtggtgagcgaaatcgttgttaagacccttgaagaaatgaacccaaaatacccgacggtgactaaagagacattggaacgctttgaaggctatcgtaccaaactgctggaggagtataattatgacttagataccattcgccctattgaaaagggccaaactggccaccatcaccatcaccattag

SEQ ID NO: 60 - protein, 308 aa, synthetic construct
MINIYKIDKLNNFNLNNHKTDDYSLCKDKDTALELTQKNIQKIYDYQQKLYAEKKEGLIIAFQAMDAAGKDGTIREVLKALAPQGVHEKPFKSPSSTELAHDYLWRVHNAVPEKGEITIFNRSHYEDVLIGKVKELYKFQNKADRIDENTVVDNRYEDIRNFEKYLYNNSVRIIKIFTNVSKKEQAERFLSRIEEPEKNWKFSDSDFEERVYWDKYQQAFEDAINATSTKDCPWYVVPADRKWYMRYVVSEIVVKTLEEMNPKYPTVTKETLERFEGYRTKLLEEYNYDLDTIRPIEKGQTGHHHHHH

SEQ ID NO: 61 - skipped

SEQ ID NO: 62 - protein, 308 aa, synthetic construct
MINIYKIDKLNNFNLNNHKTDDYSLCKDKDTALELTQKNIQKIYDYQQKLYAEKKEGLIIAFQAMDAAGKDGTIREVLKALAPQGVHEKPFKSPSSTELAHDYLWRVHNAVPEKGEITIFNRSHYEDVLIGKVKELYKFQNKADRIDENTVVDNRYEDIRNFEKYLYNNSVRIIKIFLNVSKKEQAERFLSRIEEPEKNWKFSDSDFEERVYWDKYQQAFEDAINATSTKDCPWYVVPADRKWYMRYVVSEIVVKTLEEMNPKYPTVTKETLERFEGYRTKLLEEYNYGLDTIRPIEKGQTGHHHHHH

SEQ ID NO: 63 - DNA, 927 bp, synthetic construct
  source 1..927: mol_type = other DNA
atgattaacatctataaaattgataaactgaataattttaacttgaacaatcataagaccgacgactacagcctgtgcaaggataaagatactgcgctcgaactgacccagaagaacatccagaagatttatgactaccaacagaaactgtatgccgagaagaaagaaggccttatcatcgcattccaagctatggatgcagcgggtaaagatggaacgattcgcgaagtgttaaaagcgttagctccgcagggtgttcacgagaaaccatttaaaagtccgtcgtcgacagaactggcccatgattacttgtggcgtgtccacaatgccgtacctgagaaaggcgaaattaccatcttcaaccgctcccattatgaagacgtgctgattgggaaagttaaagaactgtacaagtttcagaacaaagcggatcgtattgacgaaaatacggtggtggataaccgctatgaggatatccgtaatttcgagaaatatctctacaacaactctgtccgcatcatcaaaatctttctgaacgtcagcaagaaagaacaagcagaacgttttctgagccgcattgaagaaccggagaagaattggaaattcagtgactcagattttgaagagcgtgtttattgggacaaatatcagcaggcgttcgaagatgccattaatgcgacctccacgaaagattgtccctggtacgtagtaccggctgatcgcaaatggtacatgcgttatgtggtgagcgaaatcgttgttaagacccttgaagaaatgaacccaaaatacccgacggtgactaaagagacattggaacgctttgaaggctatcgtaccaaactgctggaggagtataattatgacgtggataccattcgccctattgaaaagggccaaactggccaccatcaccatcaccattag

SEQ ID NO: 64 - protein, 308 aa, synthetic construct
MINIYKIDKLNNFNLNNHKTDDYSLCKDKDTALELTQKNIQKIYDYQQKLYAEKKEGLIIAFQAMDAAGKDGTIREVLKALAPQGVHEKPFKSPSSTELAHDYLWRVHNAVPEKGEITIFNRSHYEDVLIGKVKELYKFQNKADRIDENTVVDNRYEDIRNFEKYLYNNSVRIIKIFLNVSKKEQAERFLSRIEEPEKNWKFSDSDFEERVYWDKYQQAFEDAINATSTKDCPWYVVPADRKWYMRYVVSEIVVKTLEEMNPKYPTVTKETLERFEGYRTKLLEEYNYDVDTIRPIEKGQTGHHHHHH

SEQ ID NO: 65 - DNA, 927 bp, synthetic construct
  source 1..927: mol_type = other DNA
atgattaacatctataaaattgataaactgaataattttaacttgaacaatcataagaccgacgactacagcctgtgcaaggataaagatactgcgctcgaactgacccagaagaacatccagaagatttatgactaccaacagaaactgtatgccgagaagaaagaaggccttatcatcgcattccaagctatggatgcagcgggtaaagatggaacgattcgcgaagtgttaaaagcgttagctccgcagggtgttcacgagaaaccatttaaaagtccgtcgtcgacagaactggcccatgattacttgtggcgtgtccacaatgccgtacctgagaaaggcgaaattaccatcttcaaccgctcccattatgaagacgtgctgattgggaaagttaaagaactgtacaagtttcagaacaaagcggatcgtattgacgaaaatacggtggtggataaccgctatgaggatatccgtaatttcgagaaatatctctacaacaactctgtccgcatcatcaaaatctttctgaacgtcagcaagaaagaacaagcagaacgttttctgagccgcattgaagaaccggagaagaattggaaattcagtgactcagattttgaagagcgtgtttattgggacaaatatcagcaggcgtttgaagatgccattaatgcgacctccacgaaagatgccccctggtacgtagtaccggctgatcgcaaatggtacatgcgttatgtggtgagcgaaatcgttgttaagacccttgaagaaatgaacccaaaatacccgacggtgactaaagagacattggaacgcttcgaaggctatcgtaccaaactgctggaggagtataattatgacttagataccattcgccctattgaaaagggccaaactggccaccatcaccatcaccattag

SEQ ID NO: 66 - protein, 308 aa, synthetic construct
MINIYKIDKLNNFNLNNHKTDDYSLCKDKDTALELTQKNIQKIYDYQQKLYAEKKEGLIIAFQAMDAAGKDGTIREVLKALAPQGVHEKPFKSPSSTELAHDYLWRVHNAVPEKGEITIFNRSHYEDVLIGKVKELYKFQNKADRIDENTVVDNRYEDIRNFEKYLYNNSVRIIKIFLNVSKKEQAERFLSRIEEPEKNWKFSDSDFEERVYWDKYQQAFEDAINATSTKDAPWYVVPADRKWYMRYVVSEIVVKTLEEMNPKYPTVTKETLERFEGYRTKLLEEYNYDLDTIRPIEKGQTGHHHHHH

SEQ ID NO: 67 - DNA, 927 bp, synthetic construct
  source 1..927: mol_type = other DNA
atgattaacatctataaaattgataaactgaataattttaacttgaacaatcataagaccgacgactacagcctgtgcaaggataaagatactgcgctcgaactgacccagaagaacatccagaagatttatgactaccaacagaaactgtatgccgagaagaaagaaggccttatcatcgcattccaagctatggatgcagcgggtaaagatggaacgattcgcgaagtgttaaaagcgttagctccgcagggtgttcacgagaaaccatttaaaagtccgtcgtcgacagaactggcccatgattacttgtggcgtgtccacaatgccgtacctgagaaaggcgaaattaccatcttcaaccgctcccattatgaagacgtgctgattgggaaagttaaagaactgtacaagtttcagaacaaagcggatcgtattgacgaaaatacggtggtggataaccgctatgaggatatccgtaatttcgagaaatatctctacaacaactctgtccgcatcatcaaaatctttctgaacgtcagcaagaaagaacaagcagaacgttttctgagccgcattgaagaaccggagaagaattggaaattcagtgactcagattttgaagagcgtgtttattgggacaaatatcagcaggcgttcgaagatgccattaatgcgacctccacgaaagattgtccctggtacgtagtaccggctgatcgcaaatggtacatgcgttatgtggtgagcgaaatcgttgttaagacccttgaagaaatgaacccaaaatacccgacggtgactaaagagacattggaacgctttgaaggctatcgtaccaaactgctggaggagtatgtgtatgacttagataccattcgccctattgaaaagggccaaactggccaccatcaccatcaccattag

SEQ ID NO: 68 - protein, 308 aa, synthetic construct
MINIYKIDKLNNFNLNNHKTDDYSLCKDKDTALELTQKNIQKIYDYQQKLYAEKKEGLIIAFQAMDAAGKDGTIREVLKALAPQGVHEKPFKSPSSTELAHDYLWRVHNAVPEKGEITIFNRSHYEDVLIGKVKELYKFQNKADRIDENTVVDNRYEDIRNFEKYLYNNSVRIIKIFLNVSKKEQAERFLSRIEEPEKNWKFSDSDFEERVYWDKYQQAFEDAINATSTKDCPWYVVPADRKWYMRYVVSEIVVKTLEEMNPKYPTVTKETLERFEGYRTKLLEEYVYDLDTIRPIEKGQTGHHHHHH

SEQ ID NO: 69 - DNA, 927 bp, synthetic construct
  source 1..927: mol_type = other DNA
atgattaacatctataaaattgataaactgaataattttaacttgaacaatcataagaccgacgactacagcctgtgcaaggataaagatactgcgctcgaactgacccagaagaacatccagaagatttatgactaccaacagaaactgtatgccgagaagaaagaaggccttatcatcgcattccaagctatggatgcagcgggtaaagatggaacgattcgcgaagtgttaaaagcgttagctccgcagggtgttcacgagaaaccatttaaaagtccgtcgtcgacagaactggcccatgattacttgtggcgtgtccacaatgccgtacctgagaaaggcgaaattaccatcttcaaccgctcccattatgaagacgtgctgattgggaaagttaaagaactgtacaagtttcagaacaaagcggatcgtattgacgaaaatacggtggtggataaccgctatgaggatatccgtaatttcgagaaatatctctacaacaactctgtccgcatcatcaaaatctttctgaacgtcagcaagaaagaacaagcagaacgttttctgagccgcattgaagaaccggagaagaattggaaattcagtgactcagattttgaagagcgtgtttattgggacaaatatcagcaggcgttcgaagatgccattaatgcgacctccacgaaagattgtccctggtacgtagtaccggctgatcgcaaatggtacatgcgttatgtggtgagcgaaatcgttgttaagacccttgaagaaatgaacccaaaatacccgacggtgactaaagagacattggaacgctttgaaggctatcgtaccaaactgctggaggagtataattatgacttagataccattcgccctattgaaaagggccaacgtggccaccatcaccatcaccattag

SEQ ID NO: 70 - protein, 308 aa, synthetic construct
MINIYKIDKLNNFNLNNHKTDDYSLCKDKDTALELTQKNIQKIYDYQQKLYAEKKEGLIIAFQAMDAAGKDGTIREVLKALAPQGVHEKPFKSPSSTELAHDYLWRVHNAVPEKGEITIFNRSHYEDVLIGKVKELYKFQNKADRIDENTVVDNRYEDIRNFEKYLYNNSVRIIKIFLNVSKKEQAERFLSRIEEPEKNWKFSDSDFEERVYWDKYQQAFEDAINATSTKDCPWYVVPADRKWYMRYVVSEIVVKTLEEMNPKYPTVTKETLERFEGYRTKLLEEYNYDLDTIRPIEKGQRGHHHHHH

SEQ ID NO: 71 - DNA, 927 bp, synthetic construct
  source 1..927: mol_type = other DNA
atgattaacatctataaaattgataaactgaataattttaacttgaacaatcataagaccgacgactacagcctgtgcaaggataaagatactgcgctcgaactgacccagaagaacatccagaagatttatgactaccaacagaaactgtatgccgagaagaaagaaggccttatcatcgcattccaagctatggatgcagcgggtaaagatggaacgattcgcgaagtgttaaaagcgttagctccgcagggtgttcacgagaaaccatttaaaagtccgtcgtcgacagaactggcccatgattacttgtggcgtgtccacaatgccgtacctgagaaaggcgaaattaccatcttcaaccgctcccattatgaagacgtgctgattgggaaagttaaagaactgtacaagtttcagaacaaagcggatcgtattgacgaaaatacggtggtggataaccgctatgaggatatccgtaatttcgagaaatatctctacaacaactctgtccgcatcatcaaaatctttctgaacgtcagcaagaaagaacaagcagaacgttttctgagccgcattgaagaaccggagaagaattggaaattcagtgactcagattttgaagagcgtgtttattgggacaaatatcagcaggcgttcgaagatgccattaatgcgacctccacgaaagattgtccctggtacgtagtaccggctgatcgcaaatggtacatgcgttatgtggtgagcgaaatcgttgttaagacccttgaagaaatgaacccaaaatacccgacggtgactaaagagacattggaacgctttgaaggctatcgtaccaaactgctggaggagtataattatgacggggataccattcgccctattgaaaagggccaaactggccaccatcaccatcaccattag

SEQ ID NO: 72 - protein, 308 aa, synthetic construct
MINIYKIDKLNNFNLNNHKTDDYSLCKDKDTALELTQKNIQKIYDYQQKLYAEKKEGLIIAFQAMDAAGKDGTIREVLKALAPQGVHEKPFKSPSSTELAHDYLWRVHNAVPEKGEITIFNRSHYEDVLIGKVKELYKFQNKADRIDENTVVDNRYEDIRNFEKYLYNNSVRIIKIFLNVSKKEQAERFLSRIEEPEKNWKFSDSDFEERVYWDKYQQAFEDAINATSTKDCPWYVVPADRKWYMRYVVSEIVVKTLEEMNPKYPTVTKETLERFEGYRTKLLEEYNYDGDTIRPIEKGQTGHHHHHH

SEQ ID NO: 73 - DNA, 927 bp, synthetic construct
  source 1..927: mol_type = other DNA
atgattaacatctataaaattgataaactgaataattttaacttgaacaatcataagaccgacgactacagcctgtgcaaggataaagatactgcgctcgaactgacccagaagaacatccagaagatttatgactaccaacagaaactgtatgccgagaagaaagaaggccttatcatcgcattccaagctatggatgcagcgggtaaagatggaacgattcgcgaagtgttaaaagcgttagctccgcagggtgttcacgagaaaccatttaaaagtccgtcgtcgacagaactggcccatgattacttgtggcgtgtccacaatgccgtacctgagaaaggcgaaattaccatcttcaaccgctcccattatgaagacgtgctgattgggaaagttaaagaactgtacaagtttcagaacaaagcggatcgtattgacgaaaatacggtggtggataaccgctatgaggatatccgtaatttcgagaaatatctctacaacaactctgtccgcatcatcaaaatctttctgaacgtcagcaagaaagaacaagcagaacgttttctgagccgcattgaagaaccggagaagaattggaaattcagtgactcagattttgaagagcgtgtttattgggacaaatatcagcaggcgttcgaagatgccattaatgcgacctccacgaaagattgtccctggtacgtagtaccggctgatcgcaaatggtacatgcgttatgtggtgagcgaaatcgttgttaagacccttgaagaaatgaacccaaaatacccgacggtgactaaagagacattggaacgctttgaaggctatcgtaccaaactgctggaggagtataattatgacttagatacccagcgccctattgaaaagggccaaactggccaccatcaccatcaccattag

SEQ ID NO: 74 - protein, 308 aa, synthetic construct
MINIYKIDKLNNFNLNNHKTDDYSLCKDKDTALELTQKNIQKIYDYQQKLYAEKKEGLIIAFQAMDAAGKDGTIREVLKALAPQGVHEKPFKSPSSTELAHDYLWRVHNAVPEKGEITIFNRSHYEDVLIGKVKELYKFQNKADRIDENTVVDNRYEDIRNFEKYLYNNSVRIIKIFLNVSKKEQAERFLSRIEEPEKNWKFSDSDFEERVYWDKYQQAFEDAINATSTKDCPWYVVPADRKWYMRYVVSEIVVKTLEEMNPKYPTVTKETLERFEGYRTKLLEEYNYDLDTQRPIEKGQTGHHHHHH

SEQ ID NO: 75 - DNA, 927 bp, synthetic construct
  source 1..927: mol_type = other DNA
atgattaacatctataaaattgataaactgaataattttaacttgaacaatcataagaccgacgactacagcctgtgcaaggataaagatactgcgctcgaactgacccagaagaacatccagaagatttatgactaccaacagaaactgtatgccgagaagaaagaaggccttatcatcgcattccaagctatggatgcagcgggtaaagatggaacgattcgcgaagtgttaaaagcgttagctccgcagggtgttcacgagaaaccatttaaaagtccgtcgtcgacagaactggcccatgattacttgtggcgtgtccacaatgccgtacctgagaaaggcgaaattaccatcttcaaccgctcccattatgaagacgtgctgattgggaaagttaaagaactgtacaagtttcagaacaaagcggatcgtattgacgaaaatacggtggtggataaccgctatgaggatatccgtaatttcgagaaatatctctacaacaactctgtccgcatcatcaaaatctttctgaacgtcagcaagaaagaacaagcagaacgttttctgagccgcattgaagaaccggagaagaattggaaattcagtgactcagattttgaagagcgtgtttattgggacaaatatcagcaggcgttcgaagatgccattaatgcgacctccacgaaagattgtccctggtacgtagtaccggctgatcgcaaatggtacatgcgttatgtggtgagcgaaatcgttgttaagacccttgaagaaatgaacccaaaatacccgacggtgactaaagagacattggaacgctttgaaggctatcgtaccaaactgctggaggagtataatcaggacttagataccattcgccctattgaaaagggccaaactggccaccatcaccatcaccattag

SEQ ID NO: 76 - protein, 308 aa, synthetic construct
MINIYKIDKLNNFNLNNHKTDDYSLCKDKDTALELTQKNIQKIYDYQQKLYAEKKEGLIIAFQAMDAAGKDGTIREVLKALAPQGVHEKPFKSPSSTELAHDYLWRVHNAVPEKGEITIFNRSHYEDVLIGKVKELYKFQNKADRIDENTVVDNRYEDIRNFEKYLYNNSVRIIKIFLNVSKKEQAERFLSRIEEPEKNWKFSDSDFEERVYWDKYQQAFEDAINATSTKDCPWYVVPADRKWYMRYVVSEIVVKTLEEMNPKYPTVTKETLERFEGYRTKLLEEYNQDLDTIRPIEKGQTGHHHHHH

SEQ ID NO: 77 - DNA, 927 bp, synthetic construct
  source 1..927: mol_type = other DNA
atgattaacatctataaaattgataaactgaataattttaacttgaacaatcataagaccgacgactacagcctgtgcaaggataaagatactgcgctcgaactgacccagaagaacatccagaagatttatgactaccaacagaaactgtatgccgagaagaaagaaggccttatcatcgcattccaagctatggatgcagcgggtaaagatggaacgattcgcgaagtgttaaaagcgttagctccgcagggtgttcacgagaaaccatttaaaagtccgtcgtcgacagaactggcccatgattacttgtggcgtgtccacaatgccgtacctgagaaaggcgaaattaccatcttcaaccgctcccattatgaagacgtgctgattgggaaagttaaagaactgtacaagtttcagaacaaagcggatcgtattgacgaaaatacggtggtggataaccgctatgaggatatccgtaatttcgagaaatatctctacaacaactctgtccgcatcatcaaaatctttctgaacgtcagcaagaaagaacaagcagaacgttttctgagccgcattgaagaaccggagaagaattggaaattcagtgactcagattttgaagagcgtgtttattgggacaaatatcagcaggcgttcgaagatgccattaatgcgacctccacgaaagattgtccctggtacgtagtaccggctgatcgcaaatggtacatgcgttatgtggtgagcgaaatcgttgttaagacccttgaagaaatgaacccaaaatacccgacggtgactaaagagacattggaacgctttgaaggctatcgtaccaaactgctggaggagtataattatgacttatggaccattcgccctattgaaaagggccaaactggccaccatcaccatcaccattag

SEQ ID NO: 78 - protein, 308 aa, synthetic construct
MINIYKIDKLNNFNLNNHKTDDYSLCKDKDTALELTQKNIQKIYDYQQKLYAEKKEGLIIAFQAMDAAGKDGTIREVLKALAPQGVHEKPFKSPSSTELAHDYLWRVHNAVPEKGEITIFNRSHYEDVLIGKVKELYKFQNKADRIDENTVVDNRYEDIRNFEKYLYNNSVRIIKIFLNVSKKEQAERFLSRIEEPEKNWKFSDSDFEERVYWDKYQQAFEDAINATSTKDCPWYVVPADRKWYMRYVVSEIVVKTLEEMNPKYPTVTKETLERFEGYRTKLLEEYNYDLWTIRPIEKGQTGHHHHHH

SEQ ID NO: 79 - DNA, 927 bp, synthetic construct
  source 1..927: mol_type = other DNA
atgattaacatctataaaattgataaactgaataattttaacttgaacaatcataagaccgacgactacagcctgtgcaaggataaagatactgcgctcgaactgacccagaagaacatccagaagatttatgactaccaacagaaactgtatgccgagaagaaagaaggccttatcatcgcattccaagctatggatgcagcgggtaaagatggaacgattcgcgaagtgttaaaagcgttagctccgcagggtgttcacgagaaaccatttaaaagtccgtcgtcgacagaactggcccatgattacttgtggcgtgtccacaatgccgtacctgagaaaggcgaaattaccatcttcaaccgctcccattatgaagacgtgctgattgggaaagttaaagaactgtacaagtttcagaacaaagcggatcgtattgacgaaaatacggtggtggataaccgctatgaggatatccgtaatttcgagaaatatctctacaacaactctgtccgcatcatcaaaatctttctgaacgtcagcaagaaagaacaagcagaacgttttctgagccgcattgaagaaccggagaagaattggaaattcagtgactcagattttgaagagcgtgtttattgggacaaatatcagcaggcgttcgaagatgccattaatgcgacctccacgaaagattgtccctggtacgtagtaccggctgatcgcaaatggtacatgcgttatgtggtgagcgaaatcgttgttaagacccttgaagaaatgaacccaaaatacccgacggtgactaaagagacattggaacgctttgaaggctatcgtaccaaactgctggaggagtataattatgacttagataccattcgccctattgaatatggccaaactggccaccatcaccatcaccattag

SEQ ID NO: 80 - protein, 308 aa, synthetic construct
MINIYKIDKLNNFNLNNHKTDDYSLCKDKDTALELTQKNIQKIYDYQQKLYAEKKEGLIIAFQAMDAAGKDGTIREVLKALAPQGVHEKPFKSPSSTELAHDYLWRVHNAVPEKGEITIFNRSHYEDVLIGKVKELYKFQNKADRIDENTVVDNRYEDIRNFEKYLYNNSVRIIKIFLNVSKKEQAERFLSRIEEPEKNWKFSDSDFEERVYWDKYQQAFEDAINATSTKDCPWYVVPADRKWYMRYVVSEIVVKTLEEMNPKYPTVTKETLERFEGYRTKLLEEYNYDLDTIRPIEYGQTGHHHHHH

SEQ ID NO: 81 - DNA, 927 bp, synthetic construct
  source 1..927: mol_type = other DNA
atgattaacatctataaaattgataaactgaataattttaacttgaacaatcataagaccgacgactacagcctgtgcaaggataaagatactgcgctcgaactgacccagaagaacatccagaagatttatgactaccaacagaaactgtatgccgagaagaaagaaggccttatcatcgcattccaagctatggatgcagcgggtaaagatggaacgattcgcgaagtgttaaaagcgttagctccgcagggtgttcacgagaaaccatttaaaagtccgtcgtcgacagaactggcccatgattacttgtggcgtgtccacaatgccgtacctgagaaaggcgaaattaccatcttcaaccgctcccattatgaagacgtgctgattgggaaagttaaagaactgtacaagtttcagaacaaagcggatcgtattgacgaaaatacggtggtggataaccgctatgaggatatccgtaatttcgagaaatatctctacaacaactctgtccgcatcatcaaaatctttctgaacgtcagcaagaaagaacaagcagaacgttttctgagccgcattgaagaaccggagaagaattggaaattcagtgactcagattttgaagagcgtgtttattgggacaaatatcagcaggcgttcgaagatgccattaatgcgacctccacgaaagattgtccctggtacgtagtaccggctgatcgcaaatggtacatgcgttatgtggtgagcgaaatcgttgttaagacccttgaagaaatgaacccaaaatacccgacggtgactaaagagacattggaacgctttgaaggctatcgtaccaaactgctggaggagtataattaggacttagataccattcgccctattgaaaagggccaaactggccaccatcaccatcaccattag

SEQ ID NO: 82 - protein, 287 aa, synthetic construct
MINIYKIDKLNNFNLNNHKTDDYSLCKDKDTALELTQKNIQKIYDYQQKLYAEKKEGLIIAFQAMDAAGKDGTIREVLKALAPQGVHEKPFKSPSSTELAHDYLWRVHNAVPEKGEITIFNRSHYEDVLIGKVKELYKFQNKADRIDENTVVDNRYEDIRNFEKYLYNNSVRIIKIFLNVSKKEQAERFLSRIEEPEKNWKFSDSDFEERVYWDKYQQAFEDAINATSTKDCPWYVVPADRKWYMRYVVSEIVVKTLEEMNPKYPTVTKETLERFEGYRTKLLEEYN

SEQ ID NO: 83 - DNA, 927 bp, synthetic construct
  source 1..927: mol_type = other DNA
atgattaacatctataaaattgataaactgaataattttaacttgaacaatcataagaccgacgactacagcctgtgcaaggataaagatactgcgctcgaactgacccagaagaacatccagaagatttatgactaccaacagaaactgtatgccgagaagaaagaaggccttatcatcgcattccaagctatggatgcagcgggtaaagatggaacgattcgcgaagtgttaaaagcgttagctccgcagggtgttcacgagaaaccatttaaaagtccgtcgtcgacagaactggcccatgattacttgtggcgtgtccacaatgccgtacctgagaaaggcgaaattaccatcttcaaccgctcccattatgaagacgtgctgattgggaaagttaaagaactgtacaagtttcagatgaaagcggatcgtattgacgaaaatacggtggtggataaccgctatgaggatatccgtaatttcgagaaatatctctacaacaactctgtccgcatcatcaaaatctttctgaacgtcagcaagaaagaacaagcagaacgttttctgagccgcattgaagaaccggagaagaattggaaattcagtgactcagattttgaagagcgtgtttattgggacaaatatcagcaggcgttcgaagatgccattaatgcgacctccacgaaagattgtccctggtacgtagtaccggctgatcgcaaatggtacatgcgttatgtggtgagcgaaatcgttgttaagacccttgaagaaatgaacccaaaatacccgacggtgactaaagagacattggaacgctttgaaggctatcgtaccaaactgctggaggagtataattatgacttagataccattcgccctattgaaaagggccaaactggccaccatcaccatcaccattag

SEQ ID NO: 84 - protein, 308 aa, synthetic construct
MINIYKIDKLNNFNLNNHKTDDYSLCKDKDTALELTQKNIQKIYDYQQKLYAEKKEGLIIAFQAMDAAGKDGTIREVLKALAPQGVHEKPFKSPSSTELAHDYLWRVHNAVPEKGEITIFNRSHYEDVLIGKVKELYKFQMKADRIDENTVVDNRYEDIRNFEKYLYNNSVRIIKIFLNVSKKEQAERFLSRIEEPEKNWKFSDSDFEERVYWDKYQQAFEDAINATSTKDCPWYVVPADRKWYMRYVVSEIVVKTLEEMNPKYPTVTKETLERFEGYRTKLLEEYNYDLDTIRPIEKGQTGHHHHHH

SEQ ID NO: 85 - DNA, 927 bp, synthetic construct
  source 1..927: mol_type = other DNA
atgattaacatctataaaattgataaactgaataattttaacttgaacaatcataagaccgacgactacagcctgtgcaaggataaagatactgcgctcgaactgacccagaagaacatccagaagatttatgactaccaacagaaactgtatgccgagaagaaagaaggccttatcatcgcattccaagctatggatgcagcgggtaaagatggaacgattcgcgaagtgttaaaagcgttagctccgcagggtgttcacgagaaaccatttaaaagtccgtcgtcgacagaactggcccatgattacttgtggcgtgtccacaatgccgtacctgagaaaggcgaaattaccatcttcaaccgctcccattatgaagacgtgctgattgggaaagttaaagaactgtacaagtttcagaacaaagcggatcgtattgacgaaaatacggtggtggataaccgctatgaggatatccgtaatttcgagaaatatctctacaacaactctgtccgcatcatcaaaatctttctgaacgtcagcaagaaagaacaagcagaacgttttctgagccgcattgaagaaccggagaagaattggaaattcagtgactcagattttgaagagcgtgtttattgggacaaatatcagcaggcgttcgaagatgccattaatgcgacctccacgaaagattgtccctggtacgtagtaccggctgatcgcaaatggtacatgcgttatgtggtgagcgaaatcgttgttaagacccttgaagaaatgaacccaaaatacccgacggtgactaaagagacattggaacgctttgaaggctatcgtaccaaactgctggaggagtataattatgacttagataccattcgccctgttgaaaagggccaaactggccaccatcaccatcaccattag

SEQ ID NO: 86 - protein, 308 aa, synthetic construct
MINIYKIDKLNNFNLNNHKTDDYSLCKDKDTALELTQKNIQKIYDYQQKLYAEKKEGLIIAFQAMDAAGKDGTIREVLKALAPQGVHEKPFKSPSSTELAHDYLWRVHNAVPEKGEITIFNRSHYEDVLIGKVKELYKFQNKADRIDENTVVDNRYEDIRNFEKYLYNNSVRIIKIFLNVSKKEQAERFLSRIEEPEKNWKFSDSDFEERVYWDKYQQAFEDAINATSTKDCPWYVVPADRKWYMRYVVSEIVVKTLEEMNPKYPTVTKETLERFEGYRTKLLEEYNYDLDTIRPVEKGQTGHHHHHH

SEQ ID NO: 87 - DNA, 1041 bp, synthetic construct
  source 1..1041: mol_type = other DNA
atgcatcatcatcaccatcacgaaaatctgtattttcagagcatgttcaagaaatactcctccttggagaaccactataactcgaaattcattgaaagtctgtacaccaatggccttacgacgggtgtgtgggttgcgcgcgagaagatccatgggaccaactttagcctgattatcgaacgtgataatgtcacttgcgccaaacgtacaggcccgattctcccagctgaagacttctatggttacgaaatcgtgctgaagaaatacgataaagcaattaagaccgtccaaaagtttatgtatacagcacgcgccgtgtcttaccaggtatttggagaatttgctggtggcggcatccagaaaggtgttgattatggcgagaaagacttctatgtgttcgatattttaatcaacaccgaatcaggtgacaatacgtatctgaccgattacgagatgcaagacttctgtaacgaatttgggtttaagatggcgccgatgttaggacgcggcaccttcgatagtctgattatgattcctaacgatttggatagtgtacttgcggcgtataatgccactgcaagcgaagacctggttgaagcgaacaactgcgtctttgatgccaatgtgatcggtgacaatacggctgagggctacgttctgaaaccgtgttttcccaaatggctcccgaacggtacccgtgtggcgatcaaatgcaagaactcgaaattcagcgagaaaaagaaatctgacaaaccgattaagactcaggtaccactgacggaaatcgataagaatctgttggatgtcctggcctgttatgtgaccttaaaccgcgtcaataacgtgatttcgaagattggcacagttaccccgaaagattttgggaaagttatgggcctgactgtgcaggatatcctcgaagaaacctcacgtgaagggattgtacttacgacgagtgacaaccctaatctggtgaagaaagaattggttcgcatggtccaagatgtgttacgtcccgcatggatcgagctggtttcctaa

SEQ ID NO: 88 - protein, 346 aa, synthetic construct
MHHHHHHENLYFQSMFKKYSSLENHYNSKFIESLYTNGLTTGVWVAREKIHGTNFSLIIERDNVTCAKRTGPILPAEDFYGYEIVLKKYDKAIKTVQKFMYTARAVSYQVFGEFAGGGIQKGVDYGEKDFYVFDILINTESGDNTYLTDYEMQDFCNEFGFKMAPMLGRGTFDSLIMIPNDLDSVLAAYNATASEDLVEANNCVFDANVIGDNTAEGYVLKPCFPKWLPNGTRVAIKCKNSKFSEKKKSDKPIKTQVPLTEIDKNLLDVLACYVTLNRVNNVISKIGTVTPKDFGKVMGLTVQDILEETSREGIVLTTSDNPNLVKKELVRMVQDVLRPAWIELVS

SEQ ID NO: 89 - DNA, 1980 bp, synthetic construct
  source 1..1980: mol_type = other DNA
atgcaccatcaccatcaccatgaaaatctgtattttcagagcattaacatctataaaattgataaactgaataattttaacttgaacaatcataagaccgacgactacagcctgtgcaaggataaagatactgcgctcgaactgacccagaagaacatccagaagatttatgactaccaacagaaactgtatgccgagaagaaagaaggccttatcatcgcattccaagctatggatgcagcgggtaaagatggaacgattcgcgaagtgttaaaagcgttagctccgcagggtgttcacgagaaaccatttaaaagtccgtcgtcgacagaactggcccatgattacttgtggcgtgtccacaatgccgtacctgagaaaggcgaaattaccatcttcaaccgctcccattatgaagacgtgctgattgggaaagttaaagaactgtacaagtttcagaacaaagcggatcgtattgacgaaaatacggtggtggataaccgctatgaggatatccgtaatttcgagaaatatctctacaacaactctgtccgcatcatcaaaatctttctgaacgtcagcaagaaagaacaagcagaacgttttctgagccgcattgaagaaccggagaagaattggaaattcagtgactcagattttgaagagcgtgtttattgggacaaatatcagcaggcgttcgaagatgccattaatgcgacctccacgaaagattgtccctggtacgtagtaccggctgatcgcaaatggtacatgcgttatgtggtgagcgaaatcgttgttaagacccttgaagaaatgaacccaaaatacccgacggtgactaaagagacattggaacgctttgaaggctatcgtaccaaactgctggaggagtataattatgggttagataccattcgccctattgaaaagggccaaactggcggtagcgcgggctctgcggcgggttctggcgaatttatgttcaagaaatactcctccttggagaaccactataactcgaaattcattgaaagtctgtacaccaatggccttacgacgggtgtgtgggttgcgcgcgagaagatccatgggaccaactttagcctgattatcgaacgtgataatgtcacttgcgccaaacgtacaggcccgattctcccagctgaagacttctatggttacgaaatcgtgctgaagaaatacgataaagcaattaagaccgtccaaaagtttatgtatacagcacgcgccgtgtcttaccaggtatttggagaatttgctggtggcggcatccagaaaggtgttgattatggcgagaaagacttctatgtgttcgatattttaatcaacaccgaatcaggtgacaatacgtatctgaccgattacgagatgcaagacttctgtaacgaatttgggtttaagatggcgccgatgttaggacgcggcaccttcgatagtctgattatgattcctaacgatttggatagtgtacttgcggcgtataatgccactgcaagcgaagacctggttgaagcgaacaactgcgtctttgatgccaatgtgatcggtgacaatacggctgagggctacgttctgaaaccgtgttttcccaaatggctcccgaacggtacccgtgtggcgatcaaatgcaagaactcgaaattcagcgagaaaaagaaatctgacaaaccgattaagactcaggtaccactgacggaaatcgataagaatctgttggatgtcctggcctgttatgtgaccttaaaccgcgtcaataacgtgatttcgaagattggcacagttaccccgaaagattttgggaaagttatgggcctgactgtgcaggatatcctcgaagaaacctcacgtgaagggattgtacttacgacgagtgacaaccctaatctggtgaagaaagaattggttcgcatggtccaagatgtgttacgtcccgcatggatcgagctggtttcctaa

SEQ ID NO: 90 - protein, 659 aa, synthetic construct
MHHHHHHENLYFQSINIYKIDKLNNFNLNNHKTDDYSLCKDKDTALELTQKNIQKIYDYQQKLYAEKKEGLIIAFQAMDAAGKDGTIREVLKALAPQGVHEKPFKSPSSTELAHDYLWRVHNAVPEKGEITIFNRSHYEDVLIGKVKELYKFQNKADRIDENTVVDNRYEDIRNFEKYLYNNSVRIIKIFLNVSKKEQAERFLSRIEEPEKNWKFSDSDFEERVYWDKYQQAFEDAINATSTKDCPWYVVPADRKWYMRYVVSEIVVKTLEEMNPKYPTVTKETLERFEGYRTKLLEEYNYGLDTIRPIEKGQTGGSAGSAAGSGEFMFKKYSSLENHYNSKFIESLYTNGLTTGVWVAREKIHGTNFSLIIERDNVTCAKRTGPILPAEDFYGYEIVLKKYDKAIKTVQKFMYTARAVSYQVFGEFAGGGIQKGVDYGEKDFYVFDILINTESGDNTYLTDYEMQDFCNEFGFKMAPMLGRGTFDSLIMIPNDLDSVLAAYNATASEDLVEANNCVFDANVIGDNTAEGYVLKPCFPKWLPNGTRVAIKCKNSKFSEKKKSDKPIKTQVPLTEIDKNLLDVLACYVTLNRVNNVISKIGTVTPKDFGKVMGLTVQDILEETSREGIVLTTSDNPNLVKKELVRMVQDVLRPAWIELVS

SEQ ID NO: 91 - DNA, 1980 bp, synthetic construct
  source 1..1980: mol_type = other DNA
atgcaccatcaccatcaccatgaaaatctgtattttcagagcattaacatctataaaattgataaactgaataattttaacttgaacaatcataagaccgacgactacagcctgtgcaaggataaagatactgcgctcgaactgacccagaagaacatccagaagatttatgactaccaacagaaactgtatgccgagaagaaagaaggccttatcatcgcattccaagctatggatgcagcgggtaaagatggaacgattcgcgaagtgttaaaagcgttagctccgcagggtgttcacgagaaaccatttaaaagtccgtcgtcgacagaactggcccatgattacttgtggcgtgtccacaatgccgtacctgagaaaggcgaaattaccatcttcaaccgctcccattatgaagacgtgctgattgggaaagttaaagaactgtacaagtttcagaacaaagcggatcgtattgacgaaaatacggtggtggataaccgctatgaggatatccgtaatttcgagaaatatctctacaacaactctgtccgcatcatcaaaatctttctgaacgtcagcaagaaagaacaagcagaacgttttctgagccgcattgaagaaccggagaagaattggaaattcagtgactcagattttgaagagcgtgtttattgggacaaatatcagcaggcgttcgaagatgccattaatgcgacctccacgaaagattgtccctggtacgtagtaccggctgatcgcaaatggtacatgcgttatgtggtgagcgaaatcgttgttaagacccttgaagaaatgaacccaaaatacccgacggtgactaaagagacattggaacgctttgaaggctatcgtaccaaactgctggaggagtataattatgggttagataccattcgccctattgaaaagggccaaactggcggtagcgcgggctctgcggcgggttctggcgaatttatgttcaagaaatactcctccttggagaaccactataactcgaaattcattgaaagtctgtacaccaatggccttacgacgggtgtgtgggttgcgcgcgagaagatccatggggttaactttagcctgattatcgaacgtgataatgtcacttgcgccaaacgtacaggcccgattctcccagctgaagacttctatggttacgaaatcgtgctgaagaaatacgataaagcaattaagaccgtccaaaagtttatgtatacagcacgcgccgtgtcttaccaggtatttggagaatttgctggtggcggcatccagaaaggtgttgattatggcgagaaagacttctatgtgttcgatattttaatcaacaccgaatcaggtgacaatacgtatctgaccgattacgagatgcaagacttctgtaacgaatttgggtttaagatggcgccgatgttaggacgcggcaccttcgatagtctgattatgattcctaacgatttggatagtgtacttgcggcgtataatgccactgcaagcgaagacctggttgaagcgaacaactgcgtctttgatgccaatgtgatcggtgacaatacggctgagggctacgttctgaaaccgtgttttcccaaatggctcccgaacggtacccgtgtggcgatcaaatgcaagaactcgaaattcagcgagaaaaagaaatctgacaaaccgattaagactcaggtaccactgacggaaatcgataagaatctgttggatgtcctggcctgttatgtgaccttaaaccgcgtcaataacgtgatttcgaagattggcacagttaccccgaaagattttgggaaagttatgggcctgactgtgcaggatatcctcgaagaaacctcacgtgaagggattgtacttacgacgagtgacaaccctaatctggtgaagaaagaattggttcgcatggtccaagatgtgttacgtcccgcatggatcgagctggtttcctaa

SEQ ID NO: 92 - protein, 659 aa, synthetic construct
MHHHHHHENLYFQSINIYKIDKLNNFNLNNHKTDDYSLCKDKDTALELTQKNIQKIYDYQQKLYAEKKEGLIIAFQAMDAAGKDGTIREVLKALAPQGVHEKPFKSPSSTELAHDYLWRVHNAVPEKGEITIFNRSHYEDVLIGKVKELYKFQNKADRIDENTVVDNRYEDIRNFEKYLYNNSVRIIKIFLNVSKKEQAERFLSRIEEPEKNWKFSDSDFEERVYWDKYQQAFEDAINATSTKDCPWYVVPADRKWYMRYVVSEIVVKTLEEMNPKYPTVTKETLERFEGYRTKLLEEYNYGLDTIRPIEKGQTGGSAGSAAGSGEFMFKKYSSLENHYNSKFIESLYTNGLTTGVWVAREKIHGVNFSLIIERDNVTCAKRTGPILPAEDFYGYEIVLKKYDKAIKTVQKFMYTARAVSYQVFGEFAGGGIQKGVDYGEKDFYVFDILINTESGDNTYLTDYEMQDFCNEFGFKMAPMLGRGTFDSLIMIPNDLDSVLAAYNATASEDLVEANNCVFDANVIGDNTAEGYVLKPCFPKWLPNGTRVAIKCKNSKFSEKKKSDKPIKTQVPLTEIDKNLLDVLACYVTLNRVNNVISKIGTVTPKDFGKVMGLTVQDILEETSREGIVLTTSDNPNLVKKELVRMVQDVLRPAWIELVS

SEQ ID NO: 93 - DNA, 1980 bp, synthetic construct
  source 1..1980: mol_type = other DNA
atgcaccatcaccatcaccatgaaaatctgtattttcagagcattaacatctataaaattgataaactgaataattttaacttgaacaatcataagaccgacgactacagcctgtgcaaggataaagatactgcgctcgaactgacccagaagaacatccagaagatttatgactaccaacagaaactgtatgccgagaagaaagaaggccttatcatcgcattccaagctatggatgcagcgggtaaagatggaacgattcgcgaagtgttaaaagcgttagctccgcagggtgttcacgagaaaccatttaaaagtccgtcgtcgacagaactggcccatgattacttgtggcgtgtccacaatgccgtacctgagaaaggcgaaattaccatcttcaaccgctcccattatgaagacgtgctgattgggaaagttaaagaactgtacaagtttcagaacaaagcggatcgtattgacgaaaatacggtggtggataaccgctatgaggatatccgtaatttcgagaaatatctctacaacaactctgtccgcatcatcaaaatctttctgaacgtcagcaagaaagaacaagcagaacgttttctgagccgcattgaagaaccggagaagaattggaaattcagtgactcagattttgaagagcgtgtttattgggacaaatatcagcaggcgttcgaagatgccattaatgcgacctccacgaaagattgtccctggtacgtagtaccggctgatcgcaaatggtacatgcgttatgtggtgagcgaaatcgttgttaagacccttgaagaaatgaacccaaaatacccgacggtgactaaagagacattggaacgctttgaaggctatcgtaccaaactgctggaggagtataattatgggttagataccattcgccctattgaaaagggccaaactggcggtagcgcgggctctgcggcgggttctggcgaatttatgttcaagaaatactcctccttggagaaccactataactcgaaattcattgaaagtctgtacaccaatggccttacgacgggtgtgtgggttgcgcgcgagaagatccatgggatgaactttagcctgattatcgaacgtgataatgtcacttgcgccaaacgtacaggcccgattctcccagctgaagacttctatggttacgaaatcgtgctgaagaaatacgataaagcaattaagaccgtccaaaagtttatgtatacagcacgcgccgtgtcttaccaggtatttggagaatttgctggtggcggcatccagaaaggtgttgattatggcgagaaagacttctatgtgttcgatattttaatcaacaccgaatcaggtgacaatacgtatctgaccgattacgagatgcaagacttctgtaacgaatttgggtttaagatggcgccgatgttaggacgcggcaccttcgatagtctgattatgattcctaacgatttggatagtgtacttgcggcgtataatgccactgcaagcgaagacctggttgaagcgaacaactgcgtctttgatgccaatgtgatcggtgacaatacggctgagggctacgttctgaaaccgtgttttcccaaatggctcccgaacggtacccgtgtggcgatcaaatgcaagaactcgaaattcagcgagaaaaagaaatctgacaaaccgattaagactcaggtaccactgacggaaatcgataagaatctgttggatgtcctggcctgttatgtgaccttaaaccgcgtcaataacgtgatttcgaagattggcacagttaccccgaaagattttgggaaagttatgggcctgactgtgcaggatatcctcgaagaaacctcacgtgaagggattgtacttacgacgagtgacaaccctaatctggtgaagaaagaattggttcgcatggtccaagatgtgttacgtcccgcatggatcgagctggtttcctaa

SEQ ID NO: 94 - protein, 659 aa, synthetic construct
MHHHHHHENLYFQSINIYKIDKLNNFNLNNHKTDDYSLCKDKDTALELTQKNIQKIYDYQQKLYAEKKEGLIIAFQAMDAAGKDGTIREVLKALAPQGVHEKPFKSPSSTELAHDYLWRVHNAVPEKGEITIFNRSHYEDVLIGKVKELYKFQNKADRIDENTVVDNRYEDIRNFEKYLYNNSVRIIKIFLNVSKKEQAERFLSRIEEPEKNWKFSDSDFEERVYWDKYQQAFEDAINATSTKDCPWYVVPADRKWYMRYVVSEIVVKTLEEMNPKYPTVTKETLERFEGYRTKLLEEYNYGLDTIRPIEKGQTGGSAGSAAGSGEFMFKKYSSLENHYNSKFIESLYTNGLTTGVWVAREKIHGMNFSLIIERDNVTCAKRTGPILPAEDFYGYEIVLKKYDKAIKTVQKFMYTARAVSYQVFGEFAGGGIQKGVDYGEKDFYVFDILINTESGDNTYLTDYEMQDFCNEFGFKMAPMLGRGTFDSLIMIPNDLDSVLAAYNATASEDLVEANNCVFDANVIGDNTAEGYVLKPCFPKWLPNGTRVAIKCKNSKFSEKKKSDKPIKTQVPLTEIDKNLLDVLACYVTLNRVNNVISKIGTVTPKDFGKVMGLTVQDILEETSREGIVLTTSDNPNLVKKELVRMVQDVLRPAWIELVS

SEQ ID NO: 95 - DNA, 1980 bp, synthetic construct
  source 1..1980: mol_type = other DNA
atgcaccatcaccatcaccatgaaaatctgtattttcagagcattaacatctataaaattgataaactgaataattttaacttgaacaatcataagaccgacgactacagcctgtgcaaggataaagatactgcgctcgaactgacccagaagaacatccagaagatttatgactaccaacagaaactgtatgccgagaagaaagaaggccttatcatcgcattccaagctatggatgcagcgggtaaagatggaacgattcgcgaagtgttaaaagcgttagctccgcagggtgttcacgagaaaccatttaaaagtccgtcgtcgacagaactggcccatgattacttgtggcgtgtccacaatgccgtacctgagaaaggcgaaattaccatcttcaaccgctcccattatgaagacgtgctgattgggaaagttaaagaactgtacaagtttcagaacaaagcggatcgtattgacgaaaatacggtggtggataaccgctatgaggatatccgtaatttcgagaaatatctctacaacaactctgtccgcatcatcaaaatctttctgaacgtcagcaagaaagaacaagcagaacgttttctgagccgcattgaagaaccggagaagaattggaaattcagtgactcagattttgaagagcgtgtttattgggacaaatatcagcaggcgttcgaagatgccattaatgcgacctccacgaaagattgtccctggtacgtagtaccggctgatcgcaaatggtacatgcgttatgtggtgagcgaaatcgttgttaagacccttgaagaaatgaacccaaaatacccgacggtgactaaagagacattggaacgctttgaaggctatcgtaccaaactgctggaggagtataattatgggttagataccattcgccctattgaaaagggccaaactggcggtagcgcgggctctgcggcgggttctggcgaatttatgttcaagaaatactcctccttggagaaccactataactcgactttcattgaaagtctgtacaccaatggccttacgacgggtgtgtgggttgcgcgcgagaagatccatgggaccaactttagcctgattatcgaacgtgataatgtcacttgcgccaaacgtacaggcccgattctcccagctgaagacttctatggttacgaaatcgtgctgaagaaatacgataaagcaattaagaccgtccaaaagtttatgtatacagcacgcgccgtgtcttaccaggtatttggagaatttgctggtggcggcatccagaaaggtgttgattatggcgagaaagacttctatgtgttcgatattttaatcaacaccgaatcaggtgacaatacgtatctgaccgattacgagatgcaagacttctgtaacgaatttgggtttaagatggcgccgatgttaggacgcggcaccttcgatagtctgattatgattcctaacgatttggatagtgtacttgcggcgtataatgccactgcaagcgaagacctggttgaagcgaacaactgcgtctttgatgccaatgtgatcggtgacaatacggctgagggctacgttctgaaaccgtgttttcccaaatggctcccgaacggtacccgtgtggcgatcaaatgcaagaactcgaaattcagcgagaaaaagaaatctgacaaaccgattaagactcaggtaccactgacggaaatcgataagaatctgttggatgtcctggcctgttatgtgaccttaaaccgcgtcaataacgtgatttcgaagattggcacagttaccccgaaagattttgggaaagttatgggcctgactgtgcaggatatcctcgaagaaacctcacgtgaagggattgtacttacgacgagtgacaaccctaatctggtgaagaaagaattggttcgcatggtccaagatgtgttacgtcccgcatggatcgagctggtttcctaa

SEQ ID NO: 96 - protein, 659 aa, synthetic construct
MHHHHHHENLYFQSINIYKIDKLNNFNLNNHKTDDYSLCKDKDTALELTQKNIQKIYDYQQKLYAEKKEGLIIAFQAMDAAGKDGTIREVLKALAPQGVHEKPFKSPSSTELAHDYLWRVHNAVPEKGEITIFNRSHYEDVLIGKVKELYKFQNKADRIDENTVVDNRYEDIRNFEKYLYNNSVRIIKIFLNVSKKEQAERFLSRIEEPEKNWKFSDSDFEERVYWDKYQQAFEDAINATSTKDCPWYVVPADRKWYMRYVVSEIVVKTLEEMNPKYPTVTKETLERFEGYRTKLLEEYNYGLDTIRPIEKGQTGGSAGSAAGSGEFMFKKYSSLENHYNSTFIESLYTNGLTTGVWVAREKIHGTNFSLIIERDNVTCAKRTGPILPAEDFYGYEIVLKKYDKAIKTVQKFMYTARAVSYQVFGEFAGGGIQKGVDYGEKDFYVFDILINTESGDNTYLTDYEMQDFCNEFGFKMAPMLGRGTFDSLIMIPNDLDSVLAAYNATASEDLVEANNCVFDANVIGDNTAEGYVLKPCFPKWLPNGTRVAIKCKNSKFSEKKKSDKPIKTQVPLTEIDKNLLDVLACYVTLNRVNNVISKIGTVTPKDFGKVMGLTVQDILEETSREGIVLTTSDNPNLVKKELVRMVQDVLRPAWIELVS

SEQ ID NO: 97 - DNA, 1980 bp, synthetic construct
  source 1..1980: mol_type = other DNA
atgcaccatcaccatcaccatgaaaatctgtattttcagagcattaacatctataaaattgataaactgaataattttaacttgaacaatcataagaccgacgactacagcctgtgcaaggataaagatactgcgctcgaactgacccagaagaacatccagaagatttatgactaccaacagaaactgtatgccgagaagaaagaaggccttatcatcgcattccaagctatggatgcagcgggtaaagatggaacgattcgcgaagtgttaaaagcgttagctccgcagggtgttcacgagaaaccatttaaaagtccgtcgtcgacagaactggcccatgattacttgtggcgtgtccacaatgccgtacctgagaaaggcgaaattaccatcttcaaccgctcccattatgaagacgtgctgattgggaaagttaaagaactgtacaagtttcagaacaaagcggatcgtattgacgaaaatacggtggtggataaccgctatgaggatatccgtaatttcgagaaatatctctacaacaactctgtccgcatcatcaaaatctttctgaacgtcagcaagaaagaacaagcagaacgttttctgagccgcattgaagaaccggagaagaattggaaattcagtgactcagattttgaagagcgtgtttattgggacaaatatcagcaggcgttcgaagatgccattaatgcgacctccacgaaagattgtccctggtacgtagtaccggctgatcgcaaatggtacatgcgttatgtggtgagcgaaatcgttgttaagacccttgaagaaatgaacccaaaatacccgacggtgactaaagagacattggaacgctttgaaggctatcgtaccaaactgctggaggagtataattatgggttagataccattcgccctattgaaaagggccaaactggcggtagcgcgggctctgcggcgggttctggcgaatttatgttcaagaaatactcctccttggagaaccactataactcgaaattcattgaaagtctgtacaccaatggccttacgacgggtgtgtgggttgcgcgcgagaagatccatggggctaactttagcctgattatcgaacgtgataatgtcacttgctataaacgtacaggcccgattctcccagctgaagacttctatggttacgaaatcgtgctgaagaaatacgataaagcaattaagaccgtccaaaagtttatgtatacagcacgcgccgtgtcttaccaggtatttggagaatttgctggtggcggcatccagaaaggtgttgattatggcgagaaagacttctatgtgttcgatattttaatcaacaccgagtcaggtgactacacgtatctgaccgattacgagatgcaagacttctgtaacgaatttggctttaagttggcgccgatgttaggacgcggcacctgggatagtctgattatgattcctaacgatttggatagtgtacttgcggcgtataatgcccgcgcaagcgaagacctggttgaagcgaacaactgcgtctttgatgccaatgtgatcggtgacaatacggctgagggctacgttctgaaaccgtgttttcccaaatggctcccgaacggtacccgtgtggcgatcaaatgcaagaactcgaaattcagcgagaaaaagaaatctgacaaaccgattaagactcaggtaccactgacggaaatcgataagaatctgttggattgcctggcctgttatgtgaccttaaaccgcgtcaataacgtgatttcgaagattggcacagttaccccgaaagattttgggaaagttatgggcctgactgtgcaggatatcctcgaagaaacctcacgtgaagggattgtacttacgacgagtgacaaccctaatctggtgaagaaagaattggttcgcatggtccaagatgtgttacgtcccgcatggatcgagctggtttcctaa

SEQ ID NO: 98 - protein, 659 aa, synthetic construct
MHHHHHHENLYFQSINIYKIDKLNNFNLNNHKTDDYSLCKDKDTALELTQKNIQKIYDYQQKLYAEKKEGLIIAFQAMDAAGKDGTIREVLKALAPQGVHEKPFKSPSSTELAHDYLWRVHNAVPEKGEITIFNRSHYEDVLIGKVKELYKFQNKADRIDENTVVDNRYEDIRNFEKYLYNNSVRIIKIFLNVSKKEQAERFLSRIEEPEKNWKFSDSDFEERVYWDKYQQAFEDAINATSTKDCPWYVVPADRKWYMRYVVSEIVVKTLEEMNPKYPTVTKETLERFEGYRTKLLEEYNYGLDTIRPIEKGQTGGSAGSAAGSGEFMFKKYSSLENHYNSKFIESLYTNGLTTGVWVAREKIHGANFSLIIERDNVTCYKRTGPILPAEDFYGYEIVLKKYDKAIKTVQKFMYTARAVSYQVFGEFAGGGIQKGVDYGEKDFYVFDILINTESGDYTYLTDYEMQDFCNEFGFKLAPMLGRGTWDSLIMIPNDLDSVLAAYNARASEDLVEANNCVFDANVIGDNTAEGYVLKPCFPKWLPNGTRVAIKCKNSKFSEKKKSDKPIKTQVPLTEIDKNLLDCLACYVTLNRVNNVISKIGTVTPKDFGKVMGLTVQDILEETSREGIVLTTSDNPNLVKKELVRMVQDVLRPAWIELVS

SEQ ID NO: 99 - RNA, 23 nt, synthetic construct
  source 1..23: mol_type = other RNA
  modified_base 1: mod_base = OTHER; note = 2'-O-methyl modified nucleotide with an internucleotide phosphorothioate linkage
  modified_base 2: mod_base = OTHER; note = 2'-fluoro modified nucleotide with an internucleotide phosphorothioate linkage
  modified_base 3: mod_base = OTHER; note = 2'-O-methyl modified nucleotide
  modified_base 4: mod_base = OTHER; note = 2'-fluoro modified nucleotide
  modified_base 5: mod_base = OTHER; note = 2'-fluoro modified nucleotide
  modified_base 6: mod_base = OTHER; note = 2'-fluoro modified nucleotide
  modified_base 7: mod_base = OTHER; note = 2'-O-methyl modified nucleotide
  modified_base 8: mod_base = OTHER; note = 2'-fluoro modified nucleotide
  modified_base 9: mod_base = OTHER; note = 2'-O-methyl modified nucleotide
  modified_base 10: mod_base = OTHER; note = 2'-fluoro modified nucleotide
  modified_base 11: mod_base = OTHER; note = 2'-O-methyl modified nucleotide
  modified_base 12: mod_base = OTHER; note = 2'-fluoro modified nucleotide
  modified_base 13: mod_base = OTHER; note = 2'-O-methyl modified nucleotide
  modified_base 14: mod_base = OTHER; note = 2'-fluoro modified nucleotide
  modified_base 15: mod_base = OTHER; note = 2'-O-methyl modified nucleotide
  modified_base 16: mod_base = OTHER; note = 2'-fluoro modified nucleotide
  modified_base 17: mod_base = OTHER; note = 2'-O-methyl modified nucleotide
  modified_base 18: mod_base = OTHER; note = 2'-fluoro modified nucleotide
  modified_base 19: mod_base = OTHER; note = 2'-O-methyl modified nucleotide
  modified_base 20: mod_base = OTHER; note = 2'-O-methyl modified nucleotide
  modified_base 21: mod_base = OTHER; note = 2'-O-methyl modified nucleotide with an internucleotide phosphorothioate linkage
  modified_base 22: mod_base = OTHER; note = 2'-O-methyl modified nucleotide with an internucleotide phosphorothioate linkage
  modified_base 23: mod_base = OTHER; note = 2'-O-methyl modified nucleotide
acaaaagcaaaacaggtctagaa

SEQ ID NO: 100 - DNA, 21 bp, synthetic construct
  source 1..21: mol_type = other DNA
  modified_base 1: mod_base = OTHER; note = 2'-O-methyl modified nucleotide with an internucleotide phosphorothioate linkage
  modified_base 2: mod_base = OTHER; note = 2'-O-methyl modified nucleotide with an internucleotide phosphorothioate linkage
  modified_base 3: mod_base = OTHER; note = 2'-O-methyl modified nucleotide
  modified_base 4: mod_base = OTHER; note = 2'-O-methyl modified nucleotide
  modified_base 5: mod_base = OTHER; note = 2'-O-methyl modified nucleotide
  modified_base 6: mod_base = OTHER; note = 2'-O-methyl modified nucleotide
  modified_base 7: mod_base = OTHER; note = 2'-fluoro modified nucleotide
  modified_base 8: mod_base = OTHER; note = 2'-O-methyl modified nucleotide
  modified_base 9: mod_base = OTHER; note = 2'-fluoro modified nucleotide
  modified_base 10: mod_base = OTHER; note = 2'-O-methyl modified nucleotide
  modified_base 12: mod_base = OTHER; note = 2'-O-methyl modified nucleotide
  modified_base 13: mod_base = OTHER; note = 2'-O-methyl modified nucleotide
  modified_base 14: mod_base = OTHER; note = 2'-O-methyl modified nucleotide
  modified_base 15: mod_base = OTHER; note = 2'-O-methyl modified nucleotide
  modified_base 16: mod_base = OTHER; note = 2'-O-methyl modified nucleotide
  modified_base 17: mod_base = OTHER; note = 2'-O-methyl modified nucleotide
  modified_base 18: mod_base = OTHER; note = 2'-O-methyl modified nucleotide
  modified_base 19: mod_base = OTHER; note = 2'-O-methyl modified nucleotide
  modified_base 20: mod_base = OTHER; note = 2'-O-methyl modified nucleotide
  modified_base 21: mod_base = OTHER; note = 2'-O-Methyl N-acetyl galactosamine modified nucleotide
  misc_feature 1..10: note = RNA
  misc_feature 11: note = DNA
  misc_feature 12..21: note = RNA
ctagacctgttttgcttttgt